(19) United States
(12) Patent Application Publication
(10) Pub. No.: US 2025/0191689 A1
(43) Pub. Date: Jun. 12, 2025

(54) METHODS FOR SUBTYPING AND TREATMENT OF HEAD AND NECK SQUAMOUS CELL CARCINOMA

(71) Applicants: GeneCentric Therapeutics, Inc., Durham, NC (US); University of Tennessee Research Foundation, Memphis, TN (US); Washington University, St. Louis, MO (US)

(72) Inventors: Gregory M. MAYHEW, Durham, NC (US); David Neil HAYES, Memphis, TN (US); Jose P. ZEVALLOS, St. Louis, MO (US)

(21) Appl. No.: 18/840,884

(22) PCT Filed: Feb. 24, 2023
(86) PCT No.: PCT/US2023/063188

§ 371 Date: Aug. 22, 2024

Related U.S. Application Data
(60) Provisional application No. 63/313,890, filed on Feb. 25, 2022.

(51) Int. Cl. G16B 30/00; G16B 40/20

(52) U.S. Cl. CPC G16B 30/00; G16B 40/20

(57) ABSTRACT

Methods, systems and compositions are provided for determining a subtype of head and neck squamous cell carcinoma (HNSCC) of an individual by detecting the expression level of a plurality of classifier biomarkers selected from a gene signature for HNSCC presented herein. Also provided herein are methods and compositions for determining the response of an individual with a HNSCC subtype to a therapy such as radiation therapy.

### CROSS REFERENCE TO RELATED APPLICATIONS

This application claims priority from U.S. Provisional Application No. 63/313,890 filed Feb. 25, 2022, which is incorporated by reference herein in its entirety for all purposes.

### STATEMENT AS TO FEDERALLY SPONSORED RESEARCH

This invention was made with U.S. government support under grant number CA211939 awarded by the National Institutes of Health. This work was supported by the U.S. Department of Veterans Affairs, and the Federal Government has certain rights in the invention.

### FIELD OF THE INVENTION

The present invention relates to methods for determining a squamous cell carcinoma subtype of a head and neck sample (e.g., oral cavity) and for predicting the response to a treatment (e.g., radiation therapy) for a patient inflicted with specific subtypes of head and neck cancer.

### STATEMENT REGARDING SEQUENCE LISTING

The Sequence Listing XML associated with this application is provided in XML file format and is hereby incorporated by reference into the specification. The name of the XML file containing the Sequence Listing XML is GNCN_023_01WO_SeqList_ST26.xml. The XML file is 390,778 bytes, was created on Feb. 22, 2023, and is being submitted electronically via U.S. Patent Center.

### BACKGROUND OF THE INVENTION

Head and neck squamous cell carcinoma (HNSCC), including cancers of the oral cavity, oropharynx, nasopharynx, hypopharynx and larynx, is one of the most common cancers worldwide (American Cancer Society. Cancer Facts and Figures 2021. Atlanta: American Cancer Society). In the United States, it is estimated that there were approximately 66,000 new cases and 14,000 deaths in 2021 (American Cancer Society. Cancer Facts and Figures 2021. Atlanta: American Cancer Society). The majority of HNSCC are associated with heavy tobacco and alcohol use, although over the last thirty years there has been an increase in the incidence of human papillomavirus (HPV)-related cancer, primarily in the oropharynx. While the treatment of HNSCC depends on multiple tumor and patient-related factors, the three main modalities used in the management of HNSCC are surgical resection, radiation therapy, and chemotherapy. Patients with early-stage tumors are generally treated with a single modality therapy while those with advanced stage tumors often require multiple modalities. Oncologie outcomes in HNSCC are driven largely by stage at presentation: The 5-year overall survival for Stage I-II and III-IV HNSCC is approximately 70-90% and 40-60%, respectively.

While the majority of early stage HNSCC cases may be curable with surgical or radiation-based therapies, it is notable that 10-30% of HPV-negative HNSCC cases without pathologically aggressive features still experience a relapse event (Amin M B, Greene F L, Edge S B, et al. The Eighth Edition AJCC Cancer Staging Manual: Continuing to build a bridge from a population-based to a more “personalized” approach to cancer staging. CA Cancer J Clin. 2017; 67(2):93-99). Oral cavity squamous cell carcinoma (OCSCC) is the most common head and neck cancer, comprising ⅓ of all cases, with the vast majority of OCSCC cases being HPV-negative and associated with tobacco use. Dependent on clinical staging, OCSCC treatment involves surgical excision of the primary tumor with or without neck dissection, followed by radiation with or without chemotherapy. Cancers arising from the larynx and hypopharynx are also almost exclusively tobacco-associated and HPV-negative. Primary radiation-based treatments are common for early and intermediate stage cancers of the larynx and hypopharynx to preserve function, with surgical resection often reserved for locally advanced tumors or salvage after failed radiation therapy. Oropharyngeal squamous cell carcinoma (OPSCC) includes cancers arising from the tonsils, base of tongue, soft palate and lateral and posterior pharyngeal walls. While traditionally associated with heavy smoking and alcohol consumption, it is estimated that approximately 60-70% of incident OPSCC cases are attributable to human papillomavirus (HPV) (Ang K K, Harris J, Wheeler R, et al. Human papillomavirus and survival of patients with oropharyngeal cancer. N Engl J Med. 2010; 363(1):24-35; Chaturvedi A K, Engels E A, Pfeiffer R M, et al. Human papillomavirus and rising oropharyngeal cancer incidence in the United States. J Clin Oncol 2011; 29(32):4294-4301; Haughey B H, Sinha P. Prognostic factors and survival unique to surgically treated p16+ oropharyngeal cancer. Laryngoscope. 2012; 122 Suppl 2: S13-33. Treatment for OPSCC usually includes radiation+/−chemotherapy, although novel treatment paradigms including minimally invasive surgery have been investigated. In contrast to excellent oncologic outcomes associated with HPV-positive OPSCC, HPV-negative OPSCC is associated with high recurrence rates and mortality (Fakbry C, Westra W H, Li S, et al. Improved survival of patients with human papillomavirus-positive head and neck squamous cell carcinoma in a prospective clinical trial. J Natl Cancer Inst. 2008; 100(4):261-269; Gillison ML, D'Souza G, Westra W, et al. Distinct risk factor profiles for human papillomavirus type 16-positive and human papillomavirus type 16-negative head and neck cancers. J Natl Cancer Inst. 2008; 100(6):407-420; and Wu Y, Posner M R, Schumaker L M, et al. Novel biomarker panel predicts prognosis in human papillomavirus-negative oropharyngeal cancer: an analysis of the TAX 324 trial. Cancer. 2012; 118(7):1811-1817).

There have been significant advances in our understanding of the molecular biology of HNSCC and of the genomic heterogeneity across tumors. Based on earlier work in lung cancer (Wilkerson M D, Yin X, Hoadley K A, et al. Lung squamous cell carcinoma mRNA expression subtypes are reproducible, clinically important, and correspond to normal cell types. Clin Cancer Res. 2010; 16(19):4864-4875), four mRNA expression patterns (classical, atypical, basal, and mesenchymal) that demonstrate unique genomic features and prognostic significance have been described (Chung C H, Parker J S, Karaca G, et al. Molecular classification of head and neck squamous cell carcinomas using patterns of gene expression. Cancer Cell. 2004; 5(5):489-500; and Walter V, Yin X, Wilkerson M D, et al. Molecular subtypes in head and neck cancer exhibit distinct patterns of chromosomal gain and loss of canonical cancer genes. PLOS One. 2013; 8(2):e56823). These HNSCC subtypes show varied biology and may be helpful in prognostic assessments complementing other risk stratification based on HPV status, stage, anatomic site, and other characteristics. The basal subtype is characterized by over-expression of genes functioning in cell adhesion including COL17A1, and growth factor and receptor TGFA and EGFR (Walter V, Yin X, Wilkerson M D, et al. Molecular subtypes in head and neck cancer exhibit distinct patterns of chromosomal gain and loss of canonical cancer genes. PLOS One. 2013; 8(2):e56823). The mesenchymal subtype displayed over-expression of genes involved in immune response (Cancer Genome Atlas N. Comprehensive genomic characterization of head and neck squamous cell carcinomas. Nature. 2015; 517(7536):576-582; and Keck M K, Zuo Z, Kbattri A, et al. Integrative analysis of head and neck cancer identifies two biologically distinct HPV and three non-HPV subtypes. Clin Cancer Res. 2015; 21(4):870-881) and is characterized by expression of genes associated with epithelial to mesenchymal transition including VIM, DES, TWIST1, and HGF (Walter V, Yin X, Wilkerson M D, et al. Molecular subtypes in head and neck cancer exhibit distinct patterns of chromosomal gain and loss of canonical cancer genes. PLOS One. 2013; 8(2):e56823). It has been suggested previously that epithelial to mesenchymal transition pathways are important in the initiation of nodal metastasis (Wilkerson M D, Yin X, Hoadley K A, et al. Lung squamous cell carcinoma mRNA expression subtypes are reproducible, clinically important, and correspond to normal cell types. Clin Cancer Res. 2010; 16(19):4864-4875; Walter V, Yin X, Wilkerson M D, et al. Molecular subtypes in head and neck cancer exhibit distinct patterns of chromosomal gain and loss of canonical cancer genes. PLOS One. 2013; 8(2):e56823; and De Cecco L, Nicolau M, Giannoccaro M, et al. Head and neck cancer subtypes with biological and clinical relevance: Meta-analysis of gene-expression data. Oncotarget. 2015; 6(11):9627-9642). The classical subtype is characterized by over-expression of genes related to oxidative stress response and xenobiotic metabolism and is most strongly associated with tobacco exposure (Bao J, Li J, Li D, Li Z. Correlation between expression of NF-E2-related factor 2 and progression of gastric cancer. Int J Clin Exp Med. 2015; 8(8):13235-13242; Bao L J, Jaramillo M C, Zhang Z B, et al. Nrf2 induces cisplatin resistance through activation of autophagy in ovarian carcinoma. Int J Clin Exp Pathol. 2014; 7(4):1502-1513; Kawasaki Y, Ishigami S, Arigami T, et al. Clinicopathological significance of nuclear factor (erythroid-2)-related factor 2 (Nrf2) expression in gastric cancer. BMC Cancer. 2015; 15:5; and Kawasaki Y, Okumura H, Uchikado Y, et al. Nrf2 is useful for predicting the effect of chemoradiation therapy on esophageal squamous cell carcinoma. Ann Surg Oncol. 2014; 21(7):2347-2352). The atypical subtype, which includes both HPV and non-HPV tumors, is characterized by elevated expression of CDKN2A, LIG1, and RPA2. The atypical subtype was also associated with low EGFR expression (Walter V, Yin X, Wilkerson M D, et al. Molecular subtypes in head and neck cancer exhibit distinct patterns of chromosomal gain and loss of canonical cancer genes. PLOS One. 2013; 8(2):e56823). These four gene-expression based head and neck cancer subtypes have been validated in other studies, including in The Cancer Genome Atlas (TCGA) head and neck cancer cohort (Chung C H, Parker J S, Karaca G, et al. Molecular classification of head and neck squamous cell carcinomas using patterns of gene expression. Cancer Cell. 2004; 5(5):489-500; and Walter V, Yin X, Wilkerson M D, et al. Molecular subtypes in head and neck cancer exhibit distinct patterns of chromosomal gain and loss of canonical cancer genes. PLOS One. 2013; 8(2):e56823).

The present invention addresses the need in the field for a clinically useful method for improved HNSCC tumor classification that could inform prognosis, drug response and patient management based on underlying genomic and biologic tumor characteristics. The methods provided herein include evaluation of gene expression subtypes and application of an algorithm for categorization of HNSCC tumors into one of four (4) subtypes (Atypical (AT), Mesenchymal (MS), Classical (CL) and Basal (BA)) and, optionally, evaluation of the nodal status of the HNSCC tumors.

### SUMMARY OF THE INVENTION

In one aspect, provided herein is a method for determining a head and neck squamous cell carcinoma (HNSCC) subtype of a sample obtained from a subject suffering from or suspected of suffering from HNSCC, the method comprising detecting an expression level of a plurality of classifier biomarkers selected from Table 1, wherein the detection of the expression level of the plurality of the classifier biomarkers specifically identifies a basal (BA), mesenchymal (MS), atypical (AT) or classical (CL) HNSCC subtype. In some cases, the method further comprises comparing the detected levels of expression of the plurality of classifier biomarkers selected from Table 1 to the expression of the plurality of classifier biomarkers selected from Table 1 in at least one sample training set(s), wherein the at least one sample training set comprises expression data of the plurality of classifier biomarkers selected from Table 1 from a reference HNSCC BA sample, expression data of the plurality of classifier biomarkers selected from Table 1 from a reference HNSCC MS sample, expression data of the plurality of classifier biomarkers selected from Table 1 from a reference HNSCC AT sample, expression data of the plurality of classifier biomarkers selected from Table 1 from a reference HNSCC CL sample or a combination thereof; and classifying the sample as BA, MS, AT or CL subtype based on the results of the comparing step. In some cases, the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm. In some cases, the expression level of the plurality of classifier biomarkers selected from Table 1 is detected at the nucleic acid level. In some cases, the nucleic acid level is RNA or cDNA. In some cases, the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing qRT-PCR. In some cases, the detection of the expression level comprises using at least one pair of oligonucleotide primers specific for each classifier biomarker from the plurality of classifier biomarkers selected from Table 1. In some cases, the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the subject. In some cases, the bodily fluid is blood or fractions thereof, urine, saliva, or sputum. In some cases, the plurality of classifier biomarkers selected from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers selected from Table 1 comprise olfml3, poolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of classifier biomarkers selected from Table 1 comprises all the classifier biomarkers from Table 1. In some cases, the method further comprises determining the nodal status of the subject suffering from or suspected of suffering from HNSCC. In some cases, the HNSCC is oral cavity HNSCC.

In another aspect, provided herein is a method for determining a head and neck squamous cell carcinoma (HNSCC) subtype of a sample obtained from a subject suffering from or suspected of suffering from HNSCC comprising detecting an expression level of a plurality of nucleic acid molecules that each encode a classifier biomarker having a specific expression pattern in head and neck cancer cells, wherein the plurality of classifier biomarkers are selected from the classifier biomarkers in Table 1, the method comprising: (a) isolating nucleic acid material from a sample from a subject suffering from or suspected of suffering from HNSCC; (b) mixing the nucleic acid material with a plurality of oligonucleotides, wherein the plurality of oligonucleotides comprises at least one oligonucleotide that is substantially complementary to a portion of each nucleic acid molecule from the plurality of the classifier biomarkers; and (c) detecting expression of the plurality of classifier biomarkers, wherein the HNSCC subtype is selected from a basal (BA), mesenchymal (MS), atypical (AT) or classical (CL) subtype. In some cases, the method further comprises comparing the detected levels of expression of the plurality of classifier biomarkers from Table 1 to the expression of the plurality of classifier biomarkers from Table 1 in at least one sample training set(s), wherein the at least one sample training set comprises expression data of the plurality of classifier biomarkers from Table 1 from a reference HNSCC BA sample, expression data of the plurality of classifier biomarkers from Table 1 from a reference HNSCC MS sample, expression data of the plurality of classifier biomarkers from Table 1 from a reference HNSCC AT sample, expression data of the plurality of classifier biomarkers from Table 1 from a reference HNSCC CL sample or a combination thereof, and classifying the sample as BA, MS, AT or CL subtype based on the results of the comparing step. In some cases, the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm. In some cases, the detecting the expression level comprises performing qRT-PCR or any hybridization-based gene assays. In some cases, the expression level is detected by performing qRT-PCR. In some cases, the detection of the expression level comprises using at least one pair of oligonucleotide primers specific for each nucleic acid molecule from the plurality of the classifier biomarker from Table 1. In some cases, the method further comprises determining the nodal status of the subject suffering from or suspected of suffering from HNSCC. In some cases, the method further comprises predicting the response to a therapy for treating a subtype of HNSCC based on the detected expression level of the classifier biomarker. In some cases, the subtype is mesenchymal, and the therapy is radiation therapy. In some cases, the nodal status is node negative. In some cases, the sample is a formalin-fixed, paraffin-embedded (FFPE) sample from the head and neck area of the subject, fresh or a frozen tissue sample from the head and neck area of the subject, an exosome, wash fluids, cell pellets or a bodily fluid obtained from the subject. In some cases, the bodily fluid is blood or fractions thereof, urine, saliva, or sputum. In some cases, the plurality of classifier biomarkers selected from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers selected from the classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of classifier biomarkers selected from the classifier biomarkers of Table 1 comprises all the classifier biomarkers from Table 1. In some cases, the HNSCC is oral cavity HNSCC.

In still another aspect, provided herein is a method of detecting a biomarker in a sample obtained from a subject suffering from or suspected of suffering from HNSCC, the method comprising, consisting essentially of or consisting of measuring the expression level of a plurality of biomarker nucleic acids selected from Table 1 using an amplification, hybridization and/or sequencing assay. In some cases, the sample was previously diagnosed as being squamous cell carcinoma. In some cases, the previous diagnosis was by histological examination. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing qRT-PCR. In some cases, the detection of the expression level comprises using at least one pair of oligonucleotide primers per each of the plurality of biomarker nucleic acids selected from Table 1. In some cases, the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the patient. In some cases, the bodily fluid is blood or fractions thereof, urine, saliva, or sputum. In some cases, the plurality of classifier biomarkers from Table 1 comprises, consists essentially of, or consists of at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers from Table 1 comprises, consists essentially of, or consists of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers from Table 1 comprises, consists essentially of, or consists of olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of classifier biomarkers from Table 1 comprises, consists essentially of, or consists of all the classifier biomarkers from Table 1. In some cases, the HNSCC is oral cavity HNSCC.

In one aspect, provided herein is a method of determining whether a patient suffering from or suspected of suffering from HNSCC is likely to respond to radiation therapy, the method comprising: determining the HNSCC subtype of a sample obtained from the patient, wherein the HNSCC subtype is selected from the group consisting of basal, mesenchymal, atypical and classical; and based on the subtype, assessing whether the patient is likely to respond to radiation therapy. In some cases, the method further comprises determining the nodal status of the patient suffering from or suspected of suffering from HNSCC. In some cases, the patient is assessed as likely to respond to radiation therapy if the HNSCC subtype is determined to be mesenchymal, regardless of nodal status of the patient. In some cases, the patient is assessed as likely to respond to radiation therapy if the HNSCC subtype is determined to be basal, atypical or classical and nodal status of the patient is determined to be N123.

In another aspect, provided herein is a method for selecting a patient suffering from or suspected of suffering from HNSCC for radiation therapy, the method comprising, determining a HNSCC subtype of a sample obtained from the patient, based on the subtype; and selecting the patient for radiation therapy, wherein the HNSCC subtype is selected from the group consisting of basal, mesenchymal, atypical and classical. In some cases, the method further comprises determining the nodal status of the patient suffering from or suspected of suffering from HNSCC. In some cases, the patient is selected for radiation therapy if the HNSCC subtype is determined to be mesenchymal, regardless of nodal status of the patient. In some cases, the patient is selected for radiation therapy if the HNSCC subtype is determined to be basal, atypical or classical and nodal status of the patient is determined to be N123. In some cases, the HNSCC is oral cavity HNSCC. In some cases, the radiation therapy is used in combination with surgery and/or chemotherapy. In some cases, the sample is a formalin-fixed, paraffin-embedded (FFPE) sample obtained from the head and neck area of the patient, fresh or a frozen tissue sample obtained from the head and neck area of the patient, an exosome, or a bodily fluid obtained from the patient. In some cases, the bodily fluid is blood or fractions thereof, urine, saliva, or sputum. In some cases, the patient is initially determined to have HNSCC via a histological analysis of a sample. In some cases, the patient's HNSCC subtype is determined via a histological analysis of a sample obtained from the patient. In some cases, the determining the HNSCC subtype comprises determining expression levels of a plurality of classifier biomarkers. In some cases, the determining the expression levels of the plurality of classifier biomarkers is at a nucleic acid level by performing RNA sequencing, reverse transcriptase polymerase chain reaction (RT-PCR) or hybridization-based analyses. In some cases, the plurality of classifier biomarkers for determining the HNSCC subtype is selected from a publicly available HNSCC dataset. In some cases, the publicly available HNSCC dataset is TCGA HNSCC RNAseq dataset. In some cases, the plurality of classifier biomarkers for determining the HNSCC subtype is selected from Table 1. In some cases, the RT-PCR is quantitative real time reverse transcriptase polymerase chain reaction (qRT-PCR). In some cases, the RT-PCR is performed with primers specific to each of the plurality of classifier biomarkers from Table 1. In some cases, the method further comprises comparing the detected levels of expression of the plurality of classifier biomarkers from Table 1 to the levels of expression of the plurality of classifier biomarkers from Table 1 in at least one sample training set(s), wherein the at least one sample training set comprises expression level data of the plurality of classifier biomarkers from Table 1 from a reference HNSCC BA sample, expression level data of the plurality of classifier biomarkers from Table 1 from a reference HNSCC MS sample, expression level data of the plurality of classifier biomarkers from Table 1 from a reference HNSCC AT sample, expression level data of the plurality of classifier biomarkers from Table 1 from a reference HNSCC CL sample or a combination thereof; and classifying the sample obtained from the patient as BA, MS, AT or CL based on the results of the comparing step. In some cases, the comparing step comprises applying a statistical algorithm which comprises determining a correlation between the expression data obtained from the sample obtained from the patient and the expression data from the at least one training set(s); and classifying the sample obtained from the patient as a BA, MS, AT or CL subtype based on the results of the statistical algorithm. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of the classifier biomarkers comprise all of the classifier biomarkers from Table 1.

In yet another aspect, provided herein is a method of treating HNSCC in a subject, the method comprising: determining a subtype of HNSCC of a subject suffering from HNSCC by measuring a nucleic acid expression level of a plurality of classifier biomarkers in a sample obtained from a subject suffering from or suspected of suffering from HNSCC, wherein the plurality of classifier biomarkers is selected from Table 1, wherein the nucleic acid expression level of the plurality of classifier biomarkers indicates the HNSCC subtype of the subject as being basal (BA), mesenchymal (MS), atypical (AT) or classical (CL); and administering radiation therapy to the subject based on the subtype of the HNSCC. In some cases, the HNSCC is oral cavity HNSCC. In some cases, the radiation therapy is administered to the subject if the HNSCC subtype is determined to be mesenchymal, regardless of nodal status of the subject. In some cases, the radiation therapy is administered to the subject if the HNSCC subtype is determined to be basal, classical or atypical and nodal status of the subject is N123. In some cases, the radiation therapy is used in combination with surgery and/or chemotherapy. In some cases, the determining step further comprises comparing the nucleic acid expression levels of the plurality of classifier biomarkers from Table 1 to the nucleic acid expression levels of the plurality of classifier biomarkers from Table 1 in at least one sample training set(s), wherein the at least one sample training set comprises nucleic acid expression level data of the plurality of classifier biomarkers from Table 1 from a reference HNSCC BA sample, nucleic acid expression level data of the plurality of classifier biomarkers from Table 1 from a reference HNSCC MS sample, nucleic acid expression level data of the plurality of classifier biomarkers from Table 1 from a reference HNSCC AT sample, nucleic acid expression level data of the plurality of classifier biomarkers from Table 1 from a reference HNSCC CL sample or a combination thereof; and classifying the sample obtained from the subject as BA, MS, AT or CL based on the results of the comparing step. In some cases, the comparing step comprises applying a statistical algorithm which comprises determining a correlation between the expression data obtained from the sample obtained from the patient and the expression data from the at least one training set(s); and classifying the sample obtained from the subject as a BA, MS, AT or CL subtype based on the results of the statistical algorithm. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of the classifier biomarkers comprise all of the classifier biomarkers from Table 1. In some cases, the measuring the nucleic acid expression level is conducted using an amplification, hybridization and/or sequencing assay. In some cases, the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing qRT-PCR. In some cases, the sample is a formalin-fixed, paraffin-embedded (FFPE) sample obtained from the head and neck area of the subject, fresh or a frozen tissue sample obtained from the head and neck area of the subject, an exosome, wash fluids, cell pellets, or a bodily fluid obtained from the patient. In some cases, the bodily fluid is blood or fractions thereof, urine, saliva, or sputum.

In another aspect, provided herein is a system for determining a head and neck squamous cell carcinoma (HNSCC) subtype of a sample obtained from a subject suffering from HNSCC, the system comprising: (a) one or more processors; and (b) one or more memories operatively coupled to at least one of the one or more processors and having instructions stored thereon that, when executed by at least one of the one or more processors, cause the system to (i) detect an expression level of each of a plurality of classifier biomarkers from Table 1; (ii) compare the expression levels of each of the plurality of classifier biomarkers from Table 1 to the expression levels of each of the plurality of classifier biomarkers from Table 1 in a control; and (iii) classifying the sample as a basal (BA), mesenchymal (MS), atypical (AT) or classical (CL) HNSCC subtype based on the results of the comparing step. In some cases, the control comprises at least one sample training set(s), wherein the at least one sample training set comprises expression levels of each of the plurality of classifier biomarkers from Table 1 from a reference HNSCC B A sample, expression levels of each of the plurality of classifier biomarkers from Table 1 from a reference HNSCC MS sample, expression levels of each of the plurality of classifier biomarkers from Table 1 from a reference HNSCC AT sample, expression levels of each of the plurality of classifier biomarkers from Table 1 from a reference HNSCC CL sample or a combination thereof. In some cases, the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm. In some cases, the expression level of each of the plurality of classifier biomarkers from Table 1 is detected at the nucleic acid level, In some cases, the nucleic acid level is RNA or cDNA. In some cases, the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. In some cases, the expression level is detected by performing qRT-PCR. In some cases, the detecting the expression level is performed using a device that is part of the system or in communication with at least one of the one or more processors, wherein upon receipt of instructions sent by the at least one of the one or more processors, perform the detection of the expression levels. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of classifier biomarkers of Table 1 comprises all the classifier biomarkers from Table 1. In some cases, the system further comprises determining the nodal status of the subject suffering from or suspected of suffering from HNSCC. In some cases, the HNSCC is oral cavity HNSCC.

### BRIEF DESCRIPTION OF THE DRAWINGS

FIGS. 1A-1B illustrate gene expression heat maps including 838 gene classifier genes as described previously (Walter V, Yin X, Wilkerson M D, et al. Molecular subtypes in head and neck cancer exhibit distinct patterns of chromosomal gain and loss of canonical cancer genes. PLOS One. 2013; 8(2):e56823) for (FIG. 1A) all (The Cancer Genome Atlas) TCGA (Head & Neck Squamous Cell Carcinoma) HNSCC (n=520) and (FIG. 1B) OC (n=315).

FIGS. 2A-2B illustrate Kaplan-Meier overall survival (OS) curves for OCSCC patients from TCGA as stratified by node status (FIG. 2A) or stratified by subtype (i.e., mesenchymal vs. non-mesenchymal (other)) and node status (FIG. 2B).

FIGS. 3A-3C illustrate Kaplan-Meier overall survival (OS) curves for oral cavity cancer patients stratified by subtype (mesenchymal vs. non-mesenchymal (other)) and node status from Pickering et al., 2013 (FIG. 3A), non-OCSCC patients from TCGA (FIG. 3B) and Kaplan-Meier OS curve for HPV-negative, non-OCSCC patients from TCGA (FIG. 3C).

FIG. 4A illustrates feature selection from genes in the training set. FIG. 4B illustrates five-fold cross validation curves using a Clanc approach on the TCGA HNSCC dataset (n=520) to guide the selection of the number of genes per subtype to include in the signature for HNSCC subtyping provided herein.

### DETAILED DESCRIPTION OF THE INVENTION

### Overview

The present invention provides systems, kits, compositions and methods for identifying or diagnosing head and neck squamous cell carcinoma or cancer (HNSCC) subtype. In particular, the systems, kits, compositions and methods can be useful for molecularly defining subtypes of HNSCC. In one embodiment, the classifier biomarkers provided herein (e.g., the classifiers in Table 1) can classify the HNSCC patient as possessing one of four molecular subtypes selected from the group consisting of mesenchymal (MS), basal (BA), classical (CL) and atypical (AT). In another embodiment, the classifier biomarkers provided herein (e.g., the classifiers in Table 1) can classify the HNSCC patient as possessing one of four molecular subtypes selected from the group consisting of MS, BA, CL and AT or as not possessing one of four molecular subtypes selected from the group consisting of MS, BA, CL and AT. In other words, the classifiers in Table 1 may be used to a provide a binary classification of a patient's sample as being either a specific subtype or not being that same specific subtype. For example, measuring the expression levels of one or a plurality of the classifiers in Table 1 may be used to classify a sample obtained from a subject suffering from or suspected of suffering from HNSCC as possessing a mesenchymal subtype or a non-mesenchymal subtype. In some cases, the detection of the expression levels of one or a plurality of classifier biomarkers selected from Table 1 in a sample obtained from an HNSCC patient can be used to diagnose that the patient possesses a mesenchymal molecular subtype or a non-mesenchymal subtype. In some cases, the detection of the expression levels of one or a plurality of classifier biomarkers selected from Table 1 in a sample obtained from an HNSCC patient can be used to diagnose that the patient possesses a basal molecular subtype or a non-basal subtype. In some cases, the detection of the expression levels of one or a plurality of classifier biomarkers selected from Table 1 in a sample obtained from an HNSCC patient can be used to diagnose that the patient possesses a classical molecular subtype or a non-classical subtype. In some cases, the detection of the expression levels of one or a plurality of classifier biomarkers selected from Table 1 in a sample obtained from an HNSCC patient can be used to diagnose that the patient possesses an atypical molecular subtype or a non-atypical subtype. In some case, the HNSCC is oral cavity HNSCC. The systems, kits, compositions and methods can be performed to detect HNSCC in patients that are HPV negative. The systems, kits, compositions and methods provide a classification of HNSCC that can be prognostic and predictive for therapeutic response. The therapeutic response can include chemotherapy, immunotherapy, surgical intervention and radiation therapy. The methods can be also provide a prognosis with regards to overall survival for HNSCC patients according to their HNSCC subtype (e.g., AT, MS, CL and BA).

While a useful term for epidemiologic purposes, “Head and Neck Squamous Cell Carcinoma” can refer to cancers arising from the oral cavity, oropharynx, nasopharynx, hypopharynx, and larynx. Subtypes of these types of cancer as defined by underlying genomic features can have varied cell of origin, tumor drivers, proliferation, immune responses, and prognosis.

“Determining a HNSCC subtype” can include, for example, diagnosing or detecting the presence and type of HNSCC, monitoring the progression of the disease, and identifying or detecting cells or samples that are indicative of subtypes.

In one embodiment, HNSCC status is assessed through the evaluation of expression patterns, or profiles, of a plurality of classifier genes or biomarkers or classifier biomarkers in one or more subject samples alone or in combination with assessing HPV status and/or nodal status. For the purpose of discussion, the term “subject”, or “subject sample”, can refer to an individual regardless of health and/or disease status. A subject can be a subject, a study participant, a control subject, a screening subject, or any other class of individual from whom a sample is obtained and assessed in the context of the invention. Accordingly, a subject can be diagnosed with HNSCC (including subtypes, or grades thereof), can present with one or more symptoms of HNSCC, or a predisposing factor, such as a family (genetic) or medical history (medical) factor, for HNSCC, can be undergoing treatment or therapy for HNSCC, or the like. Alternatively, a subject can be healthy with respect to any of the aforementioned factors or criteria. It will be appreciated that the term “healthy” as used herein, is relative to HNSCC status, as the term “healthy” cannot be defined to correspond to any absolute evaluation or status. Thus, an individual defined as healthy with reference to any specified disease or disease criterion, can in fact be diagnosed with any other one or more diseases, or exhibit any other one or more disease criterion, including one or more other cancers.

As used herein, an “expression profile” or a “biomarker profile” or “gene signature” comprises one or more values corresponding to a measurement of the relative abundance, level, presence, or absence of expression of a discriminative or classifier gene or biomarker or classifier biomarker. An expression profile can be derived from a subject prior to or subsequent to a diagnosis of HNSCC, can be derived from a biological sample collected from a subject at one or more time points prior to or following treatment or therapy, can be derived from a biological sample collected from a subject at one or more time points during which there is no treatment or therapy (e.g., to monitor progression of disease or to assess development of disease in a subject diagnosed with or at risk for HNSCC), or can be collected from a healthy subject. The term subject can be used interchangeably with patient. The patient can be a human patient. The one or more biomarkers of the biomarker profiles provided herein can be selected from one or a plurality of biomarkers from Table 1.

As used herein, the term “determining an expression level” or “determining an expression profile” or “detecting an expression level” or “detecting an expression profile” as used in reference to a biomarker or classifier or classifier biomarker can mean the application of a biomarker specific reagent such as a probe, primer or antibody and/or a method to a sample, for example a sample of the subject or patient and/or a control sample, for ascertaining or measuring quantitatively, semi-quantitatively or qualitatively the amount of a biomarker or biomarkers, for example the amount of biomarker polypeptide or mRNA (or cDNA derived therefrom). For example, a level of a biomarker can be determined by a number of methods including for example immunoassays including for example immunohistochemistry, ELISA, Western blot, immunoprecipitation and the like, where a biomarker detection agent such as an antibody for example, a labeled antibody, specifically binds the biomarker and permits for example relative or absolute ascertaining of the amount of polypeptide biomarker, hybridization and PCR protocols where a probe or primer or primer set are used to ascertain the amount of nucleic acid biomarker, including for example probe based and amplification based methods including for example microarray analysis, RT-PCR such as quantitative RT-PCR (qRT-PCR), serial analysis of gene expression (SAGE), Northern Blot, digital molecular barcoding technology, for example Nanostring Counter Analysis, and TaqMan quantitative PCR assays. Other methods of mRNA detection and quantification can be applied, such as mRNA in situ hybridization in formalin-fixed, paraffin-embedded (FFPE) tissue samples or cells. This technology is currently offered by the QuantiGene ViewRNA (Affymetrix), which uses probe sets for each mRNA that bind specifically to an amplification system to amplify the hybridization signals; these amplified signals can be visualized using a standard fluorescence microscope or imaging system. This system for example can detect and measure transcript levels in heterogeneous samples; for example, if a sample has normal and tumor cells present in the same tissue section. As mentioned, TaqMan probe-based gene expression analysis (PCR-based) can also be used for measuring gene expression levels in tissue samples, and this technology has been shown to be useful for measuring mRNA levels in FFPE samples. In brief, TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded, and signals are measured by a detection system; these signal intensities are used to calculate the abundance of a given transcript (gene expression) in a sample.

In one embodiment, the “expression profile” or a “biomarker profile” or “gene signature” associated with the gene cassettes or classifier genes described herein (e.g., Table 1) can be useful for distinguishing between normal and tumor samples. In another embodiment, the tumor samples are Head and Neck Squamous Cell Carcinoma (HNSCC). In another embodiment, HNSCC can be further classified as atypical (AT), basal (BA), classical (CL) and mesenchymal (MS) based upon an expression profile determined using the methods provided herein. In still another embodiment, the expression of HPV genes is determined in the HNSCC sample in order to ascertain the HPV status. The HPV status can be determined prior to, in parallel or after classifying the subtype of HNSCC using the gene signatures presented herein. In another embodiment, the nodal status or presence of nodal metastasis is determined in the HNSCC sample. Expression profiles using the classifier genes disclosed herein (e.g., Table 1) can provide valuable molecular tools for specifically identifying HNSCC subtypes, and for evaluating therapeutic efficacy in treating HNSCC. Accordingly, the invention provides methods for screening and classifying a subject for molecular HNSCC subtypes and methods for monitoring efficacy of certain therapeutic treatments for HNSCC.

In some instances, a single classifier or a plurality of classifiers provided herein (e.g., from Table 1) is capable of identifying subtypes of HNSCC with a predictive success of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

In some instances, a single classifier or a plurality of classifiers as provided herein (e.g., from Table 1) is capable of determining HNSCC subtypes with a sensitivity or specificity of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%.

The present invention also encompasses a system capable of distinguishing or determining various subtypes of HNSCC not detectable using current methods. This system can be capable of processing a large number of subjects and subject variables such as expression profiles and other diagnostic criteria. The methods described herein can also be used for “pharmacometabonomics,” in analogy to pharmacogenomics, e.g., predictive of response to therapy. In this embodiment, subjects could be divided into “responders” and “nonresponders” using the expression profile as evidence of “response,” and features of the expression profile could then be used to target future subjects who would likely respond to a particular therapeutic course or therapy such as, for example, radiation therapy.

The expression profile can be used in combination with other diagnostic methods including histochemical, immunohistochemical, cytologic, immunocytologie, and visual diagnostic methods including histologic or morphometric evaluation of head and neck tissue.

In various embodiments of the present invention, the expression profile derived from a subject is compared to a reference expression profile. A “reference expression profile” or “control expression profile” can be a profile derived from the subject prior to treatment or therapy; can be a profile produced from the subject sample at a particular time point (usually prior to or following treatment or therapy but can also include a particular time point prior to or following diagnosis of HNSCC); or can be derived from a healthy individual or a pooled reference from healthy individuals. A reference expression profile can be generic for HNSCC or can be specific to different subtypes of HNSCC. The HNSCC reference expression profile can be from the oral cavity, oropharynx, nasopharynx, hypopharynx, larynx or any combination thereof.

The reference expression profile can be compared to a test expression profile. A “test expression profile” can be derived from the same subject as the reference expression profile except at a subsequent time point (e.g., one or more days, weeks or months following collection of the reference expression profile) or can be derived from a different subject. In summary, any test expression profile of a subject can be compared to a previously collected profile from a subject that has a AT, MS, BL or CL HNSCC subtype. The previously collected profile can be HPV positive or negative.

The classifier biomarkers of the invention can include nucleic acids (RNA, cDNA, and DNA) and proteins, and variants and fragments thereof. Such biomarkers can include DNA comprising the entire or partial sequence of the nucleic acid sequence encoding the biomarker, or the complement of such a sequence. The biomarkers described herein can include RNA comprising the entire or partial sequence of any of the nucleic acid sequences of interest, or their non-natural cDNA products, obtained synthetically in vitro in a reverse transcription reaction. The biomarker nucleic acids can also include any expression product or portion thereof of the nucleic acid sequences of interest. A biomarker protein can be a protein encoded by or corresponding to a DNA biomarker of the invention. A biomarker protein can comprise the entire or partial amino acid sequence of any of the biomarker proteins or polypeptides. The biomarker nucleic acid can be extracted from a cell or can be cell free or extracted from an extracellular vesicular entity such as an exosome.

A “classifier biomarker” or “biomarker” or “classifier gene” can be any gene or protein whose level of expression in a tissue or cell is altered compared to that of a normal or healthy cell or tissue. For example, a “classifier biomarker” or “biomarker” or “classifier gene” can be any gene or protein whose level of expression in a tissue or cell is altered in a specific HNSCC subtype. The detection of the biomarkers of the invention can permit the determination of the specific subtype. The “classifier biomarker” or “biomarker” or “classifier gene” may be one that is up-regulated (e.g., expression is increased) or down-regulated (e.g., expression is decreased) relative to a reference or control as provided herein. The reference or control can be any reference or control as provided herein. In some embodiments, the expression values of genes that are up-regulated or down-regulated in a particular subtype of HNSCC can be pooled into one gene cassette. The overall expression level in each gene cassette is referred to herein as the “expression profile” and is used to classify a test sample according to the subtype of HNSCC. However, it is understood that independent evaluation of expression for each of the genes disclosed herein can be used to classify tumor subtypes without the need to group up-regulated and down-regulated genes into one or more gene cassettes. In some cases, as shown in Table 2, a total of 88 biomarkers can be used for HNSCC subtype determination. For each HNSCC subtype, 11 of the 22 biomarkers can be negatively correlated genes, while 11 can be positively correlated genes which can be selected as the gene signature of a specific HNSCC subtype.

The classifier biomarkers of the invention include any gene or protein that is selectively expressed in HNSCC, as defined herein above. Sample biomarker genes are listed in Tables 1-2, below. In Table 2, the first column of the table represents the biomarker list selected for distinguishing atypical (AT). The second column of the table represents the biomarker list selected for distinguishing basal (BA). The third column of the table represents the biomarker list selected for distinguishing classical (CL). The last column of the table represents the biomarker list selected for distinguishing Mesenchymal (MS).

In one embodiment, the gene expression levels (e.g., T-statistics) of the classifier biomarkers for HNSCC subtyping are shown in Table 1. In one embodiment, a plurality of classifier biomarkers from Table 1 can be used to classify the subtypes of HNSCC. The plurality of classifier biomarkers selected from Table 1 can comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75,76, 77, 78, 79, 80, 81, 82, 83, 84, 85, 86, 87 or 88 of the biomarkers from Table 1. The plurality of classifier biomarkers selected from Table 1 can comprise at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. The plurality of classifier biomarkers selected from Table 1 can comprise from 1-11 classifier biomarkers, 12-22 classifier biomarkers, 23-33 classifier biomarkers, 34-44 classifier biomarkers, 45-55 classifier biomarkers, 56-66 classifier biomarkers, 67-77 classifier biomarkers or 78-88 classifier biomarkers. The plurality of classifier biomarkers selected from Table 1 can comprise at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the biomarkers from Table 1. The plurality of classifier biomarkers selected from Table 1 can comprise the classifier biomarkers specified for each HNSCC subtype as outlined in Table 2 or subsets thereof.

TABLE 1
Gene Centroids of 88 Classifier Biomarkers for the Head & Neck Squamous
Cell Carcinoma (HNSCC) Subtypes

| | | | | | | GenBank | SEQ |
|---|---|---|---|---|---|---|---|
| Gene | | | | | Mesenchymal | Accession | ID |
| Symbol | Gene Name | Atypical (AT) | Basal (BA) | Classical (CL) | (MS) | Number* | NO: |
| ABCC1 | ATP binding | −0.031099521 | −0.088022349 | 1.577266762 | −0.218432138 | NM_004996 | 1 |
| | cassette subfamily | | | | | | |
| | C member 1 | | | | | | |
| ABCC5 | ATP binding | 0.848165306 | −0.620695031 | 1.706850075 | −0.445549677 | NM_005688 | 2 |
| | cassette subfamily | | | | | | |
| | C member 5 | | | | | | |
| ACTN1 | actinin alpha 1 | −1.293584309 | 0.298046714 | −0.402145104 | 0.522579935 | NM_001130004 | 3 |
| ADH7 | alcohol | 1.821628068 | −1.725423951 | 2.375396314 | −3.931734615 | NM_000673 | |
| | dehydrogenase 7 | | | | | | |
| ALDH3A1 | aldehyde | 1.739405007 | −1.14227612 | 3.140240719 | −2.508052893 | NM_001135168.1 | 5 |
| | dehydrogenase 3 | | | | | | |
| | family member | | | | | | |
| | A1 | | | | | | |
| APBB2 | amyloid beta | −0.949643571 | 0.232934212 | −0.401590283 | 0.631536884 | NM_004307 | 6 |
| | precursor protein | | | | | | |
| | binding family B | | | | | | |
| | member 2 | | | | | | |
| APOL3 | apolipoprotein | 0.185011664 | 0.356891782 | −1.312029702 | 0.648715202 | NM_014349 | 7 |
| | 1.3 | | | | | | |
| AQP3 | aquaporin 3 | 0.283388192 | 1.382159215 | −2.45885437 | −1.004443776 | NM_004925 | 8 |
| ATP6V1D | ATPase H+ | −0.338252765 | 0.590353958 | −0.389255392 | −0.021203396 | NM_015994 | 9 |
| | transporting V1 | | | | | | |
| | subunit D | | | | | | |
| CABYR | calcium binding | −0.100055188 | −0.988063965 | 1.939529766 | 0.124303738 | NM_012189 | 10 |
| | tyrosine | | | | | | |
| | phosphorylation | | | | | | |
| | regulated | | | | | | |
| CASP4 | caspase 4 | −0.319195269 | 1.007963576 | −0.818460799 | 0.055167237 | NM_001225 | |
| CAV1 | caveolin 1 | −1.693362694 | 0.712991833 | −0.646447104 | 0.570953433 | NM_001753 | 12 |
| CDSN | corneodesmosin | −3.161014982 | 3.351364866 | −1.735738737 | −0.144278064 | NM_001264 | 13 |
| CHPT1 | choline | 0.8184384 | −1.172389871 | 0.443862893 | 0.052774556 | NM_020244 | 14 |
| | phosphotransferase | | | | | | |
| | 1 | | | | | | |
| CHST7 | carbohydrate | 0.01618388 | −0.744563015 | 1.471041713 | 0.01113381 | NM_019886 | 15 |
| | sulfotransferase 7 | | | | | | |
| CHTA | class II major | 0.861691256 | −0.602594558 | −1.771738528 | 0.55032782 | NM_001286402 | 16 |
| | histocompatibility | | | | | | |
| | complex | | | | | | |
| | transactivator | | | | | | |
| CMTM3 | CKLF like | −0.365194396 | −0.33427279 | −0.061274715 | 0.874011757 | NM_144601 | 17 |
| | MARVEL | | | | | | |
| | transmembrane | | | | | | |
| | domain | | | | | | |
| | containing 3 | | | | | | |
| COL6A1 | collagen type VI | −0.95091155 | −0.345006545 | 0.015156324 | 1.479593776 | NM_001848 | 18 |
| | alpha 1 chain | | | | | | |
| COL6A2 | collagen type VI | −0.877414641 | −0.392937241 | −0.008200304 | 1.393961586 | NM_001849 | 19 |
| | alpha 2 chain | | | | | | |
| CSTA | cystatin A | 0.854688493 | 1.309208931 | −0.607570577 | −1.685886632 | NM_005213 | 20 |
| CYP26A1 | ytochrome P450 | 1.843455752 | −1.941663157 | 5.466906249 | −0.776253849 | NM_000783 | 21 |
| | family 26 | | | | | | |
| | subfamily A | | | | | | |
| | member 1 | | | | | | |
| CYP2C18 | cytochrome P450 | 1.356729589 | 0.354459727 | 0.403107806 | −2.670294053 | NM_000772 | 22 |
| | family 2 | | | | | | |
| | subfamily C | | | | | | |
| | member 18 | | | | | | |
| DHRS1 | dehydrogenase/ | −0.038783854 | 1.021308448 | −0.890296642 | −0.413141678 | NM_001136050 | 23 |
| | reductase 1 | | | | | | |
| ELF3 | E74 like ETS | 1.515555672 | −0.572308251 | 0.680521993 | −1.567893679 | NM_004433 | 24 |
| | transcription | | | | | | |
| | factor 3 | | | | | | |
| EPCAM | epithelial cell | 0.759829367 | −0.778769628 | 2.235464768 | −0.537704156 | NM_002354 | 25 |
| | adhesion | | | | | | |
| | molecule | | | | | | |
| EPGN | epithelial | −0.849644824 | 2.751069624 | −1.561946089 | −0.50052785 | NM_001270989 | 26 |
| | mitogen | | | | | | |
| F2RL1 | F2R like trypsin | −1.137490546 | 0.527010052 | −0.187750552 | 0.051849021 | NM_005242 | 27 |
| | receptor 1 | | | | | | |
| FAM171A1 | family with | 1.060683199 | −1.483370036 | 0.573897987 | 0.324765224 | NM_001010924 | 28 |
| | sequence | | | | | | |
| | similarity 171 | | | | | | |
| | member A1 | | | | | | |
| FAM3B | family with | 1.060683199 | −1.483370036 | 0.573897987 | 0.324765224 | NM_058186 | 29 |
| | sequence | | | | | | |
| | similarity 3 | | | | | | |
| | member B | | | | | | |
| FAM40A | striatin | 0.038610305 | 0.276587248 | −0.435828797 | −0.031790512 | NM_033088 | 30 |
| | interacting protein | | | | | | |
| | 1 | | | | | | |
| FBLIM1 | filamin binding | −0.983094146 | 0.825431115 | −0.605490022 | 0.213253525 | NM_017556 | 31 |
| | LIM protein 1 | | | | | | |
| FOXA1 | forkhead box A1 | 2.91945945 | −1.744221617 | 1.548666747 | −1.562853306 | NM_004496 | 32 |
| FSTL3 | follistatin like 3 | −1.600890337 | 0.547486069 | −0.586648673 | 0.837816469 | NM_005860 | 33 |
| FUT6 | fucosyltransferase | 2.784333146 | −0.08954694 | −0.543062349 | −1.909670258 | NM_000150 | 34 |
| | 6 | | | | | | |
| GCNT2 | glucosaminyl (N- | 1.332068547 | −1.618593447 | 1.754231039 | −0.753190768 | NM_145649 | 35 |
| | acetyl) transferase | | | | | | |
| | 2, I-branching | | | | | | |
| | enzyme (I blood | | | | | | |
| | group) | | | | | | |
| GPX8 | glutathione | −0.529954827 | −0.227552031 | −0.047591051 | 0.903499417 | NM_001008397 | 36 |
| | peroxidase 8 | | | | | | |
| | (putative) | | | | | | |
| GRHL3 | grainy head like | 0.640291551 | 0.696898246 | −0.082486542 | −1.651684921 | NM_021180 | 37 |
| | transcription | | | | | | |
| | factor 3 | | | | | | |
| GSDMA | gasdermin A | −1.906289619 | 2.73576717 | −1.591903136 | −0.19742346 | NM_178171 | 38 |
| HLF | HLF, PAR bZIP | 2.147467678 | −1.424085584 | 1.023965686 | −1.471836156 | NM_002126 | 39 |
| | transcription | | | | | | |
| | factor | | | | | | |
| IL4R | interleukin 4 | −0.023822143 | 0.50614129 | −0.94171812 | −0.030139037 | NM_000418 | 40 |
| | receptor | | | | | | |
| INHBA | inhibin beta A | −2.321727685 | 0.76655613 | −0.376302156 | 1.295501649 | NM_002192 | 41 |
| | subunit | | | | | | |
| KIAA1609 | KIAA1609 | −0.709922727 | 0.727885172 | −0.869073748 | 0.057541023 | AB046829 | 42 |
| KLF5 | Kruppel like | 0.214024277 | 0.189189637 | 0.230622819 | −1.009211838 | NM_001730 | 43 |
| | factor 5 | | | | | | |
| KRT6B | keratin 6B | −1.128016918 | 1.912448163 | −0.79463927 | −0.275607667 | NM_005555 | 44 |
| LEPRE1 | prolyl 3- | −0.598607195 | −0.07373454 | 0.033878301 | 0.926909146 | NM_022356 | 45 |
| | hydroxylase 1 | | | | | | |
| LTBP3 | latent | 0.290345624 | −1.057031806 | 0.552016552 | 0.483311516 | NM_001130144 | 46 |
| | transforming | | | | | | |
| | growth factor beta | | | | | | |
| | binding protein 3 | | | | | | |
| MAP7D1 | MAP7 domain | −0.607395988 | 0.506161147 | −0.672818118 | 0.074151567 | NM_018067 | 47 |
| | containing 1 | | | | | | |
| MEIS1 | Meis homeobox | 1.324693348 | −0.570108622 | −0.145704397 | −0.312498724 | NM_002398 | 48 |
| | 1 | | | | | | |
| MOBKL2B | MOB kinase | −0.341540641 | 0.668236691 | −1.353477513 | 0.342254939 | NM_024761 | 49 |
| | activator 3B | | | | | | |
| MUC20 | mucin 20, cell | 2.101356837 | −1.264580747 | 0.351022262 | −1.928550867 | NM_001282506 | 50 |
| | surface associated | | | | | | |
| MUC4 | mucin 4, cell | 3.716198619 | −2.223689252 | 1.386732186 | −1.740076741 | NM_018406 | 51 |
| | surface | | | | | | |
| | associated | | | | | | |
| NNMT | nicotinamide N- | −1.020903024 | −0.293641174 | −0.503772478 | 1.571306782 | NM_024677 | 52 |
| | methyltransferase | | | | | | |
| NSUN7 | NOP2/Sun RNA | 1.914071438 | −2.054022059 | 1.062558206 | −0.7903323 | NM_006180 | 53 |
| | methyltransferase | | | | | | |
| | family member 7 | | | | | | |
| OLFML2B | olfactomedin like | −0.574224533 | −0.539134111 | −0.122494458 | 1.425018051 | NM_001297713 | 54 |
| | 2B | | | | | | |
| OLFML3 | olfactomedin like | −0.393002594 | −0.388316214 | −0.30044045 | 1.739914503 | NM_020190 | 55 |
| | 3 | | | | | | |
| P4HTM | prolyl 4- | 0.837588916 | −1.262928977 | 0.385594983 | 0.040529582 | NM_177939 | 56 |
| | hydroxylase, | | | | | | |
| | transmembrane | | | | | | |
| PATZ1 | POZ/BTB and | 0.569619498 | −0.780939229 | 0.603096762 | 0.147128874 | NM_014323 | 57 |
| | AT hook | | | | | | |
| | containing zinc | | | | | | |
| | finger 1 | | | | | | |
| PBX1 | PBX homeobox 1 | 1.41891856 | −2.065393653 | 0.582813384 | −0.289716958 | NM_002585 | 58 |
| PCOLCE | procollagen C- | −0.623866829 | −0.724947656 | 0.169461396 | 1.655170706 | NM_002593 | 59 |
| | endopeptidase | | | | | | |
| | enhancer | | | | | | |
| PHLDB1 | pleckstrin | −0.646447264 | −0.198849026 | −0.030781782 | 0.958476723 | NM_015157 | 60 |
| | homology like | | | | | | |
| | domain family B | | | | | | |
| | member 1 | | | | | | |
| PIR | pirin | 0.529711003 | −0.781884581 | 1.201463319 | −0.833053986 | NM_003662 | 61 |
| PLAC8 | placenta specific | 3.064847313 | −1.490282981 | 0.270596341 | −0.383189938 | NM_001130716 | 62 |
| | 8 | | | | | | |
| PLD2 | phospholipase | −0.219944115 | 0.831689264 | −0.512740581 | −0.225493291 | NM_002663 | 63 |
| | D2 | | | | | | |
| PPARD | peroxisome | −0.287680267 | 0.76911827 | −0.356343286 | −0.097154473 | NM_006238 | 64 |
| | proliferator | | | | | | |
| | activated receptor | | | | | | |
| | delta | | | | | | |
| PRKX | protein kinase, | 0.65819026 | −0.643335513 | 1.513787469 | −0.453801881 | NM_005044 | 65 |
| | X-linked | | | | | | |
| PTH1R | parathyroid | −0.620526783 | −0.665018743 | −0.044018096 | 1.468148537 | NM_000316 | 66 |
| | hormone 1 | | | | | | |
| | receptor | | | | | | |
| RAB6B | RAB6B, member | −0.015731951 | −0.898063489 | 2.611374968 | 0.227812876 | NM_016577 | 67 |
| | RAS oncogene | | | | | | |
| | family | | | | | | |
| RIMKLA | ribosomal | 0.825364092 | −1.702444703 | 2.485284775 | −0.328228788 | NM_173642 | 68 |
| | modification | | | | | | |
| | protein rimK like | | | | | | |
| | family member A | | | | | | |
| SERPINE1 | serpin family E | −1.858000797 | 0.306735823 | 0.243735393 | 0.897894212 | NM_000602 | 69 |
| | member 1 | | | | | | |
| SERPINH1 | serpin family H | −1.088498793 | −0.010766577 | 9.89E−05 | 0.711345224 | NM_001207014 | 70 |
| | member 1 | | | | | | |
| SFXN3 | sideroflexin 3 | −0.763412695 | 0.331651271 | −0.561650179 | 0.507585 | NM_030971 | 71 |
| SGEF | SH3-containing | 2.032561924 | −2.280754792 | 2.363347289 | −1.476907968 | AY552599 | 72 |
| | guanine | | | | | | |
| | nucleotide | | | | | | |
| | exchange factor | | | | | | |
| SLAMF7 | SLAM family | 0.458423187 | 0.112741008 | −1.618459318 | 0.163825343 | NM_021181 | 73 |
| | member 7 | | | | | | |
| SLC31A2 | solute carrier | −0.60782846 | 0.613355295 | −1.001307761 | 0.373857753 | NM_001860 | 74 |
| | family 31 member | | | | | | |
| | 2 | | | | | | |
| SLC9A3R1 | SLC9A3 | 0.541348713 | −0.108826906 | 0.266863903 | −0.904566234 | NM_004252 | 75 |
| | regulator 1 | | | | | | |
| SNAI2 | snail family | −1.01844029 | 0.244349221 | −0.001603835 | 0.38921459 | NM_003068 | 76 |
| | transcriptional | | | | | | |
| | repressor 2 | | | | | | |
| SOX2 | SRY-box | 1.509922452 | −0.920956385 | 2.467128213 | −1.616313701 | NM_003106 | 77 |
| | transcription | | | | | | |
| | factor 2 | | | | | | |
| SPRR3 | small proline rich | 2.89913386 | 0.333079744 | 0.015490942 | −4.20364091 | NM_005416 | 78 |
| | protein 3 | | | | | | |
| TGFBI | transforming | −1.631233479 | 0.535964267 | −0.480920716 | 1.019689967 | NM_000358 | 79 |
| | growth factor beta | | | | | | |
| | induced | | | | | | |
| TJP3 | tight junction | 2.740061086 | −0.983576292 | −0.207989711 | −1.456299411 | NM_001267560 | 80 |
| | protein 3 | | | | | | |
| TMEM51 | transmembrane | 0.097399701 | 0.154143771 | −0.982669471 | 0.050400488 | NM_001136216 | 81 |
| | protein 51 | | | | | | |
| TMPRSS11A | transmembrane | 3.436710203 | −0.745119028 | 0.436009077 | −3.133790366 | NM_182606 | 82 |
| | protease, serine | | | | | | |
| | 11A | | | | | | |
| TMPRSS11B | transmembrane | 4.408612343 | −0.75328622 | 0 | −2.878438108 | NM_182502 | 83 |
| | protease, setine | | | | | | |
| | 11B | | | | | | |
| TMPRSS2 | transmembrane | 3.325694152 | −2.366380237 | 0.523971501 | −2.639486469 | NM_001135099 | 84 |
| | protease, serine 2 | | | | | | |
| TXNRD1 | thioredoxin | 0.056473899 | −0.304486896 | 2.153316092 | −0.143440851 | NM_182729 | 85 |
| | reductase 1 | | | | | | |
| UBA7 | ubiquitin like | 0.320973905 | −0.09917239 | −1.239676508 | 0.324881323 | NM_003335 | 86 |
| | modifier | | | | | | |
| | activating enzyme | | | | | | |
| | 7 | | | | | | |
| WNK2 | WNK lysine | 2.451530343 | −3.101925236 | 2.344715243 | −1.040438635 | NM_001282394 | 87 |
| | deficient protein | | | | | | |
| | kinase 2 | | | | | | |
| ZDHHC2 | zinc finger | 0.796593087 | −1.94177472 | 1.428597198 | 0.048202702 | NM_016353 | 88 |
| | DHHC-type | | | | | | |
| | containing 2 | | | | | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 2
Classifier Biomarkers Selected for
AT, MS, BA and CL HNSCC Subtypes

| | Atypical | Mesenchymal | Classical | Basal |
|---|---|---|---|---|
| Number | (AT) | (MS) | (CL) | (BA) |
| 1 | ACTN1 | CYP2C18 | ABCC1 | ATP6V1D |
| 2 | APBB2 | CMTM3 | ABCC5 | CDSN |
| 3 | CAV1 | COL6A1 | APOL3 | CHPT1 |
| 4 | FAM3B | COL6A2 | AQP3 | DHRS1 |
| 5 | FOXA1 | CSTA | CABYR | EPGN |
| 6 | FSTL3 | ELF3 | CASP4 | FAM171A1 |
| 7 | FUT6 | GPX8 | CHST7 | FBLIM1 |
| 8 | HLF | GRHL3 | CIITA | GCNT2 |
| 9 | INHBA | KLF5 | CYP26A1 | GSDMA |
| 10 | MEIS1 | LEPRE1 | EPCAM | KIAA1609 |
| 11 | MUC4 | SPRR3 | FAM40A | LTBP3 |
| 12 | PLAC8 | NNMT | IL4R | MAP7D1 |
| 13 | SERPINE1 | OLFML2B | MOBKL2B | NSUN7 |
| 14 | SERPINH1 | OLFML3 | PRKX | P4HTM |
| 15 | SFXN3 | PCOLCE | RAB6B | PATZ1 |
| 16 | SNAI2 | PHLDB1 | RIMKLA | PBX1 |
| 17 | TGFBI | ADH7 | SLC31A2 | PLD2 |
| 18 | TJP3 | SLC9A3R1 | TMEM51 | PPARD |
| 19 | TMPRSS11B | TMPRSS11A | TXNRD1 | SGEF |
| 20 | TMPRSS2 | ALDH3A1 | UBA7 | ZDHHC2 |
| 21 | MUC20 | PTH1R | PIR | KRT6B |
| 22 | F2RL1 | SOX2 | SLAMF7 | WNK2 |

### Diagnostic Uses

In one embodiment, the methods and compositions provided herein allow for the differentiation of the four subtypes of HNSCC: (1) Basal (BA); (2) Mesenchymal (MS); (3) Atypical (AT); and (4) Classical (CL), with fewer genes needed than the molecular HNSCC subtyping methods known in the art

In general, the methods provided herein are used to classify a sample obtained from a subject suffering from or suspected of suffering from or at risk of suffering from HNSCC, as a particular HNSCC subtype (e.g., subtype of HNSCC). In one embodiment, the method comprises measuring, detecting or determining an expression level of at least one or a plurality of the classifier biomarkers of any publicly available HNSCC expression dataset. In one embodiment, the method comprises detecting or determining an expression level of at least one or a plurality of the classifier biomarkers of Table 1 in a sample obtained from a patient or a subject suffering from or suspected of suffering from or at risk of suffering from HNSCC. The sample for the detection or differentiation methods described herein can be a sample previously determined or diagnosed as squamous cell carcinoma (SCC) sample. The previous diagnosis can be based on a histological analysis. The histological analysis can be performed by one or more pathologists.

In one embodiment, the measuring or detecting step is at the nucleic acid level by performing RNA-seq, a reverse transcriptase polymerase chain reaction (RT-PCR) or a hybridization assay with oligonucleotides that are substantially complementary to portions of cDNA molecules of the one or a plurality of classifier biomarker(s) (such as the classifier biomarkers of Table 1) under conditions suitable for RNA-seq, RT-PCR or hybridization and obtaining expression levels of the one or plurality of classifier biomarkers based on the detecting step. The expression levels of the one or plurality of the classifier biomarkers are then compared to the expression level of the one or plurality of the classifier biomarkers in a sample obtained from a control (i.e., a control sample). In some cases, the control sample comprises reference expression levels of the one or plurality of the classifier biomarker(s) (such as the classifier biomarkers from Table 1) from at least one sample training set. The at least one sample training set can comprise, (i) expression levels of the one or plurality of classifier biomarker(s) from a sample that overexpresses the one or plurality of classifier biomarker(s), (ii) expression levels from a reference BA, MS, AT or CL sample, or (iii) expression levels from SCC free head and neck sample and classifying the head and neck tissue sample as a BA, MS, AT or CL subtype. The head and neck cancer sample can then be classified as a BA, MS, AT or CL subtype of squamous cell carcinoma based on the results of the comparing step. In one embodiment, the comparing step can comprise applying a statistical algorithm which comprises determining a correlation between the expression data obtained from the head and neck tissue or cancer sample and the expression data from the control sample; and classifying the head and neck tissue or cancer sample as a BA, MS, AT or CL sample subtype based on the results of the statistical algorithm. In some cases, the control sample comprises the at least one training set(s) as described herein.

In one embodiment, the method comprises probing the levels of one or a plurality of classifier biomarker(s) provided herein, such as the classifier biomarkers of Table 1 at the nucleic acid level, in sample obtained from the patient suffering from or suspected of suffering from a head and neck cancer. The sample can be a sample previously determined or diagnosed as a squamous cell carcinoma (SCC or SQ) sample. The previous diagnosis can be based on a histological analysis. The histological analysis can be performed by one or more pathologists. The probing step, in one embodiment, comprises mixing the sample with one or more oligonucleotides that are substantially complementary to portions of cDNA molecules of the one or each classifier biomarker from the plurality of classifier biomarker(s) provided herein, such as the classifier biomarkers of Table 1 under conditions suitable for hybridization of the one or more oligonucleotides to their complements or substantial complements; detecting whether hybridization occurs between the one or more oligonucleotides to their complements or substantial complements; and obtaining hybridization values of the one or plurality of classifier biomarker(s) based on the detecting step. The hybridization values of the one or plurality of classifier biomarker(s) are then compared to reference hybridization value(s) from at least one control. sample training set. For example, the control can be at least one training set, wherein the at least one sample training set comprises hybridization values from a reference BA SCC, MS SCC AT SCC, and/or CL SCC sample. The sample obtained from the subject can be classified, for example, as BA, MS, AT or CL based on the results of the comparing step.

The head and neck tissue sample can be any sample isolated from a human subject or patient. For example, in one embodiment, the analysis is performed on head and neck biopsies that are embedded in paraffin wax. In one embodiment, the sample can be a fresh frozen head and neck tissue sample obtained from the head and/or neck area of the subject or patient. In another embodiment, the sample can be a bodily fluid obtained from the patient. The bodily fluid can be blood or fractions thereof (i.e., serum, plasma), urine, saliva, sputum or cerebrospinal fluid (CSF). The sample can contain cellular as well as extracellular sources of nucleic acid for use in the methods provided herein. The extracellular sources can be cell-free DNA and/or exosomes. In one embodiment, the sample can be a cell pellet or a wash. This aspect of the invention provides a means to improve current diagnostics by accurately identifying the major histological types, even from small biopsies. The methods of the invention, including the RT-PCR methods, are sensitive, precise and have multi-analyte capability for use with paraffin embedded samples. See, for example, Cronin et al. (2004) Am. J Pathol. 164(1):35-42, herein incorporated by reference.

Formalin fixation and tissue embedding in paraffin wax is a universal approach for tissue processing prior to light microscopic evaluation. A major advantage afforded by formalin-fixed paraffin-embedded (FFPE) specimens is the preservation of cellular and architectural morphologic detail in tissue sections. (Fox et al. (1985) J Histochem Cytochem 33:845-853). The standard buffered formalin fixative in which biopsy specimens are processed is typically an aqueous solution containing 37% formaldehyde and 10-15% methyl alcohol. Formaldehyde is a highly reactive dipolar compound that results in the formation of protein-nucleic acid and protein-protein crosslinks in vitro (Clark et al. (1986) J Histochem Cytochem 34:1509-1512; McGhee and von Hippel (1975) Biochemistry 14:1281-1296, each incorporated by reference herein).

In one embodiment, the sample used herein is obtained from an individual, and comprises formalin-fixed paraffin-embedded (FFPE) tissue. However, other tissue and sample types are amenable for use herein. In one embodiment, the other tissue and sample types can be fresh frozen tissue, wash fluids, or cell pellets, or the like. In one embodiment, the sample can be a bodily fluid obtained from the individual. The bodily fluid can be blood or fractions thereof (e.g., serum, plasma), urine, sputum, saliva or cerebrospinal fluid (CSF). A biomarker nucleic acid as provided herein can be extracted from a cell or can be cell free or extracted from an extracellular vesicular entity such as an exosome.

Methods are known in the art for the isolation of RNA from FFPE tissue. In one embodiment, total RNA can be isolated from FFPE tissues as described by Bibikova et al. (2004) American Journal of Pathology 165:1799-1807, herein incorporated by reference. Likewise, the High Pure RNA Paraffin Kit (Roche) can be used. Paraffin is removed by xylene extraction followed by ethanol wash. RNA can be isolated from sectioned tissue blocks using the MasterPure Purification kit (Epicenter, Madison, Wis.); a DNase I treatment step is included. RNA can be extracted from frozen samples using Trizol reagent according to the supplier's instructions (Invitrogen Life Technologies, Carlsbad, Calif.). Samples with measurable residual genomic DNA can be resubjected to DNaseI treatment and assayed for DNA contamination. All purification, DNase treatment, and other steps can be performed according to the manufacturer's protocol. After total RNA isolation, samples can be stored at −80° C. until use.

General methods for mRNA extraction are well known in the art and are disclosed in standard textbooks of molecular biology, including Ausubel et al., ed., Current Protocols in Molecular Biology, John Wiley & Sons, New York 1987-1999. Methods for RNA extraction from paraffin embedded tissues are disclosed, for example, in Rupp and Locker (Lab Invest. 56: A67, 1987) and De Andres et al. (Biotechniques 18:42-44, 1995). In particular, RNA isolation can be performed using a purification kit, a buffer set and protease from commercial manufacturers, such as Qiagen (Valencia, Calif.), according to the manufacturer's instructions. For example, total RNA from cells in culture can be isolated using Qiagen RNeasy mini-columns. Other commercially available RNA isolation kits include MasterPure™. Complete DNA and RNA Purification Kit (Epicentre, Madison, Wis.) and Paraffin Block RNA Isolation Kit (Ambion, Austin, Tex.). Total RNA from tissue samples can be isolated, for example, using RNA Stat-60 (Tel-Test, Friendswood, Tex.). RNA prepared from a tumor can be isolated, for example, by cesium chloride density gradient centrifugation. Additionally, large numbers of tissue samples can readily be processed using techniques well known to those of skill in the art, such as, for example, the single-step RNA isolation process of Chomczynski (U.S. Pat. No. 4,843,155, incorporated by reference in its entirety for all purposes).

In one embodiment, a sample comprises cells harvested from a head and neck tissue sample, for example, a squamous cell carcinoma sample. Cells can be harvested from a biological sample using standard techniques known in the art. For example, in one embodiment, cells are harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract nucleic acid, e.g., messenger RNA (mRNA). All samples obtained from a subject, including those subjected to any sort of further processing, are considered to be obtained from the subject,

The sample, in one embodiment, is further processed before the detection of the biomarker levels of the combination of biomarkers set forth herein. For example, mRNA in a cell or tissue sample can be separated from other components of the sample. The sample can be concentrated and/or purified to isolate mRNA in its non-natural state, as the mRNA is not in its natural environment. For example, studies have indicated that the higher order structure of mRNA in vivo differs from the in vitro structure of the same sequence (see, e.g., Rouskin et al. (2014). Nature 505, pp. 701-705, incorporated herein in its entirety for all purposes).

mRNA from the sample in one embodiment, is hybridized to a synthetic DNA probe, which in some embodiments, includes a detection moiety (e.g., detectable label, capture sequence, barcode reporting sequence). Accordingly, in these embodiments, a non-natural mRNA-cDNA complex is ultimately made and used for detection of the biomarker. In another embodiment, mRNA from the sample is directly labeled with a detectable label, e.g., a fluorophore. In a further embodiment, the non-natural labeled-mRNA molecule is hybridized to a cDNA probe and the complex is detected.

In one embodiment, once the mRNA is obtained from a sample, it is converted to complementary DNA (cDNA) prior to the hybridization reaction or is used in a hybridization reaction together with one or more cDNA probes. cDNA does not exist in vivo and therefore is a non-natural molecule. Furthermore, cDNA-mRNA hybrids are synthetic and do not exist in vivo. Besides cDNA not existing in vivo, cDNA is necessarily different than mRNA, as it includes deoxyribonucleic acid and not ribonucleic acid. The cDNA is then amplified, for example, by the polymerase chain reaction (PCR) or other amplification method known to those of ordinary skill in the art. For example, other amplification methods that may be employed include the ligase chain reaction (LCR) (Wu and Wallace, Genomics, 4:560 (1989), Landegren et al., Science, 241:1077 (1988), incorporated by reference in its entirety for all purposes, transcription amplification (Kwoh et al., Proc. Natl. Acad. Sci. USA, 86:1173 (1989), incorporated by reference in its entirety for all purposes), self-sustained sequence replication (Guatelli et al., Proc. Nat. Acad. Sci. USA, 87:1874 (1990), incorporated by reference in its entirety for all purposes), incorporated by reference in its entirety for all purposes, and nucleic acid based sequence amplification (NASBA). Guidelines for selecting primers for PCR amplification are known to those of ordinary skill in the art. See, e.g., McPherson et al., PCR Basics: From Background to Bench, Springer-Verlag, 2000, incorporated by reference in its entirety for all purposes. The product of this amplification reaction, i.e., amplified cDNA is also necessarily a non-natural product. First, as mentioned above, cDNA is a non-natural molecule. Second, in the case of PCR, the amplification process serves to create hundreds of millions of cDNA copies for every individual cDNA molecule of starting material. The numbers of copies generated are far removed from the number of copies of mRNA that are present in vivo.

In one embodiment, cDNA is amplified with primers that introduce an additional DNA sequence (e.g., adapter, reporter, capture sequence or moiety, barcode) onto the fragments (e.g., with the use of adapter-specific primers), or mRNA or cDNA biomarker sequences are hybridized directly to a cDNA probe comprising the additional sequence (e.g., adapter, reporter, capture sequence or moiety, barcode). Amplification and/or hybridization of mRNA to a cDNA probe therefore serves to create non-natural double stranded molecules from the non-natural single stranded cDNA, or the mRNA, by introducing additional sequences and forming non-natural hybrids. Further, as known to those of ordinary skill in the art, amplification procedures have error rates associated with them. Therefore, amplification introduces further modifications into the cDNA molecules. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (i) adapter sequences are added to the ends of cDNA molecules to make DNA sequences that do not exist in vivo, (ii) the error rate associated with amplification further creates DNA sequences that do not exist in vivo, (iii) the disparate structure of the cDNA molecules as compared to what exists in nature, and (iv) the chemical addition of a detectable label to the cDNA molecules.

In some embodiments, the expression of a biomarker of interest is detected at the nucleic acid level via detection of non-natural cDNA molecules.

In some embodiments, the method for head and neck cancer SCC subtyping includes detecting expression levels of a classifier biomarker set in a sample obtained from a subject. The method can further comprise detecting expression levels of said classifier biomarker set in one or more control or reference samples. The one or more control or reference samples can be selected from a normal or HNSCC-free sample, a HNSCC AT sample, a HNSCC HPV+ AT-like sample, a HNSCC BA sample, a HNSCC MS sample, a HNSCC CL sample or any combination thereof. In some embodiments, the detecting includes all of the classifier biomarkers of Table 1 at the nucleic acid level or protein level. In another embodiment, a single or a subset or a plurality of the classifier biomarkers of Table 1 are detected, for example, from about 11 to about 22. For example, in one embodiment, from about 5 to about 11, from about 12 to about 22, from about 23 to about 33, from about 34 to about 44, from about 45 to about 55, from about 56 to about 66, from about 67 to about 77 or from about 78 to about 88 of the biomarkers in Table 1 are detected in a method to determine the Head and Neck cancer SQ subtype. In another embodiment, each of the biomarkers from Table 1 is detected in a method to determine the Head and Neck cancer subtype. In another embodiment, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75,76, 77, 78, 79, 80, 81, 82, 83, 84, 85, 86, 87 or 88 of the biomarkers from Table 1 are detected in a method to determine the Head and Neck cancer SQ subtype. In another embodiment, at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1 are detected in a method to determine the Head and Neck cancer SQ subtype. In yet another embodiment, at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the biomarkers from Table 1 are detected in a method to determine the Head and Neck cancer SQ subtype. In another embodiment, 22 of the biomarkers from Table 1 are selected as the gene signatures for a specific Head and Neck cancer SQ subtype as shown in Table 2. The detecting can be performed by any suitable technique including, but not limited to, RNA-seq, a reverse transcriptase polymerase chain reaction (RT-PCR), a microarray hybridization assay, or another hybridization assay, e.g., a NanoString assay for example, with primers and/or probes specific to the classifier biomarkers, and/or the like. In some cases, the primers useful for the amplification methods (e.g., RT-PCR or qRT-PCR) are any forward and reverse primers suitable for binding to a classifier gene provided herein, such as the classifier biomarkers listed in Table 1.

The biomarkers described herein include RNA comprising the entire or partial sequence of any of the nucleic acid sequences of interest, or their non-natural cDNA product, obtained synthetically in vitro in a reverse transcription reaction. The term “fragment” is intended to refer to a portion of the polynucleotide that generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polynucleotide will generally encode at least 15, 25, 30, 50, 100, 150, 200, or 250 contiguous amino acids, or up to the total number of amino acids present in a full-length biomarker protein of the invention.

In some embodiments, overexpression, such as of an RNA transcript or its expression product, is determined by normalization to the level of reference RNA transcripts or their expression products, which can be all measured transcripts (or their products) in the sample or a particular reference set of RNA transcripts (or their non-natural cDNA products). Normalization is performed to correct for or normalize away both differences in the amount of RNA or cDNA assayed and variability in the quality of the RNA or cDNA used. Therefore, an assay typically measures and incorporates the expression of certain normalizing genes, including well known housekeeping genes, such as, for example, GAPDH and/or β-Actin. Alternatively, normalization can be based on the mean or median signal of all of the assayed biomarkers or a large subset thereof (global normalization approach).

Isolated mRNA can be used in hybridization or amplification assays that include, but are not limited to, Southern or Northern analyses, PCR analyses and probe arrays, NanoString Assays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least 7, 15, 30, 50, 100, 250, or 500 nucleotides in length and sufficient to specifically hybridize under stringent conditions to the non-natural cDNA or mRNA biomarker of the present invention.

As explained above, in one embodiment, once the mRNA is obtained from a sample, it is converted to complementary DNA (cDNA) in a hybridization reaction. Conversion of the mRNA to cDNA can be performed with oligonucleotides or primers comprising sequence that is complementary to a portion of a specific mRNA. Conversion of the mRNA to cDNA can be performed with oligonucleotides or primers comprising random sequence. Conversion of the mRNA to cDNA can be performed with oligonucleotides or primers comprising sequence that is complementary to the poly (A) tail of an mRNA. cDNA does not exist in vivo and therefore is a non-natural molecule. In a further embodiment, the cDNA is then amplified, for example, by the polymerase chain reaction (PCR) or other amplification method known to those of ordinary skill in the art. PCR can be performed with the forward and/or reverse primers comprising sequence complementary to at least a portion of a classifier gene provided herein, such as the classifier biomarkers in Table 1. The product of this amplification reaction, i.e., amplified cDNA is necessarily a non-natural product. As mentioned above, cDNA is a non-natural molecule. Second, in the case of PCR, the amplification process serves to create hundreds of millions of cDNA copies for every individual cDNA molecule of starting material. The number of copies generated is far removed from the number of copies of mRNA that are present in vivo.

In one embodiment, cDNA is amplified with primers that introduce an additional DNA sequence (adapter sequence) onto the fragments (with the use of adapter-specific primers). The adaptor sequence can be a tail, wherein the tail sequence is not complementary to the cDNA. For example, the forward and/or reverse primers comprising sequence complementary to at least a portion of a classifier gene provided herein, such as the classifier biomarkers from Table 1 can comprise tail sequence. Amplification therefore serves to create non-natural double stranded molecules from the non-natural single stranded cDNA, by introducing barcode, adapter and/or reporter sequences onto the already non-natural cDNA. In one embodiment, during amplification with the adapter-specific primers, a detectable label, e.g., a fluorophore, is added to single strand cDNA molecules. Amplification therefore also serves to create DNA complexes that do not occur in nature, at least because (i) cDNA does not exist in vivo, (ii) adapter sequences are added to the ends of cDNA molecules to make DNA sequences that do not exist in vivo, (iii) the error rate associated with amplification further creates DNA sequences that do not exist in vivo, (iv) the disparate structure of the cDNA molecules as compared to what exists in nature, and (v) the chemical addition of a detectable label to the cDNA molecules.

In one embodiment, the synthesized cDNA (for example, amplified cDNA) is immobilized on a solid surface via hybridization with a probe, e.g., via a microarray. In another embodiment, cDNA products are detected via real-time polymerase chain reaction (PCR) via the introduction of fluorescent probes that hybridize with the cDNA products. For example, in one embodiment, biomarker detection is assessed by quantitative fluorogenic RT-PCR (e.g., with TaqMan® probes). For PCR analysis, well known methods are available in the art for the determination of primer sequences for use in the analysis.

Biomarkers provided herein in one embodiment, are detected via a hybridization reaction that employs a capture probe and/or a reporter probe. For example, the hybridization probe is a probe derivatized to a solid surface such as a bead, glass or silicon substrate. In another embodiment, the capture probe is present in solution and mixed with the patient's sample, followed by attachment of the hybridization product to a surface, e.g., via a biotin-avidin interaction (e.g., where biotin is a part of the capture probe and avidin is on the surface). The hybridization assay, in one embodiment, employs both a capture probe and a reporter probe. The reporter probe can hybridize to either the capture probe or the biomarker nucleic acid. Reporter probes e.g., are then counted and detected to determine the level of biomarker(s) in the sample. The capture and/or reporter probe, in one embodiment contain a detectable label, and/or a group that allows functionalization to a surface.

For example, the nCounter gene analysis system (see, e.g., Geiss et al. (2008) Nat. Biotechnol. 26, pp. 317-325, incorporated by reference in its entirety for all purposes, is amenable for use with the methods provided herein.

Hybridization assays described in U.S. Pat. Nos. 7,473,767 and 8,492,094, the disclosures of which are incorporated by reference in their entireties for all purposes, are amenable for use with the methods provided herein, i.e., to detect the biomarkers and biomarker combinations described herein.

Biomarker levels may be monitored using a membrane blot (such as used in hybridization analysis such as Northern, Southern, dot, and the like), or microwells, sample tubes, gels, beads, or fibers (or any solid support comprising bound nucleic acids). See, for example, U.S. Pat. Nos. 5,770,722, 5,874,219, 5,744,305, 5,677,195 and 5,445,934, each incorporated by reference in their entireties.

In one embodiment, microarrays are used to detect biomarker levels. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning hybridization intensities for each probe on the array are determined and converted to a quantitative value representing relative gene expression levels. See, for example, U.S. Pat. Nos. 6,040,138, 5,800,992 and 6,020,135, 6,033,860, and 6,344,316, each incorporated by reference in their entireties. High-density oligonucleotide arrays are particularly useful for determining the gene expression profile for a large number of RNAs in a sample.

Techniques for the synthesis of these arrays using mechanical synthesis methods are described in, for example, U.S. Pat. No. 5,384,261. Although a planar array surface is generally used, the array can be fabricated on a surface of virtually any shape or even a multiplicity of surfaces. Arrays can be nucleic acids (or peptides) on beads, gels, polymeric surfaces, fibers (such as fiber optics), glass, or any other appropriate substrate. See, for example, U.S. Pat. Nos. 5,770,358, 5,789,162, 5,708,153, 6,040,193 and 5,800,992, each incorporated by reference in their entireties. Arrays can be packaged in such a manner as to allow for diagnostics or other manipulation of an all-inclusive device. See, for example, U.S. Pat. Nos. 5,856,174 and 5,922,591, each incorporated by reference in their entireties.

Serial analysis of gene expression (SAGE) in one embodiment is employed in the methods described herein. SAGE is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags and identifying the gene corresponding to each tag. See, Velculescu et al. Science 270:484-87, 1995; Cell 88:243-51, 1997, incorporated by reference in its entirety.

An additional method of biomarker level analysis at the nucleic acid level is the use of a sequencing method, for example, RNAseq, next generation sequencing, and massively parallel signature sequencing (MPSS), as described by Brenner et al. (Nat. Biotech. 18:630-34, 2000, incorporated by reference in its entirety). This is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3.0×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

Another method of biomarker level analysis at the nucleic acid level is the use of an amplification method such as, for example, RT-PCR or quantitative RT-PCR (qRT-PCR). Methods for determining the level of biomarker mRNA in a sample may involve the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Numerous different PCR or qRT-PCR protocols are known in the art and can be directly applied or adapted for use using the presently described compositions for the detection and/or quantification of expression of discriminative genes in a sample. See, for example, Fan et al. (2004) Genome Res. 14:878-885, herein incorporated by reference. Generally, in PCR, a target polynucleotide sequence is amplified by reaction with at least one oligonucleotide primer or pair of oligonucleotide primers. The primer(s) hybridize to a complementary region of the target nucleic acid and a DNA polymerase extends the primer(s) to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target polynucleotide sequence. The reaction can be performed in any thermocycler commonly used for PCR.

Quantitative RT-PCR (qRT-PCR) (also referred as real-time RT-PCR) is preferred under some circumstances because it provides not only a quantitative measurement, but also reduced time and contamination. As used herein, “quantitative PCR” (or “real time qRT-PCR”) refers to the direct monitoring of the progress of a PCR amplification as it is occurring without the need for repeated sampling of the reaction products. In quantitative PCR, the reaction products may be monitored via a signaling mechanism (e.g., fluorescence) as they are generated and are tracked after the signal rises above a background level but before the reaction reaches a plateau. The number of cycles required to achieve a detectable or “threshold” level of fluorescence varies directly with the concentration of amplifiable targets at the beginning of the PCR process, enabling a measure of signal intensity to provide a measure of the amount of target nucleic acid in a sample in real time. A DNA binding dye (e.g., SYBR green) or a labeled probe can be used to detect the extension product generated by PCR amplification. Any probe format utilizing a labeled probe comprising the sequences of the invention may be used.

Immunohistochemistry methods are also suitable for detecting the levels of the biomarkers of the present invention. Samples can be frozen for later preparation or immediately placed in a fixative solution. Tissue samples can be fixed by treatment with a reagent, such as formalin, gluteraldehyde, methanol, or the like and embedded in paraffin. Methods for preparing slides for immunohistochemical analysis from formalin-fixed, paraffin-embedded tissue samples are well known in the art.

In one embodiment, the expression levels of the biomarkers provided herein, such as the classifier biomarkers of Table 1 (or subsets thereof, for example 11 to 22, 23 to 33, 34 to 44, 45 to 55, 56 to 66, 67 to 77, or 78 to 88 biomarkers), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, HNSCC subtypes can be evaluated using levels of protein expression of one or more of the classifier genes provided herein, such as the classifier biomarkers listed in Table 1. The level of protein expression can be measured using an immunological detection method. Immunological detection methods which can be used herein include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and the like. Such assays are routine and well known in the art (see, e.g., Ausubel et al, eds, 1994, Current Protocols in Molecular Biology, Vol. I, John Wiley & Sons, Inc., New York, which is incorporated by reference herein in its entirety).

In one embodiment, antibodies specific for biomarker proteins are utilized to detect the expression of a biomarker protein in a body sample. The method comprises obtaining a body sample from a patient or a subject, contacting the body sample with at least one antibody directed to a biomarker that is selectively expressed in Head and Neck cancer cells, and detecting antibody binding to determine if the biomarker is expressed in the patient sample. In one aspect of the present invention provided is an immunocytochemistry technique for diagnosing Head and Neck cancer subtypes. One of skill in the art will recognize that the immunocytochemistry method described herein below may be performed manually or in an automated fashion.

As provided throughout, the methods set forth herein provide a method for determining the Head and Neck cancer SCC subtype of a patient. Once the biomarker levels are determined, for example by measuring non-natural cDNA biomarker levels or non-natural mRNA-cDNA biomarker complexes, the biomarker levels are compared to reference values or a reference sample as provided herein, for example with the use of statistical methods or direct comparison of detected levels, to make a determination of the Head and Neck cancer molecular SCC subtype. Based on the comparison, the patient's Head and Neck cancer sample is SCC classified, e.g., as BA, MS, AT or CL.

In one embodiment, expression level values of the at least one or plurality of classifier biomarkers provided herein, such as the classifier biomarkers of Table 1 are compared to reference expression level value(s) from at least one sample training set, wherein the at least one sample training set comprises expression level values from a reference sample(s). In a further embodiment, the at least one sample training set comprises expression level values of the at least one or plurality of classifier biomarkers provided herein, such as the classifier biomarkers of Table 1 from a HNSCC BA, HNSCC MS, HNSCC AT, HNSCC CL, or HNSCC-free sample or a combination thereof.

In a separate embodiment, hybridization values of the at least one or plurality of classifier biomarkers provided herein, such as the classifier biomarkers of Table 1 are compared to reference hybridization value(s) from a control, which can be at least one sample training set, wherein the at least one sample training set comprises hybridization values from a reference sample(s). In a further embodiment, the at least one sample training set comprises hybridization values of the at least one or plurality of classifier biomarkers provided herein, such as the classifier biomarkers of Table 1 from a HNSCC BA, HNSCC MS, HNSCC AT, HNSCC CL, or HNSCC-free sample, or a combination thereof. Methods for comparing detected levels of biomarkers to reference values and/or reference samples are provided herein. Based on this comparison, in one embodiment a correlation between the biomarker levels obtained from the subject's sample and the reference values is obtained. An assessment of the Head and Neck cancer SCC subtype is then made.

Various statistical methods can be used to aid in the comparison of the biomarker levels obtained from the patient and reference biomarker levels, for example, from at least one sample training set.

In one embodiment, a supervised pattern recognition method is employed. Examples of supervised pattern recognition methods can include, but are not limited to, the nearest centroid methods (Dabney (2005) Bioinformatics 21(22):4148-4154 and Tibshirani et al. (2002) Proc. Natl. Acad. Sci. USA 99(10):6576-6572); soft independent modeling of class analysis (SIMCA) (see, for example, Wold, 1976); partial least squares analysis (PLS) (see, for example, Wold, 1966; Joreskog, 1982; Frank, 1984; Bro, R., 1997); linear descriminant analysis (LDA) (see, for example, Nillson, 1965); K-nearest neighbour analysis (KNN) (see, for example, Brown et al., 1996); artificial neural networks (ANN) (see, for example, Wasserman, 1989; Anker et al., 1992; Hare, 1994); probabilistic neural networks (PNNs) (see, for example, Parzen, 1962; Bishop, 1995; Speckt, 1990; Broomhead et al., 1988; Patterson, 1996); rule induction (RI) (see, for example, Quinlan, 1986); and, Bayesian methods (see, for example, Bretthorst, 1990a, 1990b, 1988). In one embodiment, the classifier for identifying tumor subtypes based on gene expression data is the centroid based method described in Mullins et al. (2007) Clin Chem. 53(7):1273-9, each of which is herein incorporated by reference in its entirety.

In other embodiments, an unsupervised training approach is employed, and therefore, no training set is used.

Referring to sample training sets for supervised learning approaches again, in some embodiments, a sample training set(s) can include expression data of a plurality or all of the classifier biomarkers (e.g., the classifier biomarkers of Table 1) as measured in a sample obtained from a HNSCC patient. The plurality of classifier biomarkers selected from Table 1 can comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75,76, 77, 78, 79, 80, 81, 82, 83, 84, 85, 86, 87 or 88 of the biomarkers from Table 1. The plurality of classifier biomarkers selected from Table 1 can comprise at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. The plurality of classifier biomarkers selected from Table 1 can comprise from 1-11 classifier biomarkers, 12-22 classifier biomarkers, 23-33 classifier biomarkers, 34-44 classifier biomarkers, 45-55 classifier biomarkers, 56-66 classifier biomarkers, 67-77 classifier biomarkers or 78-88 classifier biomarkers. The plurality of classifier biomarkers selected from Table 1 can comprise at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the biomarkers from Table 1. The plurality of classifier biomarkers selected from Table 1 can comprise the classifier biomarkers specified for each HNSCC subtype as outlined in Table 2 or subsets thereof. In some embodiments, the sample training set(s) are normalized to remove sample-to-sample variation. The normalization can be done using any housekeeping gene known in the art, such as, for example, GAPDH and/or beta-actin.

In some embodiments, comparing can include applying a statistical algorithm, such as, for example, any suitable multivariate statistical analysis model, which can be parametric or non-parametric. In some embodiments, applying the statistical algorithm can include determining a correlation between the expression data obtained from the human head and neck tissue sample and the expression data from the HNSCC training set(s). In some embodiments, cross-validation is performed, such as (for example), leave-one-out cross-validation (LOOCV). In some embodiments, integrative correlation is performed. In some embodiments, a Spearman correlation is performed. In some embodiments, a centroid based method is employed for the statistical algorithm as described in Mullins et al. (2007) Clin Chem. 53(7):1273-9, and based on gene expression data, which is herein incorporated by reference in its entirety.

Results of the gene expression performed on a sample from a subject (test sample) may be compared to a biological sample(s) or data derived from a biological sample(s) that is known or suspected to be normal (“reference sample” or “normal sample”, e.g., non-HNSCC sample). In some embodiments, a reference sample or reference gene expression data is obtained or derived from an individual known to have a particular molecular subtype of HNSCC, i.e., BA, MS, AT or CL.

The reference sample may be assayed at the same time, or at a different time from the test sample. Alternatively, the biomarker level information from a reference sample may be stored in a database or other means for access at a later date.

The biomarker level results of an assay on the test sample may be compared to the results of the same assay on a reference sample. In some cases, the results of the assay on the reference sample are from a database, or a reference value(s). In some cases, the results of the assay on the reference sample are a known or generally accepted value or range of values by those skilled in the art. In some cases, the comparison is qualitative. In other cases, the comparison is quantitative. In some cases, qualitative or quantitative comparisons may involve but are not limited to one or more of the following: comparing fluorescence values, spot intensities, absorbance values, chemiluminescent signals, histograms, critical threshold values, statistical significance values, expression levels of the genes described herein, mRNA copy numbers.

In one embodiment, an odds ratio (OR) is calculated for each biomarker level panel measurement. Here, the OR is a measure of association between the measured biomarker values for the patient and an outcome, e.g., HNSCC subtype. For example, see, J. Can. Acad. Child Adolesc. Psychiatry 2010; 19(3):227-229, which is incorporated by reference in its entirety for all purposes.

In one embodiment, a specified statistical confidence level may be determined in order to provide a confidence level regarding the Head and Neck cancer subtype. For example, it may be determined that a confidence level of greater than 90% may be a useful predictor of the Head and Neck cancer subtype. In other embodiments, more or less stringent confidence levels may be chosen. For example, a confidence level of about or at least about 50%, 60%, 70%, 75%, 80%, 85%, 90%, 95%, 97.5%, 99%, 99.5%, or 99.9% may be chosen. The confidence level provided may in some cases be related to the quality of the sample, the quality of the data, the quality of the analysis, the specific methods used, and/or the number of gene expression values (i.e., the number of genes) analyzed. The specified confidence level for providing the likelihood of response may be chosen on the basis of the expected number of false positives or false negatives. Methods for choosing parameters for achieving a specified confidence level or for identifying markers with diagnostic power include but are not limited to Receiver Operating Characteristic (ROC) curve analysis, binormal ROC, principal component analysis, odds ratio analysis, partial least squares analysis, singular value decomposition, least absolute shrinkage and selection operator analysis, least angle regression, and the threshold gradient directed regularization method.

Determining the HNSCC subtype in some cases can be improved through the application of algorithms designed to normalize and/or improve the reliability of the gene expression data. In some embodiments of the present invention, the data analysis utilizes a computer or other device, machine or apparatus for application of the various algorithms described herein due to the large number of individual data points that are processed. A “machine learning algorithm” refers to a computational-based prediction methodology, also known to persons skilled in the art as a “classifier,” employed for characterizing a gene expression profile or profiles, e.g., to determine the HNSCC subtype. The biomarker levels, determined by, e.g., microarray-based hybridization assays, sequencing assays, NanoString assays, etc., are in one embodiment subjected to the algorithm in order to classify the profile. Supervised learning generally involves “training” a classifier to recognize the distinctions among subtypes such as BA positive, MS positive, AT positive or CL positive, and then “testing” the accuracy of the classifier on an independent test set. Therefore, for new, unknown samples the classifier can be used to predict, for example, the class (e.g., BA vs. MS vs. AT vs. CL) in which the samples belong.

In some embodiments, a robust multi-array average (RMA) method may be used to normalize raw data. The RMA method begins by computing background-corrected intensities for each matched cell on a number of microarrays. In one embodiment, the background corrected values are restricted to positive values as described by Irizarry et al. (2003). Biostatistics April 4 (2):249-64, incorporated by reference in its entirety for all purposes. After background correction, the base-2 logarithm of each background corrected matched-cell intensity is then obtained. The background corrected, log-transformed, matched intensity on each microarray is then normalized using the quantile normalization method in which for each input array and each probe value, the array percentile probe value is replaced with the average of all array percentile points, this method is more completely described by Bolstad et al. Bioinformatics 2003, incorporated by reference in its entirety. Following quantile normalization, the normalized data may then be fit to a linear model to obtain an intensity measure for each probe on each microarray. Tukey's median polish algorithm (Tukey, J. W., Exploratory Data Analysis. 1977, incorporated by reference in its entirety for all purposes) may then be used to determine the log-scale intensity level for the normalized probe set data.

Various other software programs may be implemented. In certain methods, feature selection and model estimation may be performed by logistic regression with lasso penalty using glmnet (Friedman et al. (2010). Journal of statistical software 33(1): 1-22, incorporated by reference in its entirety). Raw reads may be aligned using TopHat (Trapnell et al. (2009). Bioinformatics 25(9): 1105-11, incorporated by reference in its entirety). In methods, top features (N ranging from 10 to 200) are used to train a linear support vector machine (SVM) (Suykens J A K, Vandewalle J. Least Squares Support Vector Machine Classifiers. Neural Processing Letters 1999; 9(3): 293-300, incorporated by reference in its entirety) using the e1071 library (Meyer D. Support vector machines: the interface to libsvm in package e1071. 2014, incorporated by reference in its entirety), Confidence intervals, in one embodiment, are computed using the pROC package (Robin X, Turek N, Hainard A, et al. pROC: an open-source package for R and S+ to analyze and compare ROC curves. BMC bioinformatics 2011; 12:77, incorporated by reference in its entirety).

In addition, data may be filtered to remove data that may be considered suspect. In one embodiment, data derived from microarray probes that have fewer than about 4, 5, 6, 7 or 8 guanosine+cytosine nucleotides may be considered to be unreliable due to their aberrant hybridization propensity or secondary structure issues. Similarly, data deriving from microarray probes that have more than about 12, 13, 14, 15, 16, 17, 18, 19, 20, 21, or 22 guanosine+cytosine nucleotides may in one embodiment be considered unreliable due to their aberrant hybridization propensity or secondary structure issues.

In some embodiments of the present invention, data from probe-sets may be excluded from analysis if they are not identified at a detectable level (above background).

In some embodiments of the present disclosure, probe-sets that exhibit no, or low variance may be excluded from further analysis. Low-variance probe-sets are excluded from the analysis via a Chi-Square test. In one embodiment, a probe-set is considered to be low-variance if its transformed variance is to the left of the 99 percent confidence interval of the Chi-Squared distribution with (N−l) degrees of freedom. (N−l)*Probe-set Variance/(Gene Probe-set Variance). Chi-Sq(N−l) where N is the number of input CEL files, (N−l) is the degrees of freedom for the Chi-Squared distribution, and the “probe-set variance for the gene” is the average of probe-set variances across the gene. In some embodiments of the present invention, probe-sets for a given mRNA or group of mRNAs may be excluded from further analysis if they contain less than a minimum number of probes that pass through the previously described filter steps for GC content, reliability, variance and the like. For example, in some embodiments, probe-sets for a given gene or transcript cluster may be excluded from further analysis if they contain less than about 1, 2, 3, 4, 5, 6, 7, 8, 9, 10, 11, 12, 13, 14, 15, or less than about 20 probes.

Methods of biomarker level data analysis in one embodiment, further include the use of a feature selection algorithm as provided herein. In some embodiments of the present invention, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420, incorporated by reference in its entirety for all purposes).

Methods of biomarker level data analysis, in one embodiment, include the use of a pre-classifier algorithm. For example, an algorithm may use a specific molecular fingerprint to pre-classify the samples according to their composition and then apply a correction/normalization factor. This data/information may then be fed into a final classification algorithm which would incorporate that information to aid in the final diagnosis.

Methods of biomarker level data analysis, in one embodiment, further include the use of a classifier algorithm as provided herein. In one embodiment of the present invention, a diagonal linear discriminant analysis, k-nearest neighbor algorithm, support vector machine (SVM) algorithm, linear support vector machine, random forest algorithm, or a probabilistic model-based method or a combination thereof is provided for classification of microarray data. In some embodiments, identified markers that distinguish samples (e.g., of varying biomarker level profiles, and/or varying molecular subtypes of HNSCC (e.g., basal, mesenchymal, atypical, classical)) are selected based on statistical significance of the difference in biomarker levels between classes of interest. In some cases, the statistical significance is adjusted by applying a Benjamin Hochberg or another correction for false discovery rate (FDR).

In some cases, the classifier algorithm may be supplemented with a meta-analysis approach such as that described by Fishel and Kaufman et al. 2007 Bioinformatics 23(13): 1599-606, incorporated by reference in its entirety for all purposes. In some cases, the classifier algorithm may be supplemented with a meta-analysis approach such as a repeatability analysis.

Methods for deriving and applying posterior probabilities to the analysis of biomarker level data are known in the art and have been described for example in Smyth, G. K. 2004 Stat. Appi. Genet. Mol. Biol. 3: Article 3, incorporated by reference in its entirety for all purposes. In some cases, the posterior probabilities may be used in the methods of the present invention to rank the markers provided by the classifier algorithm.

A statistical evaluation of the results of the biomarker level profiling may provide a quantitative value or values indicative of one or more of the following: molecular subtype of HNSCC (e.g., basal, mesenchymal, atypical, classical); the likelihood of the success of a particular therapeutic intervention, e.g., radiation therapy, angiogenesis inhibitor therapy, chemotherapy, or immunotherapy. In one embodiment, the data is presented directly to the physician in its most useful form to guide patient care or is used to define patient populations in clinical trials or a patient population for a given medication. In this way, the biomarker level profiling methods provided herein serve as a therapeutic response signature. The results of the molecular profiling can be statistically evaluated using a number of methods known to the art including, but not limited to: the students T test, the two sided T test, Pearson rank sum analysis, hidden Markov model analysis, analysis of q-q plots, principal component analysis, one way ANOVA, two way ANOVA, LIMMA and the like.

In some cases, accuracy may be determined by tracking the subject over time to determine the accuracy of the original diagnosis. In other cases, accuracy may be established in a deterministic manner or using statistical methods. For example, receiver operator characteristic (ROC) analysis may be used to determine the optimal assay parameters to achieve a specific level of accuracy, specificity, positive predictive value, negative predictive value, and/or false discovery rate.

In some cases, the results of the biomarker level profiling assays, are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, a computer or algorithmic analysis of the data is provided automatically. In some cases, the molecular profiling business may bill the individual, insurance provider, medical provider, researcher, or government entity for one or more of the following: molecular profiling assays performed, consulting services, data analysis, reporting of results, or database access.

In some embodiments of the present invention, the results of the biomarker level profiling assays are presented as a report on a computer screen or as a paper record. In some embodiments, the report may include, but is not limited to, such information as one or more of the following: the levels of biomarkers (e.g., as reported by copy number or fluorescence intensity, etc.) as compared to the reference sample or reference value(s); the likelihood the subject will respond to a particular therapy, based on the biomarker level values and the HNSCC subtype and proposed therapies.

In one embodiment, the results of the gene expression profiling may be classified into one or more of the following: basal positive, mesenchymal positive, atypical positive or classical positive, basal negative, mesenchymal negative, atypical negative or classical negative; likely to respond to surgery (e.g., neck dissection), radiotherapy, angiogenesis inhibitor, immunotherapy or chemotherapy; unlikely to respond to surgery (e.g., neck dissection), radiotherapy, angiogenesis inhibitor, immunotherapy or chemotherapy; or a combination thereof. In a further embodiment, the results of the gene expression profiling may be further classified into being HPV positive or HPV negative. In yet another embodiment, the results of the gene expression profiling may be further classified into being nodal positive (e.g., N123) or node negative (NO).

In some embodiments of the present invention, results are classified using a trained algorithm. Trained algorithms of the present invention include algorithms that have been developed using a reference set of known gene expression values and/or normal samples, for example, samples from individuals diagnosed with a particular molecular subtype of HNSCC. In some cases, a reference set of known gene expression values are obtained from individuals who have been diagnosed with a particular molecular subtype of HNSCC and are also known to respond (or not respond) to angiogenesis inhibitor therapy. In some cases, a reference set of known gene expression values are obtained from individuals who have been diagnosed with a particular molecular subtype of HNSCC and are also known to respond (or not respond) to immunotherapy. In some cases, a reference set of known gene expression values are obtained from individuals who have been diagnosed with a particular molecular subtype of HNSCC and are also known to respond (or not respond) to chemotherapy or radiation therapy or surgical intervention. In some cases, the reference sets described above are HPV positive. In some cases, the reference sets described above are HPV negative. In some cases, the reference sets described above are node positive (e.g., N123) or known to possess nodal metastasis. In some cases, the reference sets described above are node negative (e.g., NO) or not to possess nodal metastasis.

Algorithms suitable for categorization of samples include but are not limited to k-nearest neighbor algorithms, support vector machines, linear discriminant analysis, diagonal linear discriminant analysis, updown, naive Bayesian algorithms, neural network algorithms, hidden Markov model algorithms, genetic algorithms, or any combination thereof.

When a binary classifier is compared with actual true values (e.g., values from a biological sample), there are typically four possible outcomes. If the outcome from a prediction is p (where “p” is a positive classifier output, such as the presence of a deletion or duplication syndrome) and the actual value is also p, then it is called a true positive (TP); however, if the actual value is n then it is said to be a false positive (FP). Conversely, a true negative has occurred when both the prediction outcome and the actual value are n (where “n” is a negative classifier output, such as no deletion or duplication syndrome), and false negative is when the prediction outcome is n while the actual value is p. In one embodiment, consider a test that seeks to determine whether a person is likely or unlikely to respond to angiogenesis inhibitor therapy. A false positive in this case occurs when the person tests positive, but actually does respond. A false negative, on the other hand, occurs when the person tests negative, suggesting they are unlikely to respond, when they actually are likely to respond. The same holds true for classifying a Head and Neck cancer subtype.

The positive predictive value (PPV), or precision rate, or post-test probability of disease, is the proportion of subjects with positive test results who are correctly diagnosed as likely or unlikely to respond or diagnosed with the correct Head and Neck cancer subtype, or a combination thereof. It reflects the probability that a positive test reflects the underlying condition being tested for. Its value does however depend on the prevalence of the disease, which may vary. In one example the following characteristics are provided: FP (false positive); TN (true negative); TP (true positive); FN (false negative). False positive rate (α)=FP/(FP+TN)-specificity; False negative rate (β)=FN/(TP+FN)-sensitivity; Power=sensitivity=1−β; Likelihood-ratio positive=sensitivity/(l−specificity); Likelihood-ratio negative=(1−sensitivity)/specificity. The negative predictive value (NPV) is the proportion of subjects with negative test results who are correctly diagnosed.

In some embodiments, the results of the biomarker level analysis of the subject methods provide a statistical confidence level that a given diagnosis is correct. In some embodiments, such statistical confidence level is at least about, or more than about 85%, 90%, 91%, 92%, 93%, 94%, 95%, 96%, 97%, 98%, 99% 99.5%, or more.

In some embodiments, the method further includes classifying the Head and Neck tissue sample as a particular Head and Neck cancer subtype based on the comparison of biomarker levels in the sample and reference biomarker levels, for example, present in at least one training set. In some embodiments, the Head and Neck tissue sample is classified as a particular subtype if the results of the comparison meet one or more criterion such as, for example, a minimum percent agreement, a value of a statistic calculated based on the percentage agreement such as (for example) a kappa statistic, a minimum correlation (e.g., Pearson's correlation) and/or the like.

It is intended that the methods described herein can be performed by software (stored in memory and/or executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including Unix utilities, C, C++, Java™, Ruby, SQL, SAS®, the R programming language/software environment, Visual Basic™, and other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

Some embodiments described herein relate to devices with a non-transitory computer-readable medium (also can be referred to as a non-transitory processor-readable medium or memory) having instructions or computer code thereon for performing various computer-implemented operations and/or methods disclosed herein. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

In one embodiment, provided herein is a system comprising one or more processors, one or more memories, and/or a non-transitory computer readable medium as well as instructions and/or computer code designed to execute any of the diagnostic, prognostic or theranostic methods described herein when executed by at least one of the one or more processors in combination with any hardware devices (e.g., computers, sequencers, microfluidic handling devices) that are specifically configured to store and execute the program code and/or instructions stored in the one or more memories. In some cases, provided herein is a system for determining a head and neck squamous cell carcinoma (HNSCC) subtype of a sample obtained from a subject suffering from HNSCC. The system can be used to diagnose or determine the subtype of HNSCC of the subject. The system may also be used to predict responsive of the subject to a particular treatment modality or modalities as a result of determining the subject's HNSCC subtype. Selection of the treatment modality or treatment modalities may also be aided by the system's ability to either determine or integrate data on the subject's nodal status and/or HPV status. In some cases, the system comprises one or more processors and one or more memories operatively coupled to at least one of the one or more processors and having instructions stored thereon that, when executed by at least one of the one or more processors, cause the system to perform or integrate the results of a biomarker level profiling assay. In some cases, the results of the biomarker level profiling assay are entered into a database for access by representatives or agents of a molecular profiling business, the individual, a medical provider, or insurance provider. In some cases, assay results include sample classification, identification, or diagnosis by a representative, agent or consultant of the business, such as a medical professional. In other cases, the system is configured to perform an algorithmic analysis of the biomarker level profiling assay automatically. In some cases, the molecular profiling business may bill the individual, insurance provider, medical provider, researcher, or government entity for one or more of the following: biomarker level profiling assays performed, consulting services, data analysis, reporting of results, or database access.

In some embodiments of the present invention, the system is configured such that the results of the biomarker level profiling assays are presented as a report on a computer screen or as a paper record. In some embodiments, the report may include, but is not limited to, such information as one or more of the following: the levels of biomarkers (e.g., as reported by copy number or fluorescence intensity, etc.) as compared to the reference sample or reference value(s); the likelihood the subject will respond to a particular therapy, based on the biomarker level values and the HNSCC subtype and proposed therapies.

The biomarker level profiling assay that is utilized by the system can entail detecting an expression level of each of a plurality of classifier biomarkers from Table 1 in a sample obtained from a subject suffering from, suspected of suffering from or at risk of suffering from HNSCC. The assay can further entail comparing the expression levels of each of the plurality of classifier biomarkers from Table 1 to the expression levels of each of the plurality of classifier biomarkers from Table 1 in a control sample; and classifying the sample as a basal (BA), mesenchymal (MS), atypical (AT) or classical (CL) HNSCC subtype based on the results of the comparing step. The control can comprise at least one sample training set(s), wherein the at least one sample training set comprises expression levels of each of the plurality of classifier biomarkers from Table 1 from a reference HNSCC BA sample, expression levels of each of the plurality of classifier biomarkers from Table 1 from a reference HNSCC MS sample, expression levels of each of the plurality of classifier biomarkers from Table 1 from a reference HNSCC AT sample, expression levels of each of the plurality of classifier biomarkers from Table 1 from a reference HNSCC CL sample, expression levels of each of the plurality of classifier biomarkers from Table 1 from a reference HNSCC negative sample or a combination thereof. In some cases, the comparing step can comprise applying a statistical algorithm provided herein. The statistical algorithm can comprise determining a correlation between the expression data obtained from the sample and the expression data from the at least one training set(s); and classifying the sample as a BA, MS, AT or CL subtype based on the results of the statistical algorithm. In some cases, the expression level of each of the plurality of classifier biomarkers from Table 1 is detected at the nucleic acid level (e.g., RNA, DNA or cDNA) in the sample obtained from the subject and/or the control sample. In some cases, the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques. The detecting the expression level or performing the biomarker level profiling assay can be performed using a device that is part of the system or in communication with at least one of the one or more processors, wherein upon receipt of instructions sent by the at least one of the one or more processors, perform the biomarker level profiling assay. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof. In some cases, the plurality of classifier biomarkers of Table 1 comprises all the classifier biomarkers from Table 1. In some cases, the system is configured to determine or diagnose the subject as possessing a mesenchymal (MS) subtype of HNSCC or a non-mesenchymal subtype of HNSCC. In one embodiment, the HNSCC is oral cavity HNSCC. In one embodiment, the system is further configured to either perform or to integrate data with regard to the subject's nodal status. The nodal status in the subject can be ascertained by any method known in the art and/or provided herein for determining the nodal status. In some embodiments, the nodal status (stage) can include different status of primary tumor (T). In some embodiments, the nodal status (stage) can include different status of regional lymph nodes (N). In some embodiments, the nodal status (stage) can include different status of distant metastasis.

In some embodiments, a single biomarker, or a plurality of classifier biomarkers from Table 1 is capable of classifying subtypes of HNSCC with a predictive success of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between. In some embodiments, any combination of biomarkers disclosed herein (e.g., in Table 1) can be used to obtain a predictive success of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between. The plurality of classifier biomarkers selected from Table 1 can comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87 or 88 of the biomarkers from Table 1. The plurality of classifier biomarkers selected from Table 1 can comprise at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. The plurality of classifier biomarkers selected from Table 1 can comprise from 1-11 classifier biomarkers, 12-22 classifier biomarkers, 23-33 classifier biomarkers, 34-44 classifier biomarkers, 45-55 classifier biomarkers, 56-66 classifier biomarkers, 67-77 classifier biomarkers or 78-88 classifier biomarkers. The plurality of classifier biomarkers selected from Table 1 can comprise at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the biomarkers from Table 1. The plurality of classifier biomarkers selected from Table 1 can comprise the classifier biomarkers specified for each HNSCC subtype as outlined in Table 2 or subsets thereof.

In some embodiments, a single biomarker, or a plurality of classifier biomarkers from Table 1 is capable of classifying subtypes of HNSCC with a sensitivity or specificity of at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between. In some embodiments, any combination of biomarkers disclosed herein can be used to obtain a sensitivity or specificity of at least about 70%, at least about 71%, at least about 72%, about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, up to 100%, and all values in between. The plurality of classifier biomarkers selected from Table 1 can comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87 or 88 of the biomarkers from Table 1. The plurality of classifier biomarkers selected from Table 1 can comprise at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. The plurality of classifier biomarkers selected from Table 1 can comprise from 1-11 classifier biomarkers, 12-22 classifier biomarkers, 23-33 classifier biomarkers, 34-44 classifier biomarkers, 45-55 classifier biomarkers, 56-66 classifier biomarkers, 67-77 classifier biomarkers or 78-88 classifier biomarkers. The plurality of classifier biomarkers selected from Table 1 can comprise at least, at most, or exactly 5%, 10%, 15%, 20%, 25%, 30%, 35%, 40%, 45%, 50%, 55%, 60%, 65%, 70%, 75%, 80%, 85%, 90%, 95% or 99% of the biomarkers from Table 1. The plurality of classifier biomarkers selected from Table 1 can comprise the classifier biomarkers specified for each HNSCC subtype as outlined in Table 2 or subsets thereof.

### Clinical/Therapeutic Uses

In one embodiment, a method is provided herein for determining a disease outcome or prognosis for a patient suffering from cancer. In some cases, the cancer is head and neck squamous cell carcinoma. In some cases, the HNSCC is oral cavity squamous cell carcinoma (OCSCC). The disease outcome or prognosis can be measured by examining the overall survival for a period of time or intervals (e.g., 0 to 36 months or 0 to 60 months). In one embodiment, survival is analyzed as a function of subtype (e.g., for HNSCC (BA, MS, AT and CL)). The HNSCC subtype can be determined using the methods provided herein such as, for example, determining the expression of all or subsets of the genes in Table 1 alone or in combination with determining the HPV status and/or the nodal status. Relapse-free and overall survival can be assessed using standard Kaplan-Meier plots as well as Cox proportional hazards modeling. The gene expression based HNSCC subtyping can be performed using any of the methods provided herein such as, for example, detecting the expression of one or more of the biomarkers listed in Table 1.

In one embodiment, upon determining a patient's HNSCC subtype (e.g., by measuring the expression of all or subsets of the genes in Table 1 alone or in combination with determining the HPV status and/or nodal status), the patient is selected for suitable therapy, for example, radiotherapy (radiation therapy), surgical intervention, target therapy, chemotherapy or drug therapy with an angiogenesis inhibitor or immunotherapy or combinations thereof. In some embodiments, the suitable treatment can be any treatment or therapeutic method that can be used for a HNSCC patient. In one embodiment, upon determining a patient's HNSCC subtype, the patient is administered a suitable therapeutic agent, for example chemotherapeutic agent(s) or an angiogenesis inhibitor or immunotherapeutic agent(s). In one embodiment, the therapy is immunotherapy, and the immunotherapeutic agent is a checkpoint inhibitor, monoclonal antibody, biological response modifier, therapeutic vaccine or cellular immunotherapy. In some embodiments, the determination of a suitable treatment can identify treatment responders. In some embodiments, the determination of a suitable treatment can identify treatment non-responders. In some embodiments, upon determining a patient's HNSCC subtype, the HNSCC patients can be selected for any combination of suitable therapies. For example, chemotherapy or drug therapy with a radiotherapy, a neck dissection with an immunotherapy or a chemotherapeutic agent with a radiotherapy. In some embodiments, immunotherapy, or immunotherapeutic agent can be a checkpoint inhibitor, monoclonal antibody, biological response modifier, therapeutic vaccine or cellular immunotherapy.

The methods of present invention are also useful for evaluating clinical response to therapy, as well as for endpoints in clinical trials for efficacy of new therapies. The extent to which sequential diagnostic expression profiles move towards normal can be used as one measure of the efficacy of the candidate therapy.

In one embodiment, the methods of the invention also find use in predicting response to different lines of therapies based on the subtype of HNSCC. For example, chemotherapeutic response can be improved by more accurately assigning tumor subtypes. Likewise, treatment regimens can be formulated based on the tumor subtype.

### Angiogenesis Inhibitors

In one embodiment, upon determining a patient's HNSCC subtype, the patient is selected for drug therapy with an angiogenesis inhibitor.

In one embodiment, the angiogenesis inhibitor is a vascular endothelial growth factor (VEGF) inhibitor, a VEGF receptor inhibitor, a platelet derived growth factor (PDGF) inhibitor or a PDGF receptor inhibitor.

Each biomarker panel can include one, two, three, four, five, six, seven, eight or more biomarkers usable by a classifier (also referred to as a “classifier biomarker”) to assess whether a HNSCC patient is likely to respond to angiogenesis inhibitor therapy; to select a HNSCC patient for angiogenesis inhibitor therapy; to determine a “hypoxia score” and/or to subtype a HNSCC sample as basal, mesenchymal, atypical, or classical molecular subtype. As used herein, the term “classifier” can refer to any algorithm for statistical classification, and can be implemented in hardware, in software, or a combination thereof. The classifier can be capable of 2-level, 3-level, 4-level, or higher, classification, and can depend on the nature of the entity being classified. In one embodiment, the classifier biomarkers provided herein (e.g., the classifiers in Table 1) can classify the HNSCC patient as possessing one of four molecular subtypes selected from the group consisting of mesenchymal (MS), basal (BA), classical (CL) and atypical (AT). In another embodiment, the classifier biomarkers provided herein (e.g., the classifiers in Table 1) can classify the HNSCC patient as possessing one of four molecular subtypes selected from the group consisting of MS, BA, CL and AT or as not possessing one of four molecular subtypes selected from the group consisting of MS, BA, CL and AT. In some cases, the detection of the expression levels of one or a plurality of classifier biomarkers selected from Table 1 in a sample obtained from an HNSCC patient can be used to diagnose that the patient possesses a mesenchymal molecular subtype or a non-mesenchymal subtype. In some cases, the detection of the expression levels of one or a plurality of classifier biomarkers selected from Table 1 in a sample obtained from an HNSCC patient can be used to diagnose that the patient possesses a basal molecular subtype or a non-basal subtype. In some cases, the detection of the expression levels of one or a plurality of classifier biomarkers selected from Table 1 in a sample obtained from an HNSCC patient can be used to diagnose that the patient possesses a classical molecular subtype or a non-classical subtype. In some cases, the detection of the expression levels of one or a plurality of classifier biomarkers selected from Table 1 in a sample obtained from an HNSCC patient can be used to diagnose that the patient possesses an atypical molecular subtype or a non-atypical subtype. One or more classifiers can be employed to achieve the aspects disclosed herein.

In general, methods of determining whether a HNSCC patient is likely to respond to angiogenesis inhibitor therapy, or methods of selecting a HNSCC patient for angiogenesis inhibitor therapy are provided herein. In one embodiment, the method comprises assessing whether the patient's HNSCC subtype is basal, mesenchymal, atypical, or classical using the methods described herein (e.g., assessing the expression of one or more classifier biomarkers of Table 1 alone or in combination with assessing the expression of one or more HPV genes and/or the nodal status of the patient) and probing a HNSCC sample from the patient for the levels of at least five biomarkers selected from the group consisting of RRAGD, FABP5, UCHLI, GAL, PLOD, DDIT4, VEGF, ADM, ANGPTL4, NDRGI, NP, SLC16A3, and C14ORF58 (see Table 3) at the nucleic acid level. In a further embodiment, the probing step comprises mixing the sample with five or more oligonucleotides that are substantially complementary to portions of nucleic acid molecules of the at least five biomarkers under conditions suitable for hybridization of the five or more oligonucleotides to their complements or substantial complements, detecting whether hybridization occurs between the five or more oligonucleotides to their complements or substantial complements; and obtaining hybridization values of the sample based on the detecting steps. The hybridization values of the sample are then compared to reference hybridization value(s) from at least one sample training set, wherein the at least one sample training set comprises (i) hybridization value(s) of the at least five biomarkers from a sample that overexpresses the at least five biomarkers, or overexpresses a subset of the at least five biomarkers, (ii) hybridization values of the at least five biomarkers from a reference basal, mesenchymal, atypical, or classical sample, or (iii) hybridization values of the at least five biomarkers from a HNSCC free head and neck sample. A determination of whether the patient is likely to respond to angiogenesis inhibitor therapy, or a selection of the patient for angiogenesis inhibitor is then made based upon (i) the patient's HNSCC subtype and (ii) the results of comparison.

TABLE 3
Biomarkers for hypoxia profile

| | | GenBank |
|---|---|---|
| Name | Abbreviation | Accession No. |
| RRAGD | Ras-related GTP binding D | BC003088 |
| FABP5 | fatty acid binding protein 5 | M94856 |
| UCHL1 | ubiquitin carboxyl-terminal esterase L1 | NM_004181 |
| GAL | Galanin | BC030241 |
| PLOD | procollagen-lysine, 2-oxoglutarate | M98252 |
| | 5-dioxygenase lysine hydroxylase | |
| DDIT4 | DNA-damage-inducible transcript 4 | NM_{——}019058 |
| VEGF | vascular endothelial growth factor | M32977 |
| ADM | Adrenomedullin | NM_{——}001124 |
| ANGPTL4 | angiopoietin-like 4 | AF202636 |
| NDRG1 | N-myc downstream regulated gene 1 | NM_{——}006096 |
| NP | nucleoside phosphorylase | NM 000270 |
| SLC16A3 | solute carrier family 16 monocarboxylic | NM_{——}004207 |
| | acid transporters, member 3 | |
| C14ORF58 | chromosome 14 open reading frame 58 | AK000378 |

The aforementioned set of thirteen biomarkers, or a subset thereof, is also referred to herein as a “hypoxia profile”.

In one embodiment, the method provided herein includes determining the levels of at least five biomarkers, at least six biomarkers, at least seven biomarkers, at least eight biomarkers, at least nine biomarkers, or at least ten biomarkers, or five to thirteen, six to thirteen, seven to thirteen, eight to thirteen, nine to thirteen or ten to thirteen biomarkers selected from RRAGD, FABP5, UCHLI, GAL, PLOD, DDIT4, VEGF, ADM, ANGPTL4, NDRGI, NP, SLC16A3, and C14ORF58 in a HNSCC sample obtained from a subject. Biomarker expression in some instances may be normalized against the expression levels of all RNA transcripts or their expression products in the sample, or against a reference set of RNA transcripts or their expression products. The reference set as explained throughout, may be an actual sample that is tested in parallel with the HNSCC sample, or may be a reference set of values from a database or stored dataset. Levels of expression, in one embodiment, are reported in number of copies, relative fluorescence value or detected fluorescence value. The level of expression of the biomarkers of the hypoxia profile together with HNSCC subtype as determined using the methods provided herein can be used in the methods described herein to determine whether a patient is likely to respond to angiogenesis inhibitor therapy.

In one embodiment, the levels of expression of the thirteen biomarkers (or subsets thereof, as described above, e.g., five or more, from about five to about 13), are normalized against the expression levels of all RNA transcripts or their non-natural cDNA expression products, or protein products in the sample, or of a reference set of RNA transcripts or a reference set of their non-natural cDNA expression products, or a reference set of their protein products in the sample.

In one embodiment, angiogenesis inhibitor treatments include, but are not limited to an integrin antagonist, a selectin antagonist, an adhesion molecule antagonist, an antagonist of intercellular adhesion molecule (ICAM)-1, ICAM-2, ICAM-3, platelet endothelial adhesion molecule (PCAM), vascular cell adhesion molecule (VCAM)), lymphocyte function-associated antigen 1 (LFA-1), a basic fibroblast growth factor antagonist, a vascular endothelial growth factor (VEGF) modulator, a platelet derived growth factor (PDGF) modulator (e.g., a PDGF antagonist).

In one embodiment of determining whether a subject is likely to respond to an integrin antagonist, the integrin antagonist is a small molecule integrin antagonist, for example, an antagonist described by Paolillo et al. (Mini Rev Med Chem, 2009, volume 12, pp. 1439-1446, incorporated by reference in its entirety), or a leukocyte adhesion-inducing cytokine or growth factor antagonist (e.g., tumor necrosis factor-α (TNF-α), interleukin-1β (IL-1β), monocyte chemotactic protein-1 (MCP-1) and a vascular endothelial growth factor (VEGF)), as described in U.S. Pat. No. 6,524,581, incorporated by reference in its entirety herein.

The methods provided herein are also useful for determining whether a subject is likely to respond to one or more of the following angiogenesis inhibitors: interferon gamma 1B, interferon gamma Iβ (Actimmune®) with pirfenidone, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with salvia and Schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon a-2B, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming growth factor, β-receptor 2 oligonucleotide, VA999260, XV615 or a combination thereof.

In another embodiment, a method is provided for determining whether a subject is likely to respond to one or more endogenous angiogenesis inhibitors. In a further embodiment, the endogenous angiogenesis inhibitor is endostatin, a 20 kDa C-terminal fragment derived from type XVIII collagen, angiostatin (a 38 kDa fragment of plasmin), a member of the thrombospondin (TSP) family of proteins. In a further embodiment, the angiogenesis inhibitor is a TSP-1, TSP-2, TSP-3, TSP-4 and TSP-5. Methods for determining the likelihood of response to one or more of the following angiogenesis inhibitors are also provided a soluble VEGF receptor, e.g., soluble VEGFR-1 and neuropilin 1 (NPR1), angiopoietin-1, angiopoietin-2, vasostatin, calreticulin, platelet factor-4, a tissue inhibitor of metalloproteinase (TIMP) (e.g., TIMP1, TIMP2, TIMP3, TIMP4), cartilage-derived angiogenesis inhibitor (e.g., peptide troponin I and chrondomodulin I), a disintegrin and metalloproteinase with thrombospondin motif 1, an interferon (IFN), (e.g., IFN-α, IFN-β, IFN-γ), a chemokine, e.g., a chemokine having the C-X-C motif (e.g., CXCL10, also known as interferon gamma-induced protein 10 or small inducible cytokine B10), an interleukin cytokine (e.g., IL-4, IL-12, IL-18), prothrombin, antithrombin III fragment, prolactin, the protein encoded by the TNFSF15 gene, osteopontin, maspin, canstatin, proliferin-related protein.

In one embodiment, a method for determining the likelihood of response to one or more of the following angiogenesis inhibitors is provided is angiopoietin-1, angiopoietin-2, angiostatin, endostatin, vasostatin, thrombospondin, calreticulin, platelet factor-4, TIMP, CDAI, interferon α, interferon β, vascular endothelial growth factor inhibitor (VEGI) meth-1, meth-2, prolactin, VEGI, SPARC, osteopontin, maspin, canstatin, proliferin-related protein (PRP), restin, TSP-1, TSP-2, interferon gamma 1B, ACUHTR028, αVβ5, aminobenzoate potassium, amyloid P, ANG1122, ANG1170, ANG3062, ANG3281, ANG3298, ANG4011, anti-CTGF RNAi, Aplidin, Astragalus membranaceus extract with salvia and Schisandra chinensis, atherosclerotic plaque blocker, Azol, AZX100, BB3, connective tissue growth factor antibody, CT140, danazol, Esbriet, EXC001, EXC002, EXC003, EXC004, EXC005, F647, FG3019, Fibrocorin, Follistatin, FT011, a galectin-3 inhibitor, GKT137831, GMCT01, GMCT02, GRMD01, GRMD02, GRN510, Heberon Alfa R, interferon α-2B, ITMN520, JKB119, JKB121, JKB122, KRX168, LPA1 receptor antagonist, MGN4220, MIA2, microRNA 29a oligonucleotide, MMI0100, noscapine, PBI4050, PBI4419, PDGFR inhibitor, PF-06473871, PGN0052, Pirespa, Pirfenex, pirfenidone, plitidepsin, PRM151, Px102, PYN17, PYN22 with PYN17, Relivergen, rhPTX2 fusion protein, RXI109, secretin, STX100, TGF-β Inhibitor, transforming 2 growth factor, β-receptor oligonucleotide, VA999260, XV615 or a combination thereof.

In yet another embodiment, the angiogenesis inhibitor can include pazopanib (Votrient), sunitinib (Sutent), sorafenib (Nexavar), axitinib (Inlyta), ponatinib (Iclusig), vandetanib (Caprelsa), cabozantinib (Cometrig), ramucirumab (Cyramza), regorafenib (Stivarga), ziv-aflibercept (Zaltrap), motesanib, or a combination thereof. In another embodiment, the angiogenesis inhibitor is a VEGF inhibitor. In a further embodiment, the VEGF inhibitor is axitinib, cabozantinib, aflibercept, brivanib, tivozanib, ramucirumab or motesanib. In yet a further embodiment, the angiogenesis inhibitor is motesanib.

In one embodiment, the methods provided herein relate to determining a subject's likelihood of response to an antagonist of a member of the platelet derived growth factor (PDGF) family, for example, a drug that inhibits, reduces or modulates the signaling and/or activity of PDGF-receptors (PDGFR). For example, the PDGF antagonist, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFR-α or PDGFR-β. In one embodiment, the PDGF antagonist is the anti-PDGF-β aptamer E10030, sunitinib, axitinib, sorefenib, imatinib, imatinib mesylate, nintedanib, pazopanib HCl, ponatinib, MK-2461, dovitinib, pazopanib, crenolanib, PP-121, telatinib, imatinib, KRN 633, CP 673451, TSU-68, Ki8751, amuvatinib, tivozanib, masitinib, motesanib diphosphate, dovitinib dilactic acid, linifanib (ABT-869).

Upon making a determination of whether a patient is likely to respond to angiogenesis inhibitor therapy, or selecting a patient for angiogenesis inhibitor therapy, in one embodiment, the patient is administered the angiogenesis inhibitor. The angiogenesis in inhibitor can be any of the angiogenesis inhibitors described herein.

### Immunotherapy

In one embodiment, provided herein is a method for determining whether a HNSCC cancer patient is likely to respond to immunotherapy by determining the subtype of HNSCC of a sample obtained from the patient and based on the HNSCC subtype, assessing whether the patient is likely to respond to immunotherapy. In another embodiment, provided herein is a method of selecting a patient suffering from HNSCC for immunotherapy by determining a HNSCC subtype of a sample from the patient and, based on the HNSCC subtype, selecting the patient for immunotherapy. The determination of the HNSCC subtype of the sample obtained from the patient can be performed using any method for subtyping HNSCC known in the art. The determination of the HNSCC subtype of the sample obtained from the patient can be performed using any method for subtyping HNSCC provided herein. In one embodiment, the sample obtained from the patient has been previously diagnosed as being HNSCC, and the methods provided herein are used to determine the HNSCC subtype of the sample. The previous diagnosis can be based on a histological analysis. The histological analysis can be performed by one or more pathologists. In one embodiment, the HNSCC subtyping is performed via gene expression analysis of a set or panel of biomarkers or subsets thereof in order to generate an expression profile. The gene expression analysis can be performed on a head and neck cancer sample (e.g., HNSCC sample) obtained from a patient in order to determine the presence, absence or level of expression of one or more biomarkers selected from a publicly available head and neck cancer database described herein and/or Table 1 provided herein. The gene expression analysis can further comprise determining the HPV status of the sample obtained from the subject. The HPV status can be assessed as provided herein (e.g., detecting the expression of one or more HPV genes). The nodal status of the patient may be also be ascertained. The method for ascertaining the nodal status may entail use of any method known in the art for assessing nodal status or nodal metastasis. The HNSCC subtype can be selected from the group consisting of basal, atypical, mesenchymal or classical. The immunotherapy can be any immunotherapy provided herein. In one embodiment, the immunotherapy comprises administering one or more checkpoint inhibitors. The checkpoint inhibitors can be any checkpoint inhibitor provided herein such as, for example, a checkpoint inhibitor that targets PD-1, PD-L1 or CTLA4.

As disclosed herein, the biomarkers panels, or subsets thereof, can be those disclosed in any publicly available HNSCC gene expression dataset or datasets. In one embodiment, the head and neck cancer is SCC and the biomarker panel or subset thereof is, for example, the cancer genome atlas (TCGA) HNSCC RNAseq gene expression dataset (n=520). In one embodiment, the head and neck cancer is SCC and the biomarker panel or subset thereof is, for example, the HNSCC gene expression dataset (n=134) disclosed in Keck et al. (Clin Cancer Res. 2014; 21:870-881.), the contents of which are herein incorporated by reference in its entirety. In one embodiment, the head and neck cancer is SCC and the biomarker panel or subset thereof is, for example, the HNSCC gene expression dataset (n=138) disclosed in Von Walter et al. (PLOS One, 8(2):e56823), the contents of which are herein incorporated by reference in its entirety. In one embodiment, the head and neck cancer is SCC and the biomarker panel or subset thereof is, for example, the HNSCC gene expression dataset (n=270) disclosed in Wichman et al. (Intl Jrnl Cancer 2015; 137:2846-2857), the contents of which are herein incorporated by reference in its entirety. In one embodiment, the head and neck cancer is SCC and the biomarker panel or subset thereof is, for example, the HNSCC gene expression dataset disclosed in Table 1. In one embodiment, the head and neck cancer is SCC and the biomarker panel or subset thereof is, for example, the HNSCC gene expression dataset disclosed in Table 1 in combination with one or more biomarkers from a publicly available HNSCC expression dataset. In one embodiment, the head and neck cancer is SCC and the biomarker panel or subset thereof is, for example, the HNSCC gene expression dataset disclosed in Table 1 in combination with one or more biomarkers of HPV. In one embodiment, the head and neck cancer is SCC and the biomarker panel or subset thereof is, for example, the HNSCC gene expression dataset disclosed in Table 1 in combination with one or more biomarkers from a publicly available HNSCC expression dataset and one or more biomarkers of HPV. In Table 2, the first column of the table represents the biomarker list for distinguishing atypical. The second column of the table represents the biomarker list for basal. The third column of the table represents the biomarker list for distinguishing classical. The last column of the table represents the biomarker list for distinguishing mesenchymal. In some cases, the subset of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the subset of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the subset of classifier biomarkers of Table 1 comprise OLFML3, PCOLCE, LEPRE1, NNMT, OLFML2B, COL6A1, PHLDB1, COL6A2, CMTM3, GPX8, PTH1R, CYP2C18, GRHL3, CSTA, ELF3, SPRR3, ADH7, ALDH3A1, TMPRSS11A, KLF5, SLC9A3R1, SOX2 or any combination thereof. In some cases, the subset of classifier biomarkers of Table 1 comprises all the classifier biomarkers from Table 1.

In one embodiment, the methods provided herein further comprise determining the presence, absence or level of immune activation in a HNSCC subtype. The presence or level of immune cell activation can be determined by creating an expression profile or detecting the expression of one or more biomarkers associated with innate immune cells and/or adaptive immune cells associated with each HNSCC subtype in a sample obtained from a patient. In one embodiment, immune cell activation associated with a HNSCC subtype is determined by monitoring the immune cell signatures of Bindea et al (Immunity 2013; 39(4); 782-795), the contents of which are herein incorporated by reference in its entirety. In one embodiment, the method further comprises measuring single gene immune biomarkers, such as, for example, CTLA4, PDCD1 and CD274 (PD-L1), PDCDLG2(PD-L2) and/or IFN gene signatures. The presence or a detectable level of immune activation (Innate and/or Adaptive) associated with a HNSCC subtype can indicate or predict that a patient with said HNSCC subtype may be amendable to immunotherapy. The immunotherapy can be treatment with a checkpoint inhibitor as provided herein. In one embodiment, a method is provided herein for detecting the expression of at least one classifier biomarker provided herein in a sample (e.g., HNSCC sample) obtained from a patient further comprises administering an immunotherapeutic agent following detection of immune activation as provided herein in said sample.

In one embodiment, the method comprises determining a subtype of a HNSCC sample and subsequently determining a level of immune cell activation of said sub-type. In one embodiment, the subtype is determined by determining the expression levels of one or more classifier biomarkers using sequencing (e.g., RNASeq), amplification (e.g., qRT-PCR) or hybridization assays (e.g., microarray analysis) as described herein. The one or more biomarkers can be selected from a publicly available database (e.g., TCGA HNSCC RNASeq gene expression datasets or any other publicly available HNSCC gene expression datasets provided herein). In some embodiments, one or a plurality of the biomarkers of Table 1 can be used to specifically determine the subtype of a HNSCC sample obtained from a patient, In some embodiments, the subtyping can further comprises determining the HPV status by measuring one or more biomarkers of HPV as described herein. In some embodiments, the subtyping can be in combination with also determining the HPV status by measuring one or more biomarkers of HPV as described herein. The nodal status of the patient may also be ascertained. The method for ascertaining the nodal status may entail use of any method known in the art for assessing nodal status or nodal metastasis. In one embodiment, the level of immune cell activation is determined by measuring gene expression signatures of immunomarkers. The immunomarkers can be measured in the same and/or different sample used to subtype the HNSCC sample as described herein. The immunomarkers that can be measured can comprise, consist of, or consistently essentially of innate immune cell (IIC) and/or adaptive immune cell (AIC) gene signatures, interferon (IFN) gene signatures, individual immunomarkers, major histocompatability complex class II (MHC class II) genes or a combination thereof. The gene expression signatures for both IICs and AICs can be any known gene signatures for said cell types known in the art. For example, the immune gene signatures can be those from Bindea et al. (Immunity 2013; 39(4); 782-795). In one embodiment, the immunomarkers for use in the methods provided herein are selected from Table 4A and/or Table 4B. The individual immunomarkers can be CTLA4, PDCD1 and CD274 (PD-L1). In one embodiment, the individual immunomarkers for use in the methods provided herein are selected from Table 5. The immunomarkers can be one or more interferon (INF) genes. In one embodiment, the immunomarkers for use in the methods provided herein are selected from Table 6. The immunomarkers can be one or more MHCII genes. In one embodiment, the immunomarkers for use in the methods provided herein are selected from Table 7. In yet another embodiment, the immunomarkers for use in the methods provided herein are selected from Tables 4A, 4B, 5, 6, 7, or a combination thereof.

TABLE 4A
Adaptive immune cell (AIC) gene signature immunomarkers for
use in the methods provided herein.

| | Cell Type | | | | | |
|---|---|---|---|---|---|---|
| | B cells | T cells | T helper cells | Tcm | Tem | Th1 cells |
| Human | ABCB4 | BCL11B (B-cell | ANP32B | AQP3 | AKT3 (AKT | APBB2 |
| Gene | (ATP binding | lymphoma/ | (acidic nuclear | (aquaporine 3; | serine/threonine | (amyloid |
| (Gene | cassette | leukaemia 11B; | phosphoprotein | NM_004925.4) | kinase 3; | beta precursor |
| Name; | subfamily B | AJ404614.1) | 32 family | | NM_005465.4) | protein binding |
| GenBank | member 4; | | member B; | | | family B |
| Accession | NM_000443) | | NM_006401.2) | | | member 2; |
| No.*) | | | | | | NM_001166054.1) |
| | BACH2 (BTB | CD2 (CD2 | ASF1A (anti- | ATF7IP | C7orf54 | APOD |
| | domain and | molecule; | silencing | (activating | (staphylococcal | (apolipoprotein D; |
| | CNC homolog 2; | NM_001328609.1) | function 1A | transcription | nuclease | NM_001647.3) |
| | NM_021813.3) | | histone | factor 7 | and tudor | |
| | | | chaperone; | interacting | domain | |
| | | | NM_014034.2) | protein; | containing 1 | |
| | | | | NM_181352.1) | (SND1); | |
| | | | | | NG_051199.1) | |
| | BCL11A (B-cell | CD28 (CD28 | ATF2 | ATM (ATM | CCR2 (C—C | ATP9A |
| | CLL/lymphoma | molecule; | (activating | serine/threonine | motif | (ATPase |
| | 11A; | NM_001243078.1) | transcription | kinase; | chemokine | phospholipid |
| | NM_022893.3) | | factor 2; | NM_000051.3) | receptor 2; | transporting 9A; |
| | | | NM_001256093.1) | | NM_001123396.1) | NM_006045.2) |
| | BLK (BLK | CD3D (CD3d | BATF (basic | CASP8 | DDX17 | BST2 (bone |
| | proto- | molecule; | leucine | (caspase 8; | (DEAD-box | marrow |
| | oncogene, | NM_000732.4) | zipper ATF-like | NM_001228.4) | helicase 17; | stromal cell |
| | Src family | | transcription | | NM_006386.4) | antigen 2; |
| | tyrosine | | factor; | | | NM_004335.3) |
| | kinase; | | NM_006399.3) | | | |
| | NM_001715.2) | | | | | |
| | BLNK (B-cell | CD3E (CD3e | C13orf34 | CDC14A (cell | EWSR1 | BTG3 (BTG |
| | linker; | molecule; | (aurora | division cycle | (EWS RNA | anti- |
| | NM_013314.3) | NM_000733.3) | borealis; | 14A; | binding | proliferation |
| | | | EU834129.1) | NM_003672.3) | protein 1; | factor 3; |
| | | | | | NM_013986.3) | NM_001130914.1) |
| | CCR9 (C—C | CD3G (CD3g | CD28 (CD28 | CEP68 | FLI1 (Fli-1 | CCL4 (C—C |
| | motif | molecule; | molecule; | (centrosomal | proto- | motif |
| | chemokine | NM_000073.2) | NM_006139.3) | protein 68; | oncogene, ETS | chemokine |
| | receptor 9; | | | NM_015147.2) | transcription | ligand 4; |
| | NM_031200.2) | | | | factor; | NM_002984.3) |
| | | | | | NM_002017.4) | |
| | CD19 (CD19 | CD6 (CD6 | DDX50 | CG030 | GDPD5 | CD38 (CD38 |
| | molecule; | molecule; | (DEAD-box | (BRCA2 region, | (glycerophos- | molecule; |
| | NM_001178098.1) | NM_006725.4) | helicase 50; | mRNA sequence | phodiester | NM_001775.3) |
| | | | NM_024045.1) | CG030; | phospho- | |
| | | | | US0531.1) | diesterase | |
| | | | | | domain | |
| | | | | | containing 5; | |
| | | | | | NM_030792.6) | |
| | CD72 (CD72 | CD96 (CD96 | FAM111A | CLUAP1 | LTK | CD70 (CD70 |
| | molecule; | molecule; | (family with | (clusterin | (leukocyte | molecule; |
| | NM_001782.2) | NM_198196.2) | sequence | associated | receptor | NM_001252.4) |
| | | | similarity 111 | protein 1; | tyrosine kinase; | |
| | | | member A; | NM_015041.2) | NM_002344.5) | |
| | | | NM_022074.3) | | | |
| | COCH | GIMAPS | FRYL (FRY | CREBZF | MEFV | CMAH |
| | (cochlin; | (GTPase, | like | (CREB/ATF | (Mediterranean | (cytidine |
| | NM_001135058.1) | IMAP family | transcription | bZIP | fever; | monophospho-N- |
| | | member 5; | coactivator; | transcription | NM_000243.2) | acetylneur- |
| | | NM_018384.4) | NM_015030.1) | factor; | | aminic acid |
| | | | | NM_001039618.2) | | hydroxylase |
| | | | | | | pseudogene; |
| | | | | | | NR_002174.2) |
| | CR2 | ITM2A | FUSIP1 | CYLD (CYLD | NFATC4 | CSF2 |
| | (complement | (integral | (serine and | lysine 63 | (nuclear | (colony |
| | C3d | membrane | arginine rich | deubiquitinase; | factor of | stimulating |
| | receptor 2; | protein 2A; | splicing | NM_015247.2) | activated | factor 2; |
| | NM_001006658.2) | NM_004867.4) | factor 10; | | T-cells 4; | NM_000758.3) |
| | | | NM_006625.5) | | NM_001136022.2) | |
| | DTNB | LCK (LCK | GOLGA8A | CYorf15B | PRKY | CTLA4 |
| | (dystrobrevin | proto- | (golgin A8 | (taxilin | (protein | (cytotoxic |
| | beta; | oncogene, | family | gamma | kinase, Y- | T-lymphocyte |
| | NM_021907.4) | Src family | member A; | pseudogene, | linked, | associated |
| | | tyrosine | NM_181077.3) | Y-linked; | pseudogene; | protein 4; |
| | | kinase; | | NR 045128.1) | NR_028062.1) | NM_005214.4) |
| | | NM_001042771.2) | | | | |
| | FAM30A | NCALD | ICOS | DOCK9 | TBC1D5 | DGKI |
| | (family with | (neurocalcin | (inducible T-cell | (dedicator of | (TBC1 | (diacylglycerol |
| | sequence | delta; | costimulator; | cytokinesis 9; | domain family | kinase |
| | similarity | NM_001040624.1) | NM_012092.3) | NM_015296.2) | member 5; | iota; |
| | 30, member A; | | | | NM_001134381.1) | NM_004717.3) |
| | NR_026800.2) | | | | | |
| | FCRL2 (Fc | PRKCQ | ITM2A | FOXP1 | TBCD | DOKS |
| | receptor like 2; | (protein | (integral | (forkhead | (tubulin | (docking |
| | NM_030764.3) | kinase C theta; | membrane | box P1; | folding | protein 5; |
| | | NM_0062574) | protein 2A; | NM_032682.5) | cofactor D; | NM_018431.4) |
| | | | NM_004867.4) | | NM_005993.4) | |
| | GLDC | SH2D1A | LRBA (LPS | FYB (FYN | TRA (T cell | DPP4 |
| | (glycine | (SH2 domain | responsive | binding | receptor | (dipeptidyl |
| | decarboxylase; | containing 1A; | beige-like | protein; | alpha delta | peptidase 4; |
| | NM_000170.2) | NM_0023514) | anchor protein; | NM_001465.4) | locus; | NM_001935.3) |
| | | | NM_001199282.2) | | NG 001332.3) | |
| | GNG7 (G | SKAP1 (src | NAP1L4 | HNRPH1 | VIL2 (ezrin; | DUSP5 |
| | protein | kinase | (nucleosome | (heterogeneous | NM_003379.4) | (dual |
| | subunit | associated | assembly | nuclear | | specificity |
| | gamma 7; | phosphoprotein 1; | protein 1 like 4; | ribonucleo- | | phosphatase 5; |
| | NM_052847.2) | NM_001075099.1) | NM_005969.3) | protein H1 (H); | | NM_004419.3 |
| | | | | NM_001257293.1) | | |
| | HLA-DOB | TRA (T cell | NUP107 | INPP4B | | EGFL6 (EGF |
| | (major | receptor | (nucleoporin | (inositol | | like domain |
| | histocompatibility | alpha delta | 107; | polyphosphate-4- | | multiple 6; |
| | complex, | locus; | NM_020401.3) | phosphatase | | NM_015507.3) |
| | class II, DO beta; | NG 001332.3) | | type II B; | | |
| | NM_002120.3) | | | NM_003866.3) | | |
| | HLA-DQA1 | TRAC | PHF10 (PHD | KLF12 | | GGT1 |
| | (major | (nuclear | finger | (Kruppel like | | (gamma- |
| | histocompatibility | receptor | protein 10; | factor 12; | | glutamyl- |
| | complex, | corepressor 2; | NM_018288.3) | NM_007249.4) | | transferase 1; |
| | class II, DQ | NM_006312.5) | | | | NM_013421.2) |
| | alpha 1; | | | | | |
| | NM_002122.3) | | | | | |
| | IGHA1 | TRAT1 (T | PPP2R5C | LOC202134 | | HBEGF |
| | (immunoglobulin | cell receptor | (protein | (family with | | (heparin binding |
| | heavy locus; | associated | phosphatase 2 | sequence | | EGF like |
| | NG_001019.6) | transmembrane | regulatory | similarity 153 | | growth factor; |
| | | adaptor 1; | subunit B′, | member B; | | NM_001945.2) |
| | | NM_0163883) | gamma; | NM_001265615.1) | | |
| | | | NM_001161725.1) | | | |
| | IGHG1 | TRBC1 (T | RPA1 | MAP3K1 | | IFNG |
| | (immuno- | cell receptor | (replication | (mitogen- | | (interferon |
| | globulin heavy | beta locus; | protein A1; | activated | | gamma; |
| | locus; | NG_001333.2) | NM_002945.3) | protein | | NM_000619.2) |
| | NG_001019.6) | | | kinase kinase | | |
| | | | | kinase 1, E3 | | |
| | | | | ubiquitin | | |
| | | | | protein | | |
| | | | | ligase; | | |
| | | | | NM_005921.1) | | |
| | IGHM | | SEC24C | MLL (lysine | | IL12RB2 |
| | (immunoglobulin | | (SEC24 | (K)-specific | | (interleukin |
| | heavy locus; | | homolog C, | methyltrans- | | 12 receptor |
| | NG_001019.6) | | COPII coat | ferase 2A; | | subunit beta 2; |
| | | | complex | NM_005933. | | NM_001319233.1) |
| | | | component; | | | |
| | | | NM_004922.3) | | | |
| | IGKC | | SLC25A12 | NEFL | | L22 |
| | (immuno- | | (solute carrier | (neurofilament, | | (interleukin 22; |
| | globulin kappa | | family | light | | NM_020525.4) |
| | locus, proximal | | 25 member 12; | polypeptide; | | |
| | V-cluster and | | NM_003705.4) | NM_006158.4) | | |
| | J-C cluster; | | | | | |
| | NG_000834.1) | | | | | |
| | IGL | | TRA (T cell | NFATC3 | | LRP8 (LDL |
| | (immuno- | | receptor | (nuclear | | receptor |
| | globulin lambda | | alpha delta | factor of | | related |
| | locus; | | locus; | activated | | protein 8; |
| | NG_000002.1) | | NG_001332.3) | T-cells 3; | | NM_017522.4) |
| | | | | NM_173165.2) | | |
| | KIAA0125 | | UBE2L3 | PCM1 | | LRRN3 |
| | (family with | | (ubiquitin | (pericentriolar | | (leucine |
| | sequence | | conjugating | material 1; | | rich repeat |
| | similarity | | enzyme E2 L3; | NM_001315507.1) | | neuronal 3; |
| | 30, member A; | | NM_003347.3) | | | NM_018334.4) |
| | NR_026800.2) | | | | | |
| | MEF2C | | YME1L1 | PCNX | | LTA |
| | (myocyte | | (YME1 like 1 | (pecanex | | (lymphotox |
| | enhancer | | ATPase; | homolog 1; | | in alpha; |
| | factor 2C; | | NM_001253866.1) | NM_014982.2) | | NM_000595.3) |
| | NM_001308002.1) | | | | | |
| | MICAL3 | | | PDXDC2 | | SGCB |
| | (microtubule | | | (pyridoxal | | (sarcoglycan, |
| | associated | | | dependent | | beta |
| | monooxygenase, | | | decarboxylase | | (43 kDa |
| | calponin | | | domain | | dystrophin- |
| | and LIM | | | containing 2, | | associated |
| | domain | | | pseudogene; | | glycoproteln); |
| | containing 3; | | | NR_003610.1) | | NM_000232.4) |
| | NM_001136004.3) | | | | | |
| | MS4A1 | | | PHC3 | | SYNGR3 |
| | (membrane | | | (polyhomeotic | | (synaptogyrin 3; |
| | spanning 4- | | | homolog 3; | | NM_004209.5) |
| | domains A1; | | | NM_001308116.1) | | |
| | NM_021950.3) | | | | | |
| | OSBPL10 | | | POLR2J2 | | ZBTB32 |
| | (oxysterol | | | (RNA | | (zinc finger |
| | binding | | | polymerase II | | and BTB |
| | protein like 10; | | | subunit J2; | | domain |
| | NM_017784.4) | | | NM_032959.5) | | containing 32; |
| | | | | | | NM_014383.2) |
| | PNOC | | | PSPC1 | | |
| | (prepronociceptin; | | | (paraspeckle | | |
| | NM_001284244.1) | | | component 1; | | |
| | | | | NM_001042414.2) | | |
| | QRSL1 | | | REPS1 | | |
| | (glutaminyl- | | | (RALBP1 | | |
| | RNA | | | associated | | |
| | synthase | | | Eps domain | | |
| | (glutamine- | | | containing 1; | | |
| | hydrolyzing) | | | NM_001128617.2) | | |
| | -like 1; | | | | | |
| | NM_018292.4) | | | | | |
| | SCN3A | | | RP11- | | |
| | (sodium | | | 74E24.2 (zinc | | |
| | voltage-gated | | | finger CCCH- | | |
| | channel alpha | | | type domain- | | |
| | subunit 3; | | | containing-like; | | |
| | NM_001081677.1) | | | NM_001271675.1) | | |
| | SLC15A2 | | | RPP38 | | |
| | (solute carrier | | | (ribonuclease | | |
| | family 15 | | | P/MRP | | |
| | member 2; | | | subunit p38; | | |
| | XM_017007074.1) | | | NM_001265601.1) | | |
| | SPIB (Spi-B | | | SLC7A6 | | |
| | transcription | | | (solute | | |
| | factor; | | | carrier family | | |
| | NM_001244000.1) | | | 7 member 6; | | |
| | | | | NM_003983.5) | | |
| | TCL1A (T-cell | | | SNRPN (small | | |
| | leukemia/ | | | nuclear | | |
| | lymphoma 1A; | | | ribonucleoprotein | | |
| | NM_001098725.1) | | | polypeptide N; | | |
| | | | | NM_022807.3) | | |
| | TNFRSF17 | | | ST3GAL1 | | |
| | (TNF receptor | | | (ST3 beta- | | |
| | superfamily | | | galactoside | | |
| | member 17; | | | alpha-2,3- | | |
| | NM_0011922) | | | sialyltransferase 1; | | |
| | | | | NM_173344.2) | | |
| | | | | STX16 | | |
| | | | | (syntaxin 16; | | |
| | | | | NM_001204868.1) | | |
| | | | | TIMM8A | | |
| | | | | (translocase | | |
| | | | | of inner | | |
| | | | | mitochondrial | | |
| | | | | membrane | | |
| | | | | 8 homolog A; | | |
| | | | | NM_001145951.1) | | |
| | | | | TRAF3IP3 | | |
| | | | | (TRAF3 | | |
| | | | | interacting | | |
| | | | | protein 3; | | |
| | | | | NM_001320144.1) | | |
| | | | | TXK (TXK | | |
| | | | | tyrosine kinase; | | |
| | | | | NM_003328.2) | | |
| | | | | USP9Y | | |
| | | | | (ubiquitin | | |
| | | | | specific | | |
| | | | | peptidase 9, | | |
| | | | | Y-linked; | | |
| | | | | NG_008311.1) | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 4A
Cell Type

| | | | Th17 | | CD8 T | | Cytotoxic |
|---|---|---|---|---|---|---|---|
| | Th2 cells | TFH | cells | TReg | cells | Tgd | cells |
| Human | ADCY1 | B3GAT1 | IL17A | FOXP3 | ABT1 | C1orf61 | APBA2 |
| Gene | (adenylate | (beta-1,3- | (interleukin | (forkhead | (activator | (chromosome | (amyloid |
| (Gene | cyclase 1; | glucuronyl | 17A; | box P3; | of basal | 1 | beta |
| Name; | NM_{—} | transferase | NM_{—} | NM_{—} | transcription | open | precursor |
| GenBank | 001281768.1) | 1; | 002190.2) | 014009.3) | 1; | reading | protein |
| Accession | | NM_{—} | | | NM_{—} | frame 61; | binding |
| No.*) | | 018644.3) | | | 013375.3) | NM_{—} | family A |
| | | | | | | 006365.2) | member 2; |
| | | | | | | | NM_{—} |
| | | | | | | | 005503.3) |
| | AHI1 | BLR1 (c-x-c | IL17RA | | AES | CD160 | APOL3 |
| | (Abelson | chemokine | (interleukin | | (amino- | (CD160 | (apolipoprotein |
| | helper | receptor | 17 | | terminal | molecule; | L3; |
| | integration | type 5; | receptor | | enhancer | NM_{—} | NM_{—} |
| | site 1; | EF444957.1) | A; | | of split; | 007053.3) | 014349.2) |
| | NM_{—} | | NM_{—} | | NM_{—} | | |
| | 001134831.1) | | 014339.6) | | 198969.1) | | |
| | Al582773 | C18orf1 | RORC | | APBA2 | FEZ1 | CTSW |
| | (tn17d08.x1 | (low | (RAR | | (amyloid | (Fasciculation | (cathepsin |
| | NCl_CGAP_{—} | density | related | | beta | And | W; |
| | Brn25 | lipoprotein | orphan | | precursor | Elongation | NM_001335.3) |
| | Homo | receptor | receptor | | protein | Protein | |
| | sapiens | class A | C; | | binding | Zeta 1; | |
| | cDNA | domain | NM_{—} | | family A | AF123659.1) | |
| | clone; | containing | 001001523.1) | | member | | |
| | Al582773.1) | 4; | | | 2; NM_{—} | | |
| | | NM_181481.4) | | | 001130414.1) | | |
| | ANK1 | CDK5R1 | | | ARHGAP8 | TARP (TCR | DUSP2 |
| | (ankyrin 1; | (cyclin | | | (Rho | gamma | (dual |
| | NM_{—} | dependent | | | GTPase | alternate | specificity |
| | 020476.2) | kinase 5 | | | activating | reading | phosphatase |
| | | regulatory | | | protein 8; | frame | 2; |
| | | subunit 1; | | | NM_{—} | protein; | NM_004418.3) |
| | | NM_003885.2) | | | 001198726.1) | NM_{—} | |
| | | | | | | 001003806.1) | |
| | BIRC5 | CHGB | | | C12orf47 | TRD (T cell | GNLY |
| | (baculoviral | (chromogranin B; | | | (MAPKAPK5 | receptor | (granulysin; |
| | IAP | NM_001819.2) | | | antisense | alpha | NM_012483.3) |
| | repeat | | | | RNA 1; | delta | |
| | containing | | | | NR_015404.1) | locus; | |
| | 5; NM_{—} | | | | | NG_001332.3) | |
| | 001012271.1) | | | | | | |
| | CDC25C | CHI3L2 | | | C19orf6 | TRGV9 (T | GZMA |
| | (cell | (chitinase | | | (transmembrane | cell | (granzyme |
| | division | 3 like 2; | | | protein | receptor | A; |
| | cycle 25C; | NM_{—} | | | 259; | gamma V | NM_006144.3) |
| | NM_001318098.1) | 001025199.1) | | | NM_001033026.1) | region 9; | |
| | | | | | | X69385.1) | |
| | CDC7 (cell | CXCL13 | | | C4orf15 | | GZMH |
| | division | (C-X-C | | | (HAUS | | (granzyme |
| | cycle 7; | motif | | | augmin | | H; |
| | NM_001134420.1) | chemokine | | | like | | NM_{—} |
| | | ligand | | | complex | | 001270781.1) |
| | | 13; | | | subunit 3; | | |
| | | NM_006419.2) | | | NM_001303143.1) | | |
| | CENPF | HEY1 (hes | | | CAMLG | | KLRB1 |
| | (centromere | related | | | (calcium | | (killer cell |
| | protein | family | | | modulating | | lectin like |
| | F; | bHLH | | | ligand; | | receptor |
| | NM_016343.3) | transcription | | | NM_001745.3) | | B1; |
| | | factor | | | | | NM_002258.2) |
| | | with | | | | | |
| | | YRPW | | | | | |
| | | motif 1; | | | | | |
| | | NM_001282851.1) | | | | | |
| | CXCR6 | HIST1H4K | | | CD8A | | KLRD1 |
| | (killer cell | (histone | | | (CD8a | | (killer cell |
| | lectin like | cluster 1 | | | molecule; | | lectin like |
| | receptor | H4 family | | | NM_001768.6) | | receptor |
| | B1; | member | | | | | D1; |
| | NM_002258.2) | k; | | | | | NM_001114396.1) |
| | | NM_003541.2) | | | | | |
| | DHFR | ICA1 (islet | | | CD8B | | KLRF1 |
| | (dihydrofolate | cell | | | (CD8b | | (killer cell |
| | reductase; | autoantigen | | | molecule; | | lectin like |
| | NM_{—} | 1; | | | NM_{—} | | receptor |
| | 001290354.1) | NM_{—} | | | 001178100.1) | | F1; |
| | | 001136020.2) | | | | | NM_001291822.1) |
| | EVI5 | KCNK5 | | | CDKN2AIP | | KLRK1 |
| | (ecotropic | (potassium | | | (CDKN2A | | (killer cell |
| | viral | two | | | interacting | | lectin like |
| | integration | pore | | | protein; | | receptor |
| | site 5; | domain | | | NM_{—} | | K1; |
| | NM_{—} | channel | | | 001317343.1) | | NM_007360.3) |
| | 001308248.1) | subfamily | | | | | |
| | | K member | | | | | |
| | | 5; | | | | | |
| | | NM_003740.3) | | | | | |
| | GATA3 | KIAA1324; | | | DNAJB1 | | NKG7 |
| | (GATA | (KIAA1324 | | | (DnaJ heat | | (natural |
| | binding | NM_001284353.1) | | | shock | | killer cell |
| | protein 3; | | | | protein | | granule |
| | NM_001002295.1) | | | | family | | protein 7; |
| | | | | | (Hsp40) | | NM_005601.3) |
| | | | | | member | | |
| | | | | | B1; | | |
| | | | | | NM_001313964.1) | | |
| | GSTA4 | MAF (MAF | | | FLT3LG | | RORA (RAR |
| | (glutathione | bZIP | | | (fms | | related |
| | S- | transcription | | | related | | orphan |
| | transferase | factor; | | | tyrosine | | receptor A; |
| | alpha 4; | NM_001031804.2) | | | kinase 3 | | NM_134262.2) |
| | NM_001512.3) | | | | ligand; | | |
| | | | | | NM_001278638.1) | | |
| | HELLS | MAGEH1 | | | GADD45A | | RUNX3 |
| | (helicase, | (MAGE | | | (growth | | (runt |
| | lymphoid- | family | | | arrest and | | related |
| | specific; | member | | | DNA | | transcription |
| | NM_001289074.1) | H1; | | | damage | | factor 3; |
| | | NM_014061.4) | | | inducible | | NM_004350.2) |
| | | | | | alpha; | | |
| | | | | | NM_001199742.1) | | |
| | IL26 | MKL2 | | | GZMM | | SIGIRR |
| | (interleukin | (MKL1/myocardin | | | (granzyme | | (single lg |
| | 26; | like 2; | | | M; | | and TIR |
| | NM_018402.1) | NM_014048.4) | | | NM_001258351.1) | | domain |
| | | | | | | | containing; |
| | | | | | | | NM_001135054.1) |
| | LAIR2 | MYO6 | | | KLF9 | | WHDC1L1 |
| | (leukocyte | (myosin | | | (Kruppel | | (WAS |
| | associated | VI; | | | like factor | | protein |
| | immunoglobulin | NM_001300899.1) | | | 9; | | homolog |
| | like | | | | NM_001206.2) | | associated |
| | receptor 2, | | | | | | with actin, |
| | NM_021270.4) | | | | | | golgi |
| | | | | | | | membranes |
| | | | | | | | and |
| | | | | | | | microtubules |
| | | | | | | | pseudogene 3; |
| | | | | | | | NR_003521.1 |
| | LIMA1 | MYO7A | | | LEPROTL1 | | ZBTB16 |
| | (LIM | (myosin | | | (leptin | | (zinc finger |
| | domain | VIIA; | | | receptor | | and BTB |
| | and actin | NM_001127179.2) | | | overlapping | | domain |
| | binding 1; | | | | transcript- | | containing |
| | NM_001243775.1) | | | | like 1; | | 16; |
| | | | | | NM_001128208.1) | | NM_001018011.1) |
| | MB | PASK (PAS | | | LIME1 | | |
| | (myoglobin; | domain | | | (Lck | | |
| | NM_203377.1) | containing | | | interacting | | |
| | | serine/threonine | | | transmembrane | | |
| | | kinase; | | | adaptor 1; | | |
| | | NM_001252119.1) | | | NM_017806.3) | | |
| | MICAL2 | PDCD1 | | | MYST3 | | |
| | (microtubule | (programmed | | | (MYST | | |
| | associated | cell | | | histone | | |
| | monooxygenase, | death 1; | | | acetyltransferase | | |
| | calponin | NM_{—} | | | (monocytic | | |
| | and LIM | 005018.2) | | | leukemia) | | |
| | domain | | | | 3; | | |
| | containing | | | | NM_{—} | | |
| | 2; NM_{—} | | | | 006766.4) | | |
| | 001282663.1) | | | | | | |
| NEIL3 (nei | POMT1 | | | | PF4 | | |
| like DNA | (protein | | | | (platelet | | |
| glycosylase 3; | O- | | | | factor 4; | | |
| NM_018248.2) | mannosyltransferase | | | | NM_{—} | | |
| | 1; NM_{—} | | | | 002619.3) | | |
| | 001136114.1) | | | | | | |
| | PHEX | PTPN13 | | | PPP1R2 | | |
| | (phosphate | (protein | | | (protein | | |
| | regulating | tyrosine | | | phosphatase | | |
| | endopeptidase | phosphatase, | | | 1 | | |
| | homolog, | non- | | | regulatory | | |
| | X-linked; | receptor | | | inhibitor | | |
| | NM_000444.5) | type 13; | | | subunit 2; | | |
| | | NM_080685.2) | | | NM_001291504.1) | | |
| | PMCH | PVALB | | | PRF1 | | |
| | (pro- | (parvalbumin; | | | (perforin | | |
| | melanin | NM_{—} | | | 1; | | |
| | concentrating | 001315532.1) | | | NM_005041.4) | | |
| | hormone; | | | | | | |
| | NM_002674.3) | | | | | | |
| | PTGIS (12 | SH3TC1 | | | PRRS | | |
| | synthase; | (SH3 | | | (proline | | |
| | NM_000961.3) | domain | | | rich 5; | | |
| | | and | | | NM_181333.3) | | |
| | | tetratrico | | | | | |
| | | peptide | | | | | |
| | | repeats 1; | | | | | |
| | | NM_018986.4) | | | | | |
| | SLC39A14 | SIRPG | | | RBM3 | | |
| | (solute | (signal | | | (RNA | | |
| | carrier | regulatory | | | binding | | |
| | family 39 | protein | | | motif | | |
| | member | gamma; | | | (RNP1, | | |
| | 14; | NM_018556.3) | | | RRM) | | |
| | NM_{—} | | | | protein 3; | | |
| | 001135153.1) | | | | NM_006743.4) | | |
| | SMAD2 | SLC7A10 | | | SF1 | | |
| | (SMAD | (solute | | | (splicing | | |
| | family | carrier | | | factor 1; | | |
| | member 2; | family 7 | | | NM_004630.3) | | |
| | NM_{—} | member | | | | | |
| | 001135937.2) | 10; NM_{—} | | | | | |
| | | 019849.2) | | | | | |
| | SNRPD1 | SMAD1 | | | SFRS7 | | |
| | (small | (SMAD | | | (serine | | |
| | nuclear | family | | | and | | |
| | ribonucle | member | | | arginine | | |
| | oprotein | 1; | | | rich | | |
| | D1 | NM_{—} | | | splicing | | |
| | polypeptide; | 001003688.1) | | | factor 7; | | |
| | NM_001291916.1) | | | | NM_001031684.2) | | |
| | WDHD1 | ST8SIA1 | | | SLC16A7 | | |
| | (WD | (ST8 | | | (solute | | |
| | repeat | alpha-N- | | | carrier | | |
| | and HMG- | acetyl- | | | family 16 | | |
| | box DNA | neuraminide | | | member | | |
| | binding | alpha- | | | 7: | | |
| | protein 1; | 2,8- | | | NM_{—} | | |
| | NM_001008396.2) | sialyltransferase | | | 001270622.1) | | |
| | | 1; NM_{—} | | | | | |
| | | 001304450.1) | | | | | |
| | | STK39 | | | TBCC | | |
| | | (serine/threonine | | | (tubulin | | |
| | | kinase 39; | | | folding | | |
| | | NM_013233.2) | | | cofactor | | |
| | | | | | C; NM_{—} | | |
| | | | | | 003192.2) | | |
| | | THADA | | | THUMPD1 | | |
| | | (THADA, | | | (THUMP | | |
| | | armadillo | | | domain | | |
| | | repeat | | | containing | | |
| | | containing; | | | 1; | | |
| | | NM_001271644.1) | | | NM_017736.4) | | |
| | | TOX (thymocyte) | | | TMC6 | | |
| | | selection | | | (transmembrane | | |
| | | associated | | | channel | | |
| | | high | | | like 6; | | |
| | | mobility | | | NM_{—} | | |
| | | group | | | 001321185.1) | | |
| | | box; | | | | | |
| | | NM_014729.2) | | | | | |
| | | TSHR | | | TSC22D3 | | |
| | | (thyroid | | | (TSC22 | | |
| | | stimulating | | | domain | | |
| | | hormone | | | family | | |
| | | receptor; | | | member | | |
| | | NM_{—} | | | 3; NM_{—} | | |
| | | 000369.2) | | | 001318470.1) | | |
| | | ZNF764 | | | VAMP2 | | |
| | | (zinc | | | (vesicle | | |
| | | finger | | | associated | | |
| | | protein | | | membrane | | |
| | | 764; | | | protein | | |
| | | NM_{—} | | | 2; | | |
| | | 001172679.1) | | | NM_014232.2) | | |
| | | | | | ZEB1 (zinc | | |
| | | | | | finger E- | | |
| | | | | | box | | |
| | | | | | binding | | |
| | | | | | homeobox | | |
| | | | | | 1; NM_{—} | | |
| | | | | | 001128128.2) | | |
| | | | | | ZFP36L2 | | |
| | | | | | (ZFP36 | | |
| | | | | | ring finger | | |
| | | | | | protein | | |
| | | | | | like 2; | | |
| | | | | | NM_006887.4) | | |
| | | | | | ZNF22 | | |
| | | | | | (zinc | | |
| | | | | | finger | | |
| | | | | | protein | | |
| | | | | | 22; | | |
| | | | | | NM_006963.4) | | |
| | | | | | ZNF609 | | |
| | | | | | (zinc | | |
| | | | | | finger | | |
| | | | | | protein | | |
| | | | | | 609; | | |
| | | | | | NM_015042.1) | | |
| | | | | | ZNF91 | | |
| | | | | | (zinc | | |
| | | | | | finger | | |
| | | | | | protein | | |
| | | | | | 91; | | |
| | | | | | NM_{—} | | |
| | | | | | 001300951.1) | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 4B
Innate immune cell (IIC) gene signature immunomarkers for use in the methods provided herein.
Cell Type

| | | NK CD56dim | NK CD56bright | | |
|---|---|---|---|---|---|
| | NK cells | cells | cells | DC | iDC |
| Human Gene | ADARB1 | EDG8 | BG255923 | CCL13 (C-C | ABCG2 (ATP- |
| (Gene Name; | (adenosine | (sphingosine-1- | (lysophosphatidylcholine | motif chemokine | binding cassette, |
| GenBank | deaminase, | phosphate | acyltransferase 4; | ligand 13; | sub-family G |
| Accession | RNA specific | receptor 5; | NM_153613.2) | NM_005408.2) | (WHITE), member 2 |
| No.*) | B1; NM_001112) | NM_001166215.1) | | | (Junior blood group); |
| | | | | | NM_001257386.1) |
| | AF107846 | FLJ20699 (cDNA | DUSP4 (dual | CCL17 (C-C | BLVRB |
| | (neuroendocrine- | FLJ20699 fis, | specificity | motif chemokine | (biliverdin |
| | specific Golgi | clone KAIA2372; | phosphatase 4; | ligand 17; | reductase B; |
| | protein p55; | AK000706.1) | NM_057158.3) | NM_002987.2) | NM_000713.2) |
| | AF107846.1) | | | | |
| | AL080130 | GTF3C1 (general | FOXJ1 | CCL22 (C-C | CARD9 (caspase |
| | (cDNA | transcription | (forkhead box J1; | motif chemokine | recruitment |
| | DKFZp434E033 | factor IIIC | NM_001454.3) | ligand 22; | domain family |
| | (from clone | subunit 1; | | NM_002990.4) | member 9; |
| | DKFZp434E033); | NM_001286242.1) | | | NM_052814.3) |
| | AL080130.1) | | | | |
| | ALDH1B1 | GZMB | MADD (MAP | CD209 (CD209 | CD1A (CD1a |
| | (aldehyde | (granzyme B; | kinase activating | molecule; | molecule; |
| | dehydrogenase | NM_004131.4) | death domain; | NM_001144899.1) | NM_001763.2) |
| | 1 family | | NM_001135944.1) | | |
| | member B1; | | | | |
| | NM_000692.4) | | | | |
| | ARL6IP2 | IL21R | MPPED1 | HSD11B1 | CD1B (CD1b |
| | (atlastin | (interleukin 21 | (metallophosphoesterase | (hydroxysteroid | molecule; |
| | GTPase 2; | receptor; | domain containing 1, | 11-beta | NM_001764.2) |
| | NM_001330461.1) | NM_181079.4) | mRNA; | dehydrogenase 1; | |
| | | | NM_001044370.1) | NM_001206741.1) | |
| | BCL2 (apoptosis | KIR2DL3 (killer | MUC3B (mucin | NPR1 | CD1C (CD1c |
| | regulator (BCL2); | cell immunoglobulin | 3B cell surface | (natriuretic | molecule; |
| | NM_000633.2) | like receptor, two | associated; | peptide | NM_001765.2) |
| | | lg domains and long | JQ511939.1) | receptor 1; | |
| | | cytoplasmic tail 3; | | NM_000906.3) | |
| | | NM_015868.2) | | | |
| | CDC5L (cell | KIR2DS1 (killer | NIBP (NIK and | PPFIBP2 | CD1E (CD1e |
| | division cycle | cell immunoglobulin | IKKbetta- | (PPFIA binding | molecule; |
| | 5 like; | like receptor, two | binding protein; | protein 2; | NM_001185115.1) |
| | NM_001253.3) | lg domains and short | AY630619.1) | XR_930917.2) | |
| | | cytoplasmic tail 1; | | | |
| | | NM_014512.1) | | | |
| | FGF18 | KIR2DS2 (killer | PLA2G6 | | CH25H |
| | (fibroblast | cell immunoglobulin | (phospholipase | | (cholesterol 25- |
| | growth factor 18; | like receptor, two | A2 group VI; | | hydroxylase; |
| | NM_003862.2) | lg domains and short | NM_001004426.1) | | NM_003956.3) |
| | | cytoplasmic tail 2; | | | |
| | | NM_001291700.1) | | | |
| | FUT5 | KIR2DS5 (killer | RRAD (Ras related | | CLEC10A (C-type |
| | (fucosyltransferase | cell immunoglobulin | glycolysis inhibitor | | lectin domain |
| | 5; NM_002034.2) | like receptor, two | and calcium channel | | family 10 member A; |
| | | lg domains and short | regulator; | | NM_001330070.1) |
| | | cytoplasmic tail 5; | NM_001128850.1) | | |
| | | NM_014513.2) | | | |
| | FZR1 (fizzy/cell | KIR3DL1 (killer | SEPT6 (septin 6; | | CSF1R (colony |
| | division cycle | cell immunoglobulin | NM_145802.3) | | stimulating |
| | 20 related 1; | like receptor, three | | | factor 1 receptor; |
| | XM_005259573.4) | lg domains and long | | | NM_001288705.1) |
| | | cytoplasmic tail 1; | | | |
| | | NM_013289.2) | | | |
| | GAGE2 | KIR3DL2 (killer | XCL1 (X-C motif | | CTNS (cystinosin, |
| | (G antigen 2; | cell immunoglobulin | chemokine ligand 1; | | lysosomal cystine |
| | NM_001127212.1) | like receptor, three | NM_002995.2) | | transporter; |
| | | lg domains and long | | | NM_001031681.2) |
| | | cytoplasmic tail 2; | | | |
| | | NM_006737.3) | | | |
| | IGFBP5 (insulin | KIR3DL3 (killer | | | F13A1 (factor |
| | like growth | cell immunoglobulin | | | XIII a subunit; |
| | factor binding | like receptor, three | | | AH002691.2) |
| | protein 5; | lg domains and long | | | |
| | NM_000599.3) | cytoplasmic tail 3; | | | |
| | | NM_153443.4) | | | |
| | LDB3 (LIM | KIR3DS1 (killer | | | FABP4 (fatty acid |
| | domain binding 3; | cell immunoglobulin | | | binding protein 4; |
| | NM_001171611.1) | like receptor, three | | | NM_001442.2) |
| | | lg domains and short | | | |
| | | cytoplasmic tail 1; | | | |
| | | NM_001083539.2) | | | |
| | LOC643313 | SPON2 (spondin 2 | | | FZD2 (frizzled |
| | (similar to | NM_001199021.1) | | | class receptor 2; |
| | hypothetical | | | | NM_001466.3) |
| | protein | | | | |
| | LOC284701; | | | | |
| | XM_933043.1) | | | | |
| | LOC730096 | TMEPAI (prostate | | | GSTT1 |
| | (hypothetical | transmembrane | | | (glutathione |
| | protein | protein, androgen | | | S-transferase |
| | LOC730096; | induced 1; | | | theta 1; |
| | NC_000022.9) | NM_199169.2) | | | NM_001293814.1) |
| | MAPRE3 | | | | GUCA1A |
| | (microtubule | | | | (guanylate cyclase |
| | associated | | | | activator 1A; |
| | protein RP/EB | | | | NM_001319062.1) |
| | family member 3; | | | | |
| | NM_001303050.1) | | | | |
| | MCM3AP | | | | HS3ST2 |
| | (minichromosome | | | | (heparan sulfate |
| | maintenance | | | | (glucosamine) 3-O- |
| | complex component 3 | | | | sulfotransferase 2; |
| | associated protein; | | | | NM_006043.1) |
| | NM_003906.4) | | | | |
| | MRC2 | | | | LMAN2L (lectin, |
| | (mannose receptor | | | | mannose binding |
| | C type 2; | | | | 2 like; |
| | NM_006039.4) | | | | NM_001322355.1) |
| | NCR1 (natural | | | | MMP12 (matrix |
| | cytotoxicity | | | | metallopeptidase 12; |
| | triggering | | | | NM_002426.5) |
| | receptor 1; | | | | |
| | NM_001242357.2) | | | | |
| | NM_014114 | | | | MS4A6A |
| | (PRO0097 protein; | | | | (membrane spanning |
| | NM_014114.1) | | | | 4-domains A6A; |
| | | | | | NM_001330275.1) |
| | NM_014274 | | | | NM_021941 |
| | (transient | | | | (chromosome 21 |
| | receptor potential | | | | open reading |
| | cation channel, | | | | frame 97; |
| | subfamily V, | | | | NM_021941.1) |
| | member 6; | | | | |
| | NM_014274.3) | | | | |
| | NM_017616 | | | | NUDT9 (nudix |
| | (KN motif and | | | | hydrolase 9; |
| | ankyrin repeat | | | | NM_001248011.1) |
| | domains 2; | | | | |
| | NM_015493.6) | | | | |
| | PDLIM4 (PDZ and | | | | PPARG |
| | LIM domain 4; | | | | (peroxisome |
| | NM_003687.3) | | | | proliferator |
| | | | | | activated |
| | | | | | receptor gamma; |
| | | | | | NM_005037.5) |
| | PRX (periaxin; | | | | PREP (prolyl |
| | NM_020956.2) | | | | endopeptidase; |
| | | | | | NM_002726.4) |
| | PSMD4 | | | | RAP1GAP (RAP1 |
| | (proteasome | | | | GTPase activating |
| | 26S subunit, | | | | protein; |
| | non-ATPase 4; | | | | NM_001330383.1) |
| | NM_001330692.1) | | | | |
| | RP5-886K2.1 | | | | SLC26A6 (solute |
| | (neuronal thread | | | | carrier family 26 |
| | protein AD7c-NTP; | | | | member 6; |
| | AF010144.1) | | | | NM_001281733.1) |
| | SLC30A5 (solute | | | | SLC7A8 (solute |
| | carrier family 30 | | | | carrier family 7 |
| | member 5; | | | | member 8; |
| | NM_001251969.1) | | | | NR_049767.1) |
| | SMEK1 (protein | | | | SYT17 |
| | phosphatase 4 | | | | (synaptotagmin 17; |
| | regulatory | | | | NM_001330509.1) |
| | subunit 3A; | | | | |
| | NM_001284280.1) | | | | |
| | SPN | | | | TACSTD2 (tumor- |
| | (sialophorin; | | | | associated |
| | NM_003123.4) | | | | calcium signal |
| | | | | | transducer 2; |
| | | | | | NM_002353.2) |
| | TBXA2R | | | | TM75F4 |
| | (thromboxane | | | | (dendrocyte |
| | A2 receptor; | | | | expressed seven |
| | NM_001060.5) | | | | transmembrane |
| | | | | | protein; |
| | | | | | NM_001257317.1) |
| | TCTN2 | | | | VASH1 |
| | (tectonic family | | | | (vasohibin 1; |
| | member 2; | | | | NM_014909.4) |
| | NM_001143850.2) | | | | |
| | TINAGL1 | | | | |
| | (tubulointerstitial | | | | |
| | nephritis antigen | | | | |
| | like 1; | | | | |
| | NM_001204415.1) | | | | |
| | XCL1 (X-C motif | | | | |
| | chemokine ligand 1; | | | | |
| | NM_002995.2) | | | | |
| | XCL2 (X-C motif | | | | |
| | chemokine ligand 2; | | | | |
| | NM_003175.3) | | | | |
| | ZNF205 (zinc | | | | |
| | finger protein 205; | | | | |
| | NM_001278158.1) | | | | |
| | ZNF528 (zinc | | | | |
| | finger protein 528; | | | | |
| | NM_032423.2) | | | | |
| | ZNF747 (zinc | | | | |
| | finger protein 747; | | | | |
| | NM_023931.3) | | | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number,

TABLE 4B
Cell Type

| | aDC | pDC | Eosinophils | Macrophages | Mast cells | Neutrophils |
|---|---|---|---|---|---|---|
| Human | CCL1 | IL3RA | ABHD2 | APOE | ABCC4 (ATP | ALPL |
| Gene | (Chemokine | (interleukin | (abhydrolase | (apolipoprotein | binding | (alkaline |
| (Gene | (C-C | 3 | domain | E; | cassette | phosphatase, |
| Name; | motif) | receptor | containing 2; | NM_001302691.1) | subfamily C | liver/bone/kidney; |
| GenBank | ligand 1; | subunit | NM_007011.7) | | member 4; | NM_001127501.3) |
| Accession | NM_002981) | alpha; | | | NM_001301829.1) | |
| No.*) | | NM_001267713.1) | | | | |
| | EBI3 | | ACACB | ATG7 | ADCYAP1 | BST1 (bone |
| | (Epstein- | | (acetyl-CoA | (autophagy | (adenylate | marrow |
| | Barr virus | | carboxylase | related 7; | cyclase | stromal cell |
| | induced 3; | | beta; | NM_001144912.1) | activating | antigen 1; |
| | NM_005755.2) | | NM_001093.3) | | polypeptide | NM_004334.2) |
| | | | | | 1; | |
| | | | | | NM_001117.4) | |
| | INDO | | C9orf156 | BCAT1 | CALB2 | CD93 (CD93 |
| | (indoleamine- | | (tRNA | (branched | (calbindin 2; | molecule; |
| | pyrrole | | methyltransferase | chain | NM_001740.4) | NM_012072.3) |
| | 2,3 | | O; | amino acid | | |
| | dioxygenase; | | NM_001330725.1) | transaminase | | |
| | AY221100.1) | | | 1; | | |
| | | | | NM_001178094.1) | | |
| | LAMP3 | | CAT | CCL7 (C-C | CEACAM8 | CEACAM3 |
| | (lysosomal | | (catalase; | motif | (carcinoembryonic | (carcinoembryonic |
| | associated | | NM_001752.3) | chemokine | antigen | antigen |
| | membrane | | | ligand 7; | related cell | related cell |
| | protein 3; | | | NM_006273.3) | adhesion | adhesion |
| | NM_014398.3) | | | | molecule 8; | molecule 3; |
| | | | | | NM_001816.3) | NM_001277163.2) |
| | OAS3 (2′-5′- | | CCR3 (C-C | CD163 | CMA1 | CREB5 |
| | oligoadenylate | | motif | (CD163 | (chymase 1, | (CAMP |
| | synthetase | | chemokine | molecule; | mast cell; | responsive |
| | 3; | | receptor 3; | NM_203416.3) | NM_001308083.1) | element |
| | NM_006187.3) | | NM_178329.2) | | | binding |
| | | | | | | protein 5; |
| | | | | | | NM_001011666.2) |
| | | | CLC (Charcot- | CD68 | CPA3 | CRISPLD2 |
| | | | Leyden | (CD68 | (carboxypeptidase | (cysteine |
| | | | crystal | molecule; | A3; | rich |
| | | | galectin; | NM_001040059.1) | NM_001870.3) | secretory |
| | | | NM_001828.5) | | | protein LCCL |
| | | | | | | domain |
| | | | | | | containing 2; |
| | | | | | | NM_031476.3) |
| | | | CYSLTR2 | CD84 | CTSG | CSF3R |
| | | | (cysteinyl | (CD84 | (cathepsin | (colony |
| | | | leukotriene | molecule; | G; | stimulating |
| | | | receptor 2; | NM_001184881.1) | NM_001911.2) | factor 3 |
| | | | NM_001308471.1) | | | receptor; |
| | | | | | | NM_172313.2) |
| | | | EMR1 (EGF- | CHI3L1 | ELA2 | CYP4F3 |
| | | | like module | (chitinase 3 | (neutrophil | (cytochrome |
| | | | containing | like 1; | elastase; | P450 family |
| | | | mucin-like | NM_001276.2) | EU617980.1) | 4 subfamily F |
| | | | hormone | | | member 3; |
| | | | receptor-like | | | NM_001199209.1) |
| | | | 1; | | | |
| | | | DQ217942.1) | | | |
| | | | EPN2 (epsin | CHIT1 | GATA2 | DYSF |
| | | | 2; | (chitinase | (GATA | (dysferlin; |
| | | | NM_001102664.1) | 1; | binding | NM_001130455.1) |
| | | | | NM_001270509.1) | protein 2; | |
| | | | | | NM_001145661.1) | |
| | | | GALC | CLECSA (C- | HDC | FCAR (Fc |
| | | | (galactosylceramidase; | type lectin | (histidine | fragment of |
| | | | NM_000153.3) | domain | decarboxylase; | IgA receptor; |
| | | | | family 5 | NM_002112.3) | NM_133278.3) |
| | | | | member A; | | |
| | | | | NM_001301167.1) | | |
| | | | GPR44 | COL8A2 | HPGD | FCGR3B (Fc |
| | | | (orphan G | (collagen | (hydroxypro | fragment of |
| | | | protein- | type VIII | staglandin | IgG receptor |
| | | | coupled | alpha 2 | dehydrogenase | IIIb; |
| | | | receptor; | chain; | 15- | NM_001271035.1) |
| | | | AF118265.1) | NM_001294347.1) | (NAD); | |
| | | | | | NM_001256307.1) | |
| | | | HES1 (hes | COLEC12 | KIT (KIT | FLJ11151 |
| | | | family bHLH | (collectin | proto- | (hypothetical |
| | | | transcription | subfamily | oncogene | protein |
| | | | factor 1; | member | receptor | FUJ11151; |
| | | | NM_005524.3) | 12; | tyrosine | BC006289.2) |
| | | | | NM_130386.2) | kinase; | |
| | | | | | NM_000222.2) | |
| | | | HIST1H1C | CTSK | LOC339524 | FPR1 (formyl |
| | | | (histone | (cathepsin | (long | peptide |
| | | | cluster 1 H1 | K; | intergenic | receptor 1; |
| | | | family | NM_000396.3) | non-protein | NM_001193306.1) |
| | | | member c; | | coding RNA | |
| | | | NM_005319.3) | | 1140; | |
| | | | | | NR_026985.1) | |
| | | | HRH4 | CXCLS (C-X- | LOH11CR2A | FPRL1 |
| | | | (histamine | C motif | (BCSC-1 | (formyl |
| | | | receptor H4; | chemokine | isoform; | peptide |
| | | | NM_001143828.1) | ligand 5; | AY366508.1) | receptor-like |
| | | | | NM_002994.4) | | receptor; |
| | | | | | | M84562.1) |
| | | | IGSF2 | CYBB | MAOB | G0S2 (G0/G1 |
| | | | (immunoglobulin | (cytochrome | (monoamine | switch 2; |
| | | | superfamily, | b-245 | oxidase B; | NM_015714.3) |
| | | | member 2; | beta chain; | NM_000898.4) | |
| | | | BC130327.1) | NM_000397.3) | | |
| | | | IL5RA | DNASE2B | MLPH | HIST1H2BC |
| | | | (interleukin 5 | (deoxyribonuclease | (melanophilin; | (histone |
| | | | receptor | 2 | NM_001042467.2) | cluster 1 H2B |
| | | | subunit | beta; | | family |
| | | | alpha; | NM_058248.1) | | member c; |
| | | | NM_001243099.1) | | | NM_003526.2) |
| | | | KBTBD11 | EMP1 | MPOidase; | HPSE |
| | | | (kelch repeat | (epithelial | (myeloperox | (heparanase; |
| | | | and BTB | membrane | NM_000250.1 | NM_001098540.2) |
| | | | domain | protein 1; | | |
| | | | containing | NM_001423.2) | | |
| | | | 11; | | | |
| | | | NM_014867.2) | | | |
| | | | KCNH2 | FDX1 | MS4A2 | IL8RA |
| | | | (potassium | (ferredoxin | (membrane | (interleukin |
| | | | voltage-gated | 1; | spanning 4- | 8 receptor |
| | | | channel, | NM_004109.4) | domains A2; | alpha; |
| | | | subfamily H | | NM_001256916.1) | L19591.1) |
| | | | (eag-related), | | | |
| | | | member 2; | | | |
| | | | NM_000238.3) | | | |
| | | | LRPSL (LDL | FN1 | NM_003293 | IL8RB |
| | | | receptor | (fibronectin | (tryptase | (interleukin- |
| | | | related | 1; | alpha/beta | 8 receptor |
| | | | protein 5 like; | NM_001306131.1) | 1; | type B; |
| | | | NM_001135772.1) | | NM_003294.3) | U11878.1) |
| | | | MYO15B | GM2A | NR0B1 | KCNJ15 |
| | | | (myosin XVB; | (GM2 | (nuclear | (potassium |
| | | | NM_001309242.1) | ganglioside | receptor | voltage- |
| | | | | activator; | subfamily 0 | gated |
| | | | | NM_000405.4) | group B | channel |
| | | | | | member 1; | subfamily J |
| | | | | | NM_000475.4) | member 15; |
| | | | | | | NM_001276438.1) |
| | | | RCOR3 (REST | GPC4 | PGDS | KIAA0329 |
| | | | corepressor | (glypican 4; | (hematopoietic | (tectonin |
| | | | 3; | NM_001448.2) | prostaglandin | beta- |
| | | | NM_001136224.2) | | D | propeller |
| | | | | | | repeat |
| | | | | | synthase; | containing 2; |
| | | | | | NM_014485.2) | NM_014844.4) |
| | | | RNASE2 | KAL1 | PPM1H | LILRB2 |
| | | | (ribonuclease | (anosmin | (protein | (leukocyte |
| | | | A family | 1; | phosphatase, | immunoglobulin |
| | | | member 2; | NM_000216.3) | Mg2+/Mn2+ | like |
| | | | NM_002934.2) | | dependent | receptor B2; |
| | | | | | 1H; | NR_103521.2) |
| | | | | | NM_020700.1) | |
| | | | RNU2 (U2 | MARCO | PRG2 | MGAM |
| | | | snRNA; | (macrophage | (proteoglycan | (maltase- |
| | | | U57614.1) | receptor | 2, proeosinophil | glucoamylase; |
| | | | | with | major basic | NM_004668.2) |
| | | | | collagenous | protein; | |
| | | | | structure; | NM_001302927.1) | |
| | | | | NM_006770.3) | | |
| | | | RRP12 | ME1 (malic | PTGS1 | MME |
| | | | (ribosomal | enzyme 1; | (prostagland | (membrane |
| | | | RNA | NM_002395.5) | in- | metalloendo |
| | | | processing 12 | | endoperoxide | peptidase; |
| | | | homolog; | | synthase 1; | NM_007289.2) |
| | | | NM_001284337.1) | | NM_000962.3) | |
| | | | SIAH1 (siah | MS4A4A | SCG2 | PDE4B |
| | | | E3 ubiquitin | (membrane | (secretogranin | (phosphodiesterase |
| | | | protein ligase | spanning | II; | 4B; |
| | | | 1; | 4-domains | NM_003469.4) | NM_001297440.1) |
| | | | NM_003031.3) | A4A; | | |
| | | | | NM_001243266.1) | | |
| | | | SMPD3 | MSR1 | SIGLEC6 | S100A12 |
| | | | (sphingomyelin | (macrophage | (sialic acid | (S100 |
| | | | phosphodiesterase | scavenger | binding lg | calcium |
| | | | 3; | receptor 1; | like lectin 6; | binding |
| | | | NM_018667.3) | NM_138716.2) | NM_198845.5) | protein A12; |
| | | | | | | NM_005621.1) |
| | | | SYNJ1 | PCOLCE2 | SLC18A2 | SIGLEC5 |
| | | | (synaptojanin | (procollagen | (solute | (sialic acid |
| | | | 1; | C- | carrier | binding lg |
| | | | NM_001160302.1) | endopeptidase | family 18 | like lectin 5; |
| | | | | enhancer | member A2; | NM_003830.3) |
| | | | | 2; | NM_003054.4) | |
| | | | | NM_013363.3 | | |
| | | | TGIF1 (TGFB | PTGDS | SLC24A3 | SLC22A4 |
| | | | induced | (prostaglandin | (solute | (solute |
| | | | factor | D2 | carrier | carrier |
| | | | homeobox 1; | synthase; | family 24 | family 22 |
| | | | NM_174886.2) | NM_000954.5) | member 3; | member 4; |
| | | | | | NM_020689.3) | NM_003059.2) |
| | | | THBS1 | RAI14 | TAL1 (T-cell | SLC25A37 |
| | | | (thrombospondin | (retinoic | acute | (solute |
| | | | 1; | acid | lymphocytic | carrier |
| | | | NM_003246.3) | induced | leukemia 1; | family 25 |
| | | | | 14; | X51990.1) | member 37; |
| | | | | NM_001145525.1) | | NM_001317812.1) |
| | | | THBS4 | SCARB2 | TPSAB1 | TNFRSF10C |
| | | | (thrombospondin | (scavenger | (tryptase | (TNF |
| | | | 4; | receptor | alpha/beta | receptor |
| | | | NM_001306213.1) | class B | 1; | superfamily |
| | | | | member 2; | NM_003294.3) | member |
| | | | | NM_001204255.1) | | 10c; |
| | | | | | | NM_003841.3) |
| | | | TIPARP | SCG5 | TPSB2 | VNN3 (vanin |
| | | | (TCDD | (secretogranin | (tryptase | 3; |
| | | | inducible | V; | beta 2; | NM_001291703.1) |
| | | | poly(ADP- | NM_001144757.2) | NM_024164.5) | |
| | | | ribose) | | | |
| | | | polymerase; | | | |
| | | | NM_001184718.1) | | | |
| | | | TKTL1 | SGMS1 | | |
| | | | (transketolase | (sphingom | | |
| | | | like 1; | yelin | | |
| | | | NM_001145934.1) | synthase 1; | | |
| | | | | NM_147156.3) | | |
| | | | | SULT1C2 | | |
| | | | | (sulfotransferase | | |
| | | | | family 1C | | |
| | | | | member 2; | | |
| | | | | NM_176825.2) | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 5
Individual Immunomarkers for use in the methods provided herein.

| | | GenBank |
|---|---|---|
| Gene Name | Abbreviation | Accession No.* |
| Programmed Death Ligand 1 | PDL1 | NM_014143 |
| programmed death ligand 2 | PDL2 | AY254343 |
| programmed cell death 1 | PDCD1 | NM_005018 |
| cytotoxic T-lymphocyte | CTLA4 | NM_005214 |
| associated protein 4 | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 6
Interferon (IFN) Genes for use in the methods provided herein.

| | | GenBank |
|---|---|---|
| Gene Name | Abbreviation | Accession No.* |
| Chemokine (C-X-C Motif) Ligand 10 | CXCL10 | NM_001565 |
| C-X-C motif chemokine ligand 9 | CXCL9 | NM_002416 |
| Interferon alpha inducible protein 27 | IFI27 | NM_001130080 |
| Interferon induced protein with | IFIT1 | NM_001548 |
| tetratricopeptide repeats 1 | | |
| interferon induced protein with | IFIT2 | NM_001547 |
| tetratricopeptide repeats 2 | | |
| interferon induced protein with | IFIT3 | NM_001549 |
| totratricopeptide repeats 3 | | |
| MX dynamin like GTPase 1 | MX1 | NM_001144925 |
| MX dynamin like GTPase 2 | MX2 | XM_005260983 |
| 2′-5′-oligoadenylate synthetase 1 | OAS1 | NM_016816 |
| 2′-5′-oligoadenylate synthetase 2 | OAS2 | NM_016817 |
| signal transducer and activator | STAT1 | NM_007315 |
| of transcription 1 | | |
| signal transducer and activator | STAT2 | NM_005419 |
| of transcription 2 | | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

TABLE 7
MHC class II genes for use in the methods provided herein.

| | | GenBank |
|---|---|---|
| Name | Abbreviation | Accession No.* |
| CD74 | Homo sapiens CD74 molecule (CD74) | NM_01025159 |
| CIITA | class II major histocompatibility | NM_001286402 |
| | complex transactivator | |
| CTSH | cathepsin H | NM_004390 |
| HLA-DMA | Homo sapiens major histocompatibility | NM_006120 |
| | complex, class II, DM alpha | |
| HLA-DPA1 | Homo sapiens major histocompatibility | NM_033554 |
| | complex, class II, DP alpha 1 | |
| HLA-DPB1 | Human MHC class II lymphocyte | M83664 |
| | antigen (HLA-DP) beta chain | |
| HLA-DQA1 | Homo sapiens major histocompatibility | NM_002122 |
| | complex, class II, DQ alpha 1 | |
| HLA-DRB1 | Homo sapiens major histocompatibility | NM_002124 |
| | complex, class II, DR beta 1 | |
| HLA-DRB5 | Homo sapiens major histocompatibility | NM_002125 |
| | complex, class II, DR beta 5 | |
| HLA-DRB6 | Homo sapiens major histocompatibility | NR_001298 |
| | complex, class II, DR beta 6 | |
| NCOA1 | Homo sapiens nuclear receptor | NM_003743 |
| | coactivator 1 | |

*Each GenBank Accession Number is a representative or exemplary GenBank Accession Number for the listed gene and is herein incorporated by reference in its entirety for all purposes. Further, each listed representative or exemplary accession number should not be construed to limit the claims to the specific accession number.

In one embodiment, upon determining a patient's HNSCC cancer subtype using any of the methods and classifier biomarkers panels or subsets thereof as provided herein alone or in combination with determining expression of one or more immune cell markers as provided herein and/or expression of HPV genes and/or the patient's nodal status, the patient is selected for treatment with or administered an immunotherapeutic agent. The immunotherapeutic agent can be a checkpoint inhibitor, monoclonal antibody, biological response modifiers, therapeutic vaccine or cellular immunotherapy.

In another embodiment, the immunotherapeutic agent is a checkpoint inhibitor. In some cases, a method for determining the likelihood of response to one or more checkpoint inhibitors is provided. In one embodiment, the checkpoint inhibitor is a PD-1/PD-L1 checkpoint inhibitor. The PD-1/PD-L1 checkpoint inhibitor can be nivolumab, pembrolizumab, atezolizumab, durvalumab, lambrolizumab, or avelumab. In one embodiment, the checkpoint inhibitor is a CTLA-4 checkpoint inhibitor. The CTLA-4 checkpoint inhibitor can be ipilimumab or tremelimumab. In one embodiment, the checkpoint inhibitor is a combination of checkpoint inhibitors such as, for example, a combination of one or more PD-1/PD-L1 checkpoint inhibitors used in combination with one or more CTLA-4 checkpoint inhibitors.

In one embodiment, the immunotherapeutic agent is a monoclonal antibody. In some cases, a method for determining the likelihood of response to one or more monoclonal antibodies is provided. The monoclonal antibody can be directed against tumor cells or directed against tumor products. The monoclonal antibody can be panitumumab, matuzumab, necitumumab, trastuzumab, amatuximab, bevacizumab, ramucirumab, bavituximab, patritumab, rilotumumab, cetuximab, immu-132, or demcizumab.

In yet another embodiment, the immunotherapeutic agent is a therapeutic vaccine. In some cases, a method for determining the likelihood of response to one or more therapeutic vaccines is provided. The therapeutic vaccine can be a peptide or tumor cell vaccine. The vaccine can target MAGE-3 antigens, NY-ESO-1 antigens, p53 antigens, survivin antigens, or MUC1 antigens. The therapeutic cancer vaccine can be GVAX (GM-CSF gene-transfected tumor cell vaccine), belagenpumatucel-L (allogeneic tumor cell vaccine made with four irradiated NSCLC cell lines modified with TGF-beta2 antisense plasmid), MAGE-A3 vaccine (composed of MAGE-A3 protein and adjuvant AS15), (1)-BLP-25 anti-MUC-1 (targets MUC-1 expressed on tumor cells), CimaVax EGF (vaccine composed of human recombinant Epidermal Growth Factor (EGF) conjugated to a carrier protein), WTI peptide vaccine (composed of four Wilms' tumor suppressor gene analogue peptides), CRS-207 (live-attenuated Listeria monocytogenes vector encoding human mesothelin), Bec2/BCG (induces anti-GD3 antibodies), GV1001 (targets the human telomerase reverse transcriptase), TG4010 (targets the MUC1 antigen), racotumomab (anti-idiotypic antibody which mimicks the NGcGM3 ganglioside that is expressed on multiple human cancers), tecemotide (liposomal BLP25; liposome-based vaccine made from tandem repeat region of MUC1) or DRibbles (a vaccine made from nine cancer antigens plus TLR adjuvants).

In one embodiment, the immunotherapeutic agent is a biological response modifier. In some cases, a method for determining the likelihood of response to one or more biological response modifiers is provided. The biological response modifier can trigger inflammation such as, for example, PF-3512676 (CpG 7909) (a toll-like receptor 9 agonist), CpG-ODN 2006 (downregulates Tregs), Bacillus Calmette-Guerin (BCG), mycobacterium vaccae (SRL172) (nonspecific immune stimulants now often tested as adjuvants). The biological response modifier can be cytokine therapy such as, for example, IL-2+tumor necrosis factor alpha (TNF-alpha) or interferon alpha (induces T-cell proliferation), interferon gamma (induces tumor cell apoptosis), or Mda-7 (IL-24) (Mda-7/IL-24 induces tumor cell apoptosis and inhibits tumor angiogenesis). The biological response modifier can be a colony-stimulating factor such as, for example granulocyte colony-stimulating factor. The biological response modifier can be a multi-modal effector such as, for example, multi-target VEGFR: thalidomide and analogues such as lenalidomide and pomalidomide, cyclophosphamide, cyclosporine, denileukin diftitox, talactoferrin, trabecetedin or all-trans-retinmoic acid.

In one embodiment, the immunotherapy is cellular immunotherapy. In some cases, a method for determining the likelihood of response to one or more cellular therapeutic agents. The cellular immunotherapeutic agent can be dendritic cells (DCs) (ex vivo generated DC-vaccines loaded with tumor antigens), T-cells (ex vivo generated lymphokine-activated killer cells; cytokine-induce killer cells; activated T-cells; gamma delta T-cells), or natural killer cells.

In some cases, specific subtypes of HNSCC have different levels of immune activation (e.g., innate immunity and/or adaptive immunity) such that subtypes with elevated or detectable immune activation (e.g., innate immunity and/or adaptive immunity) are selected for treatment with one or more immunotherapeutic agents described herein. In some cases, specific subtypes of HNSCC have high or elevated levels of immune activation. In some cases, the MS subtype of AD has elevated levels of immune activation (e.g., innate immunity and/or adaptive immunity) as compared to other HNSCC subtypes. In some cases, the HPV positive, AT-like subtype of HNSCC has elevated levels of immune activation (e.g., innate immunity and/or adaptive immunity) as compared to other HNSCC subtypes. In one embodiment, HNSCC subtypes with low levels of or no immune activation (e.g., innate immunity and/or adaptive immunity) are not selected for treatment with one or more immunotherapeutic agents described herein.

### Radiotherapy

In one embodiment, provided herein is a method for determining whether a HNSCC cancer patient is likely to respond to radiotherapy or radiation therapy by determining the subtype of HNSCC of a sample obtained from the patient and based on the HNSCC subtype, assessing whether the patient is likely to respond to radiotherapy. In another embodiment, provided herein is a method of selecting a patient suffering from HNSCC for radiotherapy by determining a HNSCC subtype of a sample from the patient and, based on the HNSCC subtype, selecting the patient for radiotherapy. The determination of the HNSCC subtype of the sample obtained from the patient can be performed using any method for molecular subtyping HNSCC known in the art. The determination of the HNSCC subtype of the sample obtained from the patient can be performed using any method for molecular subtyping HNSCC provided herein. In some embodiments, the method for HNSCC subtyping includes detecting expression levels (e.g., RNA, cDNA or DNA) of a classifier biomarker in a sample obtained from a patient suffering from or suspected of suffering from HNSCC (e.g., oral cavity SCC) set alone or in combination with one or more biomarkers of HPV and/or assessing the nodal status of the patient. The method for ascertaining the nodal status may entail use of any method known in the art for assessing nodal status or nodal metastasis. The classifier biomarker set can be a set of biomarkers from a publicly available database such as, for example, TCGA HNSCC RNASeq gene expression dataset(s) or any other dataset provided herein. In some embodiments, the detecting includes the expression levels of a plurality of or all of the classifier biomarkers of Table 1 or any other dataset provided herein at the nucleic acid level or protein level. In one embodiment, from about 1 to about 5, about 5 to about 10, from about 5 to about 15, from about 5 to about 20, from about 5 to about 25, from about 5 to about 30, from about 5 to about 35, from about 5 to about 40, from about 5 to about 45, from about 5 to about 50, from about 5 to about 55, from about 5 to about 60, from about 5 to about 65, from about 5 to about 70, from about 5 to about 75, or from about 5 to about 80 of the biomarkers in any of the HNSCC gene expression datasets provided herein, including, for example, Table 1 for an HNSCC sample are detected in a method to determine the HNSCC subtype as provided herein. In another embodiment, each of the biomarkers from any one of the HNSCC gene expression datasets provided herein, including, for example, Table 1 for an HNSCC sample are detected in a method to determine the HNSCC subtype as provided herein. In another embodiment, a plurality of the biomarkers from any one of the HNSCC gene expression datasets provided herein, including, for example, Table 1 for an HNSCC sample are detected in a method to determine the HNSCC subtype as provided herein. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise OLFML3, PCOLCE, LEPRE1, NNMT, OLFML2B, COL6A1, PHLDB1, COL6A2, CMTM3, GPX8, PTH1R, CYP2C18, GRHL3, CSTA, ELF3, SPRR3, ADH7, ALDH3A1, TMPRSS11A, KLF5, SLC9A3R1, SOX2 or any combination thereof. In some cases, the plurality of classifier biomarkers of Table 1 comprises all or a subset of the classifier biomarkers outlined in each column of Table 2. Further to the above embodiments, the HPV status can be determined by measuring one or more biomarkers of HPV as described herein. Further to the above embodiments, the nodal status of the patient can be determined. In one embodiment, a patient determined to have a mesenchymal subtype of HNSCC (e.g., oral cavity SCC) is a candidate for or administered radiation therapy regardless of the nodal status of the patient. In another embodiment, a patient determined to not have a mesenchymal subtype (i.e., non-mesenchymal) of HNSCC (e.g., oral cavity SCC) is a candidate for radiation therapy if they are determined to display nodal metastasis (e.g., their nodal status is N123). Any candidate for radiation therapy may also be a candidate for or administered an additional standard of care treatment such as, for example, chemotherapy and/or surgical intervention.

In some embodiments, the radiotherapy can include but are not limited to proton therapy and external-beam radiation therapy. In some embodiments, the radiotherapy can include any types or forms of treatment that is suitable for HNSCC patients. In some embodiments, the surgery can include laser technology, excision, lymph node dissection or neck dissection, and reconstructive surgery.

In some embodiments, an HNSCC can have or display resistance to radiotherapy. Radiotherapy resistance in any HNSCC subtype can be determined by measuring or detecting the expression levels of one or more genes known in the art and/or provided herein associated with or related to the presence of radiotherapy resistance. Genes associated with radiotherapy resistance can include NFE2L2, KEAP1 and CUL3. In some embodiments, radiotherapy resistance can be associated with the alterations of KEAP1 (Kelch-like ECH-associated protein 1)/NRF2 (nuclear factor E2-related factor 2) pathway. Association of a particular gene to radiotherapy resistance can be determined by examining expression of said gene in one or more patients known to be radiotherapy non-responders and comparing expression of said gene in one or more patients known to be radiotherapy responders. In one embodiment, the HNSCC subtype that has radiotherapy resistance can be a CL subtype. In some embodiments, the HNSCC subtype that has radiotherapy resistance can be a BA subtype. In some embodiments, the HNSCC subtype that has radiotherapy resistance can be a MS subtype. In some embodiments, the HNSCC subtype that has radiotherapy resistance can be an AT subtype. In some embodiments, the HNSCC subtype that has radiotherapy resistance can be any HNSCC subtypes. In one embodiment, the HNSCC subtype is a CL subtype. The HNSCC patient can be HPV-negative or positive. The HNSCC can be nodal positive (e.g., N123) or nodal negative (e.g., NO). In some embodiments, the methods for clinical applications as described herein can determine radiotherapy resistance for surgically respectable HPV-negative or HPV-positive HNSCC cases.

### Surgical Intervention

In one embodiment, provided herein is a method for determining whether a HNSCC cancer patient is likely to respond to surgical intervention by determining the subtype of HNSCC of a sample obtained from the patient and, based on the HNSCC subtype, assessing whether the patient is likely to respond to surgery. In another embodiment, provided herein is a method of selecting a patient suffering from HNSCC for surgery by determining a HNSCC subtype of a sample from the patient and, based on the HNSCC subtype, selecting the patient for surgery. The determination of the HNSCC subtype of the sample obtained from the patient can be performed using any method for molecular subtyping HNSCC known in the art. The determination of the HNSCC subtype of the sample obtained from the patient can be performed using any method for molecular subtyping HNSCC provided herein. In some embodiments, the method for HNSCC subtyping includes detecting expression levels (e.g., RNA, cDNA or DNA) of a classifier biomarker in a sample obtained from a patient suffering from or suspected of suffering from HNSCC (e.g., oral cavity SCC) set alone or in combination with one or more biomarkers of HPV and/or assessing the nodal status of the patient. The method for ascertaining the nodal status may entail use of any method known in the art for assessing nodal status or nodal metastasis. The classifier biomarker set can be a set of biomarkers from a publicly available database such as, for example, TCGA HNSCC RNASeq gene expression dataset(s) or any other dataset provided herein. In some embodiments, the detecting includes the expression levels of a plurality of or all of the classifier biomarkers of Table 1 or any other dataset provided herein at the nucleic acid level or protein level. In one embodiment, the plurality of classifier biomarkers from Table 1 comprises from about 1 to about 5, about 5 to about 10, from about 5 to about 15, from about 5 to about 20, from about 5 to about 25, from about 5 to about 30, from about 5 to about 35, from about 5 to about 40, from about 5 to about 45, from about 5 to about 50, from about 5 to about 55, from about 5 to about 60, from about 5 to about 65, from about 5 to about 70, from about 5 to about 75, or from about 5 to about 80 of the biomarkers in any of the HNSCC gene expression datasets provided herein, including, for example, Table 1 for an HNSCC sample are detected in a method to determine the HNSCC subtype as provided herein. In another embodiment, each of the biomarkers from any one of the HNSCC gene expression datasets provided herein, including, for example, Table 1 for an HNSCC sample are detected in a method to determine the HNSCC subtype as provided herein. In another embodiment, a plurality of the biomarkers from any one of the HNSCC gene expression datasets provided herein, including, for example, Table 1 for an HNSCC sample are detected in a method to determine the HNSCC subtype as provided herein. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise OLFML3, PCOLCE, LEPRE1, NNMT, OLFML2B, COL6A1, PHLDB1, COL6A2, CMTM3, GPX8, PTH1R, CYP2C18, GRHL3, CSTA, ELF3, SPRR3, ADH7, ALDH3A1, TMPRSS11A, KLF5, SLC9A3R1, SOX2 or any combination thereof. In some cases, the plurality of classifier biomarkers of Table 1 comprises all or a subset of the classifier biomarkers outlined in each column of Table 2. Further to the above embodiments, the HPV status can be determined by measuring one or more biomarkers of HPV as described herein.

In some embodiments, surgery approaches for use herein can include but are not limited to minimally invasive or endoscopic head and neck surgery (eHNS), Transoral Robotic Surgery (TORS), Transoral Laser Microsurgery (TLM), Endoscopic Thyroid and Neck Surgery, Robotic Thyroidectomy, Minimally Invasive Video-Assisted Thyroidectomy (MIVAT), and Endoscopic Skull Base Tumor Surgery. In some embodiments, the surgery can include any types of surgical treatment that is suitable for HNSCC patients. In one embodiment, the suitable treatment is surgery.

### Prediction of Overall Survival Rate and Metastasis for HNSCC Patients

The present disclosure provides methods for predicting overall survival rate for a HNSCC patient (e.g., OCSCC). In some embodiments, the prediction of overall survival rate can involve obtaining a head and neck tissue sample for a HNSCC patient. In some embodiments, the HNSCC patients can have various stages of cancers. some embodiments, the overall survival rate can be determined by detecting the expression level of at least one or a plurality of subtype classifiers of a publicly available head and neck cancer database or dataset. In some embodiments, an overall survival rate can be determined by detecting the expression level (e.g., protein and/or nucleic acid) of any subtype classifiers that are relevant to HNSCC. In one embodiment, the subtype classifiers can be all or a subset of classifiers from Table 1. The method can further entail determining the HPV status of the HNSCC patient. The HNSCC patient or subject can be HPV-negative. The method can further entail determining the nodal status of the HNSCC patient. Nodal status can be determined as provided herein. The HNSCC patient or subject can be nodal-negative or nodal-positive.

In some embodiments, the present disclosure further provide methods of predicting overall survival in HNSCC from specific areas of the head and neck such as, for example, the oral cavity (i.e., oral cavity squamous cell carcinoma (OCSCC)). In some embodiments, the prediction includes detecting an expression level of at least one gene or a plurality of genes from an HNSCC dataset (e.g., Table 1) in a head and neck tissue sample (e.g., sample from oral cavity) obtained from a patient. In some embodiments, the detection of the expression level of a subtype classifier or a plurality of subtype classifiers from an HNSCC dataset (e.g., Table 1) using the methods provided herein specifically identifies a BA, MS, AT or CL OCSCC subtype. In some embodiments, the identification of the OCSCC subtype is indicative of the overall survival in the patient. A mesenchymal subtype of OCSCC as ascertained by measuring one or more subtype classifiers, such as, for example, from Table 1 in a sample obtained from an OCSCC patient as provided herein can indicate a poor overall survival of an OCSCC patient as compared to patients with other subtypes of OCSCC. The poor overall survival of a patient with a MS subtype of HNSCC (e.g., OCSCC) can be regardless of the patient's nodal status.

The present disclosure provides methods for predicting nodal metastasis for a HNSCC patient. In some embodiments, the prediction of nodal metastasis can involve obtaining a head and neck tissue sample for a HNSCC patient. In some embodiments, the HNSCC patients can have various stages of cancers. In some embodiments, the nodal metastasis can be determined by detecting the expression level of at least one subtype classifier from a head and neck gene set. The head and neck gene set can be a publicly available head and neck database. The publicly available head and neck gene set can be the TCGA HNSCC gene set.

### Detection Methods

In one embodiment, the methods and compositions provided herein allow for the detection of at least one nucleic acid in a HNSCC sample obtained from a subject. The at least one nucleic acid can be a classifier biomarker provided herein. In one embodiment, the at least one nucleic acid detected using the methods and compositions provided herein are selected from Table 1 alone or in combination with assessing the nodal status of the subject. In one embodiment, the methods of detecting the nucleic acid(s) (e.g., classifier biomarkers) in the HNSCC sample obtained from the subject comprises, consists essentially of, or consists of measuring the expression level of at least one or a plurality of biomarkers using any of the methods provided herein. The biomarkers can be selected from Table 1. In some cases, the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1. In some cases, the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1. In some cases, the plurality of classifier biomarkers of Table 1 comprise OLFML3, PCOLCE, LEPRE1, NNMT, OLFML2B, COL6A1, PHLDB1, COL6A2, CMTM3, GPX8, PTH1R, CYP2C18, GRHL3, CSTA, ELF3, SPRR3, ADH7, ALDH3AI, TMPRSS11A, KLF5, SLC9A3RI, SOX2 or any combination thereof. In some cases, the plurality of classifier biomarkers of Table 1 comprises all or a subset of the classifier biomarkers outlined in each column of Table 2. The detection can be at the nucleic acid level. The detection can be by using any amplification, hybridization and/or sequencing assay disclosed herein.

In another embodiment, the methods and compositions provided herein allow for the detection of at least one nucleic acid or a plurality of nucleic acids in a head and neck cancer sample (e.g. HNSCC sample) obtained from a subject such that the at least one nucleic acid is or the plurality of nucleic acids are selected from the biomarkers listed in Table 1 alone or in combination with the detection of at least one biomarker from a set of biomarkers whose presence, absence and/or level of expression is indicative of immune activation. The set of biomarkers for indicating immune activation can be gene expression signatures of and/or Adaptive Immune Cells (AIC) (e.g., Table 4A) and/or Innate Immune Cells (IIC) (e.g., Table 4B), individual immune biomarkers (e.g., Table 5), interferon genes (e.g., Table 6), major histocompatibility complex, class II (MHC II) genes (e.g., Table 7) or a combination thereof. The gene expression signatures of both IIC and AIC can be any gene signatures known in the art such as, for example, the gene signature listed in Bindea et al. (Immunity 2013; 39(4); 782-795). The detection can be at the nucleic acid level. The detection can be by using any amplification, hybridization and/or sequencing assay disclosed herein.

### Kits

Kits for practicing the methods of the invention can be further provided. By “kit” can encompass any manufacture (e.g., a package or a container) comprising at least one reagent, e.g., an antibody, a nucleic acid probe or primer, etc., for specifically detecting the expression of a biomarker of the invention. The kit may be promoted, distributed, or sold as a unit for performing the methods of the present invention. Additionally, the kits may contain a package insert describing the kit and methods for its use.

In one embodiment, kits for practicing the methods of the invention are provided. Such kits are compatible with both manual and automated immunocytochemistry techniques (e.g., cell staining). These kits comprise at least one antibody directed to a biomarker of interest, chemicals for the detection of antibody binding to the biomarker, a counterstain, and, optionally, a bluing agent to facilitate identification of positive staining cells. Any chemicals that detect antigen-antibody binding may be used in the practice of the invention. The kits may comprise at least 2, at least 3, at least 4, at least 5, at least 6, at least 7, at least 8, at least 9, at least 10, or more antibodies for use in the methods of the invention.

In one embodiment, kits for practicing the methods of the invention are provided. Such kits are compatible with both manual and automated gene sequencing or hybridization techniques. These kits comprise at least one probe or primer pair directed to a biomarker of interest and means for detecting the amplification of, sequencing of or hybridization to said biomarker of interest. The kits may comprise at least 2, at least 3, at least 4, at least 5, at least 6, at least 7, at least 8, at least 9, at least 10, or more probes or primer pairs for use in the methods of the invention.

### EXAMPLES

The present invention is further illustrated by reference to the following Examples. However, it should be noted that these Examples, like the embodiments described above, are illustrative and are not to be construed as restricting the scope of the invention in any way.

### Example 1—Development and Validation of the Head and Neck Squamous Cell Carcinoma (HNSCC) Subtyping Signature

### BACKGROUND

Head and neck squamous cell carcinoma (HNSCC), including cancers of the oral cavity, oropharynx, nasopharynx, hypopharynx, and larynx, is one of the most common cancers worldwide¹. In the United States, it is estimated that there were approximately 66,000 new cases and 14,00 deaths in 20211. The majority of HNSCC are associated with heavy tobacco and alcohol use, although over the last thirty years there has been an increase in the incidence of human papillomavirus (HPV)-related cancer, primarily in the oropharynx. While the treatment of HNSCC depends on multiple tumor and patient-related factors, the three main modalities used in the management of HNSCC are surgical resection, radiation therapy, and chemotherapy. Patients with early-stage tumors are generally treated with a single modality therapy while those with advanced stage tumors often require multiple modalities. Oncologic outcomes in HNSCC are driven largely by stage at presentation: The 5-year overall survival for Stage I-II and III-IV HNSCC is approximately 70-90% and 40-60%, respectively.

While the majority of early stage HNSCC cases may be curable with surgical or radiation-based therapies, it is notable that 10-30% of HPV-negative HNSCC cases without pathologically aggressive features can still experience a relapse event². Oral cavity squamous cell carcinoma (OCSCC) is the most common head and neck cancer, comprising ⅓ of all cases, with the vast majority of OCSCC cases being HPV-negative and associated with tobacco use. Dependent on clinical staging, OCSCC treatment can involve surgical excision of the primary tumor with or without neck dissection, followed by radiation with or without chemotherapy. Cancers arising from the larynx and hypopharynx are also almost exclusively tobacco-associated and HPV-negative. Primary radiation-based treatments are common for early and intermediate stage cancers of the larynx and hypopharynx to preserve function, with surgical resection often reserved for locally advanced tumors or salvage after failed radiation therapy. Oropharyngeal squamous cell carcinoma (OPSCC) includes cancers arising from the tonsils, base of tongue, soft palate and lateral and posterior pharyngeal walls. While traditionally associated with heavy smoking and alcohol consumption, it is estimated that approximately 60-70% of incident OPSCC cases may be attributable to human papillomavirus (HPV)³⁻⁵. Treatment for OPSCC usually includes radiation+/−chemotherapy, although novel treatment paradigms including minimally invasive surgery have been investigated. In contrast to excellent oncologic outcomes associated with HPV-positive OPSCC, HPV-negative OPSCC can be associated with high recurrence rates and mortality⁶⁻⁸

There have been significant advances in our understanding of the molecular biology of HNSCC and of the genomic heterogeneity across tumors. Based on earlier work in lung cancer⁹, four mRNA expression patterns (classical, atypical, basal, and mesenchymal) have been described that demonstrate unique genomic features and prognostic significance^{10,11}. These HNSCC subtypes show varied biology and may be helpful in prognostic assessments complementing other risk stratification based on HPV status, stage, anatomic site, and other characteristics^{10,11}. The basal subtype is characterized by over-expression of genes functioning in cell adhesion including COL17A1, and growth factor and receptor TGFA and EGFR¹¹. The mesenchymal subtype displayed over-expression of genes involved in immune response^{12,13} and is characterized by expression of genes associated with epithelial to mesenchymal transition including VIM, DES, TWIST1, and HGF¹¹. It has been suggested previously that epithelial to mesenchymal transition pathways may be important in the initiation of nodal metastasis^{9,11,14}. The classical subtype is characterized by over-expression of genes related to oxidative stress response and xenobiotic metabolism and is most strongly associated with tobacco exposure¹⁵⁻¹⁸. The atypical subtype, which includes both HPV and non-HPV tumors, is characterized by elevated expression of CDKN2A, LIG1, and RPA2. The atypical subtype was also associated with low EGFR expression¹¹. These four gene-expression based head and neck cancer subtypes have been validated in other studies, including in The Cancer Genome Atlas (TCGA) head and neck cancer cohort^{10,11}. Head and Neck Squamous Cell Carcinoma (HNSCC) is comprised of cancers arising from the oral cavity, oropharynx, nasopharynx, hypopharynx, and larynx are responsible for approximately 3% of all malignancies (NCI HNSCC www.cancer.gov/types/head-and-neck/hp accessed Jun. 7, 2017).

### Objective

In this study, the potential clinical utility of gene expression subtyping in HNSCC was examined, with an emphasis on evaluating this biomarker among early-stage HPV-negative cancers. The findings were expected to provide further evidence to support the clinical utility of gene expression subtyping in HNSCC within the context of clinical site, stage, and treatment as well as introduce the potential for predictive applications of gene expression subtyping analysis in HPV-negative HNSCC.

### Methods

### Patients and Data

The study was conducted in accordance with the Declaration of Helsinki and the International Conference on Harmonization Good Clinical Practice guidelines and was approved by Institutional Review Boards of Washington University in St. Louis (IRB #201706088) and the University of Tennessee Health Science Center (IRB #17-05549-XP).

### Datasets

Two datasets were identified in the public record: 1) TCGA HNSCC (n=520)¹² and 2) large institutional cohort (n=42)¹⁹. For statistical analyses, cases were considered if they had clinical parameters of N stage and overall survival. Model fits and analyses used all available patients having complete relevant data. TCGA data were sourced from the Broad Institute Genome Data Commons (GDC)²⁰ and the institutional cohort was obtained from the gene expression omnibus (GEO)²¹ (GSE41116).

### Gene Expression Analysis

For TOGA, upper quantile normalized RNA-seq expression values by expected maximization (RSEM)²² was downloaded from GDC (gdac.broadinstitute.org/, accessed Dec. 4, 2015) and log 2-transformed. All samples were assigned a molecular subtype using the centroid classification method as previously reported^{35, 36}, describing each sample as belonging to one of four molecular subtypes (basal, mesenchymal, atypical or classical).

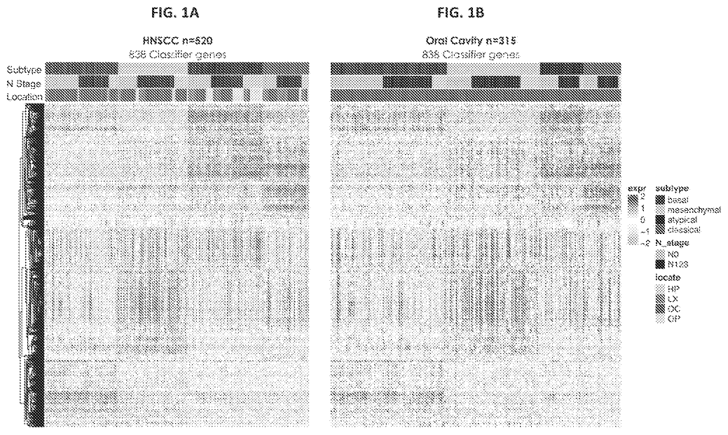

Briefly, the gold standard HNSCC centroid predictor is a vector-based algorithm based on the median gene expression of a set of 838 feature genes selected to distinguish the four molecular subtypes¹¹. By calculating distance (1−pearson correlation coefficient) between each sample and each centroid, the algorithm determines the class to which a sample obtained from an HNSCC patient is most similar based on the predictor gene set. Each sample is then uniquely assigned to the class for which the distance was shortest. For the purpose of developing a more parsimonious and potentially clinically relevant predictor, a reduced gene centroid predictor was developed in this example. To do this, a 5-fold cross-validation (CV) on the entire TCGA HNSCC dataset (n=520) as well as the ClaNC software package³⁵ was used to identify the number of genes required to provide strong separation of the subtypes and sufficient agreement with the previously developed gold standard (i.e., aforementioned ˜840-gene classifier) as shown in FIG. 1. Candidates for the reduced set were all genes in the gold standard classifier and an additional set of genes (348) chosen for high observed mean and variance in the entire data set. Here, the standard ClaNC approach was modified by requiring an equal proportion of high and low genes per subtype (i.e., select an equal number of negatively and positively correlated genes for each HNSCC subtype) in the final model rather than selecting genes based on extreme absolute values of the ClaNC t-statistic. Additionally, calculation of the coefficients in the final nearest centroid classifier excluded samples with low gold standard classifier call strength (20% per subtype were excluded), where call strength was the commonly used silhouette score, and the coefficients themselves were within-subtype gene medians after each gene had been centered by its overall median. Heat maps displaying expression profile patterns by subtype calls were generated using the Complexheatmap package in R.

### Clinical Data and Statistical Analysis

Paired clinic data was obtained from GDC (gdac.broadinstitute.org/accessed Dec. 4, 2015)²⁰. To account for limitations in the median follow up, and for the purposes of comparison to prior work, all survival times longer than 36 months were truncated and censored at 36 months. In general, clinical parameters were represented as presented in downloaded clinical datasets. HPV positivity was assessed by RNAseq evaluation of HPV aligned sequences in HPV types 16, 18, 33, and 35 at levels >1000 counts. HPV reference sequence data was based on the PaVe website: pave.niaid.nih.gov/. Read Counts>1000 for HPV RNAseq (TCGA) or HPV E6 gene expression⁴ were used as the criterion for ongoing HPV replication and an HPV positive tumor designation. Other parameters of interest included gender, age, smoking, T stage, N stage, and overall stage. Associations between two categorical variables were evaluated using Fisher's exact test and the chi-square test. Associations between categorical variables and continuous variables were evaluated using the Kruskal-Wallis test. Kaplan-Meier plots and the logrank test were used to assess univariate associations between survival and study parameters. Cox models were used to check associations with adjustment for potential confounders. The R survival package was used for all statistical analysis.

### Results

### Clinical and Molecular Groups

The clinical characteristics of 418 patients were first considered from the TCGA dataset meeting our eligibility criteria in order to understand the generalizability of the results obtained to the greater population of head and neck cancer patients. The median age of the TCGA was 60 years, which is only slightly younger than that reported in the American population for this disease, 63 years¹. Twenty five percent of patients were female compared to 27% of patients in the overall American population of HNSCC. Seventy eight percent of patients in the dataset admitted to some degree of smoking, which is consistent with prior reports (www.cancer.net/cancer-types/head-and-neck-cancer/risk-factors-and-prevention) Eighty one percent of patients presented with at least stage III disease, consistent with most datasets studied by molecular profiling. Consistent with head and neck cancer disease course, only 1 patient was known to have metastatic disease at presentation, although this data field was missing for more than half of patients. Larger and more advanced tumors were more amenable to providing tissue for molecular profiling. Nonetheless, nearly 20% of patients presented with stage I and II tumors, offering a unique opportunity to assess risk profiles in early-stage patients. Somewhat unexpectedly, only 63% of patients had a record of radiation in the dataset, a percentage that seems low considering that most of the 81% of advanced stage patients might be expected to receive radiation as part of a multidisciplinary treatment of their cancer. Considering subgroups, it was noted that 71% of node-positive oral cavity patients reported radiation versus 85% of node-positive non-oral cavity. Forty three percent of node-negative oral cavity patients were radiated, compared to 50% of node-negative non-oral cavity. Whether the low percentage represents underutilization of the standard of care based on patient-specific factors or under-reporting of radiation in the database was not known. That said, the trends were as expected in which the highest rates of radiation were found in node-positive non-OC patients, for whom nearly all patients would have a recommendation for radiation based on NCCN guidelines, either as part of concurrent chemoradiation or surgery followed by radiation. The lowest rates were for node-negative oral cavity patients, many of whom could be treated with single modality surgery, again according to NCCN guidelines.

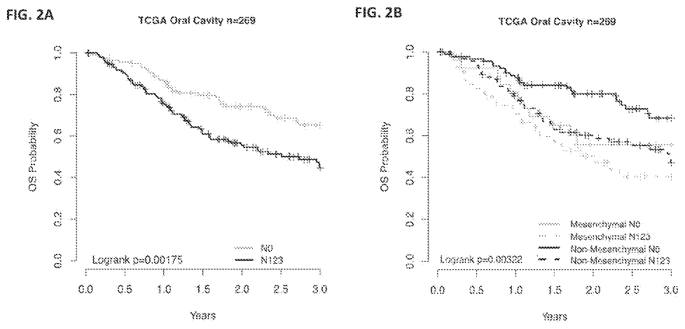

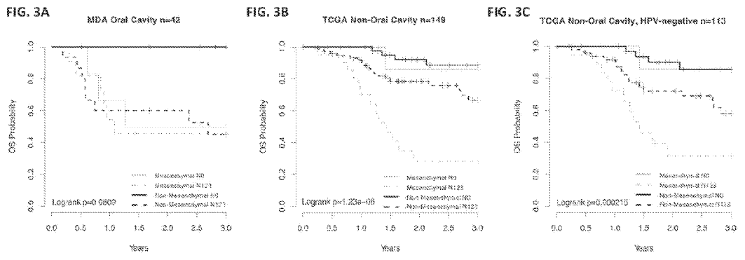

The molecular aspect of the cohort was then interrogated for the distribution of molecular subtypes as a function of anatomic site. Molecular subtypes were determined by applying the centroid subtype predictor described herein (i.e., Table 1) to all samples. For the 418 TCGA samples meeting eligibility criteria for the current study, the distribution of molecular subtypes was nearly identical to that of the original TCGA HNSCC report of 279 cases published in 2015: 30% basal, 26% mesenchymal, 18% classical and 26% atypical versus 31% basal, 27% mesenchymal, 16% classical and 26% atypical in the 2015 publication¹² (FIG. 1A and Table 8). As previously reported by multiple authors, there is a strong association of subtype with anatomic site. Consistent with other reports, an enrichment was observed in oral cavity tumors for the mesenchymal and basal subtypes, atypical samples in the oropharynx primarily and to a lesser extent larynx, and classical subtype in the larynx. In an unexpected finding, although patients with lymph node involvement is observed in all molecular and anatomic sites as expected, a statistically significant association with lymph node positivity in the mesenchymal tumors was observed. A significant association was found between subtype (mesenchymal versus other) and overall survival in OCSCC, which had previously been reported in smaller cohorts (i.e., a statistically significant association for overall survival and mesenchymal subtype) 10-12,19. This finding was again observed in this cohort Table 9, FIG. 2B and FIG. 3. Since nodal status is a component of overall cancer stage, itself defining of patient prognosis, lymph node involvement might be considered either to be confounding for the worse prognosis for mesenchymal tumors, or even more interestingly, be in the causal pathway of poor prognosis.

TABLE 8
Descriptive Statistics of Clinical and Demographic Variable by Molecular Subtype (n = 418
Oral Cavity

| | | | | | | | |
|---|---|---|---|---|---|---|---|
| | | Non- | | | | Non- | |
| Mesenchymal | | Mesenchymal | P- | Mesenchymal | | Mesenchymal | P- |
| (n = 26) | N0 | (n = 93) | Value¹ | (n = 53) | N+ | (n = 97) | Value² |

| | | | | | | |
|---|---|---|---|---|---|---|
| Location | | | | | | |
| Oral | 26(100%) | 93(100%) | | 53(100%) | 97(100%) | |
| Cavity | | | | | | |
| Larynx | | | | | | |
| Oropharynx | | | | | | |
| Hypopharnyx | | | | | | |
| Age | | | | | | |
| (years) | | | | | | |
| Median | 66 | 62 | | 64 | 58 | 0.014 |
| Gender | | | | | | |
| F | 9(35%) | 36(39%) | | 15(28%) | 22(23%) | |
| M | 17(65%) | 57(61%) | | 38(72%) | 75(77%) | |
| Smoker | | | | | | |
| (ever) | | | | | | |
| Yes | 16(67% | 61(66%) | | 41(80%) | 73(77%) | |
| No | 8(33%) | 31(34%) | | 10(20%) | 22(23%) | |
| HPV | | | | | | |
| Status | | | | | | |
| Positive | 1(4%) | 7(8%) | | 5(9%) | 14(14%) | |
| Negative | 25(96%) | 86(92%) | | 48(91%) | 83(86%) | |
| Radiation | | | | | | |
| Yes | 10(40%) | 40(45%) | | 30(62%) | 65(76%) | |
| No | 15(60%) | 49(55%) | | 18(38%) | 20(24%) | |
| T stage | | | | | | |
| T1 | 3(12%) | 16(17%) | 0.034 | 3(6%) | 5(5%) | |
| T2 | 15(58%) | 24(26%) | | 14(26%) | 26(27%) | |
| T3 | 3(12%) | 18(19%) | | 14(26%) | 23(24%) | |
| T4 | 5(19%) | 35(38%) | | 22(42%) | 43(44%) | |
| N stage | | | | | | |
| N0 | 26(100%) | 93(100%) | | 0(0%) | 0(0%) | |
| N1 | 0(0%) | 0(0%) | | 16(30%) | 31(32%) | |
| N2 | 0(0%) | 0(0%) | | 36(68%) | 64(66%) | |
| N3 | 0(0%) | 0(0%) | | 1(2%) | 2(2%) | |
| M stage | | | | | | |
| M0 | 9(100%) | 40(100%) | | 22(100%) | 42(100%) | |
| M1 | 0(0%) | 0(0%) | | 0(0%) | 1(4%) | |
| Overall | | | | | | |
| stage | | | | | | |
| I | 3(12%) | 15(16%) | 0.037 | 0(0%) | 0(0%) | |
| II | 15(58%) | 24(26%) | | 0(0%) | 0(0%) | |
| III | 3(12%) | 18(20%) | | 11(21%) | 23(24%) | |
| IV | 5(19%) | 35(38%) | | 42(79%) | 73(76%) | |
| Subtype | | | | | | |
| Basal | | 57(61%) | | | 52(54%) | |
| Mesenchymal | 26(100%) | | | 53(100%) | | |
| Atypical | | 20(22%) | | | 23(24%) | |
| Classical | | 16(17%) | | | 22(23%) | |

TABLE 8
Descriptive Statistics of Clinical and Demographic Variable by Molecular Subtype (n = 418)

| Non-Oral Cavity | | | | | | | | |
|---|---|---|---|---|---|---|---|---|
| | | Non- | | | | Non- | | All |
| Mesenchymal | | Mesenchymal | P- | Mesenchymal | | Mesenchymal | P- | Samples |
| (n = 10) | N0 | (n = 46) | Value³ | (n = 20) | N+ | (n = 73) | Value⁴ | Total |

| | | | | | |
|---|---|---|---|---|---|
| Location | | | | | |
| Oral | | | | | 269 |
| Cavity | | | | | |
| Larynx | 6 (60%) | 34 (74%) | 13 (65%) | 42 (58%) | 95 |
| Oropharynx | 3 (30%) | 12 (26%) | 4 (20%) | 28 (38%) | 47 |
| Hypopharnyx | 1 (10%) | 0 (0%) | 3 (15%) | 3 (4%) | 7 |
| Age | | | | | |
| (years) | | | | | |
| Median | 61.5 | 60.5 | 60.5 | 59 | 60 |
| Gender | | | | | |
| F | 1(10%) | 9 (20%) | 2(10%) | 13(18%) | 107 |
| M | 9(90%) | 37(80%) | 18(90%) | 60(82%) | 311 |
| Smoker | | | | | |
| (ever) | | | | | |
| Yes | 9(90%) | 41(91%) | 15(79%) | 65(89%) | 321 |
| No | 1(10%) | 4(9%) | 4(21%) | 8(11%) | 88 |
| HPV | | | | | |
| Status | | | | | |
| Positive | 1(10%) | 9(20%) | 2(10%) | 24(33%) | 63 |
| Negative | 9(90%) | 37(80%) | 18(90%) | 49(67%) | 355 |
| Radiation | | | | | |
| Yes | 5(56%) | 19(49%) | 11(73%) | 57(88%) | 237 |
| No | 4(44%) | 20(51%) | 4(27%) | 8(12%) | 138 |
| T stage | | | | | |
| T1 | 3(30%) | 4(9%) | 1(5%) | 7(10%) | 42 |
| T2 | 1(10%) | 12(27%) | 4(20%) | 16(22%) | 112 |
| T3 | 2(20%) | 7(16%) | 3(15%) | 25(35%) | 95 |
| T4 | 4(40%) | 22(49%) | 12(60%) | 24(33%) | 167 |
| N stage | | | | | |
| N0 | 10(100%) | 46(100%) | 0(0%) | 0(0%) | 175 |
| N1 | 0(0%) | 0(0%) | 5(25%) | 15(21%) | 67 |
| N2 | 0(0%) | 0(0%) | 14(70%) | 54(74%) | 168 |
| N3 | 0(0%) | 0(0%) | 1(5%) | 4(5%) | 8 |
| M stage | | | | | |
| M0 | 8(100%) | 24(100%) | 9(100%) | 24(96%) | 178 |
| M1 | 0(0%) | 0(0%) | 0(0%) | 1(4%) | 1 |
| Overall | | | | | |
| stage | | | | | |
| I | 3(30%) | 3(7%) | 0(0%) | 0(0%) | 24 |
| II | 1(10%) | 13(29%) | 0(0%) | 1(1%) | 54 |
| III | 2(20%) | 7(16%) | 2(10%) | 11(15%) | 77 |
| IV | 4(40%) | 22(49%) | 18(90%) | 59(83%) | 258 |
| Subtype | | | | | |
| Basal | | 9(20%) | | 6(8%) | 124 |
| Mesenchymal | 10(100%) | | 20(100%) | | 109 |
| Atypical | | 26(57%) | | 40(55%) | 109 |
| Classical | | 11(24%) | | 27(37%) | 76 |

Statistical comparisons:
¹Oral Cavity, N0, Mesenchymal vs. Oral Cavity, N0, Non-Mesenchymal;
²Oral cavity, N+, Mesenchymal vs. Oral Cavity, N+, Non-Mesenchymal;
³Non-Oral Cavity, N0, Mesenchymal vs. Non-Oral Cavity, N0, Non-Mesenchymal;
⁴Non-Oral Cavity, N+, Mesenchymal vs. Non-Oral Cavity, N+, Non-Mesenchymal.
Some variables do not sum to total due to missing data.

TABLE 9
Univariate and multivariate survival analysis within Oral Cavity N0 and N+ subgroups.

| | | Univariate | | | Multivariate | | |
|---|---|---|---|---|---|---|---|
| | | | | P- | | | P- |
| Reference | n | HR | CI | Value | HR | CI | Value |

Oral Cavity N0

| | | | | | | | | |
|---|---|---|---|---|---|---|---|---|
| Gender | Female | 119 | 0.98 | (0.49, 1.99) | 0.96 | | | |
| Smoker | No | 116 | 0.78 | (0.38, 1.59 | 0.49 | | | |
| HPV | Positive | 119 | 1.24 | (0.3, 5.17) | 0.77 | | | |
| Status | | | | | | | | |
| Radiation | Yes | 114 | 1.94 | (0.92, 4.1) | 0.082 | | | |
| T stage | 1, 2 | 119 | 2.3 | (1.12, 4.72) | 0.023 | 2.77 | (1.32, 5.82) | 0.0072 |
| Overall | I, II | 118 | 2.48 | (1.18, 5.21) | 0.017 | | | |
| stage | | | | | | | | |
| Subtype | Non- | 119 | 1.83 | (0.89, 3.76) | 0.099 | 2.4 | (1.14, 5.06) | 0.021 |
| | mesenchymal | | | | | | | |
| Age | 19-61 | 119 | 0.95 | (0.48, 1.88) | 0.88 | | | |

Oral Cavity N+

| | | | | | | | | |
|---|---|---|---|---|---|---|---|---|
| Gender | Female | 150 | 1.06 | (0.61, 1.84) | 0.83 | | | |
| Smoker | No | 146 | 2.09 | (1.03, 4.22) | 0.041 | | | |
| HPV | Positive | 150 | 1 | (0.49, 2.01) | 0.99 | | | |
| Status | | | | | | | | |
| Radiation | Yes | 133 | 2.12 | (1.25, 3.6) | 0.0053 | | | |
| T stage | 1, 2 | 150 | 2.33 | (1.25, 4.34) | 0.008 | | | |
| Overall | I, II | 149 | 2.44 | (1.17, 5.11) | 0.018 | | | |
| stage | | | | | | | | |
| Subtype | Non- | 150 | 1.36 | (0.84, 2.21) | 0.22 | 1.3 | (0.79, 2.14) | 0.3 |
| | mesenchymal | | | | | | | |
| Age | 19-61 | 150 | 1.3 | (0.81, 2.1) | 0.28 | 1.24 | (0.76, 2.01) | 0.39 |

HR = hazard ratio, CI = confidence

### Oral Cavity Cohort:

For the purposes of defining a cohort in which questions of clinical management and prognosis might be more explicitly considered, patients with oral cavity squamous cancers were isolated, a group generally treated by a more explicitly clinical pathway (FIG. 1B). In general, patients with oral cavity cancer are treated primarily with surgery in all cases where a tumor is expected to be resected with negative margins. Early-stage patients, such as stage I and II can be managed with surgery only or surgery plus adjuvant radiation. Patients with more advanced tumors generally receive surgery followed by radiation or concurrent chemoradiation. Patients with oral cavity cancer were primarily basal, n=109 (41%) and mesenchymal n=79 (29%), with minority contributions of atypical n=43 (16%) and classical n=38(14%). Overall, 67% (53 of 79) of the mesenchymal patients were node-positive versus 48% (52 of 109) of basal, 53% atypical (23 of 43), and 58% (22 of 38) of classical. A finding of higher probability of node involvement, would be consistent with higher clinical stage and the overall worse prognosis observed for mesenchymal patients in multiple datasets. Interestingly, although the mesenchymal patients were overall of higher nodal status, the association with T stage was less clear. Among node-positive OCSCC patients, mesenchymal and non-mesenchymal patients had nearly identical nodal stage distribution of N1, N2, and N3. By contrast, among node-negative patients, 69 (18 of 26) % of mesenchymal patients were T1 or T2, whereas only 43% (40 of 93) of non-mesenchymal patients were TI or T2. Only 19% (5 of 26) of node-negative mesenchymal patients were T4 compared to 38% (35 of 93) of non-mesenchymal patients. Of T1-T2 mesenchymal tumors, 49% (17 of 35) were node-positive, compared to 44% (31 of 71) of T1-T2 non-mesenchymal patients. By contrast, of mesenchymal T3-T4 tumors, 82% (36 of 44) were node-positive compared to 55% (66 of 119) of non-mesenchymal T3-T4 tumors. Summarizing the associations for OC patients, mesenchymal patients are both more likely to develop nodal metastasis, and they are more likely to do this at earlier T stage. At higher T stage, mesenchymal patients were much more likely to be node-positive.

Given the association between three clinical prognostic factors (anatomic site, T and N stage) with a validated molecular marker (mesenchymal subtype) both stratified and multivariate models of prognosis were considered to investigate potential subgroups as well as the independent contribution of each factor (Table 9). As expected, it was demonstrated that clinical outcomes differ as a function of anatomic site, T stage, and N stage. Substrata as a function of nodal status was then considered, observing a striking finding in which those patients who were node-negative mesenchymal subtype demonstrated identical risk of death to patients who were node-positive mesenchymal or node-positive non-mesenchymal (HR=2.4, p=0.021). In other words, mesenchymal molecular subtype conveyed all the risk of positive nodes whether nodes were clinically present or not. Among node-positive mesenchymal patients, the added risk of mesenchymal subtype was no longer observed (HR=1.3, p=0.3). Given the unexpected nature of this finding, independent datasets of oral cavity cancer were sought for the purposes of validation, noting perhaps the largest being a set of well-characterized tumors from MD Anderson. Quite strikingly, the results were nearly identical, with patients of the non-mesenchymal OC group showing overall excellent survival and node-negative mesenchymal patients, node-positive mesenchymal patients, and node-positive non-mesenchymal patients all with similarly poor survival (FIGS. 3A-3C).

The remaining mesenchymal patients from non-OC sites were then considered, of which there were only 30 out of 418 TCGA patients, divided roughly equally between larynx and oropharynx. The non-OC mesenchymal patients were ⅓ node-negative and ⅔ node-positive. Unlike OC patients, the node-negative patients did extremely well overall. Although the sample number was only 10 patients, there was no suggestion that non-OC mesenchymal patients did worse than non-mesenchymal patients in node-negative state. By contrast, node-positive mesenchymal patients did terribly overall compared to node-positive non-OC patients. We then excluded HPV (+) patients, showing that the results were somewhat attenuated, but mesenchymal patients still fared worse than non-mesenchymal patients.

### Role of Radiation:

Possible explanations for the poor survival experienced by mesenchymal patients in some strata but not others was then considered. The overall inferior survival of patients with EMT signature, the most prominent component of the mesenchymal has been suggested by many prior reports²³⁻²⁶. Mesenchymal tumors are characterized by EMT programs of gene expression, as well as inflammatory signatures that might be associated with worse outcomes. However, such programs might not be expected to have differential outcomes with respect to node-positive versus node-negative disease. One possible explanation would be differential treatment, especially radiation. In node-negative patients, radiation was administered at overall similar rates between mesenchymal and non-mesenchymal patients, 40% and 45% respectively, suggesting that differential radiation usage alone would not explain differential outcomes. The role of chemotherapy in node-negative patients would be in conjunction with radiation and would only be limited to patients with positive margins, and as such, differential chemotherapy usages is also an unlikely explanation of differential outcome. As expected, radiation usage is more common in node-positive patients. Among node-positive patients, mesenchymal patients were radiated at slightly lower rates overall, 62% versus 76%. Similarly, in the non-OC sites (larynx and oropharynx), node-negative mesenchymal and non-mesenchymal patients were radiated at somewhat higher rates, 56% and 49% respectively, consistent with higher rates of radiation-based treatment of these disease sites. Node-positive non-OC mesenchymal and non-OC, non-mesenchymal patients were radiated at the highest rates of 73% and 88%, respectively, likely a combination of primary chemoradiation and adjuvant radiation cases. Although speculative, it is at least possible that part of the difference between patient groups might be due to the use of radiation, in which the poor prognosis of early-stage OC mesenchymal patients can be at least somewhat attenuated in higher stage compared to non-mesenchymal patients when they are radiated. This would argue that increased radiation of node-negative OC mesenchymal patients might be beneficial.

### Nodal Predictor

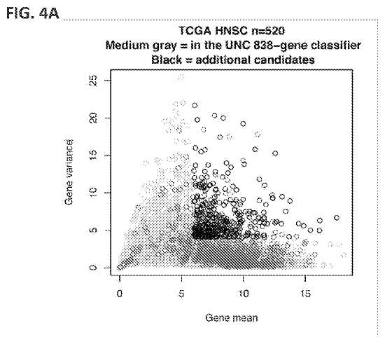

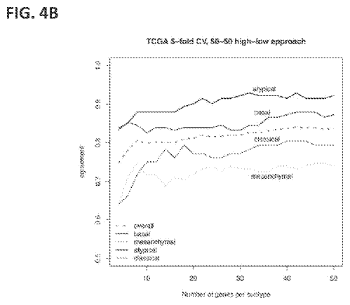

The possibility that the risk conveyed by Mesenchymal subtype, might simply be replaced in risk models by lymph node status alone was then considered. Controlling for node status did not eliminate the independent risk of the Mesenchymal subtype. Interestingly, correlations between both T stage and N stage and molecular subtype was observed, suggesting that molecular subtype was likely in the causal pathway for risk conveyed by these canonical stratification variables. Mesenchymal tumors overall appeared to be smaller than non-Mesenchymal tumors (45% (44%) of total versus 37% of total) and despite this, demonstrate much higher fraction of node positive tumors (68% (67%) versus 51%). To investigate this further, a predictor of lymph node metastasis was constructed similar to those which have been reported multiple times²⁷. The predictor performed reasonably well with an accuracy of around 63% (FIGS. 4A-4B), although modestly lower than the most favorable prior reports in the low 70% accuracy range. However, when considering the false positives and negatives of the nodal predictor as a function of gene expression subtypes, a remarkable pattern was observed. Of 54 mesenchymal samples, 53 were predicted to be node positive, including 18 that were pathologically negative, representing an overall accuracy of 65%. These 18 patients were at increased risk for recurrence and death as described in the prior figures. In other words, the predictor of nodal spread was inherent to the gene expression pattern of the mesenchymal subtype. In parallel, it was observed that tumors of the Basal subtype were much more likely to be predicted as node negative, including a sizable fraction of patients who were clinically node positive, documenting an overall accuracy of only 58%. Importantly, the Basal subjects who were node positive by pathologic results and predicted to be node negative by molecular profiling, still retained their risk of negative outcomes. For the remaining cases classified (Classical and Atypical), the predictor had an accuracy of 77%. In summary, it was documented that lymph node status can be predicted using gene expression but demonstrate for the first time that errors are differential as a function of molecular subtypes, which include gene expression patterns previously associated with lymph node metastasis. Importantly, the misclassifications made by the nodal classifier appear to have clinical relevance. Specifically, Mesenchymal subtypes that are pathologically node negative, but predicted to be node positive convey the risk of node positivity. Basal tumors predicted to be node negative do not demonstrate lower risk. The prediction of Mesenchymal subtype appears to be the preferred method for identification of risk in early-stage tumors, rather than through the use of gene expression subtypes to predict node status DISCUSSION

This study confirms several important previous findings regarding gene expression subtype in OCSCC and HNSCC more broadly. First, the mesenchymal and basal subtypes were demonstrated to comprise the majority of OCSCC cases. Second, this example confirms previous reports suggesting that the mesenchymal subtype is associated with worse outcomes across all HNSCC sites. Importantly, novel and more nuanced associations were identified between gene expression subtypes, stage, site, and treatment with important implications for future treatment stratification. Remarkably, it was demonstrated that the mesenchymal subtype is associated with poor survival even in the setting of early-stage, node-negative OCSCC treated with surgical resection. In contrast, the data presented herein demonstrates that mesenchymal subtype cases have favorable outcomes compared to other gene expression subtypes in early stage, non-OCSCC cases, the majority of which were treated with definitive radiation therapy. These findings highlight the potential value of gone expression subtyping as an adjunct to pathology for treatment decision-making.

Gene expression subtypes provide an objective method of molecular classification of HNSCC based on unsupervised clustering and are reflective of important differences in tumor biology. The four gene expression subtypes in HNSCC have been validated in multiple datasets, and similar classifications have been developed for lung cancer^{9-11,28}. In the present study, differences in gene expression subtype distribution by anatomic site were demonstrated. As previously reported, OCSCC is comprised primarily of mesenchymal and basal subtypes, while classical is the predominant subtype in LSCC. It has been previously demonstrated the prognostic value of the mesenchymal subtype in HNSCC¹⁹. The mesenchymal subtype is associated with epithelial to mesenchymal transition (EMT) and predisposes to increased tumor invasiveness and lymph node metastases^{11,14,24,25}. Recently, the partial EMT signature has shown further evidence of the importance of a mesenchymal phenotype in OCSCC, suggesting that the transition from epithelial to mesenchymal phenotype represents a spectrum.

The present study provides a more refined examination of the prognostic value of gene expression subtype in HNSCC that is specific to early-stage HNSCC. While the mesenchymal subtype is prognostic of worse survival in early-stage OCSCC, there is no significant difference in outcomes between mesenchymal and other subtypes in non-OCSCC early-stage tumors. Therapeutic decision-making and treatment dilemmas in HNSCC are anatomic site specific, and our data suggest that the potential clinical application of molecular subtyping should be considered within this context. These data also highlight the potential predictive application of gene expression subtype analysis in HNSCC. OCSCC is generally treated surgically, with adjuvant radiation and chemotherapy reserved for advanced stage tumors or adverse pathologic features such as positive margins and extra-nodal extension. Yet there is a subset of OCSCC patients that recur even with early-stage disease and in the absence of adverse pathologic features². It has been previously shown that the mesenchymal subtype is associated with an increased risk of occult nodal metastasis in the setting of clinically node-negative disease and suggest that a neck dissection should be considered even if other clinicopathologic criteria are not met²⁹. The present study suggests that patients with early-stage, node-negative OCSCC and mesenchymal subtype tumors could potentially benefit from treatment escalation through the addition of adjuvant radiation therapy, even when traditional pathologic criteria are not met. The argument for escalation with radiation therapy is strengthened when one considers that non-OCSCC early-stage tumors treated primarily with definitive radiation therapy showed no difference in survival by subtype. Taken together, the data suggest that the addition of radiation therapy may attenuate the poor prognosis associated with early-stage mesenchymal subtype tumors.

The data presented herein challenges existing paradigms that a mesenchymal phenotype characterized by EMT is associated with resistance to radiation therapy³⁰. Ionizing radiation therapy has also been shown to paradoxically induce a mesenchymal phenotype in multiple in vitro models. However, clinical support for this hypothesis is lacking in the literature and more specifically for head and neck cancer. In fact, among early-stage, non-OCSCC cases treated primarily with radiation therapy, the data presented herein demonstrated that the mesenchymal subtype had equal or more favorable outcomes compared to the other subtypes. The association between radiation resistance and EMT is based in vitro studies showing activation of multiple pathways but fail to account for the role of radiation has on the tumor microenvironment. EMT and mesenchymal tumors in general have been shown to be highly immunosuppressed and with high expression of PD-L1. We hypothesize that in HNSCC, radiation therapy serves as important immunomodulatory function that allows cells to overcome the immune evasion mechanisms associated with EMT and the mesenchymal subtype³¹. Radiation promotes immunogenic cell death through tumor antigen release, increased trafficking of immune cells, and ultimately results in improved outcomes in these otherwise immunosuppressed tumors^{32,33}.

Furthermore, it has been demonstrated at mesenchymal tumors have high expression of immune checkpoint targets including PD-L1, and that EMT phenotype may in fact be used to determine candidacy for immune checkpoint inhibition³⁴.

Predicting recurrence or relapse events in early-stage, HPV-negative HNSCC remains a significant challenge for clinicians. Despite significant improvements in our understanding of HNSCC molecular biology and prognostication, there is a paucity of biomarkers that have been developed to address this specific issue. Our data suggest that a gene expression classifier applied to early stage HNSCC could potentially be used to assist in therapeutic decision-making.

### INCORPORATION BY REFERENCE

The following references are incorporated by reference in their entireties for all purposes.
1. American Cancer Society. Cancer Facts and Figures 2021. Atlanta: American Cancer Society.
2. Amin M B, Greene F L, Edge S B, et al. The Eighth Edition AJCC Cancer Staging Manual: Continuing to build a bridge from a population-based to a more “personalized” approach to cancer staging. C A Cancer J Clin. 2017; 67(2):93-99.
3. Ang K K, Harris J, Wheeler R, et al. Human papillomavirus and survival of patients with oropharyngeal cancer. N Engl J Med. 2010; 363(1):24-35.
4. Chaturvedi A K, Engels E A, Pfeiffer R M, et al. Human papillomavirus and rising oropharyngeal cancer incidence in the United States. J Clin Oncol. 2011; 29(32):4294-4301.
5. Haughey B H, Sinha P. Prognostic factors and survival unique to surgically treated p16+ oropharyngeal cancer. Laryngoscope. 2012; 122 Suppl 2: S13-33.
6. Fakhry C, Westra W H, Li S, et al. Improved survival of patients with human papillomavirus-positive head and neck squamous cell carcinoma in a prospective clinical trial. J Natl Cancer Inst. 2008; 100(4):261-269.
7. Gillison M L, D'Souza G, Westra W, et al. Distinct risk factor profiles for human papillomavirus type 16-positive and human papillomavirus type 16-negative head and neck cancers. J Natl Cancer Inst. 2008; 100(6):407-420.
8. Wu Y, Posner M R, Schumaker L M, et al. Novel biomarker panel predicts prognosis in human papillomavirus-negative oropharyngeal cancer: an analysis of the TAX 324 trial. Cancer. 2012; 118(7):1811-1817.
9. Wilkerson M D, Yin X, Hoadley K A, et al. Lung squamous cell carcinoma mRNA expression subtypes are reproducible, clinically important, and correspond to normal cell types. Clin Cancer Res. 2010; 16(19):4864-4875.
10. Chung C H, Parker J S, Karaca G, et al. Molecular classification of head and neck squamous cell carcinomas using patterns of gene expression. Cancer Cell. 2004; 5(5):489-500.
11. Walter V, Yin X, Wilkerson M D, et al. Molecular subtypes in head and neck cancer exhibit distinct patterns of chromosomal gain and loss of canonical cancer genes. PLOS One. 2013; 8(2):e56823.
12. Cancer Genome Atlas N. Comprehensive genomic characterization of head and neck squamous cell carcinomas. Nature. 2015; 517(7536):576-582.
13. Keck M K, Zuo Z, Khattri A, et al. Integrative analysis of head and neck cancer identifies two biologically distinct HPV and three non-HPV subtypes. Clin Cancer Res. 2015; 21(4):870-881.
14. De Cecco L, Nicolau M, Giannoccaro M, et al. Head and neck cancer subtypes with biological and clinical relevance: Meta-analysis of gene-expression data. Oncotarget. 2015; 6(11):9627-9642.
15. Bao J, Li J, Li D, Li Z. Correlation between expression of NF-E2-related factor 2 and progression of gastric cancer. Int J Clin Exp Med. 2015; 8(8):13235-13242.
16. Bao L J, Jaramillo M C, Zhang Z B, et al. Nrf2 induces cisplatin resistance through activation of autophagy in ovarian carcinoma. Int J Clin Exp Pathol. 2014; 7(4):1502-1513.
17. Kawasaki Y, Ishigami S, Arigami T, et al. Clinicopathological significance of nuclear factor (erythroid-2)-related factor 2 (Nrf2) expression in gastric cancer. BMC Cancer. 2015; 15:5.
18. Kawasaki Y, Okumura H, Uchikado Y, et al. Nrf2 is useful for predicting the effect of chemoradiation therapy on esophageal squamous cell carcinoma. Ann Surg Oncol. 2014; 21(7):2347-2352.
19. Pickering C R, Zhang J, Yoo S Y, et al. Integrative genomic characterization of oral squamous cell carcinoma identifies frequent somatic drivers. Cancer Discov. 2013; 3(7):770-781.
20. Broad Institute TCGA Genome Data Analysis Center (2015). Head and Neck Squamous Carcinoma (HNSC). Broad Institute of MIT and Harvard doi:107908/C1571BB1.
21. Edgar R, Domrachev M, Lash A E. Gene Expression Omnibus: NCBI gene expression and hybridization array data repository. Nucleic Acids Res. 2002; 30(1):207-210.
22. Li B, Dewey C N. RSEM: accurate transcript quantification from RNA-Seq data with or without a reference genome. BMC Bioinformatics. 2011; 12:323.
23. da Silva S D, Morand G B, Alobaid F A, et al. Epithelial-mesenchymal transition (EMT) markers have prognostic impact in multiple primary oral squamous cell carcinoma. Clin Exp Metastasis. 2015; 32(1):55-63.
24. Jung A R, Jung C H, Noh J K, Lee Y C, Eun Y G. Epithelial-mesenchymal transition gene signature is associated with prognosis and tumor microenvironment in head and neck squamous cell carcinoma. Sci Rep. 2020; 10(1):3652.
25. van der Heijden M, Essers P B M, Verhagen C V M, et al. Epithelial-to-mesenchymal transition is a prognostic marker for patient outcome in advanced stage HNSCC patients treated with chemoradiotherapy. Radiother Oncol. 2020; 147:186-194.
26. Wangmo C, Charoen N, Jantharapattana K, Dechapbunkul A, Thongsuksai P. Epithelial-Mesenchymal Transition Predicts Survival in Oral Squamous Cell Carcinoma. Pathol Oncol Res. 2020; 26(3):1511-1518.
27. van Hooff S R, Leusink F K, Roepman P, et al. Validation of a gene expression signature for assessment of lymph node metastasis in oral squamous cell carcinoma. J Clin Oncol. 2012; 30(33):4104-4110.
28. Hayes D N, Monti S, Parmigiani G, et al. Gene expression profiling reveals reproducible human lung adenocarcinoma subtypes in multiple independent patient cohorts. J Clin Oncol. 2006; 24(31):5079-5090.
29. Zevallos J P, Mazul A L, Walter V, Hayes D N. Gene Expression Subtype Predicts Nodal Metastasis and Survival in Human Papillomavirus-Negative Head and Neck Cancer. Laryngoscope. 2019; 129(1):154-161.
30. Zhou S, Zhang M, Zhou C, Wang W, Yang H, Ye W. The role of epithelial-mesenchymal transition in regulating radioresistance. Crit Rev Oncol Hematol. 2020; 150:102961.
31. Jiang Y, Zhan H. Communication between EMT and PD-L1 signaling: New insights into tumor immune evasion. Cancer Lett. 2020; 468:72-81.
32. Menon H, Ramapriyan R, Cushman T R, et al. Role of Radiation Therapy in Modulation of the Tumor Stroma and Microenvironment. Front Immunol. 2019; 10:193.
33. Portella L, Scala S. Ionizing radiation effects on the tumor microenvironment. Semin Oncol. 2019; 46(3):254-260.
34. Mak M P, Tong P, Diao L, et al. A Patient-Derived, Pan-Cancer EMT Signature Identifies Global Molecular Alterations and Immune Target Enrichment Following Epithelial-to-Mesenchymal Transition. Clin Cancer Res. 2016; 22(3):609-620.
35. Dabney A R. ClaNC: Point-and-click software for classifying microarrays to nearest centroids. Bioinformatics. 2006; 22:122-123. doi:10.1093/bioinformatics/bti756
36. Dabney A R. Classification of microarrays to nearest centroids.

### Bioinformatics. 2005; 21:4148-4154.

### Further Numbered Embodiments of the Disclosure

Other subject matter contemplated by the present disclosure is set out in the following numbered embodiments:
1. A method for determining a head and neck squamous cell carcinoma (HNSCC) subtype of a sample obtained from a subject suffering from or suspected of suffering from HNSCC, the method comprising detecting an expression level of a plurality of classifier biomarkers selected from Table 1, wherein the detection of the expression level of the plurality of the classifier biomarkers specifically identifies a basal (BA), mesenchymal (MS), atypical (AT) or classical (CL) HNSCC subtype.
2. The method of embodiment 1, wherein the method further comprises comparing the detected levels of expression of the plurality of classifier biomarkers selected from Table 1 to the expression of the plurality of classifier biomarkers selected from Table 1 in at least one sample training set(s), wherein the at least one sample training set comprises expression data of the plurality of classifier biomarkers selected from Table 1 from a reference HNSCC BA sample, expression data of the plurality of classifier biomarkers selected from Table 1 from a reference HNSCC MS sample, expression data of the plurality of classifier biomarkers selected from Table 1 from a reference HNSCC AT sample, expression data of the plurality of classifier biomarkers selected from Table 1 from a reference HNSCC CL sample or a combination thereof; and classifying the sample as BA, MS, AT or CL subtype based on the results of the comparing step.
3. The method of embodiment 2, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.
4. The method of any one of the above embodiments, wherein the expression level of the plurality of classifier biomarkers selected from Table 1 is detected at the nucleic acid level.
5. The method of embodiment 4, wherein the nucleic acid level is RNA or cDNA.
6. The method embodiment 4, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.
7. The method of embodiment 6, wherein the expression level is detected by performing qRT-PCR.
8. The method of embodiment 7, wherein the detection of the expression level comprises using at least one pair of oligonucleotide primers specific for each classifier biomarker from the plurality of classifier biomarkers selected from Table 1.
9. The method of embodiment 1, wherein the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the subject.
10. The method of embodiment 9, wherein the bodily fluid is blood or fractions thereof, urine, saliva, or sputum.
11. The method of any one of the above embodiments, wherein the plurality of classifier biomarkers selected from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
12. The method of any one of embodiments 1-10, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
13. The method of any one of embodiments 1-10, wherein the plurality of classifier biomarkers selected from Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
14. The method of any one of embodiments 1-10, wherein the plurality of classifier biomarkers selected from Table 1 comprises all the classifier biomarkers from Table 1.
15. The method of any one of the above embodiments, further comprising determining the nodal status of the subject suffering from or suspected of suffering from HNSCC.
16. The method of any one of the above embodiments, wherein the HNSCC is oral cavity HNSCC.
17. A method for determining a head and neck squamous cell carcinoma (HNSCC) subtype of a sample obtained from a subject suffering from or suspected of suffering from HNSCC comprising detecting an expression level of a plurality of nucleic acid molecules that each encode a classifier biomarker having a specific expression pattern in head and neck cancer cells, wherein the plurality of classifier biomarkers are selected from the classifier biomarkers in Table 1, the method comprising: (α) isolating nucleic acid material from a sample from a subject suffering from or suspected of suffering from HNSCC; (b) mixing the nucleic acid material with a plurality of oligonucleotides, wherein the plurality of oligonucleotides comprises at least one oligonucleotide that is substantially complementary to a portion of each nucleic acid molecule from the plurality of the classifier biomarkers; and (c) detecting expression of the plurality of classifier biomarkers, wherein the HNSCC subtype is selected from a basal (BA), mesenchymal (MS), atypical (AT) or classical (CL) subtype.
18. The method of embodiment 17, wherein the method further comprises comparing the detected levels of expression of the plurality of classifier biomarkers from Table 1 to the expression of the plurality of classifier biomarkers from Table 1 in at least one sample training set(s), wherein the at least one sample training set comprises expression data of the plurality of classifier biomarkers from Table 1 from a reference HNSCC BA sample, expression data of the plurality of classifier biomarkers from Table 1 from a reference HNSCC MS sample, expression data of the plurality of classifier biomarkers from Table 1 from a reference HNSCC AT sample, expression data of the plurality of classifier biomarkers from Table 1 from a reference HNSCC CL sample or a combination thereof; and classifying the sample as BA, MS, AT or CL subtype based on the results of the comparing step.
19. The method of embodiment 18, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.
20. The method of any of embodiments 17-19, wherein the detecting the expression level comprises performing qRT-PCR or any hybridization-based gene assays.
21. The method of embodiment 20, wherein the expression level is detected by performing qRT-PCR.
22. The method of embodiment 21, wherein the detection of the expression level comprises using at least one pair of oligonucleotide primers specific for each nucleic acid molecule from the plurality of the classifier biomarker from Table 1.
23. The method of any one of embodiments 17-22, further comprising determining the nodal status of the subject suffering from or suspected of suffering from HNSCC.
24. The method of any one of embodiments 17-23, further comprising predicting the response to a therapy for treating a subtype of HNSCC based on the detected expression level of the classifier biomarker.
25. The method of embodiment 24, wherein the subtype is mesenchymal, and the therapy is radiation therapy.
26. The method of embodiment 25, wherein the nodal status is node negative.
27. The method of any one of embodiments 17-26, wherein the sample is a formalin-fixed, paraffin-embedded (FFPE) sample from the head and neck area of the subject, fresh or a frozen tissue sample from the head and neck area of the subject, an exosome, wash fluids, cell pellets or a bodily fluid obtained from the subject.
28. The method of embodiment 27, wherein the bodily fluid is blood or fractions thereof, urine, saliva, or sputum.
29. The method of any one of embodiments 17-28, wherein the plurality of classifier biomarkers selected from Table 1 comprises at least two classifier biomarkers, at least S classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
30. The method of any one of embodiments 17-28, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
31. The method of any one of embodiments 17-28, wherein the plurality of classifier biomarkers selected from the classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
32. The method of any one of embodiments 17-28, wherein the plurality of classifier biomarkers selected from the classifier biomarkers of Table 1 comprises all the classifier biomarkers from Table 1.
33. The method of any one of embodiments 17-32, wherein the HNSCC is oral cavity HNSCC.
34. A method of detecting a biomarker in a sample obtained from a subject suffering from or suspected of suffering from HNSCC, the method comprising, consisting essentially of or consisting of measuring the expression level of a plurality of biomarker nucleic acids selected from Table 1 using an amplification, hybridization and/or sequencing assay.
35. The method of embodiment 34, wherein the sample was previously diagnosed as being squamous cell carcinoma.
36. The method of embodiment 35, wherein the previous diagnosis was by histological examination.
37. The method of any one of embodiments 34-36, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.
38. The method of embodiment 37, wherein the expression level is detected by performing qRT-PCR.
39. The method of any one of embodiments 34-38, wherein the detection of the expression level comprises using at least one pair of oligonucleotide primers per each of the plurality of biomarker nucleic acids selected from Table 1.
40. The method of any one of embodiments 34-39, wherein the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the patient.
41. The method of embodiment 40, wherein the bodily fluid is blood or fractions thereof, urine, saliva, or sputum,
42. The method of any one of embodiments 34-41, wherein the plurality of classifier biomarkers from Table 1 comprises, consists essentially of, or consists of at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
43. The method of any one of embodiments 34-41, wherein the plurality of classifier biomarkers from Table 1 comprises, consists essentially of, or consists of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
44. The method of any one of embodiments 34-41, wherein the plurality of classifier biomarkers from Table 1 comprises, consists essentially of, or consists of olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
45. The method of any one of embodiments 34-41 wherein the plurality of classifier biomarkers from Table 1 comprises, consists essentially of, or consists of all the classifier biomarkers from Table 1.
46. The method of any one of embodiments 34-45, wherein the HNSCC is oral cavity HNSCC.
47. A method of determining whether a patient suffering from or suspected of suffering from HNSCC is likely to respond to radiation therapy, the method comprising, determining the HNSCC subtype of a sample obtained from the patient, wherein the HNSCC subtype is selected from the group consisting of basal, mesenchymal, atypical and classical; and
   based on the subtype, assessing whether the patient is likely to respond to radiation therapy.
48. The method of embodiment 47, further comprising determining the nodal status of the patient suffering from or suspected of suffering from HNSCC.
49. The method of embodiment 47 or 48, wherein the patient is assessed as likely to respond to radiation therapy if the HNSCC subtype is determined to be mesenchymal, regardless of nodal status of the patient.
50. The method of embodiment 47 or 48, wherein the patient is assessed as likely to respond to radiation therapy if the HNSCC subtype is determined to be basal, atypical or classical and nodal status of the patient is determined to be N123.
51. A method for selecting a patient suffering from or suspected of suffering from HNSCC for radiation therapy, the method comprising, determining a HNSCC subtype of a sample obtained from the patient, based on the subtype; and selecting the patient for radiation therapy, wherein the HNSCC subtype is selected from the group consisting of basal, mesenchymal, atypical and classical.
52. The method of embodiment 51, further comprising determining the nodal status of the patient suffering from or suspected of suffering from HNSCC.
53. The method of embodiment 51 or 52, wherein the patient is selected for radiation therapy if the HNSCC subtype is determined to be mesenchymal, regardless of nodal status of the patient.
54. The method of embodiment 51 or 52, wherein the patient is selected for radiation therapy if the HNSCC subtype is determined to be basal, atypical or classical and nodal status of the patient is determined to be N123.
55. The method of any one of embodiments 47-54, wherein the HNSCC is oral cavity HNSCC.
56. The method of any one of embodiments 47-55, wherein the radiation therapy is used in combination with surgery and/or chemotherapy.
57. The method of any one of embodiments 47-56, wherein the sample is a formalin-fixed, paraffin-embedded (FFPE) sample obtained from the head and neck area of the patient, fresh or a frozen tissue sample obtained from the head and neck area of the patient, an exosome, or a bodily fluid obtained from the patient.
58. The method of embodiment 57, wherein the bodily fluid is blood or fractions thereof, urine, saliva, or sputum.
59. The method of any one of embodiments 47-58, wherein the patient is initially determined to have HNSCC via a histological analysis of a sample.
60. The method of any one of embodiments 47-59, wherein the patient's HNSCC subtype is determined via a histological analysis of a sample obtained from the patient.
61. The method of any one of embodiments 47-59, wherein the determining the HNSCC subtype comprises determining expression levels of a plurality of classifier biomarkers.
62. The method of embodiment 61, wherein the determining the expression levels of the plurality of classifier biomarkers is at a nucleic acid level by performing RNA sequencing, reverse transcriptase polymerase chain reaction (RT-PCR) or hybridization-based analyses.
63. The method of embodiment 61 or 62, wherein the plurality of classifier biomarkers for determining the HNSCC subtype is selected from a publicly available HNSCC dataset.
64. The method of embodiment 63, wherein the publicly available HNSCC dataset is TCGA HNSCC RNAseq dataset.
65. The method of embodiment 61 or 62, wherein the plurality of classifier biomarkers for determining the HNSCC subtype is selected from Table 1.
66. The method of embodiment 65, wherein the RT-PCR is quantitative real time reverse transcriptase polymerase chain reaction (qRT-PCR).
67. The method of embodiment 66, wherein the RT-PCR is performed with primers specific to each of the plurality of classifier biomarkers from Table 1.
68. The method of any one of embodiments 65-67, further comprising comparing the detected levels of expression of the plurality of classifier biomarkers from Table 1 to the levels of expression of the plurality of classifier biomarkers from Table 1 in at least one sample training set(s), wherein the at least one sample training set comprises expression level data of the plurality of classifier biomarkers from Table 1 from a reference HNSCC BA sample, expression level data of the plurality of classifier biomarkers from Table 1 from a reference HNSCC MS sample, expression level data of the plurality of classifier biomarkers from Table 1 from a reference HNSCC AT sample, expression level data of the plurality of classifier biomarkers from Table 1 from a reference HNSCC CL sample or a combination thereof; and classifying the sample obtained from the patient as BA, MS, AT or CL based on the results of the comparing step.
69. The method of embodiment 68, wherein the comparing step comprises applying a statistical algorithm which comprises determining a correlation between the expression data obtained from the sample obtained from the patient and the expression data from the at least one training set(s); and classifying the sample obtained from the patient as a BA, MS, AT or CL subtype based on the results of the statistical algorithm.
70. The method of any one of embodiments 65-69, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
71. The method of any one of embodiments 65-69, wherein the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
72. The method of any one of embodiments 65-69, wherein the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
73. The method of any one of embodiments 65-69, wherein the plurality of the classifier biomarkers comprise all of the classifier biomarkers from Table 1.
74. A method of treating HNSCC in a subject, the method comprising:
   determining a subtype of HNSCC of a subject suffering from HNSCC by measuring a nucleic acid expression level of a plurality of classifier biomarkers in a sample obtained from a subject suffering from or suspected of suffering from HNSCC, wherein the plurality of classifier biomarkers is selected from Table 1, wherein the nucleic acid expression level of the plurality of classifier biomarkers indicates the HNSCC subtype of the subject as being basal (BA), mesenchymal (MS), atypical (AT) or classical (CL); and
   administering radiation therapy to the subject based on the subtype of the HNSCC.
75. The method of embodiment 74, wherein the HNSCC is oral cavity HNSCC.
76. The method of embodiment 74 or 75, wherein the radiation therapy is administered to the subject if the HNSCC subtype is determined to be mesenchymal, regardless of nodal status of the subject.
77. The method of embodiment 74 or 75, wherein the radiation therapy is administered to the subject if the HNSCC subtype is determined to be basal, classical or atypical and nodal status of the subject is N123.
78. The method of any one of embodiments 74-77, wherein the radiation therapy is used in combination with surgery and/or chemotherapy.
79. The method of any one of embodiments 74-78, wherein the determining step further comprises comparing the nucleic acid expression levels of the plurality of classifier biomarkers from Table 1 to the nucleic acid expression levels of the plurality of classifier biomarkers from Table 1 in at least one sample training set(s), wherein the at least one sample training set comprises nucleic acid expression level data of the plurality of classifier biomarkers from Table 1 from a reference HNSCC BA sample, nucleic acid expression level data of the plurality of classifier biomarkers from Table 1 from a reference HNSCC MS sample, nucleic acid expression level data of the plurality of classifier biomarkers from Table 1 from a reference HNSCC AT sample, nucleic acid expression level data of the plurality of classifier biomarkers from Table 1 from a reference HNSCC CL sample or a combination thereof; and classifying the sample obtained from the subject as BA, MS, AT or CL based on the results of the comparing step.
80. The method of embodiment 79, wherein the comparing step comprises applying a statistical algorithm which comprises determining a correlation between the expression data obtained from the sample obtained from the patient and the expression data from the at least one training set(s); and classifying the sample obtained from the subject as a BA, MS, AT or CL subtype based on the results of the statistical algorithm.
81. The method of any one of embodiments 74-80, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
82. The method of any one of embodiments 74-80, wherein the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
83. The method of any one of embodiments 74-80, wherein the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
84. The method of any one of embodiments 74-80, wherein the plurality of the classifier biomarkers comprise all of the classifier biomarkers from Table 1.
85. The method of any one of embodiments 74-84, wherein the measuring the nucleic acid expression level is conducted using an amplification, hybridization and/or sequencing assay.
86. The method of embodiment 85, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.
87. The method of embodiment 86, wherein the expression level is detected by performing qRT-PCR.
88. The method of any one of embodiments 74-87, wherein the sample is a formalin-fixed, paraffin-embedded (FFPE) sample obtained from the head and neck area of the subject, fresh or a frozen tissue sample obtained from the head and neck area of the subject, an exosome, wash fluids, cell pellets, or a bodily fluid obtained from the patient.
89. The method of embodiment 88, wherein the bodily fluid is blood or fractions thereof, urine, saliva, or sputum.
90. A system for determining a head and neck squamous cell carcinoma (HNSCC) subtype of a sample obtained from a subject suffering from HNSCC, the system comprising:
(a) one or more processors; and
(b) one or more memories operatively coupled to at least one of the one or more processors and having instructions stored thereon that, when executed by at least one of the one or more processors, cause the system to
   (i) detect an expression level of each of a plurality of classifier biomarkers from Table 1;
   (ii) compare the expression levels of each of the plurality of classifier biomarkers from Table 1 to the expression levels of each of the plurality of classifier biomarkers from Table 1 in a control; and
   (iii) classifying the sample as a basal (BA), mesenchymal (MS), atypical (AT) or classical (CL) HNSCC subtype based on the results of the comparing step.
91. The system of embodiment 90, wherein the control comprises at least one sample training set(s), wherein the at least one sample training set comprises expression levels of each of the plurality of classifier biomarkers from Table 1 from a reference HNSCC BA sample, expression levels of each of the plurality of classifier biomarkers from Table 1 from a reference HNSCC MS sample, expression levels of each of the plurality of classifier biomarkers from Table 1 from a reference HNSCC AT sample, expression levels of each of the plurality of classifier biomarkers from Table 1 from a reference HNSCC CL sample or a combination thereof.
92. The system of embodiment 91, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.
93. The system of any one of embodiments 90-92, wherein the expression level of each of the plurality of classifier biomarkers from Table 1 is detected at the nucleic acid level.
94. The system of embodiment 93, wherein the nucleic acid level is RNA or cDNA.
95. The system of any one of embodiments 90-94, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.
96. The system of embodiment 95, wherein the expression level is detected by performing qRT-PCR.
97. The system of embodiment 95 or 96, wherein the detecting the expression level is performed using a device that is part of the system or in communication with at least one of the one or more processors, wherein upon receipt of instructions sent by the at least one of the one or more processors, perform the detection of the expression levels.
98. The system of any one of embodiments 90-97, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.
99. The system of any one of embodiments 90-97, wherein the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.
100. The system of any one of embodiments 90-97, wherein the plurality of classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.
101. The system of any one of embodiments 90-97, wherein the plurality of classifier biomarkers of Table 1 comprises all the classifier biomarkers from Table 1.
102. The system of any one of embodiments 90-101, further comprising determining the nodal status of the subject suffering from or suspected of suffering from HNSCC.
103. The system of any one of embodiments 90-102, wherein the HNSCC is oral cavity HNSCC.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method for determining a head and neck squamous cell carcinoma (HNSCC) subtype of a sample obtained from a subject suffering from or suspected of suffering from HNSCC, the method comprising detecting an expression level of a plurality of classifier biomarkers selected from Table 1, wherein the detection of the expression level of the plurality of the classifier biomarkers specifically identifies a basal (BA), mesenchymal (MS), atypical (AT) or classical (CL) HNSCC subtype.

2. The method of claim 1, wherein the method further comprises comparing the detected levels of expression of the plurality of classifier biomarkers selected from Table 1 to the expression of the plurality of classifier biomarkers selected from Table 1 in at least one sample training set(s), wherein the at least one sample training set comprises expression data of the plurality of classifier biomarkers selected from Table 1 from a reference HNSCC BA sample, expression data of the plurality of classifier biomarkers selected from Table 1 from a reference HNSCC MS sample, expression data of the plurality of classifier biomarkers selected from Table 1 from a reference HNSCC AT sample, expression data of the plurality of classifier biomarkers selected from Table 1 from a reference HNSCC CL sample or a combination thereof; and classifying the sample as BA, MS, AT or CL subtype based on the results of the comparing step.

3. The method of claim 2, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.

4. The method of claim 1, wherein the expression level of the plurality of classifier biomarkers selected from Table 1 is detected at the nucleic acid level.

5. The method of claim 4, wherein the nucleic acid level is RNA or cDNA.

6. The method claim 4, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

7. The method of claim 6, wherein the expression level is detected by performing qRT-PCR.

8. The method of claim 7, wherein the detection of the expression level comprises using at least one pair of oligonucleotide primers specific for each classifier biomarker from the plurality of classifier biomarkers selected from Table 1.

9. The method of claim 1, wherein the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the subject.

10. The method of claim 9, wherein the bodily fluid is blood or fractions thereof, urine, saliva, or sputum.

11. The method of claim 1, wherein the plurality of classifier biomarkers selected from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

12. The method of claim 1, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

13. The method of claim 1, wherein the plurality of classifier biomarkers selected from Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

14. The method of claim 1, wherein the plurality of classifier biomarkers selected from Table 1 comprises all the classifier biomarkers from Table 1.

15. The method of claim 1, further comprising determining the nodal status of the subject suffering from or suspected of suffering from HNSCC.

16. The method of claim 1, wherein the HNSCC is oral cavity HNSCC.

17. A method for determining a head and neck squamous cell carcinoma (HNSCC) subtype of a sample obtained from a subject suffering from or suspected of suffering from HNSCC comprising detecting an expression level of a plurality of nucleic acid molecules that each encode a classifier biomarker having a specific expression pattern in head and neck cancer cells, wherein the plurality of classifier biomarkers are selected from the classifier biomarkers in Table 1, the method comprising: (a) isolating nucleic acid material from a sample from a subject suffering from or suspected of suffering from HNSCC; (b) mixing the nucleic acid material with a plurality of oligonucleotides, wherein the plurality of oligonucleotides comprises at least one oligonucleotide that is substantially complementary to a portion of each nucleic acid molecule from the plurality of the classifier biomarkers; and (c) detecting expression of the plurality of classifier biomarkers, wherein the HNSCC subtype is selected from a basal (BA), mesenchymal (MS), atypical (AT) or classical (CL) subtype.

18. The method of claim 17, wherein the method further comprises comparing the detected levels of expression of the plurality of classifier biomarkers from Table 1 to the expression of the plurality of classifier biomarkers from Table 1 in at least one sample training set(s), wherein the at least one sample training set comprises expression data of the plurality of classifier biomarkers from Table 1 from a reference HNSCC BA sample, expression data of the plurality of classifier biomarkers from Table 1 from a reference HNSCC MS sample, expression data of the plurality of classifier biomarkers from Table 1 from a reference HNSCC AT sample, expression data of the plurality of classifier biomarkers from Table 1 from a reference HNSCC CL sample or a combination thereof; and classifying the sample as BA, MS, AT or CL subtype based on the results of the comparing step.

19. The method of claim 18, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.

20. The method of claim 17, wherein the detecting the expression level comprises performing qRT-PCR or any hybridization-based gene assays.

21. The method of claim 20, wherein the expression level is detected by performing qRT-PCR.

22. The method of claim 21, wherein the detection of the expression level comprises using at least one pair of oligonucleotide primers specific for each nucleic acid molecule from the plurality of the classifier biomarker from Table 1.

23. The method of claim 17, further comprising determining the nodal status of the subject suffering from or suspected of suffering from HNSCC.

24. The method of claim 17, further comprising predicting the response to a therapy for treating a subtype of HNSCC based on the detected expression level of the classifier biomarker.

25. The method of claim 24, wherein the subtype is mesenchymal, and the therapy is radiation therapy.

26. The method of claim 25, wherein the nodal status is node negative.

27. The method of claim 17, wherein the sample is a formalin-fixed, paraffin-embedded (FFPE) sample from the head and neck area of the subject, fresh or a frozen tissue sample from the head and neck area of the subject, an exosome, wash fluids, cell pellets or a bodily fluid obtained from the subject.

28. The method of claim 27, wherein the bodily fluid is blood or fractions thereof, urine, saliva, or sputum.

29. The method of claim 17, wherein the plurality of classifier biomarkers selected from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

30. The method of claim 17, wherein the plurality of classifier biomarkers selected from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

31. The method of claim 17, wherein the plurality of classifier biomarkers selected from the classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

32. The method of claim 17, wherein the plurality of classifier biomarkers selected from the classifier biomarkers of Table 1 comprises all the classifier biomarkers from Table 1.

33. The method of claim 17, wherein the HNSCC is oral cavity HNSCC.

34. A method of detecting a biomarker in a sample obtained from a subject suffering from or suspected of suffering from HNSCC, the method comprising, consisting essentially of or consisting of measuring the expression level of a plurality of biomarker nucleic acids selected from Table 1 using an amplification, hybridization and/or sequencing assay.

35. The method of claim 34, wherein the sample was previously diagnosed as being squamous cell carcinoma.

36. The method of claim 35, wherein the previous diagnosis was by histological examination.

37. The method of claim 34, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

38. The method of claim 37, wherein the expression level is detected by performing qRT-PCR.

39. The method of claim 34, wherein the detection of the expression level comprises using at least one pair of oligonucleotide primers per each of the plurality of biomarker nucleic acids selected from Table 1.

40. The method of claim 34, wherein the sample is a formalin-fixed, paraffin-embedded (FFPE) tissue sample from the head and neck area of the subject, fresh or a frozen tissue sample from the head and neck area of the subject, an exosome, wash fluids, cell pellets, or a bodily fluid obtained from the patient.

41. The method of claim 40, wherein the bodily fluid is blood or fractions thereof, urine, saliva, or sputum.

42. The method of claim 34, wherein the plurality of classifier biomarkers from Table 1 comprises, consists essentially of, or consists of at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

43. The method of claim 34, wherein the plurality of classifier biomarkers from Table 1 comprises, consists essentially of, or consists of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

44. The method of claim 34, wherein the plurality of classifier biomarkers from Table 1 comprises, consists essentially of, or consists of olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

45. The method of claim 34, wherein the plurality of classifier biomarkers from Table 1 comprises, consists essentially of, or consists of all the classifier biomarkers from Table 1.

46. The method of claim 34, wherein the HNSCC is oral cavity HNSCC.

47. A method of determining whether a patient suffering from or suspected of suffering from HNSCC is likely to respond to radiation therapy, the method comprising, determining the HNSCC subtype of a sample obtained from the patient, wherein the HNSCC subtype is selected from the group consisting of basal, mesenchymal, atypical and classical; and
based on the subtype, assessing whether the patient is likely to respond to radiation therapy.

48. The method of claim 47, further comprising determining the nodal status of the patient suffering from or suspected of suffering from HNSCC.

49. The method of claim 47 or 48, wherein the patient is assessed as likely to respond to radiation therapy if the HNSCC subtype is determined to be mesenchymal, regardless of nodal status of the patient.

50. The method of claim 47 or 48, wherein the patient is assessed as likely to respond to radiation therapy if the HNSCC subtype is determined to be basal, atypical or classical and nodal status of the patient is determined to be N123.

51. A method for selecting a patient suffering from or suspected of suffering from HNSCC for radiation therapy, the method comprising, determining a HNSCC subtype of a sample obtained from the patient, based on the subtype; and selecting the patient for radiation therapy, wherein the HNSCC subtype is selected from the group consisting of basal, mesenchymal, atypical and classical.

52. The method of claim 51, further comprising determining the nodal status of the patient suffering from or suspected of suffering from HNSCC.

53. The method of claim 51 or 52, wherein the patient is selected for radiation therapy if the HNSCC subtype is determined to be mesenchymal, regardless of nodal status of the patient.

54. The method of claim 51 or 52, wherein the patient is selected for radiation therapy if the HNSCC subtype is determined to be basal, atypical or classical and nodal status of the patient is determined to be N123.

55. The method of any one of claims 47-54, wherein the HNSCC is oral cavity HNSCC.

56. The method of any one of claims 47-55, wherein the radiation therapy is used in combination with surgery and/or chemotherapy.

57. The method of any one of claims 47-56, wherein the sample is a formalin-fixed, paraffin-embedded (FFPE) sample obtained from the head and neck area of the patient, fresh or a frozen tissue sample obtained from the head and neck area of the patient, an exosome, or a bodily fluid obtained from the patient.

58. The method of claim 57, wherein the bodily fluid is blood or fractions thereof, urine, saliva, or sputum.

59. The method of claim 47 or 51, wherein the patient is initially determined to have HNSCC via a histological analysis of a sample.

60. The method of claim 47 or 51, wherein the patient's HNSCC subtype is determined via a histological analysis of a sample obtained from the patient.

61. The method of claim 47 or 51, wherein the determining the HNSCC subtype comprises determining expression levels of a plurality of classifier biomarkers.

62. The method of claim 61, wherein the determining the expression levels of the plurality of classifier biomarkers is at a nucleic acid level by performing RNA sequencing, reverse transcriptase polymerase chain reaction (RT-PCR) or hybridization-based analyses.

63. The method of claim 61, wherein the plurality of classifier biomarkers for determining the HNSCC subtype is selected from a publicly available HNSCC dataset.

64. The method of claim 63, wherein the publicly available HNSCC dataset is TCGA HNSCC RNAseq dataset.

65. The method of claim 61, wherein the plurality of classifier biomarkers for determining the HNSCC subtype is selected from Table 1.

66. The method of claim 65, wherein the RT-PCR is quantitative real time reverse transcriptase polymerase chain reaction (qRT-PCR).

67. The method of claim 66, wherein the RT-PCR is performed with primers specific to each of the plurality of classifier biomarkers from Table 1.

68. The method of claim 65, further comprising comparing the detected levels of expression of the plurality of classifier biomarkers from Table 1 to the levels of expression of the plurality of classifier biomarkers from Table 1 in at least one sample training set(s), wherein the at least one sample training set comprises expression level data of the plurality of classifier biomarkers from Table 1 from a reference HNSCC BA sample, expression level data of the plurality of classifier biomarkers from Table 1 from a reference HNSCC MS sample, expression level data of the plurality of classifier biomarkers from Table 1 from a reference HNSCC AT sample, expression level data of the plurality of classifier biomarkers from Table 1 from a reference HNSCC CL sample or a combination thereof; and classifying the sample obtained from the patient as BA, MS, AT or CL based on the results of the comparing step.

69. The method of claim 68, wherein the comparing step comprises applying a statistical algorithm which comprises determining a correlation between the expression data obtained from the sample obtained from the patient and the expression data from the at least one training set(s); and classifying the sample obtained from the patient as a BA, MS, AT or CL subtype based on the results of the statistical algorithm.

70. The method of claim 65, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

71. The method of claim 65, wherein the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

72. The method of claim 65, wherein the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

73. The method of claim 65, wherein the plurality of the classifier biomarkers comprise all of the classifier biomarkers from Table 1.

74. A method of treating HNSCC in a subject, the method comprising:
determining a subtype of HNSCC of a subject suffering from HNSCC by measuring a nucleic acid expression level of a plurality of classifier biomarkers in a sample obtained from a subject suffering from or suspected of suffering from HNSCC, wherein the plurality of classifier biomarkers is selected from Table 1, wherein the nucleic acid expression level of the plurality of classifier biomarkers indicates the HNSCC subtype of the subject as being basal (BA), mesenchymal (MS), atypical (AT) or classical (CL); and
administering radiation therapy to the subject based on the subtype of the HNSCC.

75. The method of claim 74, wherein the HNSCC is oral cavity HNSCC.

76. The method of claim 74 or 75, wherein the radiation therapy is administered to the subject if the HNSCC subtype is determined to be mesenchymal, regardless of nodal status of the subject.

77. The method of claim 74 or 75, wherein the radiation therapy is administered to the subject if the HNSCC subtype is determined to be basal, classical or atypical and nodal status of the subject is N123.

78. The method of claim 74, wherein the radiation therapy is used in combination with surgery and/or chemotherapy.

79. The method of claim 74, wherein the determining step further comprises comparing the nucleic acid expression levels of the plurality of classifier biomarkers from Table I to the nucleic acid expression levels of the plurality of classifier biomarkers from Table 1 in at least one sample training set(s), wherein the at least one sample training set comprises nucleic acid expression level data of the plurality of classifier biomarkers from Table 1 from a reference HNSCC BA sample, nucleic acid expression level data of the plurality of classifier biomarkers from Table 1 from a reference HNSCC MS sample, nucleic acid expression level data of the plurality of classifier biomarkers from Table 1 from a reference HNSCC AT sample, nucleic acid expression level data of the plurality of classifier biomarkers from Table 1 from a reference HNSCC CL sample or a combination thereof; and classifying the sample obtained from the subject as BA, MS, AT or CL based on the results of the comparing step.

80. The method of claim 79, wherein the comparing step comprises applying a statistical algorithm which comprises determining a correlation between the expression data obtained from the sample obtained from the patient and the expression data from the at least one training set(s); and classifying the sample obtained from the subject as a BA, MS, AT or CL subtype based on the results of the statistical algorithm.

81. The method of claim 74, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

82. The method of claim 74, wherein the plurality of classifier biomarkers from Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

83. The method of claim 74, wherein the plurality of classifier biomarkers from Table 1 comprises olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

84. The method of claim 74, wherein the plurality of the classifier biomarkers comprise all of the classifier biomarkers from Table 1.

85. The method of claim 74, wherein the measuring the nucleic acid expression level is conducted using an amplification, hybridization and/or sequencing assay.

86. The method of claim 85, wherein the amplification, hybridization and/or sequencing assay comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

87. The method of claim 86, wherein the expression level is detected by performing qRT-PCR.

88. The method of any one of claims 74-87, wherein the sample is a formalin-fixed, paraffin-embedded (FFPE) sample obtained from the head and neck area of the subject, fresh or a frozen tissue sample obtained from the head and neck area of the subject, an exosome, wash fluids, cell pellets, or a bodily fluid obtained from the patient.

89. The method of claim 88, wherein the bodily fluid is blood or fractions thereof, urine, saliva, or sputum.

90. A system for determining a head and neck squamous cell carcinoma (HNSCC) subtype of a sample obtained from a subject suffering from HNSCC, the system comprising:
(a) one or more processors; and
(b) one or more memories operatively coupled to at least one of the one or more processors and having instructions stored thereon that, when executed by at least one of the one or more processors, cause the system to
(i) detect an expression level of each of a plurality of classifier biomarkers from Table 1;
(ii) compare the expression levels of each of the plurality of classifier biomarkers from Table 1 to the expression levels of each of the plurality of classifier biomarkers from Table 1 in a control; and
(iii) classifying the sample as a basal (BA), mesenchymal (MS), atypical (AT) or classical (CL) HNSCC subtype based on the results of the comparing step.

91. The system of claim 90, wherein the control comprises at least one sample training set(s), wherein the at least one sample training set comprises expression levels of each of the plurality of classifier biomarkers from Table 1 from a reference HNSCC BA sample, expression levels of each of the plurality of classifier biomarkers from Table 1 from a reference HNSCC MS sample, expression levels of each of the plurality of classifier biomarkers from Table 1 from a reference HNSCC AT sample, expression levels of each of the plurality of classifier biomarkers from Table 1 from a reference HNSCC CL sample or a combination thereof.

92. The system of claim 91, wherein the comparing step comprises applying a statistical algorithm which comprises determining a correlation between the expression data obtained from the sample and the expression data from the at least one training set(s); and classifying the sample as a BA, MS, AT or CL subtype based on the results of the statistical algorithm.

93. The system of any one of claims 90-92, wherein the expression level of each of the plurality of classifier biomarkers from Table 1 is detected at the nucleic acid level.

94. The system of claim 93, wherein the nucleic acid level is RNA or cDNA.

95. The system of any one of claims 90-94, wherein the detecting the expression level comprises performing quantitative real time reverse transcriptase polymerase chain reaction (qRT-PCR), RNAseq, microarrays, gene chips, nCounter Gene Expression Assay, Serial Analysis of Gene Expression (SAGE), Rapid Analysis of Gene Expression (RAGE), nuclease protection assays, Northern blotting, or any other equivalent gene expression detection techniques.

96. The system of claim 95, wherein the expression level is detected by performing qRT-PCR.

97. The system of claim 95 or 96, wherein the detecting the expression level is performed using a device that is part of the system or in communication with at least one of the one or more processors, wherein upon receipt of instructions sent by the at least one of the one or more processors, perform the detection of the expression levels.

98. The system of any one of claims 90-97, wherein the plurality of classifier biomarkers from Table 1 comprises at least two classifier biomarkers, at least 5 classifier biomarkers, at least 11 classifier biomarkers, at least 22 classifier biomarkers, at least 33 classifier biomarkers, at least 44 classifier biomarkers, at least 55 classifier biomarkers, at least 66 classifier biomarkers, at least 77 classifier biomarkers or at least 88 classifier biomarkers from Table 1.

99. The system of any one of claims 90-97, wherein the plurality of classifier biomarkers of Table 1 comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% of the classifier biomarkers from Table 1.

100. The system of any one of claims 90-97, wherein the plurality of classifier biomarkers of Table 1 comprise olfml3, pcolce, lepre1, nnmt, olfml2b, col6a1, phldb1, col6a2, cmtm3, gpx8, pth1r, cyp2c18, grhl3, csta, elf3, sprr3, adh7, aldh3a1, tmprss11a, klf5, slc9a3r1, sox2 or any combination thereof.

101. The system of any one of claims 90-97, wherein the plurality of classifier biomarkers of Table 1 comprises all the classifier biomarkers from Table 1.

102. The system of any one of claims 90-101, further comprising determining the nodal status of the subject suffering from or suspected of suffering from HNSCC.

103. The system of any one of claims 90-102, wherein the HNSCC is oral cavity HNSCC.


SEQUENCE LISTING (88 sequences)

SEQ ID NO: 1 - DNA, 6504 bp, Homo sapiens
  source 1..6504: mol_type = genomic DNA
gccgccgccgccgccgccagcgctagcgccagcagccgggcccgatcacccgccgcccggtgcccgccgccgcccgcgccagcaaccgggcccgatcacccgccgcccggtgcccgccgccgcccgcgccaccggcatggcgctccggggcttctgcagcgccgatggctccgacccgctctgggactggaatgtcacgtggaataccagcaaccccgacttcaccaagtgctttcagaacacggtcctcgtgtgggtgccttgtttttacctctgggcctgtttccccttctacttcctctatctctcccgacatgaccgaggctacattcagatgacacctctcaacaaaaccaaaactgccttgggatttttgctgtggatcgtctgctgggcagacctcttctactctttctgggaaagaagtcggggcatattcctggccccagtgtttctggtcagcccaactctcttgggcatcaccatgctgcttgctacctttttaattcagctggagaggaggaagggagttcagtcttcagggatcatgctcactttctggctggtagccctagtgtgtgccctagccatcctgagatccaaaattatgacagccttaaaagaggatgcccaggtggacctgtttcgtgacatcactttctacgtctacttttccctcttactcattcagctcgtcttgtcctgtttctcagatcgctcacccctgttctcggaaaccatccacgaccctaatccctgcccagagtccagcgcttccttcctgtcgaggatcaccttctggtggatcacagggttgattgtccggggctaccgccagcccctggagggcagtgacctctggtccttaaacaaggaggacacgtcggaacaagtcgtgcctgttttggtaaagaactggaagaaggaatgcgccaagactaggaagcagccggtgaaggttgtgtactcctccaaggatcctgcccagccgaaagagagttccaaggtggatgcgaatgaggaggtggaggctttgatcgtcaagtccccacagaaggagtggaacccctctctgtttaaggtgttatacaagacctttgggccctacttcctcatgagcttcttcttcaaggccatccacgacctgatgatgttttccgggccgcagatcttaaagttgctcatcaagttcgtgaatgacacgaaggccccagactggcagggctacttctacaccgtgctgctgtttgtcactgcctgcctgcagaccctcgtgctgcaccagtacttccacatctgcttcgtcagtggcatgaggatcaagaccgctgtcattggggctgtctatcggaaggccctggtgatcaccaattcagccagaaaatcctccacggtcggggagattgtcaacctcatgtctgtggacgctcagaggttcatggacttggccacgtacattaacatgatctggtcagcccccctgcaagtcatccttgctctctacctcctgtggctgaatctgggcccttccgtcctggctggagtggcggtgatggtcctcatggtgcccgtcaatgctgtgatggcgatgaagaccaagacgtatcaggtggcccacatgaagagcaaagacaatcggatcaagctgatgaacgaaattctcaatgggatcaaagtgctaaagctttatgcctgggagctggcattcaaggacaaggtgctggccatcaggcaggaggagctgaaggtgctgaagaagtctgcctacctgtcagccgtgggcaccttcacctgggtctgcacgccctttctggtggccttgtgcacatttgccgtctacgtgaccattgacgagaacaacatcctggatgcccagacagccttcgtgtctttggccttgttcaacatcctccggtttcccctgaacattctccccatggtcatcagcagcatcgtgcaggcgagtgtctccctcaaacgcctgaggatctttctctcccatgaggagctggaacctgacagcatcgagcgacggcctgtcaaagacggcgggggcacgaacagcatcaccgtgaggaatgccacattcacctgggccaggagcgaccctcccacactgaatggcatcaccttctccatccccgaaggtgctttggtggccgtggtgggccaggtgggctgcggaaagtcgtccctgctctcagccctcttggctgagatggacaaagtggaggggcacgtggctatcaagggctccgtggcctatgtgccacagcaggcctggattcagaatgattctctccgagaaaacatcctttttggatgtcagctggaggaaccatattacaggtccgtgatacaggcctgtgccctcctcccagacctggaaatcctgcccagtggggatcggacagagattggcgagaagggcgtgaacctgtctgggggccagaagcagcgcgtgagcctggcccgggccgtgtactccaacgctgacatttacctcttcgatgatcccctctcagcagtggatgcccatgtgggaaaacacatctttgaaaatgtgattggccccaaggggatgctgaagaacaagacgcggatcttggtcacgcacagcatgagctacttgccgcaggtggacgtcatcatcgtcatgagtggcggcaagatctctgagatgggctcctaccaggagctgctggctcgagacggcgccttcgctgagttcctgcgtacctatgccagcacagagcaggagcaggatgcagaggagaacggggtcacgggcgtcagcggtccagggaaggaagcaaagcaaatggagaatggcatgctggtgacggacagtgcagggaagcaactgcagagacagctcagcagctcctcctcctatagtggggacatcagcaggcaccacaacagcaccgcagaactgcagaaagctgaggccaagaaggaggagacctggaagctgatggaggctgacaaggcgcagacagggcaggtcaagctttccgtgtactgggactacatgaaggccatcggactcttcatctccttcctcagcatcttccttttcatgtgtaaccatgtgtccgcgctggcttccaactattggctcagcctctggactgatgaccccatcgtcaacgggactcaggagcacacgaaagtccggctgagcgtctatggagccctgggcatttcacaagggatcgccgtgtttggctactccatggccgtgtccatcggggggatcttggcttcccgctgtctgcacgtggacctgctgcacagcatcctgcggtcacccatgagcttctttgagcggacccccagtgggaacctggtgaaccgcttctccaaggagctggacacagtggactccatgatcccggaggtcatcaagatgttcatgggctccctgttcaacgtcattggtgcctgcatcgttatcctgctggccacgcccatcgccgccatcatcatcccgccccttggcctcatctacttcttcgtccagaggttctacgtggcttcctcccggcagctgaagcgcctcgagtcggtcagccgctccccggtctattcccatttcaacgagaccttgctgggggtcagcgtcattcgagccttcgaggagcaggagcgcttcatccaccagagtgacctgaaggtggacgagaaccagaaggcctattaccccagcatcgtggccaacaggtggctggccgtgcggctggagtgtgtgggcaactgcatcgttctgtttgctgccctgtttgcggtgatctccaggcacagcctcagtgctggcttggtgggcctctcagtgtcttactcattgcaggtcaccacgtacttgaactggctggttcggatgtcatctgaaatggaaaccaacatcgtggccgtggagaggctcaaggagtattcagagactgagaaggaggcgccctggcaaatccaggagacagctccgcccagcagctggccccaggtgggccgagtggaattccggaactactgcctgcgctaccgagaggacctggacttcgttctcaggcacatcaatgtcacgatcaatgggggagaaaaggtcggcatcgtggggcggacgggagctgggaagtcgtccctgaccctgggcttatttcggatcaacgagtctgccgaaggagagatcatcatcgatggcatcaacatcgccaagatcggcctgcacgacctccgcttcaagatcaccatcatcccccaggaccctgttttgttttcgggttccctccgaatgaacctggacccattcagccagtactcggatgaagaagtctggacgtccctggagctggcccacctgaaggacttcgtgtcagcccttcctgacaagctagaccatgaatgtgcagaaggcggggagaacctcagtgtcgggcagcgccagcttgtgtgcctagcccgggccctgctgaggaagacgaagatccttgtgttggatgaggccacggcagccgtggacctggaaacggacgacctcatccagtccaccatccggacacagttcgaggactgcaccgtcctcaccatcgcccaccggctcaacaccatcatggactacacaagggtgatcgtcttggacaaaggagaaatccaggagtacggcgccccatcggacctcctgcagcagagaggtcttttctacagcatggccaaagacgccggcttggtgtgagccccagagctggcatatctggtcagaactgcagggcctatatgccagcgcccagggaggagtcagtacccctggtaaaccaagcctcccacactgaaaccaaaacataaaaaccaaacccagacaaccaaaacatattcaaagcagcagccaccgccatccggtcccctgcctggaactggctgtgaagacccaggagagacagagatgcgaaccacccaaaacacgcacaccctgcccctggtgccctgagacagacacacagcctcacgcccccaggaatgcaagtggtttcctggtgcttcccacggaggagttttggcagccagacttctggaggaattggttgtatagaagatcctagtgaccaaattcagcctactgcctcggatctctccagccgaagtctgtggactgcaagtctttgagatgcttctggctcccatcacctctaacatccttgtctgggtctaccaggaacgcttcatttccttggggctgcagttttgtggttgaggggcctggagaaaatcattttctccccttggcagtgtcccagggccctggatggtcctcttaccaacatctggtcttccaggcactcaaaagctgggaaccagcatctcagcgccagctctaccagttctcgttttgggccagaggcagcctctgcactcccacgcctgtcctcctggaagggacctggttggactaacggctaacctggacctggaactgtagggccaggggattgtctcagggccgacgttccacctggggcttccctccccacccaccccgactccaggctttcccttttttcttttgttcaacattgtaagaacaatcaatgctgttattactgttcccaccatgattgatgtggggtaaatattaaggagatggcctcatgggaatttgaccttgactagaaatagagactgagagtgagcaaccagctggaaggtactatgccagtcctagcagaaaaatgtgttaggggcctggcccaaagcagtgttggttgcttacagtgttgattgattttgttcttttttcttaccacctcttttctttccctctcatggtacctgctcatggttatgaagctttcaaagtaaagaacacgaaatacctcccaagtattaccagtgggtaccaaaaaaatgtccccttgagtcttttccttgtttttagatgttaattctctcccttggcatccggttagccccccagggggggcagcattgtggagaacttgatatttagttactgatgctcttccaggacacgaaaagaacccatctttgaatatcaatgattttttttttttaagtactgttccggggagaaaaacagtctcaaaacttgaacttcttgggaatagaagtgttgggctgagaagtaacattcccaggaaatagtgagaagctcgccctgtgtttgaaaccgtgttggtctctgtgttcctggaagaaaacagggaagcagcatcttttaaagcctgttctttaaggtgtctcgttagagcccaaagtggaatccggaaggcagccagagctgaggctgccccaagactcagacttgctaagaattacgccgccgacttcaaacccagagagcatctttcttttaggcgaaaacgcatatatttattttttgtaagttataccattctttcacattagataaactaagttttgggggatccttttgtaatgacttacactggaaatgcgaacatttgcagtaaaaaaatatatatatatcta

SEQ ID NO: 2 - DNA, 5790 bp, Homo sapiens
  source 1..5790: mol_type = genomic DNA
atgctcgggagcgtggttgagcggctggcgcggttgtcctggagcaggggcgcaggaattctgatgtgaaactaacagtctgtgagccctggaacctccactcagagaagatgaaggatatcgacataggaaaagagtatatcatccccagtcctgggtatagaagtgtgagggagagaaccagcacttctgggacgcacagagaccgtgaagattccaagttcaggagaactcgaccgttggaatgccaagatgccttggaaacagcagcccgagccgagggcctctctcttgatgcctccatgcattctcagctcagaatcctggatgaggagcatcccaagggaaagtaccatcatggcttgagtgctctgaagcccatccggactacttccaaacaccagcacccagtggacaatgctgggcttttttcctgtatgactttttcgtggctttcttctctggcccgtgtggcccacaagaagggggagctctcaatggaagacgtgtggtctctgtccaagcacgagtcttctgacgtgaactgcagaagactagagagactgtggcaagaagagctgaatgaagttgggccagacgctgcttccctgcgaagggttgtgtggatcttctgccgcaccaggctcatcctgtccatcgtgtgcctgatgatcacgcagctggctggcttcagtggaccagccttcatggtgaaacacctcttggagtatacccaggcaacagagtctaacctgcagtacagcttgttgttagtgctgggcctcctcctgacggaaatcgtgcggtcttggtcgcttgcactgacttgggcattgaattaccgaaccggtgtccgcttgcggggggccatcctaaccatggcatttaagaagatccttaagttaaagaacattaaagagaaatccctgggtgagctcatcaacatttgctccaacgatgggcagagaatgtttgaggcagcagccgttggcagcctgctggctggaggacccgttgttgccatcttaggcatgatttataatgtaattattctgggaccaacaggcttcctgggatcagctgtttttatcctcttttacccagcaatgatgtttgcatcacggctcacagcatatttcaggagaaaatgcgtggccgccacggatgaacgtgtccagaagatgaatgaagttcttacttacattaaatttatcaaaatgtatgcctgggtcaaagcattttctcagagtgttcaaaaaatccgcgaggaggagcgtcggatattggaaaaagctgggtacttccagagcatcactgtgggtgtggctcccattgtggtggtgattgccagcgtggtgaccttctctgttcatatgaccctgggcttcgatctgacagcagcacaggctttcacagtggtgacagtcttcaattccatgacttttgctttgaaagtaacaccgttttcagtaaagtccctctcagaagcctcagtggctgttgacagatttaagagtttgtttctaatggaagaggttcacatgataaagaacaaaccagccagtcctcacatcaagatagagatgaaaaatgccaccttggcatgggactcctcccactccagtatccagaactcgcccaagctgacccccaaaatgaaaaaagacaagagggcttccaggggcaagaaagagaaggtgaggcagctgcagcgcactgagcatcaggcggtgctggcagagcagaaaggccacctcctcctggacagtgacgagcggcccagtcccgaagaggaagaaggcaagcacatccacctgggccacctgcgcttacagaggacactgcacagcatcgatctggagatccaagagggtaaactggttggaatctgtggcagtgtgggaagtggaaaaacctctctcatttcagccattttaggccagatgacgcttctagagggcagcattgcaatcagtggaaccttcgcttatgtggcccagcaggcctggatcctcaatgctactctgagagacaacatcctgtttgggaaggaatatgatgaagaaagatacaactctgtgctgaacagctgctgcctgaggcctgacctggccattcttcccagcagcgacctgacggagattggagagcgaggagccaacctgagcggtgggcagcgccagaggatcagccttgcccgggccttgtatagtgacaggagcatctacatcctggacgaccccctcagtgccttagatgcccatgtgggcaaccacatcttcaatagtgctatccggaaacatctcaagtccaagacagttctgtttgttacccaccagttacagtacctggttgactgtgatgaagtgatcttcatgaaagagggctgtattacggaaagaggcacccatgaggaactgatgaatttaaatggtgactatgctaccatttttaataacctgttgctgggagagacaccgccagttgagatcaattcaaaaaaggaaaccagtggttcacagaagaagtcacaagacaagggtcctaaaacaggatcagtaaagaaggaaaaagcagtaaagccagaggaagggcagcttgtgcagctggaagagaaagggcagggttcagtgccctggtcagtatatggtgtctacatccaggctgctgggggccccttggcattcctggttattatggcccttttcatgctgaatgtaggcagcaccgccttcagcacctggtggttgagttactggatcaagcaaggaagcgggaacaccactgtgactcgagggaacgagacctcggtgagtgacagcatgaaggacaatcctcatatgcagtactatgccagcatctacgccctctccatggcagtcatgctgatcctgaaagccattcgaggagttgtctttgtcaagggcacgctgcgagcttcctcccggctgcatgacgagcttttccgaaggatccttcgaagccctatgaagttttttgacacgacccccacagggaggattctcaacaggttttccaaagacatggatgaagttgacgtgcggctgccgttccaggccgagatgttcatccagaacgttatcctggtgttcttctgtgtgggaatgatcgcaggagtcttcccgtggttccttgtggcagtggggccccttgtcatcctcttttcagtcctgcacattgtctccagggtcctgattcgggagctgaagcgtctggacaatatcacgcagtcacctttcctctcccacatcacgtccagcatacagggccttgccaccatccacgcctacaataaagggcaggagtttctgcacagataccaggagctgctggatgacaaccaagctcctttttttttgtttacgtgtgcgatgcggtggctggctgtgcggctggacctcatcagcatcgccctcatcaccaccacggggctgatgatcgttcttatgcacgggcagattcccccagcctatgcgggtctcgccatctcttatgctgtccagttaacggggctgttccagtttacggtcagactggcatctgagacagaagctcgattcacctcggtggagaggatcaatcactacattaagactctgtccttggaagcacctgccagaattaagaacaaggctccctcccctgactggccccaggagggagaggtgacctttgagaacgcagagatgaggtaccgagaaaacctccctctcgtcctaaagaaagtatccttcacgatcaaacctaaagagaagattggcattgtggggcggacaggatcagggaagtcctcgctggggatggccctcttccgtctggtggagttatctggaggctgcatcaagattgatggagtgagaatcagtgatattggccttgccgacctccgaagcaaactctctatcattcctcaagagccggtgctgttcagtggcactgtcagatcaaatttggaccccttcaaccagtacactgaagaccagatttgggatgccctggagaggacacacatgaaagaatgtattgctcagctacctctgaaacttgaatctgaagtgatggagaatggggataacttctcagtgggggaacggcagctcttgtgcatagctagagccctgctccgccactgtaagattctgattttagatgaagccacagctgccatggacacagagacagacttattgattcaagagaccatccgagaagcatttgcagactgtaccatgctgaccattgcccatcgcctgcacacggttctaggctccgataggattatggtgctggcccagggacaggtggtggagtttgacaccccatcggtccttctgtccaacgacagttcccgattctatgccatgtttgctgctgcagagaacaaggtcgctgtcaagggctgactcctccctgttgacgaagtctcttttctttagagcattgccattccctgcctggggcgggcccctcatcgcgtcctcctaccgaaaccttgcctttctcgattttatctttcgcacagcagttccggattggcttgtgtgtttcacttttagggagagtcatattttgattattgtatttattccatattcatgtaaacaaaatttagtttttgttcttaattgcactctaaaaggttcagggaaccgttattataattgtatcagaggcctataatgaagctttatacgtgtagctatatctatatataattctgtacatagcctatatttacagtgaaaatgtaagctgtttattttatattaaaataagcactgtgctaataacagtgcatattcctttctatcatttttgtacagtttgctgtactagagatctggttttgctattagactgtaggaagagtagcatttcattcttctctagctggtggtttcacggtgccaggttttctgggtgtccaaaggaagacgtgtggcaatagtgggccctccgacagccccctctgccgcctccccacggccgctccaggggtggctggagacgggtgggcggctggagaccatgcagagcgccgtgagttctcagggctcctgccttctgtcctggtgtcacttactgtttctgtcaggagagcagcggggcgaagcccaggccccttttcactccctccatcaagaatggggatcacagagacattcctccgagccggggagtttctttcctgccttcttctttttgctgttgtttctaaacaagaatcagtctatccacagagagtcccactgcctcaggttcctatggctggccactgcacagagctctccagctccaagacctgttggttccaagccctggagccaactgctgctttttgaggtggcactttttcatttgcctattcccacacctccacagttcagtggcagggctcaggatttcgtgggtctgttttcctttctcaccgcagtcgtcgcacagtctctctctctctctcccctcaaagtctgcaactttaagcagctcttgctaatcagtgtctcacactggcgtagaagtttttgtactgtaaagagacctacctcaggttgctggttgctgtgtggtttggtgtgttcccgcaaaccccctttgtgctgtggggctggtagctcaggtgggcgtggtcactgctgtcatcaattgaatggtcagcgttgcatgtcgtgaccaactagacattctgtcgccttagcatgtttgctgaacaccttgtggaagcaaaaatctgaaaatgtgaataaaattattttggattttgtaaaa

SEQ ID NO: 3 - DNA, 3722 bp, Homo sapiens
  source 1..3722: mol_type = genomic DNA
agtgtgcgtggagattaggtccaagcgcccgcccagaggcaggcagtccgcgagcccagccgccgctgtcgccgccagtagcagccttcgccagcagcgccgcggcggaaccgggcgcaggggagcgagcccggccccgccagcccagcccagcccagccctactccctccccacgccagggcagcagccgttgctcagagagaaggtggaggaagaaatccagaccctagcacgcgcgcaccatcatggaccattatgattctcagcaaaccaacgattacatgcagccagaagaggactgggaccgggacctgctcctggacccggcctgggagaagcagcagagaaagacattcacggcatggtgtaactcccacctccggaaggcggggacacagatcgagaacatcgaagaggacttccgggatggcctgaagctcatgctgctgctggaggtcatctcaggtgaacgcttggccaagccagagcgaggcaagatgagagtgcacaagatctccaacgtcaacaaggccctggatttcatagccagcaaaggcgtcaaactggtgtccatcggagccgaagaaatcgtggatgggaatgtgaagatgaccctgggcatgatctggaccatcatcctgcgctttgccatccaggacatctccgtggaagagacttcagccaaggaagggctgctcctgtggtgtcagagaaagacagccccttacaaaaatgtcaacatccagaacttccacataagctggaaggatggcctcggcttctgtgctttgatccaccgacaccggcccgagctgattgactacgggaagctgcggaaggatgatccactcacaaatctgaatacggcttttgacgtggcagagaagtacctggacatccccaagatgctggatgccgaagacatcgttggaactgcccgaccggatgagaaagccatcatgacttacgtgtctagcttctaccacgccttctctggagcccagaaggcggagacagcagccaatcgcatctgcaaggtgttggccgtcaaccaggagaacgagcagcttatggaagactacgagaagctggccagtgatctgttggagtggatccgccgcacaatcccgtggctggagaaccgggtgcccgagaacaccatgcatgccatgcaacagaagctggaggacttccgggactaccggcgcctgcacaagccgcccaaggtgcaggagaagtgccagctggagatcaacttcaacacgctgcagaccaagctgcggctcagcaaccggcctgccttcatgccctctgagggcaggatggtctcggacatcaacaatgcctggggctgcctggagcaggtggagaagggctatgaggagtggttgctgaatgagatccggaggctggagcgactggaccacctggcagagaagttccggcagaaggcctccatccacgaggcctggactgacggcaaagaggccatgctgcgacagaaggactatgagaccgccaccctctcggagatcaaggccctgctcaagaagcatgaggccttcgagagtgacctggctgcccaccaggaccgtgtggagcagattgccgccatcgcacaggagctcaatgagctggactattatgactcacccagtgtcaacgcccgttgccaaaagatctgtgaccagtgggacaatctgggggccctaactcagaagcgaagggaagctctggagcggaccgagaaactgctggagaccattgaccagctgtacttggagtatgccaagcgggctgcacccttcaacaactggatggagggggccatggaggacctgcaggacaccttcattgtgcacaccattgaggagatccagggactgaccacagcccatgagcagttcaaggccaccctccctgatgccgacaaggagcgcctggccatcctgggcatccacaatgaggtgtccaagattgtccagacctaccacgtcaatatggcgggcaccaacccctacacaaccatcacgcctcaggagatcaatggcaaatgggaccacgtgcggcagctggtgcctcggagggaccaagctctgacggaggagcatgcccgacagcagcacaatgagaggctacgcaagcagtttggagcccaggccaatgtcatcgggccctggatccagaccaagatggaggagatcgggaggatctccattgagatgcatgggaccctggaggaccagctcagccacctgcggcagtatgagaagagcatcgtcaactacaagccaaagattgatcagctggagggcgaccaccagctcatccaggaggcgctcatcttcgacaacaagcacaccaactacaccatggagcacatccgtgtgggctgggagcagctgctcaccaccatcgccaggaccatcaatgaggtagagaaccagatcctgacccgggatgccaagggcatcagccaggagcagatgaatgagttccgggcctccttcaaccactttgaccgggatcactccggcacactgggtcccgaggagttcaaagcctgcctcatcagcttgggttatgatattggcaacgacccccagaagaagacaggcatgatggacacggatgatttccgcgcctgcctgatctccatgggttacaacatgggagaagcagaatttgcccgcatcatgagcattgtggaccccaaccgcctgggggtagtgacattccaggccttcattgacttcatgtcccgcgagacagccgacacagatacagcagaccaagtcatggcttccttcaagatcctggctggggacaagaactacattaccatggacgagctgcgccgcgagctgccacccgaccaggctgagtactgcatcgcgcggatggccccctacaccggccccgactccgtgccaggtgctctggactacatgtccttctccacggcgctgtacggcgagagtgacctctaatccaccccgcccggccgccctcgtcttgtgcgccgtgccctgccttgcacctccgccgtcgcccatctcctgcctgggttcggtttcagctcccagcctccacccgggtgagctggggcccacgtggcatcgatcctccctgcccgcgaagtgacagtttacaaaattattttctgcaaaaaagaaaaaaaagttacgttaaaaaccaaaaaactacatattttattatagaaaaagtattttttctccaccagacaaatggaaaaaaagaggaaagattaactatttgcaccgaaatgtcttgttttgttgcgacataggaaaataaccaagcacaaagttatattccatcctttttactgatttttttttcttctatctgttccatctgctgtattcatttctccaatctcatgtccattttggtgtgggagtcggggtagggggtactcttgtcaaaaggcacattggtgcatgtgtgtttgctagctcacttgtccatgaaaatattttatgatattaaagaaaatcttttgaaatggctgttttttaaggaagagaatttatgtggcttctcatttttaaatcccctcagaggtgtgactagtctctttatcagcacacacttaaaaaatttttaatattgtctattaaaaataggacaaacttggagagtatggacaactttgatattgcttggcacagatggtattaaaaaaaccacactccta

SEQ ID NO: 4 - DNA, 2119 bp, Homo sapiens
  source 1..2119: mol_type = genomic DNA
agactatctatgtgaaggcacaagctgctgttatatacaacagagtgaactgagcatcagtcagaaaaagtctatgtttgcagaaatacagatccaagacaaagacaggatgggcactgctggaaaagttattaaatgcaaagcagctgtgctttgggagcagaagcaacccttctccattgaggaaatagaagttgccccaccaaagactaaagaagttcgcattaagattttggccacaggaatctgtcgcacagatgaccatgtgataaaaggaacaatggtgtccaagtttccagtgattgtgggacatgaggcaactgggattgtagagagcattggagaaggagtgactacagtgaaaccaggtgacaaagtcatccctctctttctgccacaatgtagagaatgcaatgcttgtcgcaacccagatggcaacctttgcattaggagcgatattactggtcgtggagtactggctgatggcaccaccagatttacatgcaagggcaaaccagtccaccacttcatgaacaccagtacatttaccgagtacacagtggtggatgaatcttctgttgctaagattgatgatgcagctcctcctgagaaagtctgtttaattggctgtgggttttccactggatatggcgctgctgttaaaactggcaaggtcaaacctggttccacttgcgtcgtctttggcctgggaggagttggcctgtcagtcatcatgggctgtaagtcagctggtgcatctaggatcattgggattgacctcaacaaagacaaatttgagaaggccatggctgtaggtgccactgagtgtatcagtcccaaggactctaccaaacccatcagtgaggtgctgtcagaaatgacaggcaacaacgtgggatacacctttgaagttattgggcatcttgaaaccatgattgatgccctggcatcctgccacatgaactatgggaccagcgtggttgtaggagttcctccatcagccaagatgctcacctatgacccgatgttgctcttcactggacgcacatggaagggatgtgtctttggaggtttgaaaagcagagatgatgtcccaaaactagtgactgagttcctggcaaagaaatttgacctggaccagttgataactcatgttttaccatttaaaaaaatcagtgaaggatttgagctgctcaattcaggacaaagcattcgaacggtcctgacgttttgagatccaaagtggcaggaggtctgtgttgtcatggtgaactggagtttctcttgtgagagttccctcatctgaaatcatgtatctgtctcacaaatacaagcataagtagaagatttgttgaagacatagaacccttataaagaattattaacctttataaacatttaaagtcttgtgagcacctgggaattagtataataacaatgttaatatttttgatttacattttgtaaggctataattgtatcttttaagaaaacatacacttggatttctatgttgaaatggagatttttaagagttttaaccagctgctgcagatatataactcaaaacagatatagcgtataaagatatagtaaatgcatctcctagagtaatattcacttaacacattgaaactattattttttagatttgaatataaatgtattttttaaacacttgttatgagttaagttggattacattttgaaatcagttcattccatgatgcatattactggattagattaagaaagacagaaaagattaagggacgggcacatttttcaacgattaagaatcatcattacataacttggtgaaactgaaaaagtatatcatatgggtacacaaggctatttgccagcatatattaatattttagaaaatattccttttgtaatactgaatataaacatagagctagaatcatattatcatacttatcataatgttcaatttgatacagtagaattgcaagtccctaagtccctattcactgtgcttagtagtgactccatttaataaaaagtgtttttagtttttaacaactacactgatgtatctatatatatctataacatgttaaaaattcttaagaaaattaaaaattatataaaa

SEQ ID NO: 5 - DNA, 1944 bp, Homo sapiens
  source 1..1944: mol_type = genomic DNA
atcagctttgcaagcaagtaagggagcggaaaaggccgggaaaggccctgccgcgagcacgctgccaagagcccccagcagcagttcggcttaggactcgggttgcggcgggtgtcaccttctcaggggctagcaaggcagccagggcccaggcgtctgagtgaggggcgggagaggaggcgaggcagaaagtggaccttccagcggaaaggccattttccccaaggccgagcccagggaagtcccttcctatagaattcaggcagggtgggaggcagggcgcgctcgtgcccctcagccagctgcaggtgctctctgtccccaggcgccatgagcaagatcagcgaggccgtgaagcgcgcccgcgccgccttcagctcgggcaggacccgtccgctgcagttccggatccagcagctggaggcgctgcagcgcctgatccaggagcaggagcaggagctggtgggcgcgctggccgcagacctgcacaagaatgaatggaacgcctactatgaggaggtggtgtacgtcctagaggagatcgagtacatgatccagaagctccctgagtgggccgcggatgagcccgtggagaagacgccccagactcagcaggacgagctctacatccactcggagccactgggcgtggtcctcgtcattggcacctggaactaccccttcaacctcaccatccagcccatggtgggcgccatcgctgcagggaactcagtggtcctcaagccctcggagctgagtgagaacatggcgagcctgctggctaccatcatcccccagtacctggacaaggatctgtacccagtaatcaatgggggtgtccctgagaccacggagctgctcaaggagaggttcgaccatatcctgtacacgggcagcacgggggtggggaagatcatcatgacggctgctgccaagcacctgacccctgtcacgctggagctgggagggaagagtccctgctacgtggacaagaactgtgacctggacgtggcctgccgacgcatcgcctgggggaaattcatgaacagtggccagacctgcgtggcccctgactacatcctctgtgacccctcgatccagaaccaaattgtggagaagctcaagaagtcactgaaagagttctacggggaagatgctaagaaatcccgggactatggaagaatcattagtgcccggcacttccagagggtgatgggcctgattgagggccagaaggtggcttatgggggcaccggggatgccgccactcgctacatagcccccaccatcctcacggacgtggacccccagtccccggtgatgcaagaggagatcttcgggcctgtgctgcccatcgtgtgcgtgcgcagcctggaggaggccatccagttcatcaaccagcgtgagaagcccctggccctctacatgttctccagcaacgacaaggtgattaagaagatgattgcagagacatccagtggtggggtggcggccaacgatgtcatcgtccacatcaccttgcactctctgcccttcgggggcgtggggaacagcggcatgggatcctaccatggcaagaagagcttcgagactttctctcaccgccgctcttgcctggtgaggcctctgatgaatgatgaaggcctgaaggtcagataccccccgagcccggccaagatgacccagcactgaggaggggttgctccgcctggcctggccatactgtgtcccatcggagtgcggaccaccctcactggctctcctggccctgggagaatcgctcctgcagccccagcccagccccactcctctgctgacctgctgacctgtgcacaccccactcccacatgggcccaggcctcaccattccaagtctccacccctttctagaccaataaagagacgaatacaattttctaactcagcaaaaaaaaaaaaaaaaaa

SEQ ID NO: 6 - DNA, 8899 bp, Homo sapiens
  source 1..8899: mol_type = genomic DNA
agttgccggagcccgttctggtctcaagtaggaagctagtgcgctgtaaccgcatctgatctgggcgctccgggaagggcgagactggagcagagccgctgggcgccggagccgaggcgagcgccgcgcgcaccactggttggagttgctgtgggtgagctgctgtggtctgtagccaagcatgctgtggtcggatctgcccagccgtggaacagaaacatttgctggatggaaaatccataaaagaaagctcctgtgaaaagctgaggctgacaataatttaagcaaaatcagatcgatctctttgggctgcctgacctccttgggtgcttgctattaattaacagactttgtggggaaaaaaaggagcttgccttctgagctttgtaccaaagacctgggaaaactaaccatctcagtctttcctgaggacttgggaactgccgaggcctctgccaatgtgttgactgtcgctatgggctcactgttgtccaggcagctcatatttcaaattataacctatttcctgcaccattgctgacgcctggtgatccatgtcagaagtacttccagctgactcaggtgttgacaccttggcagtgtttatggccagcagcggaactacagacgtcacaaatcggaacagcccagccacaccaccaaacacccttaacctccgatcctcccacaatgaactgttgaacgctgaaataaaacacacagaaaccaagaacagcacacctcccaaatgcaggaaaaaatatgcactaactaacatccaggcggccatgggcctctcggatccagctgcacagcccctgctgggaaatggctctgccaacatcaagctggtgaaaaatggggagaaccagctccgtaaggctgcagagcaagggcagcaggaccccaacaaaaacctgagccccactgcagtcatcaacataacttctgagaagttagagggtaaagagccccacccacaggattcctcgagctgtgagattttaccctcccagcccaggagaactaagagcttcctaaattactatgcagatctggaaacctcagccagagaactagagcagaaccgaggcaatcaccatgggactgcggaagagaaatcccagccagtccagggccaggcctccaccatcattgggaatggcgatttgctgctgcagaaaccaaacagaccccagtccagccctgaagacggccaagtagccacagtgtcatccagcccagaaaccaagaaggatcatccgaaaacaggggccaaaaccgactgtgcactgcaccggatccagaacctggcaccgagcgatgaggagtccagctggacaacgttgtcccaagacagtgcctcacccagctccccggatgaaacagcagatatatggagtgatcactcatttcagactgatccagatttgccgcctggctggaaaagagtcagtgacattgccgggacctattattggcacatcccaacaggaacgactcagtgggaacggcccgtctccatcccagcagatctccagggttctaggaaagggtcacttagttctgtaacgccatctcccaccccagagaacgagaaacagccatggagtgattttgctgttctgaatgggggaaagattaatagtgacatttggaaggatttgcatgcagccactgttaacccggaccccagtttaaaagagtttgaaggagcaaccctacgctatgcatctttgaaactcagaaatgccccacaccctgatgatgatgattcttgtagtatcaacagtgacccagaagccaagtgttttgctgtgcgttctctgggatgggtagagatggcagaagaggacctcgcccccggtaaaagtagtgttgcggtcaacaactgcatcaggcaactttcctactgcaaaaatgacatccgagacacagtcgggatttggggagaggggaaagacatgtacctgatcctggagaatgacatgctcagcctggtggaccccatggaccgcagcgtgctgcactcgcagcccatcgtcagcatccgcgtgtggggcgtgggccgcgacaatggccgggattttgcttatgtagcaagagataaagatacaagaattttgaaatgtcatgtatttcgatgtgacacaccagcaaaagccattgccacaagtctccacgagatctgctccaagattatggctgaacggaagaatgccaaagcgctggcctgcagctccttacaggaaagggccaatgtgaacctcgatgtccctttgcaagtagattttccaacaccaaagactgagctggtccagaagttccacgtgcagtacttgggcatgttacctgtagacaaaccagtcggaatggatattttgaacagtgccatagaaaatcttatgacctcatccaacaaggaggactggctgtcagtgaacatgaacgtggctgatgccactgtgactgtcatcagtgaaaagaatgaagaggaagtcttagtggaatgtcgtgtgcgattcctgtccttcatgggtgttgggaaggacgtccacacatttgccttcatcatggacacggggaaccagcgctttgagtgccacgttttctggtgcgagcctaatgctggtaacgtgtctgaggcggtgcaggccgcctgcatgttacgatatcagaagtgcttggtagccaggccgccttctcagaaagttcgaccacctccaccgccagcagattcagtaaccagaagagtcacaaccaatgtaaaacgaggggtcttatccctcattgacactttgaaacagaaacgccctgtcaccgaaatgccatagctgcacatgcaaaaggactcggctatttacctgaagattgactagctacactaaagaaaatgaactccgccatccgaccttccatccagttgctgatgctttgtcttcagagaatttacccttaaccaagcagtgttagacaagcatgttctctcgtcttgccaccatcatgtgatatgaaaagaagcatgaataattttttttgctgtaagttacatcatgcgcagtggaaggtctttttcttattgtaaatattgtgaacattacttaacttcacacacacacagagaagagtgtggccccacccctcctagtgaactaacgctgcgtccttggaatgaatgatgcgtgagttagtttcactgtcttcttggctggacctgtcacaagcaacctttaagtcctacagcactttgccctgttttcaacattggagtaggcactgcatagcagataccattgaattgctgtaaaaataggatggcgagtttgtgttttaatttttcataaaattgaacctgttggttgacaaaattggctgttggcatcagtatagaaaccaactggcagctttccctgacaagctctttgacacatggacaccatttcatgtctacagctgtttgtgggatgttggaaaaaaatgaaacttcaaaattgatgaaaaactaaattcgaggaattaaaatcgaacaaaacatagcctttcttttccgatggttttcaaactgattatttttaaaagagattaataaaatcataatgcattttgggtgggacatatttcaaacttctgccttatattgtacggtgcagctagagaattatagttcactatggccattctctacataaacattaagatgaaatactcctcatcagcctttcatccttagtttgagaattagctgatatgcaatttgaagttgaggaaatatcattgatatttctatcatgcacgattattttagatttctaccaccgtgtgatttttgctagtccatgtgctagaggtaaacgttctgctggaattctgcatccagctctatccccctctgatgctttttgcccagaaagctgtctgtccatcatgtattgtccatggcaacaaattacattaggttgaacctttccttgattttatgtatttaatattagaatttgttggactcaactagatatattttttaatttatattttttccattttactttgaagatttgaaatgttcatacctgagcaaagtctacacaggagtaatggactgtttaacaagtttcccaaaacagcattttcctgctccttcgtatgtaggtgagaaacttagctggaaagacatacaaatttagactctcgttgacattgtcgttttaaaaggaagttgctaaggcgatcaatctcaatattagtcttgtttacttcttcttaatgtcaaaattaacatttacaacatccaattataaaagtaatgctttatgtttatacactgctatgtacttgtcaaaatggtttccacattcttatcacatctgagccttaccaggtagagaaggtactaaatacactttagaagtaaaaatatgaagtaccgagaggctaaacccactggcctaagatctcaccaaagttcatgaaaaccaggactaggacccacggctcccaaagcccgttcttgctgtgttgtgctgcctccatatccgtcaggaagagcctttccagaatgattctgggcatatactaagaagagcaggtatggaaagatctattgtcagggaatcttagaattccctacacgagtgggagaaagatgtccaaattccttacgcagtggtattcatgatggtgccctatctaagtccaggactgttttcctacagcgtgcctcaaaagtgttgtagagggcaggattctacattcacagcctgttccatctacgagattttccagatgctacttgtggtagacattcctaactcatggtacttagccaccagagatcatgatggaatgagtgggtggcttttctacctgccattccctcagaattcatgaggggtgggggacagggggaccggaattgtcttagcaccccaatgttatgacaaaactatgctactttagaaacgcagtctgtttttcaccaattgacatactactgatctgaagtaaccagtgccatcataagaaattactgcattaagaaaatccttgctgtgccctttgaaaagctgttcagaaatcatttacagtgatctttcatctcggtcgctgtagtgaaacattttagtgtgataaatttcaaaattctaaacaaattacccacttttatattggaaatctctaccagaactccctcttcattttttaaggcatacatttgcttgttttcaagatcaagaattctgagctagctttaagtagcaaactgatttatatgtgcaattataggatgcattaagatgaatgatagcctttacatattgaaaactttgcagacgttttgttttgaaaatggcattgtatagtaaatgcaaattaattttgtaaaattatgttaaagagtatgttcagacactttctgccatggccaaaaagtatgtatgaaagtatgtgtgtatttgtttgtaaaaggatgccaatgttttacctgatatcttagtgacacttcagttatctatgcattctttagatctgtgattcggtaaacaggcagccatgttcacgatgccttctatgtcttaccatatttttaattaacctgttaaatacagcttaaaatatttttattttatttattctatttttactgaaatatactgcattattgtgttaatgtattatctttcctggatattatctcccagtgtatccagatctaagtaatctcagtgaactatacattgcctaaaaagtggttttgtaatgatttgtagtcacatttctattgggatatgtagaagaaaaggcaaaatgcttaaagttccttttattttttaaaagcagctagatagacacagacttgccacctcatacatctgctccttggcaacatcaaggggaacgactagccaacatgcctatggctaaaaactttcctttgcagactaaagcactgcttggtgcttcgtttttctacccttcacaacatgtgtgatttcatctaagagatatatacatgtacacatgccctttgtttccacctggatacaagatcactcatagctaattaggaccattgttttttgttcatctgtcttgttgcatgaagggacattagacccatttccattaaaataagttcttggtgataaactgtggcactgctacttctttttaaatccactttatgatttcaagatggacacttgtaagatgactcgacacaaggccattgcctggaagccccagagctttcctctgtttgtatggcccgttcatgtcccaggcattgcaacacaaactcctcaagatttcaccacaacatgacaagcattttcctaactgatattagcacaatttaactaataagccccttcgctctctagttggccaggcttaacctaatacacatctaacgtgtgtgccacacggccagtagaaagtttaacttcagcttcagggcaaagatacccactcacaccgtgtcaacgcaagcagtagttcctggcctccagagcagcttacttcccctgaaagaacgctttgttttcctttatgcccttttcctgttgaccacttttacacatttaaatgtaatttgttgtgagaataaatttagctgcataaaacgttcggctcatttatctgacatcttagtcacatatacaaggaatagaaatagaaactcggtgtctctagttatttttaaattattcttacctcagacttcttagaaatcactttagtaatggagcattttgctttgattagttactacatatttctgcctggtaagaactaggaagtaacttcaaattttgagtaatcaccctgtacttatttggtgatcaggaaggccagctggccttccggacatagaagcctatttagtcaccaactcgagtcttttgtaagcggtcttgctaggattgtgatattttagcacgaagaagtttatcacttcctttaagaacctgacatcaaagaataaagaatagaggtgtacacacactaaatccaaaatgaaaggtaactagagaaatcagttgaatctggtttagcttaactgttaggcgcaggaaggcagataaacagaatttaaagtatgtccccgctttttgttcatcttgcacttccacagtggtttctctctagtcagtaacaaaatttcattatggtttcaggcattatatggtggtaaataatttcagattaaaaatgtgtttgctattggagtatctgaatactagtaatttcattatttagaattttgcagcacttttatctcaagaagaagtccaagaatgtaaaatgccaaatgaaacatgtcagtggaatcaatattctccttcattagaattccctcatattgctttttttttttttcttcagacagaggagtcttactctggagtgcagtggtgtaatttcagctcaccacaacctccacctcccaagttcaagcaattctcgcctcagcctcctgagtagctgggattacaggcatgcaccgccacgcctggccaatttgtatatttttagtagagacagggtttcgccacgttggccaggctggtctcgaactcctgacctcaggtgatccgcccgccccaacctcccaaagtatgtgagccaccacgtccggcctcatattgcttttatccaaaattcttttcccttttcactctaccaaagtatttaaataatcctgtccttcatagaagattctcaaagaagaaaactgcagtgtaattaatgaatggtttaattcagaatcttcatatacttctaaagagaaaaataatttagtgccaaatgcatgttaggagataatcaatgtaagtggcaacaaattgtgacttcacatgctactgtagagatcagaaaattatcctaaactattccataacaatgagacaacatcacagaaaatacacttgaaaataaaaatctcaagaccagctacttctggacaatggaatacttttcagtctggtatggtggagggcccgaaaaggataagggattcttatgatacacaatgggattctttactgaacaatatgttaaattaagctgcaccgccttccttgaggcatggactaccctaaccaaccagatagaaatctgggtgggataagaggatgagccacacgctataattttagggcaaggagatagtgtttgattttcaaaatcagcaaaataagctgagcactttatatctttctgtacaagagtgataacatgaagaattcttcttcagggatttaaaatacaataagcctggttcaactataaaaagtcttgtttcctttcttcattgacacttttttttttttttttttttttttgaggcaaggtctcactctgcttcccaggctggagtgcagtggggcaatattggctcactgcaacctgcacctcctggactcaagagatcctcgtacctcagcctcctaagtagctgggactacaggcgtgtcccaccacacccagctaatttttgtattttttgtagagatggggttttggggtttcgccatgttgtccaggctcgtctggaactccggtgctcaagtggcgtgcccacctcagcctcccaaactgctgagattacagatgtgagccactgcacccagcccactgacacgttttactgataaatgtaaatctaagctaaaataaaaataatgtattaccgctataatacaattcaccattctcttttctcacttcaagtaagaaagtaaaaatagaatatcagagctgaagtagacctaagtattcatcttgaagaagataatattctaaaaatcatgccacctgaattgagcatttaggaatttatgtaacatttctatacaactgaattgcaaaaataaaactttaaattcaaacttta

SEQ ID NO: 7 - DNA, 2270 bp, Homo sapiens
  source 1..2270: mol_type = genomic DNA
attatgcagatgcacggctggaggtgggatccacacagctcagaacagctggatcttgctcacactctttcaagagaagcttccttggacaaaaggaccctgccttggtgtgagagtgagggcagagggagctggagcaagtagaatttctctaaataccagctggctggggcccaggagattaaaaaacaccgggctaggttggtcttggcatttgctgacacgcaaagggattgcagagatccagcccctccaacctccctctgtccacaggtggctcacattcagtcccacaatttgctttctcctcctcaagggttaagaaaaaaaacgaacccttccagtcaggtcagtgactggagagctccatggaaagtctctcagtgacctggctgctggcaccatggactcagaaaagaaacgctttactgaagaggccaccaaatacttccgggagagagtcagcccagtgcatctgcaaatcctgctgactaacaatgaagcctggaagagattcgtgactgcggctgaattgcccagggatgaggcagatgctctctacgaagctctgaagaagcttagaacatatgcagctattgaggacgaatatgtgcagcagaaagatgagcagtttagggaatggtttttgaaagagtttccccaagtcaagaggaagatccaggagtccatagaaaagcttcgtgcccttgcaaatggtattgaagaggtccacagaggctgcaccatctccaatgtggtgtccagctccactggcgctgcctctggcatcatgtcccttgctggtcttgttttggcaccatttacagcagggacgagtctggcccttactgcagctggggtagggctgggagcagcgtctgctgtgactgggatcaccaccagcatcgtggagcactcatacacatcatcagcagaagctgaagccagcaggctgactgcaaccagcattgaccgattgaaggtatttaaggaagttatgcgtgacatcacacccaacttactttcccttcttaataattattacgaagccacacaaaccattgggagtgaaatccgtgccatcaggcaagccagagccagggcccgactccctgtgaccacctggcgaatctcagctggaagtggtggtcaagcagagagaacgattgcaggcaccacccgggcagtgagcagaggagcccggatcctgagtgcgaccacttcaggcatcttccttgcactggatgtggtcaaccttgtatacgagtcaaagcacttgcatgagggggcaaagtctgcatctgctgaggagctgaggcggcaggctcaggagctggaggagaatctaatggagctcactcagatctatcagcgtctgaatccatgccatacccactgaccccagaccagtgcagccagcaggggaggtgagccatacacaggccacgacaaaatgcaggcattttattagggggataaagagggcaaggtaaagtttatggagctgagtgttagtgactttggcatttctgtagctgagcacagcaggggaggggttaatgcagatggcaagtgcaccaaggagaaggcaggaatgctggagcctggaataagggaggagaggggactggagagtgtggggaataggaagaagaaatttcctttagactaacgaatatattggggggaggaatagaggggaggtgtgcaggaaccagcaatgagaaggccaggaaaagaaagagctgaaaatgcagaaagccgaagagttagaacttttggatacagcagaagaaacagcggctccactaccgacctgcccccggttcgatgtccttccaagaatgaagtctttccctggtgatggtcccctgccctgtctttccagcatccactctgtcttgtcctcctggaagtgtatctcagtcagccagtggcttcttgatgatggcggtggaggtggtggttgtagtgtgatggatcccctttaggttatttaggggtatatgtcccctgcttgaaccctgaaggccaggtaatgagccatggccattgtccccagctgaggaccaggtgtctctaaaaacccaaacatcctggagagtatgcgagaacctaccaagaaaaacagtctcattactcatatacagcaggcaaagagacagaaaattaactgaaaagcagtttagagactgggggaggccggatctctagagccatcctgctgagtgccctgtgtgtaagtcctaataaactcacctactcaccaa

SEQ ID NO: 8 - DNA, 1825 bp, Homo sapiens
  source 1..1825: mol_type = genomic DNA
gctcgctgcgccaccgcctcccgccacccctgcccgcccgacagcgccgccgcctgccccgccatgggtcgacagaaggagctggtgtcccgctgcggggagatgctccacatccgctaccggctgctccgacaggcgctggccgagtgcctggggaccctcatcctggtgatgtttggctgtggctccgtggcccaggttgtgctcagccggggcacccacggtggtttcctcaccatcaacctggcctttggctttgctgtcactctgggcatcctcatcgctggccaggtctctggggcccacctgaaccctgccgtgacctttgccatgtgcttcctggctcgtgagccctggatcaagctgcccatctacaccctggcacagacgctgggagccttcttgggtgctggaatagtttttgggctgtattatgatgcaatctggcacttcgccgacaaccagctttttgtttcgggccccaatggcacagccggcatctttgctacctacccctctggacacttggatatgatcaatggcttctttgaccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctgagtgggcaggggccatctccccactccgctgccctggccttgagcatccactgactgtccaagggccactcccaagaagcccccttcacgatccaccctttcaggctaaggagctccctatctaccctcaccccacgagacagccccttcaggatttccactggaccttgcccaaatagcaccttaggccactgcccctaagctggggtggaaccggaatttgggtcaatacatccttttgtctcccaagggaagagaatgggcagcaggtatgtgtgtgtgtgcatgtgtgtgcatgtgtgtgcatgtgtgtgcaggggtgtgtgtgtgtggggggggttcccagatattcagggcaagggaccagtcggaagggattctggctattgggggagcccagagacaggggaaggcagcctgtccatctgtgcataaggagaggaaagttccagggtgtgtatgtttcaggggcttcacatggaggagctgcagatagatatgtgtttctgtgtatgtgtatgtctgcctttttttctaagtgggggcttctacaggcttttgggaagtagggtggatgtgggtagggctgggaggagggggccacagcttaggtttggagctctggatgtacatacataagtaggagcagtgggacgtgtttctgtcataatgcaggcatgaagggtggagtgaagtcaggtcataagtttcatgtttgcttttgttttgttttgtttttaatgtatgtagcagatgttacagtcttagggatccgggatgggagaccccactttagaaagggtcgtcactcctttaatcctctactcaacaatgtactcttttacttttatattaaaaaaaataaaataaatatgtgcctaaaa

SEQ ID NO: 9 - DNA, 1599 bp, Homo sapiens
  source 1..1599: mol_type = genomic DNA
acaaccagctgactcccgtagaggaagacactgtggaggccagttctggagctattgcagcctcggttgcccggccggggacccgagccgaaaagttatcgtcagaatgtcgggcaaagaccgaattgaaatctttccctcgcgaatggcacagaccatcatgaaggctcgtttaaagggagcacagacaggtcgaaacctcctgaagaaaaaatctgatgccttaactcttcgatttcgacagatcctaaagaagataatagagactaaaatgttgatgggcgaagtgatgagagaagctgccttttcactagctgaagccaagttcacagcaggtgacttcagcactacagttatccaaaatgtcaataaagcgcaagtgaagattcgagcgaagaaagataatgtagcaggtgttactttgccagtatttgaacattaccatgaaggaactgacagttatgaactgactggtttagccagaggtggggaacagttggctaaattaaagaggaattatgccaaagcagtggaactactggtggaactagcttctctgcagacttcttttgttactttggatgaagctattaagataaccaacaggcgtgtaaatgccattgaacatgtcatcattccccggattgaacgtactcttgcttatatcatcacagagctggatgagagagagcgagaagagttctataggttaaagaaaatacaagagaagaaaaagattctaaaggaaaaatctgagaaggacttggagcaaaggagagcagctggagaggtgttggagcctgctaatcttctggctgaagagaaggacgaggatcttctatttgaataatctttcctgttctggttctttgagaaaccctaacactggcttcattttaattcacagtgtgtaggtttgatttgtgtggctatttattttttggcctaagaatttcactggttgtaaaatttacctagatgtctatttatgggattacttttgcagaatcataatttagcaaccatttatcatggatgaaagagatctgtaaaacctgcccaggaacttacagaatttactttgcagaagcgttatcatactccatttacatctgtgttacacgtgatctgcttaccaagcatattaggaaatacctcttaggaagcattagcggtctcaggccaattactgtggagcagctttcattcctacccacttgcaaaccttggcgctgttgtctgagattgctgcagccattcttgttaccatggtacttctcaaactttgtgaaaacctgcacttttccttgcatgacaggttcctgtcttgtctgtcatgggagccattctgccaatttaaatgcgactgtggtataaacagtaaaatgatttaaaagtaagtcattccgtttttattaatttactgttaagtcatgttctcatgctcagatcagtagtgtcagccagagctttctctgcagacatgtaggaagtgggtagctatttttcccactccatgtattagagttttacaaaaaggcttacttttgagacaactgttgcattttggggtactaataaatgattgccgatgagtta

SEQ ID NO: 10 - DNA, 2262 bp, Homo sapiens
  source 1..2262: mol_type = genomic DNA
aggagccgccggcaagggggcaacgaggaagctcttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttaggtaaagtttcatctattcattctgatcaatctgatgtgttaatggtggatgtggcaaccagtatgcctgttgttatcaaggaggtgccaagctcagaggctgctgaagatgtcatggtggctgctcctcttgtgtgttctggaaaggtgctagaagtgcaggttgtgaaccaaacatctgtccatgtagatttgggttctcaacctaaagaaaatgaggctgaaccatcaacggcttcctcagtccccttgcaggatgaacaagaacctcctgcttatgatcaagctcctgaggtcactttgcaggctgatattgaggttatgtcaactgttcatatatcatctgtctataacgatgtgcctgtgactgaaggagttgtttatatcgagcaactgccagaacaaatagttatcccttttactgatcaagttgcttgtcttaaagaaaatgagcagtcaaaagaaaatgagcagtcaccacgagttagtcccaaatctgtagtagaaaagaccacctctggcatgtctaaaaaatctgtagagtctgtaaaacttgcacagttggaggagaatgcaaaatattcctcagtatatatggaggcagaagcaacagctctgctctctgacacatctttgaaaggtcagcctgaggtacctgcacaactcctggatgcagaaggtgctatcaaaataggctctgaaaaatctctgcaccttgaagtggagatcacttcaatagtctctgacaatactgggcaggaggagtctggggaaaactctgtaccccaggagatggaaggcaaacctgtgctctctggggaagctgcagaagcagtgcactcaggtacatctgtaaagtcatctagtggccccttccctcctgctccagaaggccttactgcaccagaaattgaaccagaaggggaatcaacagctgaataaggtttgatgaagccagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcctggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagcata

SEQ ID NO: 11 - DNA, 1296 bp, Homo sapiens
  source 1..1296: mol_type = genomic DNA
actctgaggctctttccaacgctgtaaaaaaggacagaggctgttccctatggcagaaggcaaccacagaaaaaagccacttaaggtgttggaatccctgggcaaagatttcctcactggtgttttggataacttggtggaacaaaatgtactgaactggaaggaagaggaaaaaaagaaatattacgatgctaaaactgaagacaaagttcgggtcatggcagactctatgcaagagaagcaacgtatggcaggacaaatgcttcttcaaaccttttttaacatagaccaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctagaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattgaaaatggaagccacaagcagcccagccctccttaatcaacttcaaggagcaccttcattagtacagcttgcatatttaacattttgtatttcaataaaagtgaagacaaacgaa

SEQ ID NO: 12 - DNA, 2456 bp, Homo sapiens
  source 1..2456: mol_type = genomic DNA
acagttttcatccagccacgggccagcatgtctgggggcaaatacgtagactcggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacgaaatactggttttaccgcttgctgtctgccctctttggcatcccgatggcactcatctggggcatttacttcgccattctctctttcctgcacatctgggcagttgtaccatgcattaagagcttcctgattgagattcagtgcatcagccgtgtctattccatctacgtccacaccgtctgtgacccactctttgaagctgttgggaaaatattcagcaatgtccgcatcaacttgcagaaagaaatataaatgacatttcaaggatagaagtatacctgattttttttccttttaattttcctggtgccaatttcaagttccaagttgctaatacagcaacaatttatgaattgaattatcttggttgaaaataaaaagatcactttctcagttttcataagtattatgtctcttctgagctatttcatctatttttggcagtctgaatttttaaaacccatttaaatttttttccttacctttttatttgcatgtggatcaaccatcgctttattggctgagatatgaacatattgttgaaaggtaatttgagagaaatatgaagaactgaggaggaaaaaaaaaaaaaagaaaagaaccaacaacctcaactgcctactccaaaatgttggtcattttatgttaagggaagaattccagggtatggccatggagtgtacaagtatgtgggcagattttcagcaaactcttttcccactgtttaaggagttagtggattactgccattcacttcataatccagtaggatccagtgatccttacaagttagaaaacataatcttctgccttctcatgatccaactaatgccttactcttcttgaaattttaacctatgatattttctgtgcctgaatatttgttatgtagataacaagacctcagtgccttcctgtttttcacattttccttttcaaatagggtctaactcagcaactcgctttaggtcagcagcctccctgaagaccaaaattagaatatccatgacctagttttccatgcgtgtttctgactctgagctacagagtctggtgaagctcacttctgggcttcatctggcaacatctttatccgtagtgggtatggttgacactagcccaatgaaatgaattaaagtggaccaatagggctgagctctctgtgggctggcagtcctggaagccagctttccctgcctctcatcaactgaatgaggtcagcatgtctattcagcttcgtttattttcaagaataatcacgctttcctgaatccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggagctttggacctaatccaagcatccctttgcccagaaagaagatgggggaggaggcagtaataaaaagattgaagtattttgctggaataagttcaaattcttctgaactcaaactgaggaatttcacctgtaaacctgagtcgtacagaaagctgcctggtatatccaaaagctttttattcctcctgctcatattgtgattctgcctttggggacttttcttaaaccttcagttatgatttttttttcatacacttattggaactctgcttgatttttgcctcttccagtcttcctgacactttaattaccaacctgttacctactttgactttttgcatttaaaacagacactggcatggatatagttttacttttaaactgtgtacataactgaaaatgtgctatactgcatactttttaaatgtaaagatatttttatctttatatgaagaaaatcacttaggaaatggctttgtgattcaatctgtaaactgtgtattccaagacatgtctgttctacatagatgcttagtccctcatgcaaatcaattactggtccaaaagattgctgaaattttatatgcttactgatatattttacaattttttatcatgcatgtcctgtaaaggttacaagcctgcacaataaaaatgtttaacggttaaa

SEQ ID NO: 13 - DNA, 2555 bp, Homo sapiens
  source 1..2555: mol_type = genomic DNA
atcagtcaggaggccgtgcagtccgagatgggctcgtctcgggcaccctggatggggcgtgtgggtgggcacgggatgatggcactgctgctggctggtctcctcctgccagggaccttggctaagagcattggcaccttctcagacccttgtaaggaccccacgcgtatcacctcccctaacgacccctgcctcactgggaagggtgactccagcggtttcagtagctacagtggctccagcagttctggcagctccatttccagtgccagaagctctggtggtggctccagtggtagctccagcggatccagcattgcccagggtggttctgcaggatcttttaagccaggaacggggtattcccaggtcagctactcctccggatctggctctagtctacaaggtgcatccggttcctcccagctggggagcagcagctctcactcgggaaacagcggctctcactcgggaagcagcagctctcattcgagcagcagcagcagctttcagttcagcagcagcagcttccaagtagggaatggctctgctctgccaaccaatgacaactcttaccgcggaatactaaacccttcccagcctggacaaagctcttcctcttcccagacctttggggtatccagcagtggccaaagcgtcagctccaaccagcgtccctgtagttcggacatccccgactctccctgcagtggagggcccatcgtctcgcactccggcccctacatccccagctcccactctgtgtcagggggtcagaggcctgtggtggtggtggtggaccagcacggttctggtgcccctggagtggttcaaggtcccccctgtagcaatggtggccttccaggcaagccctgtcccccaatcacctctgtagacaaatcctatggtggctacgaggtggtgggtggctcctctgacagttatctggttccaggcatgacctacagtaagggtaaaatctaccctgtgggctacttcaccaaagagaaccctgtgaaaggctctccaggggtcccttcctttgcagctgggccccccatctctgagggcaaatacttctccagcaaccccatcatccccagccagtcggcagcttcctcggccattgcattccagccagtggggactggtggggtccagctctgtggaggcggctccacgggctccaagggaccctgctctccctccagttctcgagtccccagcagttctagcatttccagcagctccggtttaccctaccatccctgcggcagtgcttcccagagcccctgctccccaccaggcaccggctccttcagcagcagctccagttcccaatccagtggcaaaatcatccttcagccttgtggcagcaagtccagctcttctggtcacccttgcatgtctgtctcctccttgacactgactgggggccccgatggctctccccatcctgatccctccgctggtgccaagccctgtggctccagcagtgctggaaagatcccctgccgctccatccgggatatcctagcccaagtgaagcctctggggccccagctagctgaccctgaagttttcctaccccaaggagagttactcaacagtccataagaagtcaactgttgtgtgtgtgcatgccttgggcacaaacaagcacatacactatatcccatatgggagaagtccagtgcccaggcatagggttagctcagtttccctccttcccaaaagagtggttctgctttctccactaccccaaggttgcagactctctcttatcaccccttcctccttcctcttctcaaaatggtagattcaaagctcctctcttgattctctcctactgtttaaattcccattccaccacagtgcccctcagccagatcaccaccccttacaattccctctactgtgtggaaatggtccattgagtaacacccccatcagcttctcaactgggaaacccctgaaatgctctcagagcacctctgacgcctgaagaagttataccttcctcttcccctttaccaaataaagcaaagtcaaaccatcatctggaaacagtggccacttttcactgacctttcttcgacatctagtcaacccacccaatatgccactgggctctcgctcccaattccaccccaccctccattacagagctcaccacgccctcctagatcaccgtccccaacacacccattgcctctcaaggcccttatctcagccccttcctgtggccacttccctcagtgctcagatgattccctgggtgagggagacactggggcaccctcagaggttgaagcaggctccctgctgtccctggatcctggacagatggctcagtaaactgtggggactaggtgcagactttttgccttcttggagtcctgggtctcctctgagagtctgggtggtgctcttcctacgcctctagaggtctctgtgttcctcattttccttcaaaagcgggctgtgtttctcttctaccttccagctgctcccacagaggaggaagacaataaatatttgttgaactgaaagca

SEQ ID NO: 14 - DNA, 1572 bp, Homo sapiens
  source 1..1572: mol_type = genomic DNA
gccccacccgcgagccgcagccgcggccccacagcttctggggctggggccccggcagccgggcaggccggcctgacctcgacctccgccgtgcgggcccgaccggtgagtccagcccggcagtcgcaggacccggccgccagcctctccctccacctctccctgcccccagcgccaggcgcgggctgcgctcggtggcggcggcggggccctcaggcggccatggcggcaggcgccggggccgggtccgcgccgcgctggctgagggcgctgagcgagccgctgagcgcggcgcagctgcggcgactggaggagcaccgctacagcgcggcgggcgtctcgctgctcgagccgccgctgcagctctactggacctggctgctccagtggatcccgctctggatggcccccaactccatcaccctgctggggctcgccgtcaacgtggtcaccacgctcgtgctcatctcctactgtcccacggccaccgaagaggcaccatactggacataccttttatgtgcactgggactttttatttaccagtcactggatgctattgatgggaaacaagccagaagaacaaactcttgttcccctttaggggagctctttgaccatggctgtgactctctttccacagtatttatggcagtgggagcttcaattgccgctcgcttaggaacttatcctgactggttttttttctgctcttttattgggatgtttgtgttttattgcgctcattggcagacttatgtttcaggcatgttgagatttggaaaagtggatgtaactgaaattcagatagctttagtgattgtctttgtgttgtctgcatttggaggagcaacaatgtgggactatacgattcctattctagaaataaaattgaagatccttccagttcttggatttctaggtggagtaatattttcctgttcaaattatttccatgttatcctccatggtggtgttggcaagaatggatccactatagcaggcaccagtgtcttgtcacctggactccacataggactaattattatactggcaataatgatctataaaaagtcagcaactgatgtgtttgaaaagcatccttgtctttatatcctaatgtttggatgtgtctttgctaaagtctcacaaaaattagtggtagctcacatgaccaaaagtgaactatatcttcaagacactgtctttttggggccaggtcttttgtttttagaccagtactttaataactttatagacgaatatgttgttctatggatggcaatggtgatttcttcatttgatatggtgatatactttagtgctttgtgcctgcaaatttcaagacaccttcatctaaatatattcaagactgcatgtcatcaagcacctgaacaggttcaagttctttcttcaaagagtcatcagaataacatggattgaagagacttccgaacacttgctatctcttgctgctgctgtttcatggaaggagatattaaacatttgtttaatttttatttaagtgttatacctatttcagcaaataaaatatttcattgcttatattaa

SEQ ID NO: 15 - DNA, 2396 bp, Homo sapiens
  source 1..2396: mol_type = genomic DNA
agtagcaccaccgccttccaagtttccccttgtggatgcgcggccccgcggctctgctcctcccggcgcagaggggccgggagaggccacaggagcggacctggcacgggatttctgaggaacgggagaagactggcgcccgacccgctctggagggtcggtgaacgatgaagggccggcggcggcgacgccgagagtactgcaagttcgcgctgctgttggtgctgtacacgctggtgctgttgctcgtcccctccgtattggacggcggccgcgacggggacaagggcgccgagcactgccccggcctgcagcgcagcctgggagtgtggagcctggaggcggcggcggccggcgaacgcgagcagggagcggaggcgcgggccgccgaggaagggggcgcgaaccagtctcctcggttcccaagcaacctcagcggcgctgtcggggaggcagtgtctcgcgagaagcagcacatctacgtgcatgccacctggcgcaccggctcgtccttcctgggcgaactctttaaccagcacccggacgttttctacttgtatgagcccatgtggcatctatggcaggcgctgtatccgggcgacgccgagagcttgcagggcgcgctgcgcgacatgctgcgttcgctcttccgctgcgacttctccgtgctgcggctgtacgcgccgccgggggaccccgctgcgcgcgccccggacacggccaatcttaccacggccgccctcttccgctggcggactaacaaggtcatctgctcgccgccactgtgtcctggcgcaccccgtgcccgggccgaggtgggcctcgtcgaggacaccgcctgcgagcgcagctgcccacccgtggcgatacgcgccctggaggccgagtgccgaaagtacccggtggtggtcatcaaggacgtgcgcctgctcgatctgggcgtgctggtgcccctgttgcgtgatccaggcctcaacctgaaggtggtgcagcttttccgcgacccgagggcggtgcacaactcgcgcctcaagtctaggcagggactgctgcgcgagagcatccaggtgctgcgcacccgccagaggggcgaccgcttccaccgtgtgctgctggcgcacggcgtgggtgctcgccccgggggccagtctcgcgcgctgcccgccgcgccgcgcgccgatttcttcctgaccggtgcgctcgaggtgatctgcgaagcctggctgcgcgatctgcttttcgcgcgcggcgcgcccgcctggctgcggcgccgctacctgaggctgcgctatgaggacctggtgcggcagccacgcgcccagctgcgccgcctgctgcgcttctccgggctacgcgcgctcgcagcgctcgatgccttcgcgctcaacatgactcgcggcgcggcctacggcgccgaccggcccttccacctgtcagcgcgcgacgcccgggaggcggtgcacgcctggcgcgagcgcctgagccgagagcaggtgcgccaggtggaggccgcctgcgctccagccatgcgtctgctcgcctaccctcgcagcggagaggagggcgacgcggagcagcccagggaaggggagacgccgctggagatggatgccgacggcgccacgtagcctcccatccctgtccccggcacggatccgggtcagtcaccacgaacaggggcactcggcatgctgccccagcactggagaagcagcgctgtgggggcaatctgtcacactctcagagtctgggacttgacttgctaccaacaactgctgtgcaattctgctgagcaggaatatcatgagctgttcaataatgacggacgcattggttgagatgaagtttccagtaaggaagtgacagtgcaatgtggatatttatggctgtaaaataggaagagctttagttcccaggctgaacctgccactgctggagccatttcaacaaggcatcctcacaacaaagaagagatgtgatttggtaccatttcacaccagcaggtgtctggacgaaaacatcaatgtgaataagggccaagtgcagtcctgtcttgattaaattacttaataatattattaaataataataggtctgggcagtattgtttttaacctgactcatccagctgtccttcaaatagctccgtctccctctacccagaactgatttttaaaaagaagtaatttttctccctgggctgggaaaaccctaatgaactgaaacacacttttactttaaaatttttctgtctggcgtttttgtaatcatactattaaatgactctggagtcatgttaatgacaggatttgttttgtttggatgcagttcaattgcatggtttgggtaaaagctagcctacatacaaaggaatatgaagactgtggaagaaactg

SEQ ID NO: 16 - DNA, 4657 bp, Homo sapiens
  source 1..4657: mol_type = genomic DNA
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcatagagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 17 - DNA, 2434 bp, Homo sapiens
  source 1..2434: mol_type = genomic DNA
agacgctctctagatgtgcacgcatgtgcgtgtggagtgtgtgtgtgtgtgtgtgtgagagagagagatgtgttggggtggggggactgcaggtcaggaagtcctgcagatgtgcgtgcctgtgtgtgtttgcgcgcgcgcgcgcgtgtgtgtgtgtctgtgtgtgtgcctggagctgcgcttgcaggtctgagagtcctctgagaatgggttgggggaggcgggtgcaggaccgaggcctggaacagccaacaaagaggccctggaggagggtggggctggccggggacaggaggggtggccaagaaagtcgcaagaaaactccggcccccaagaaaaagagagggcatgggttgcggagccgacatcacggccggggtctttgctgtttagacgcctgggttcccggatcccagacacgcgcacgggcaggaagttagaccggagacagcgagtatagacagcccttctggataggggcagacgggtgggagctccgaaggggtgccggggcggtgatcgggaggtctccagcggcccgaaccagggtgaagaagaagcccgggtttccgcttccctccgggcgcgagaagaggggagccaggccgagccccggccctaccgccgccgccgccatgtggcccccagaccccgaccccgacccggaccccgagcctgccggcggctcccgtcccggccccgcggtccccgggctccgcgccctgctgccggcgcgggctttcctctgctctctcaaaggccgcctcctgctggccgagtcgggtctctcattcatcacttttatctgctatgtggcgtcctcagcatctgccttcctcacagcgcctctgctggagttcctgctggccttgtacttcctctttgctgatgccatgcagctgaatgacaagtggcagggcttgtgctggcccatgatggacttcctgcgctgtgtcaccgcggccctcatctactttgctatctccatcacggccatcgccaagtactcggatggggcttccaaagccgctggggtgtttggcttctttgctaccatcgtgtttgcaactgatttctacctgatctttaacgacgtggccaaattcctcaaacaaggggactctgcagatgagaccacagcccacaagacagaagaagagaattccgactcggactctgactgaaggcctggcgggtgccttggcaacctgagccacacaggcctccacccctgcgcctcacaggggtcgctggcgttggagcggaggcctggacttctgagttgcagagggggctgcggacacagcaggccccctacagcctcaggttctgcctgagcccagcctaccaggcttgcccctcagctcagcactgttgaccacgctgcgtatgagggcatcttgggtatcccactccttctccccatttctgtcccacaggccttcagccctttaacgtctctgccaaaaaccagcacaaggagacaaagcagagccttgtctgtatctgggcagcaggtgttccatgctgctaggtggcgggggtcgggggtcttctgtttcactaacaggaacaaagacagaaaccatgacagggctgccccgccaggccccggtgggtttgtctgcacttggtgctcctgcccacaccagccactttggtgacaatgacccttccaagaatctttggttcaaggagcaccagttccctcttcattcttgaagcagggagaaattgacctttgccttgtcgcccaggaagtggggctcggcacccataactaacacctcccacccttggaaaccatgtcttctgggggtgagatgaccattctgggtctaagactgtttcaaagaagagctcatagactgactggtccagaagacagagggtacaacagtggcatcacagtgacagtgtcatggggagctgggcgggcccagccaaaccctccttcttcctagagcccagccagcaggcaggagttcctggaccctcaggacagtgaacttccagacctcagggcaggtctatgggccactgcaggagatgagaccagccttctgtgttcacctaacgatttatactgtgtatctgtctttgatggaattttgtaactttttatatttttttatgcaaaagcagcttcttaacagatggcattttctgtgactctaggcctcacaaaagagccagagttctggacccatgtttggagcatttgtagccttattctcttgcgtgtgaatctcttaccctgaaaaaaagccataatgaattaagccagactgaccacttgcttggagtgtgtgcttgaaaaaaccagagcaatactgttgggtattgtatcaggcttcagtacaaactggtaacaccaatgtggatcctgacagctttcagttttagcaaaaatacacgtgaaatctgactacca

SEQ ID NO: 18 - DNA, 4203 bp, Homo sapiens
  source 1..4203: mol_type = genomic DNA
agaaggcagcctcggtctctgggcggcggcggcggcccactctgccctggccgcgctgtgtggtgaccgcaggccccagacatgagggcggcccgtgctctgctgcccctgctgctgcaggcctgctggacagccgcgcaggatgagccggagaccccgagggccgtggccttccaggactgccccgtggacctgttctttgtgctggacacctctgagagcgtggccctgaggctgaagccctacggggccctcgtggacaaagtcaagtccttcaccaagcgcttcatcgacaacctgagggacaggtactaccgctgtgaccgaaacctggtgtggaacgcaggcgcgctgcactacagtgacgaggtggagatcatccaaggcctcacgcgcatgcctggcggccgcgacgcactcaaaagcagcgtggacgcggtcaagtactttgggaagggcacctacaccgactgcgctatcaagaaggggctggagcagctcctcgtggggggctcccacctgaaggagaataagtacctgattgtggtgaccgacgggcaccccctggagggctacaaggaaccctgtggggggctggaggatgctgtgaacgaggccaagcacctgggcgtcaaagtcttctcggtggccatcacacccgaccacctggagccgcgtctgagcatcatcgccacggaccacacgtaccggcgcaacttcacggcggctgactggggccagagccgcgacgcagaggaggccatcagccagaccatcgacaccatcgtggacatgatcaaaaataacgtggagcaagtgtgctgctccttcgaatgccagcctgcaagaggacctccggggctccggggcgaccccggctttgagggagaacgaggcaagccggggctcccaggagagaagggagaagccggagatcctggaagacccggggacctcggacctgttgggtaccagggaatgaagggagaaaaagggagccgtggggagaagggctccaggggacccaagggctacaagggagagaagggcaagcgtggcatcgacggggtggacggcgtgaagggggagatggggtacccaggcctgccaggctgcaagggctcgcccgggtttgacggcattcaaggaccccctggccccaagggagaccccggtgcctttggactgaaaggagaaaagggcgagcctggagctgacggggaggcggggagaccagggagctcgggaccatctggagacgagggccagccgggagagcctgggccccccggagagaaaggagaggcgggcgacgaggggaacccaggacctgacggtgcccccggggagcggggtggccctggagagagaggaccacgggggaccccaggcacgcggggaccaagaggagaccctggtgaagctggcccgcagggtgatcagggaagagaaggccccgttggtgtccctggagacccgggcgaggctggccctatcggacctaaaggctaccgaggcgatgagggtcccccagggtccgagggtgccagaggagccccaggacctgccggaccccctggagacccggggctgatgggtgaaaggggagaagacggccccgctggaaatggcaccgagggcttccccggcttccccgggtatccgggcaacaggggcgctcccgggataaacggcacgaagggctaccccggcctcaagggggacgagggagaagccggggaccccggagacgataacaacgacattgcaccccgaggagtcaaaggagcaaaggggtaccggggtcccgagggcccccagggacccccaggacaccaaggaccgcctgggccggacgaatgcgagattttggacatcatcatgaaaatgtgctcttgctgtgaatgcaagtgcggccccatcgacctcctgttcgtgctggacagctcagagagcattggcctgcagaacttcgagattgccaaggacttcgtcgtcaaggtcatcgaccggctgagccgggacgagctggtcaagttcgagccagggcagtcgtacgcgggtgtggtgcagtacagccacagccagatgcaggagcacgtgagcctgcgcagccccagcatccggaacgtgcaggagctcaaggaagccatcaagagcctgcagtggatggcgggcggcaccttcacgggggaggccctgcagtacacgcgggaccagctgctgccgcccagcccgaacaaccgcatcgccctggtcatcactgacgggcgctcagacactcagagggacaccacaccgctcaacgtgctctgcagccccggcatccaggtggtctccgtgggcatcaaagacgtgtttgacttcatcccaggctcagaccagctcaatgtcatttcttgccaaggcctggcaccatcccagggccggcccggcctctcgctggtcaaggagaactatgcagagctgctggaggatgccttcctgaagaatgtcaccgcccagatctgcatagacaagaagtgtccagattacacctgccccatcacgttctcctccccggctgacatcaccatcctgctggacggctccgccagcgtgggcagccacaactttgacaccaccaagcgcttcgccaagcgcctggccgagcgcttcctcacagcgggcaggacggaccccgcccacgacgtgcgggtggcggtggtgcagtacagcggcacgggccagcagcgcccagagcgggcgtcgctgcagttcctgcagaactacacggccctggccagtgccgtcgatgccatggactttatcaacgacgccaccgacgtcaacgatgccctgggctatgtgacccgcttctaccgcgaggcctcgtccggcgctgccaagaagaggctgctgctcttctcagatggcaactcgcagggcgccacgcccgctgccatcgagaaggccgtgcaggaagcccagcgggcaggcatcgagatcttcgtggtggtcgtgggccgccaggtgaatgagccccacatccgcgtcctggtcaccggcaagacggccgagtacgacgtggcctacggcgagagccacctgttccgtgtccccagctaccaggccctgctccgcggtgtcttccaccagacagtctccaggaaggtggcgctgggctagcccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctc

SEQ ID NO: 19 - DNA, 3445 bp, Homo sapiens
  source 1..3445: mol_type = genomic DNA
ccctgctgcttactcggcgcccgcgcctcgggccgtcgggagcggagcctcctcgggaccaggacttcagggccacaggtgctgccaagatgctccagggcacctgctccgtgctcctgctctggggaatcctgggggccatccaggcccagcagcaggaggtcatctcgccggacactaccgagagaaacaacaactgcccagagaagaccgactgccccatccacgtgtacttcgtgctggacacctcggagagcgtcaccatgcagtcccccacggacatcctgctcttccacatgaagcagttcgtgccgcagttcatcagccagctgcagaacgagttctacctggaccaggtggcgctgagctggcgctacggcggcctgcacttctctgaccaggtggaggtgttcagcccaccgggcagcgaccgggcctccttcatcaagaacctgcagggcatcagctccttccgccgcggcaccttcaccgactgcgcgctggccaacatgacggagcagatccggcaggaccgcagcaagggcaccgtccacttcgccgtggtcatcaccgacggccacgtcaccggcagcccctgcgggggcatcaagctgcaggccgagcgggcccgcgaggagggcatccggctcttcgccgtggcccccaaccagaacctgaaggagcagggcctgcgggacatcgccagcacgccgcacgagctctaccgcaacgactacgccaccatgctgcccgactccaccgagatcgaccaggacaccatcaaccgcatcatcaaggtcatgaaacacgaagcctacggagagtgctacaaggtgagctgcctggaaatccctgggccctctggccccaagggctaccgtggacagaagggtgccaagggcaacatgggtgagccgggagagcctggccagaagggaagacagggagacccgggcatcgaaggccccattggattcccaggacccaagggcgttcctggcttcaaaggagagaagggtgaatttggagccgacggtcgcaagggggcccctggcctggctggcaagaacgggaccgatggacagaagggcaagctggggcgcatcggacctcctggctgcaagggagaccctggaaaccggggccccgacggttacccgggggaagcagggagtccaggggagcgaggagaccaaggcggcaagggggaccctggccgcccaggacgcagagggcccccgggagaaatcggggccaagggaagcaaggggtatcaaggcaacagtggagccccaggaagtcctggtgtgaaaggagccaagggcgggcctgggccccgcggacccaaaggcgagccggggcgcaggggagaccccggcaccaagggcagcccaggcagcgatggccccaagggggagaagggggaccctggccctgaggggccccgcggcctggctggagaggttggcaacaaaggagccaagggagaccgaggcttgcctggacccagaggcccccagggagctcttggggagcccggaaagcagggatctcggggagaccccggtgatgcaggaccccgtggagactcaggacagccaggccccaagggagaccccggcaggcctggattcagctacccaggaccccgaggagcacccggagaaaaaggcgagcccggcccacgcggccccgagggaggccgaggcgactttggcttgaaaggagaacctgggaggaaaggagagaaaggagagcctgcggatcctggtccccctggtgagccaggccctcgggggccaagaggagtcccaggacccgagggtgagcccggcccccctggagaccccggtctcacggagtgtgacgtcatgacctacgtgagggagacctgcgggtgctgcgactgtgagaagcgctgtggcgccctggacgtggtcttcgtcatcgacagctccgagagcattgggtacaccaacttcacactggagaagaacttcgtcatcaacgtggtcaacaggctgggtgccatcgctaaggaccccaagtccgagacagggacgcgtgtgggcgtggtgcagtacagccacgagggcacctttgaggccatccagctggacgacgaacgtatcgactccctgtcgagcttcaaggaggctgtcaagaacctcgagtggattgcgggcggcacctggacaccctcagccctcaagtttgcctacgaccgcctcatcaaggagagccggcgccagaagacacgtgtgtttgcggtggtcatcacggacgggcgccacgaccctcgggacgatgacctcaacttgcgggcgctgtgcgaccgcgacgtcacagtgacggccatcggcatcggggacatgttccacgagaagcacgagagtgaaaacctctactccatcgcctgcgacaagccacagcaggtgcgcaacatgacgctgttctccgacctggtcgctgagaagttcatcgatgacatggaggacgtcctctgcccggaccctcagatcgtgtgcccagaccttccctgccaaacagagctgtccgtggcacagtgcacgcagcggcccgtggacatcgtcttcctgctggacggctccgagcggctgggtgagcagaacttccacaaggcccggcgcttcgtggagcaggtggcgcggcggctgacgctggcccggagggacgacgaccctctcaacgcacgcgtggcgctgctgcagtttggtggccccggcgagcagcaggtggccttcccgctgagccacaacctcacggccatccacgaggcgctggagaccacacaatacctgaactccttctcgcacgtgggcgcaggcgtggtgcacgccatcaatgccatcgtgcgcagcccgcgtggcggggcccggaggcacgcagagctgtccttcgtgttcctcacggacggcgtcacgggcaacgacagtctgcacgagtcggcgcactccatgcgcaagcagaacgtggtacccaccgtgctggccttgggcagcgacgtggacatggacgtgctcaccacgctcagcctgggtgaccgcgccgccgtgttccacgagaaggactatgacagcctggcgcaacccggcttcttcgaccgcttcatccgctggatctgctagcgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaaccca

SEQ ID NO: 20 - DNA, 744 bp, Homo sapiens
  source 1..744: mol_type = genomic DNA
actttggttccagcatcctgtccagcaaagaagcaatcagccaaaatgatacctggaggcttatctgaggccaaacccgccactccagaaatccaggagattgttgataaggttaaaccacagcttgaagaaaaaacaaatgagacttacggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttccaaagaaattatttcttcaattatttctcatttattgtattaagcagaaattaccttttctttctcaaaatcagtgttattgctttagagtataaactccatataaattgatggcaattggaaatcttataaaaactagtcaagcctaatgcaactggctaaaggatagtaccaccctcacccccaccataggcaggctggatcgtggactatcaattcaccagcctccttgttccctgtggctgctgataacccaacattccatctctaccctcatacttcaaaattaaatcaagtattttacaaa

SEQ ID NO: 21 - DNA, 2117 bp, Homo sapiens
  source 1..2117: mol_type = genomic DNA
gctggcggcggcggcaggtggcgcgggaggtcgcggcgcgccatggggctcccggcgctgctggccagtgcgctctgcaccttcgtgctgccgctgctgctcttcctggctgcgatcaagctctgggacctgtactgcgtgagcggccgcgaccgcagttgtgccctcccattgccccccgggactatgggcttccccttctttggggaaaccttgcagatggtactgcagcggaggaagttcctgcagatgaagcgcaggaaatacggcttcatctacaagacgcatctgttcgggcggcccaccgtacgggtgatgggcgcggacaatgtgcggcgcatcttgctcggagagcaccggctggtgtcggtccactggccagcgtcggtgcgcaccattctgggatctggctgcctctctaacctgcacgactcctcgcacaagcagcgcaagaaggtgattatgcgggccttcagccgcgaggcactcgaatgctacgtgccggtgatcaccgaggaagtgggcagcagcctggagcagtggctgagctgcggcgagcgcggcctcctggtctaccccgaggtgaagcgcctcatgttccgaatcgccatgcgcatcctactgggctgcgaaccccaactggcgggcgacggggactccgagcagcagcttgtggaggccttcgaggaaatgacccgcaatctcttctcgctgcccatcgacgtgcccttcagcgggctgtaccggggcatgaaggcgcggaacctcattcacgcgcgcatcgagcagaacattcgcgccaagatctgcgggctgcgggcatccgaggcgggccagggctgcaaagacgcgctgcagctgttgatcgagcactcgtgggagaggggagagcggctggacatgcaggcactaaagcaatcttcaaccgaactcctctttggaggacacgaaaccacggccagtgcagccacatctctgatcacttacctggggctctacccacatgttctccagaaagtgcgagaagagctgaagagtaagggtttactttgcaagagcaatcaagacaacaagttggacatggaaattttggaacaacttaaatacatcgggtgtgttattaaggagacccttcgactgaatcccccagttccaggagggtttcgggttgctctgaagacttttgaattaaatggataccagattcccaagggctggaatgttatctacagtatctgtgatactcatgatgtggcagagatcttcaccaacaaggaagaatttaatcctgaccgattcatgctgcctcacccagaggatgcatccaggttcagcttcattccatttggaggaggccttaggagctgtgtaggcaaagaatttgcaaaaattcttctcaaaatatttacagtggagctggccaggcattgtgactggcagcttctaaatggacctcctacaatgaaaaccagtcccaccgtgtatcctgtggacaatctccctgcaagattcacccatttccatggggaaatctgatgagcttgaatgttcaaacctgagacttattggaagtgtacatatgagtttttaaggagtgttgtgttgactttatatttaatttctaaatgtatattataatatttatgtgttttgactatactaccacaatctttaaatattaaaataatgaatttgtatcatttccaaataaagtaaaatttgaaggtacttttctggtattttaagattcctgttgggtaaaactcaccagtttagtattttcttagtgtatttaaccagattttacaatgcctacctggacttatttgtcatctttgcatctgttttctgtgagaagaaatcttagctgttttttatgttaacagttattagaaaatatatgtctgtgtgtgttattccagacgtatctctgtaaattcttctacagtcacttagattccctatttggaaaattgatccaagttaatttaatttttttttggtttgctgtactttagggaaagatgaacctgaaaaggtaacactgagaactgtcactctaacctctccagcttatctaacatgtcataaacataataaatctgtgttgtccaata

SEQ ID NO: 22 - DNA, 2310 bp, Homo sapiens
  source 1..2310: mol_type = genomic DNA
agagtcagaatcacaggtggattagtagggagtgttataaaagccttgaagtgaaagcccgcagttgtcttactaagaagagaagccttcaatggatccagctgtggctctggtgctctgtctctcctgtttgtttctcctttcactctggaggcagagctctggaagagggaggctcccgtctggccccactcctctcccgattattggaaatatcctgcagttagatgttaaggacatgagcaaatccttaaccaatttctcaaaagtctatggccctgtgttcactgtgtattttggcctgaagcccattgtggtgttgcatggatatgaagcagtgaaggaggccctgattgatcatggagaggagttttctggaagaggaagttttccagtggctgaaaaagttaacaaaggacttggaatccttttcagcaatggaaagagatggaaggagatccggcgtttctgcctcatgactctgcggaattttgggatggggaagaggagcatcgaggaccgtgttcaagaggaagcccgctgccttgtggaggagttgagaaaaaccaatgcctcaccctgtgatcccactttcatcctgggctgtgctccctgcaatgtgatctgctctgttattttccatgatcgatttgattataaagatcagaggtttcttaacttgatggaaaaattcaatgaaaacctcaggattctgagctctccatggatccaggtctgcaataatttccctgctctcatcgattatctcccaggaagtcataataaaatagctgaaaattttgcttacattaaaagttatgtattggagagaataaaagaacatcaagaatccctggacatgaacagtgctcgggactttattgattgtttcctgatcaaaatggaacaggaaaagcacaatcaacagtctgaatttactgttgaaagcttgatagccactgtaactgatatgtttggggctggaacagagacaacgagcaccactctgagatatggactcctgctcctgctgaagtacccagaggtcacagctaaagtccaggaagagattgaatgtgtagttggcagaaaccggagcccctgtatgcaggacaggagtcacatgccctacacagatgctgtggtgcacgagatccagagatacattgacctcctccccaccaacctgccccatgcagtgacctgtgatgttaaattcaaaaactacctcatccccaagggcacgaccataataacatccctgacttctgtgctgcacaatgacaaagaattccccaacccagagatgtttgaccctggccactttctggataagagtggcaactttaagaaaagtgactacttcatgcctttctcagcaggaaaacggatgtgtatgggagagggcctggcccgcatggagctgtttttattcctgaccaccattttgcagaactttaacctgaaatctcaggttgacccaaaggatattgacatcacccccattgccaatgcatttggtcgtgtgccacccttgtaccagctctgcttcattcctgtctgaagaagggcagatagtttggctgctcctgtgctgtcacctgcaattctcccttatcagggccattggcctctcccttctctctgtgagggatattttctctgacttgtcaatccacatcttcccattccctcaagatccaatgaacatccaacctccattaaagagagtttcttgggtcacttcctaaatatatctgctattctccatactctgtatcacttgtattgaccaccacatatgctaatacctatctactgctgagttgtcagtatgttatcactagaaaacaaagaaaaatgattaataaatgacaattcagagccatttattctctgcatgctctagataaaaatgattattatttactgggtcagttcttagatttctttcttttgagtaaaatgaaagtaagaaatgaaagaaaatagaatgtgaagaggctgtgctggccctcatagtgttaagcacaaaaagggagaaaggtaagagggtaggaaagctgttttagctaaatgccacctagagttattggaggtctgaatttggaaaaaaaaactatgtccaggagcagctgtaacctgtagggaaatactggaacaatcatccataagagggatgaacattaagtgtttgaattcatgctctgcttttgtgttactgtaaacacaagatcaagatttggataatctttttcctttgtgtttccaacttagatcatgtctaaatatatgctttcatatggctaa

SEQ ID NO: 23 - DNA, 1427 bp, Homo sapiens
  source 1..1427: mol_type = genomic DNA
agatcgctccgcccccatccgcaggttctaactttggcctgggactctgcccctctacctcagcacagaatcgccccgggtcctactacagaatcaatccttgaacactgcctccacgtcgccggctcaatctgggcgagaacccagacttccaccgcagccccgcaatctgcagacctcagcggcagcgcaggtggcagacctgcctcctttgcctgtgagtcatggcagctcccatgaatggccaagtgtgtgtggtgactggtgcctccaggggtattggccgtggcattgccttgcagctctgcaaagcaggcgccacagtttacatcactggccgccatctggacacccttcgcgttgttgctcaggaggcacaatccctcgggggccaatgtgtgcctgtggtgtgcgattcaagccaggagagtgaagtgcgaagcctgtttgagcaagtggatcgggaacagcaagggcgtctagatgtgctggtcaacaatgcttatgcaggggtccagacgatcctgaacaccaggaataaggcattctgggaaacccctgcctccatgtgggatgatatcaacaacgtcggactcagaggccactacttttgctcagtgtatggggcacggctgatggtaccagctggccaggggctcatcgtggtcatctcctccccaggaagcctgcagtatatgttcaatgtcccctatggtgtgggcaaagctgcgtgtgacaagctggctgctgactgtgcccacgagctgcggcgccatggggtcagctgtgtgtctctgtggccggggattgtgcagacagaactgctgaaggagcatatggcaaaggaggaggtcctgcaggatcctgtgttgaagcagttcaaatcagccttctcatctgcggaaaccacagaattgagtggcaaatgtgtggtggctttggcaacagatcccaatatcctgagcctgagtggtaaggtgctgccatcctgtgaccttgctcgacgctatggccttcgggatgtggacggccgccccgtccaagactatttgtctttgagctctgttctctcacacgtgtccggcctgggctggctggcctcctacctgccctccttcctccgtgtgcccaagtggattattgccctctacactagcaagttctaaccctcctggtctgacactacgtctctgcttgtcttctcatttggacttggtggttcgtcctgtctcagtgaaacagcagcctttcttgtttacccatacccttgatatgaagagaagccctctgctgtgtgtccgtggtgagttctggggtgcgcctaggtcccttctttgtgccttggttttccttgtccttctttttactttttgccttagtattgaaaaatgctcttggagctaataaaagtctcatttctctttcaa

SEQ ID NO: 24 - DNA, 3104 bp, Homo sapiens
  source 1..3104: mol_type = genomic DNA
aggctctatttagagccgggtaggggagcgcagcggccagatacctcagcgctacctggcggaactggatttctctcccgcctgccggcctgcctgccacagccggactccgccactccggtagcctcatggctgcaacctgtgagattagcaacatttttagcaactacttcagtgcgatgtacagctcggaggactccaccctggcctctgttccccctgctgccacctttggggccgatgacttggtactgaccctgagcaacccccagatgtcattggagggtacagagaaggccagctggttgggggaacagccccagttctggtcgaagacgcaggttctggactggatcagctaccaagtggagaagaacaagtacgacgcaagcgccattgacttctcacgatgtgacatggatggcgccaccctctgcaattgtgcccttgaggagctgcgtctggtctttgggcctctgggggaccaactccatgcccagctgcgagacctcacttccagctcttctgatgagctcagttggatcattgagctgctggagaaggatggcatggccttccaggaggccctagacccagggccctttgaccagggcagcccctttgcccaggagctgctggacgacggtcagcaagccagcccctaccaccccggcagctgtggcgcaggagccccctcccctggcagctctgacgtctccaccgcagggactggtgcttctcggagctcccactcctcagactccggtggaagtgacgtggacctggatcccactgatggcaagctcttccccagcgatggttttcgtgactgcaagaagggggatcccaagcacgggaagcggaaacgaggccggccccgaaagctgagcaaagagtactgggactgtctcgagggcaagaagagcaagcacgcgcccagaggcacccacctgtgggagttcatccgggacatcctcatccacccggagctcaacgagggcctcatgaagtgggagaatcggcatgaaggcgtcttcaagttcctgcgctccgaggctgtggcccaactatggggccaaaagaaaaagaacagcaacatgacctacgagaagctgagccgggccatgaggtactactacaaacgggagatcctggaacgggtggatggccggcgactcgtctacaagtttggcaaaaactcaagcggctggaaggaggaagaggttctccagagtcggaactgagggttggaactatacccgggaccaaactcacggaccactcgaggcctgcaaaccttcctgggaggacaggcaggccagatggcccctccactggggaatgctcccagctgtgctgtggagagaagctgatgttttggtgtattgtcagccatcgtcctgggactcggagactatggcctcgcctccccaccctcctcttggaattacaagccctggggtttgaagctgactttatagctgcaagtgtatctccttttatctggtgcctcctcaaacccagtctcagacactaaatgcagacaacaccttcctcctgcagacacctggactgagccaaggaggcctggggaggccctaggggagcaccgtgatggagaggacagagcaggggctccagcaccttctttctggactggcgttcacctccctgctcagtgcttgggctccacgggcaggggtcagagcactccctaatttatgtgctatataaatatgtcagatgtacatagagatctattttttctaaaacattcccctccccactcctctcccacagagtgctggactgttccaggccctccagtgggctgatgctgggacccttaggatggggctcccagctcctttctcctgtgaatggaggcagagacctccaataaagtgccttctgggctttttctaacctttgtcttagctacctgtgtactgaaatttgggcctttggatcgaatatggtcaagaggttggaggggaggaaaatgaaggtctaccaggctgagggtgagggcaaaggctgacgaagaggggagttacagatttcctgtagcaggtgtgggcttacagacacatggactgggctgggaggcgagcaaaggaagcagctgagactgttggagaacgcttacaagacttcatgcaagcaaggacatgaactcagaacactgaggtcagaagcatcctgctgtcatgacaccgctcgagtgaccttgaccttgaccaagtctgtcctgtttaggactgatttttcctattaggctagggtttggacctgatgttctcaagatgtctagaattgcatggctggccttgtggaatagatggttttgcattccagccaagtgtgctgtaaactgtatatctgtaatatgaatcccagcttttgagtctgacaaaatcagagttaggatcttgtaaaggaaaaaaaaaaaaaaacaaaacaaaatggagatgagtacttgctgagaaagaatgagggaaggagttggcatttgttgaaagtgtagtctttttctcttttttttttaattgcaacttttactttagatttaggaggtcgtgcgcaggtttgttacatgggtatattgtgtgatgctgagcttgggatgcgaatgatcctgtcacccaggtagtgagtatagcacccagtgaaactgtagtctcatgccaggcactgtgctagcccactctggctcatttaatcctctcctaagaagagaggagacacagcgtccccatttgacagatgcagaaagaggttccacaggtgtgccttgattctgtcctaaaaccgtttcccggaagcttttcctggtgtgggcgcttctaacctaatcctcaatcgattccagaactattactctgtttccacagtgatactgtgtctaggttttagggaggacagttcattgatgttacttaagaatgctttccaggtggaaagttccttaagtttgaggcttcaaattccatacagcacattaaaatcccattcatgagtttgaaatactgctctgttgtcttggaaataccaatcagattgttggctgaagtgatgtggataaagaagggatcttagaaaaa

SEQ ID NO: 25 - DNA, 1547 bp, Homo sapiens
  source 1..1547: mol_type = genomic DNA
acagagcgctagtccttcggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactgaa

SEQ ID NO: 26 - DNA, 2681 bp, Homo sapiens
  source 1..2681: mol_type = genomic DNA
aataaaaaccttccacccgtcagtctagaaggataagagaaagaaagttaagcaactacaggaaatggctttgggagttccaatatcagtctatcttttattcaacgcaatgacagcactgaccgaagaggcagccgtgactgtaacacctccaatcacagcccagcaaggtaactggacagttaacaaaacagaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtgtctaaaattgaaatcgccttacaatgtctgttctggagaaagacgaccactgtgaggcctttgtgaagaattttcatcaaggcatctgtagagatcagtgagcccaaaattaaagttttcagatgaaacaacaaaacttgtcaagctgactagactcgaaaataatgaaagttgggatcacaatgaaatgagaagataaaattcagcgttggcctttagactttgccatccttaaggagtgatggaagccaagtgaacaagcctcagtgacacaagtcaaattcatagtttcactctgggttttttgttgttgtgtggttattattctcactacagaaagactgagtttcatgctcctggctatgtcagatgtgaattttcatgggaataataatcaaccttgcagcaagccaaagcaatgcctcgcttgggttcttcatgttcttactacccagcgttttttaccacctagatgggcctctctaagtctatttgctcaatgaaccttatcccaaacttgttgttttcatggtgtctgaaagaatgtgatgtcagcttttagaatgaaatcagtgttaaggtacctccagtgaaccaaacatgtgtcttaaatctaagccactgaaaacagaagggaatgtccaaggcaaatacaaatcatacacagcttgtaacaacatacagcctttctatgtgaaataatggaataaactaaagagtttctgaagactgaagctatctggatatcaagtctggagtaggcaaagcatttccatttctacatggattataaaactttgtgttggactcattggtccctaatgcttttgttcatcacttcttccagttatcagtggaattacctggtgaccattcatttggaccgaaatcctggaagtctcctactgaataaaagtctacaattggccctaaaatagaaactgaaaaacaggacatagaattttttcaccagaccacagcatgtggaaactttctttatcatttttgaacacttgttaacagatttgcacatagagggagagaaaaaaaatggagtaacagtcaaaataataataatcagtatccaggccaggcgtggtggttcacgcctgtaatcctagcactttgggaggccgaggcggatggagcacctgaggtcaggagtttgagaccagcctggccaacatggtgaaaccctgtctctactaaaaatacaaaaattagctgggcgtggtggtgcatgcctgtaatcccagctactccagaggctgacacaggacaatcacttgaacccaggaggcagaggttgtagtgagccgagatggcaaccctgcactccaacctgggtgacaagagcgaaactccatctcaaaaaaaaaaaaaaaaatcagtatccttcctcttagttatatttctgccacttaattcaaacagtcatatgcaggtcgcttaatttatttgtgcttttgtttcatcttctacaaggccctcttagctctaaaacttgacagtggaataaggaaatgtttttccaaatctgcattgccggtgagatcctcaacatcagcatgttgagatggacctcaaccccacctctaaccctgaaacacactactcgatattatcttaggtatgttttagggtttagtttgtaaaataataatttatttttgaaggaaatataaaatattaaagagtaataatagctatcattttttaagattcaatctaaaacaatggactctttttttttccatttgtgatgtagataagcaagacaattttgatcatgagtggtgaaaagaggatcaaacttgactattcttgcaatggcagtccagcaacaagcctttcatttacattaaattataacttttcattcattcctaaaccaaacttaaaattctgctttcctttgagtagaaggtatttaacttgttttgtttttccttcagaaggaatttaatgcaaacggattgcagtcagcactttctgaatgttttcacacagtatgcaaagcttacatcataccaaggagtggagagttgaagtttcctcccagtgactccagtgacagaccacacctagaaagcgtttctcttcctgagtatttcaaaaagatgtaaaagagctggggagagtatgggaagaaacaatacaggattgcctttaattaattaagaattgcctcctgataaaaggaaaaa

SEQ ID NO: 27 - DNA, 2861 bp, Homo sapiens
  source 1..2861: mol_type = genomic DNA
gaaacctaacccgccctggggaggcgcgcagcagaggctccgattcggggcaggtgagaggctgactttctctcggtgcgtccagtggagctctgagtttcgaatcggcggcggcggattccccgcgcgcccggcgtcggggcttccaggaggatgcggagccccagcgcggcgtggctgctgggggccgccatcctgctagcagcctctctctcctgcagtggcaccatccaaggaaccagtagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggccttggctgacctcctctctgtcatctggttccccttgaagattgcctatcacatacatggcaacaactggatttatggggaagctctttgtaatgtgcttattggctttttctatggcaacatgtactgttccattctcttcatgacctgcctcagtgtgcagaggtattgggtcatcgtgaaccccatggggcactccaggaagaaggcaaacattgccattggcatctccctggcaatatggctgctgattctgctggtcaccattcctttgtatgtcgtgaagcagaccatcttcattcctgccctgaacatcacgacctgtcatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaagagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgttatttcctaatcaaaaaggtctcaccacataccatgtggatgcagcacctctcaggattgctaggagctcccctgtttgcatgagaaaagtagtcccccaaattaacatcagtgtctgtttcagaatctctctactcagatgaccccagaaactgaaccaacagaagcagacttttcagaagatggtgaagacagaaacccagtaacttgcaaaaagtagacttggtgtgaagactcacttctcagctgaaattatatatatacacatatatatattttacatctgggatcatgatagacttgttagggcttcaaggccctcagagatgatcagtccaactgaacgaccttacaaatgaggaaaccaagataaatgagctgccagaatcaggtttccaatcaacagcagtgagttgggattggacagtagaatttcaatgtccagtgagtgaggttcttgtaccacttcatcaaaatcatggatcttggctgggtgcggtgcctcatgcctgtaatcctagcactttgggaggctgaggcaggcaatcacttgaggtcaggagttcgagaccagcctggccatcatggcgaaacctcatctctactaaaaatacaaaagttaaccaggtgtgtggtgcacgtttgtaatcccagttactcaggaggctgaggcacaagaattgagtatcactttaactcaggaggcagaggttgcagtgagccgagattgcaccactgcactccagcttgggtgataaaataaaataaaatagtcgtgaatcttgttcaaaatgcagattcctcagattcaataatgagagctcagactgggaacagggcccaggaatctgtgtggtacaaacctgcatggtgtttatgcacacagagatttgagaaccattgttctgaatgctgcttccatttgacaaagtgccgtgataatttttgaaaagagaagcaaacaatggtgtctcttttatgttcagcttataatgaaatctgtttgttgacttattaggactttgaattatttctttattaaccctctgagtttttgtatgtattattattaaagaaaaatgcaatcaggattttaaacatgtaaatacaaattttgtataacttttgatgacttcagtgaaattttcaggtagtctgagtaatagattgttttgccacttagaatagcatttgccacttagtattttaaaaaataattgttggagtatttattgtcagttttgttcacttgttatctaatacaaaattataaagccttcagagggtttggaccacatctctttggaaaatagtttgcaacatatttaagagatacttgatgccaaaatgactttatacaacgattgtatttgtgacttttaaaaataattattttattgtgtaattgatttataaataacaaaattttttttacaactta

SEQ ID NO: 28 - DNA, 4182 bp, Homo sapiens
  source 1..4182: mol_type = genomic DNA
aaggagctggcggagcagcagcagccgccgccgccgccgccgccgcggctcttcggggaaacccggcgcggggcgcgggagggagcggccggggcggcggggccgggccaatcgggcggcgggcccggcgcgcggagccagcgacatggaggggggcgcggcgcccggccgcccgtgacgcgccgccgcccggcccgctctcggcgagcccgagccgccgccggccccgcggcggagatgagcaggtccgcgacgctgctgctgtgcctgctgggctgccacgtctggaaggcggtgaccaagacgctgcgggagcccggcgccggagcccaagaggtgacgttaaaggtgcacatcagcgacgccagcacccaccagcccgtagcagatgcgctcatcgagatcttcaccaaccaggcctccatagcctctggcacctcggggactgatggcgtcgcctttatcaagttccagtataagctgggcagtcagttgattgtcaccgcctcgaagcatgcctacgtgccaaactctgccccatggaagccaatccggttacctgtattttcctctctgagccttggcctgcttccagaacgctctgccactctaatggtatatgaagatgtcgtccaaatagtatcaggattccaaggtgcccggccacagcctcgcgttcatttccagagaagggctctgaggttgcctgagaacaccagctacagtgacctgaccgcgtttctcacggccgccagctccccttcggaggtggacagttttccttatttgcgaggattagacggaaatggaacaggaaacagcaccaggcatgacctgaccccagtcacagccgtcagcgtccacttgctgagcagtaatggaacgccggtgctggtggatggtcccatctatgtcactgtgcccctggccacgcagagcagcctgaggcacaatgcctatgtcgcggcgtggcggtttgaccagaagctgggaacgtggctgaagagcggtctgggtcttgtgcaccaggaaggcagccagctgacgtggacatacattgccccccagttggggtactgggtggccgccatgtcccctcccatcccaggtcccgttgtaacacaggacattaccacgtatcacacggtgtttcttttggccattttaggaggaatggctttcatacttttggttttgctgtgtctccttttatattattgcaggaggaagtgcttgaaacctcgtcagcaccacagaaaactgcagctccctgcaggactggagagttccaaaagagaccagtccacgtccatgtcacacattaacttgctgttttcacgccgagcgtcagaattccctggcccgctgtccgtcaccagccacggccgccccgaggcccccggcacgaaggaactgatgagtggagtccatttggaaatgatgtctccgggcggcgaaggggacctgcacacccccatgctcaagctctcctacagcacctcccaggaatttagctcccgggaggagctcctctcttgcaaggaagaggataaaagccagatctcctttgataacctcactccaagtgggacgctggggaaagactaccataagtcagtggaggtttttcccttaaaggcaagaaaatctatggaaagagaaggctacgagtcctcgggcaatgatgactacaggggtagttacaacaccgtgctctcacagcctttatttgaaaagcaggacagagaaggtccagcctccacgggaagcaaactcaccattcaggaacatctgtaccccgcgccttcatcacctgagaaagaacagctgctggaccgcagacccactgaatgtatgatgtcgcgatcagtagatcacctcgagagacctacgtccttcccacggcccggccagttaatctgctgcagttctgtcgaccaggtcaatgacagcgtttacaggaaagtactgcctgccttggtcatcccggctcattatatgaaactccccggggaccactcctatgtcagccagcccctcgtcgtcccggctgatcagcagcttgagatagaaagactacaggctgagctgtccaatccccatgccgggatcttcccacacccgtcctcacagatccagccccagcccctgtcttcccaggccatctctcagcagcacctgcaggatgcgggcacccgggagtggagccctcagaacgcatccatgtcggagtctctctccatcccagcttccctgaacgacgcggctttggctcagatgaacagtgaggtgcagctcctgactgaaaaggccctgatggagcttgggggtgggaagccgcttccgcacccccgggcgtggttcgtctccttggatggcaggtccaacgctcacgttagacattcatacattgatctccaaagagctggaaggaacggaagtaatgatgccagtttggactctggcgtagatatgaatgaaccaaaatcagcccggaagggaaggggagatgctttgtctctgcagcagaactacccgcccgtccaagagcaccagcagaaagagcctcgagccccagacagcacggcctacacgcagctcgtgtacctggatgacgtggaacagagtggtagcgaatgtgggaccacggtctgtacccccgaggacagtgccctgcgatgcttgttggaggggtcgagtcggagaagtggtggccagctgcccagcctgcaggaggagacgaccagacggactgcggatgccccctcggagccagcagccagcccccaccagagaagatctgcccacgaggaagaggaagacgatgatgatgatgaccaaggagaagacaagaaaagcccctggcagaaacgggaggagaggcccctgatggcgtttaacattaaatgagctatcgcagacccacctgactgtggaatataaaattgccaaatatcctttctcatggaagcgcgtacccgttcgtggaggaaacggaacggcagcccagccgtgggacggacgtggacgtttactgcattcctgtttgccgtgtaaatgttagaaaggaattaaagttattactcggaataaaggatgactttggcggatgtcgcccctgcaaggaggtggctgaaagtggtgtccagatgtccttccgaggactcggcgtatccgccaccagggacattaagaaaccgcacgtgatgtcgctatgctctaacgatcacctcagttctccctcggattctgggaacagatgaaactttttgcatcgcttgagtcatttttatcacaataatcctactgtgaagctgtcgttgagaacttaggttggcacgtagcgtctcaaggtatgcgttctctcaaaggaaagctatgcatcgctgcttcgttgtctgattttgcttagattttgctttggttaggttgcgttttggggtttgcctttttttgttgtcgcttaaatgcaatttggttgtaaagatttgattcctttgtgttcatctgttccgcttctcagcggtccatctcagcgtctcccttcaggaaccgctgagtgtcctctcttaacatccaagccttttaatgaaatcgtactgaaatctgtatcagctaagagtcctccaatcctggtcccattaactccaagtgcctttttgacagtgacaacagacagtccctcgctttttgttgttgttggttttcttaacccctttaatggaactgcctggattttatacagttattaaaggatgtctcttttgctttaaactgcatgctgccaagtgccatttggggtcagcatcctcgtttcaacacagtgtgctctctagttatcatgtgtaacgtgggttctgtttagcgaagatagactagaggacacgttagagatgcccttccctgctccatccctgtggcaccattatggttttttggctgtttgtatatacggttacgtattaactctggaatcctatgggctcatcttgctcacccaatgtgggagtctggtttgagcaagcgagctgaatgtgactattaaaaaaaatttaaaaaaaaaaaagaaaatcttatgtactatccaaaagtgccagaatgactcttctgtgcattcttcttaaagagctgcttggttatccaaaaatgaaaattcaaaataaactctgaagaaaaggaaaa

SEQ ID NO: 29 - DNA, 1317 bp, Homo sapiens
  source 1..1317: mol_type = genomic DNA
cttcctgacccaggggctccgctggctgcggtcgcctgggagctgccgccagggccaggaggggagcggcacctggaagatgcgcccattggctggtggcctgctcaaggtggtgttcgtggtcttcgcctccttgtgtgcctggtattcggggtacctgctcgcagagctcattccagatgcacccctgtccagtgctgcctatagcatccgcagcatcggggagaggcctgtcctcaaagctccagtccccaaaaggcaaaaatgtgaccactggactccctgcccatctgacacctatgcctacaggttactcagcggaggtggcagaagcaagtacgccaaaatctgctttgaggataacctacttatgggagaacagctgggaaatgttgccagaggaataaacattgccattgtcaactatgtaactgggaatgtgacagcaacacgatgttttgatatgtatgaaggtgataactctggaccgatgacaaagtttattcagagtgctgctccaaaatccctgctcttcatggtgacctatgacgacggaagcacaagactgaataacgatgccaagaatgccatagaagcacttggaagtaaagaaatcaggaacatgaaattcaggtctagctgggtatttattgcagcaaaaggcttggaactcccttccgaaattcagagagaaaagatcaaccactctgatgctaagaacaacagatattctggctggcctgcagagatccagatagaaggctgcatacccaaagaacgaagctgacactgcagggtcctgagtaaatgtgttctgtataaacaaatgcagctggaatcgctcaagaatcttatttttctaaatccaacagcccatatttgatgagtattttgggtttgttgtaaaccaatgaacatttgctagttgtatcaaatcttggtacgcagtatttttataccagtattttatgtagtgaagatgtcaattagcaggaaactaaaatgaatggaaattcttaaagggaatgatgtgattcaagctggaaagagggttgggagaaacagcttgtccaggtggagctatgttatgatcagatcgaagtgtgacccctgtgtggtccagacagccctgcagagagaaaacctttattccattatcaccaagcacctcctagtttccgacagtcatctccttctgctgggagaattagcagcagttcagggggcttatgttatgtccttgttcaactcaacttgagctcttgaactcctcctgtgggcctgtgaatgtattcattcattccacaactctgggtg

SEQ ID NO: 30 - DNA, 3257 bp, Homo sapiens
  source 1..3257: mol_type = genomic DNA
gtggagcagccaagatggagccggcagtcggcggtccgggcccactgatcgtgaacaacaaacagccccagcccccgccacctccgccgccggcagccgcacagccaccacccggggcaccgcgggccgccgcgggcctcctgcctgggggcaaagcccgcgagttcaaccgcaaccagcgcaaagactcagagggctattcggagtcaccagacctggagtttgagtatgctgacacagacaagtgggctgcagagctctcggagctttacagctacacggaagggccagaattcctgatgaatcgaaaatgctttgaggaggacttccggatccatgtgacagacaagaagtggactgagctggataccaaccagcaccggacccatgccatgaggctcctggatggcttggaagtcactgccagggagaagagactcaaggtggctcgagcaattctctatgttgctcaaggcacgtttggggagtgcagctcggaggcagaggtgcagtcctggatgcgctacaacatctttctcctcctggaggtgggcacgttcaatgctttggtggagcttctgaacatggaaatagacaacagtgccgcctgcagcagtgctgtgaggaagcctgccatctccctggctgacagcacagacctcagggtcctgctcaacatcatgtacctgatagtggagaccgttcatcaggagtgtgagggtgacaaggctgagtggaggaccatgcggcagaccttcagagccgagctgggctccccgctgtacaacaatgagccatttgccatcatgctgtttgggatggtgaccaaattttgcagtggtcacgcccctcactttcccatgaagaaagttctcttgctgctctggaagacagtattgtgcacgctaggcggctttgaggagctgcagagcatgaaggctgagaagcgcagcatcctgggcctccccccgcttcctgaggacagcatcaaagtgattcgcaacatgagagcagcctctccaccagcatctgcttcagacttgattgagcagcagcagaaacggggccgccgagagcacaaggctctgataaagcaggacaacctagatgccttcaacgagcgggatccctacaaggctgatgactctcgagaagaggaagaggagaatgatgatgacaacagtctggagggggagacgtttcccctggaacgggatgaagtgatgcctcccccgctacagcacccacagactgacaggctgacttgccccaaagggctcccgtgggctcccaaggtcagagagaaagacattgagatgttccttgagtccagccgcagcaaatttataggttacactctaggcagtgacacgaacacagtggtggggctgcccaggccaatccacgaaagcatcaagactctgaaacagcacaagtacacgtcgattgcagaggtccaggcacagatggaggaggaatacctccgctcccctctctcagggggagaagaagaagttgagcaagtccctgcagaaaccctctaccaaggcttgctccccagcctgcctcagtatatgattgccctcctgaagatcctgttggctgcagcacccacctcaaaagccaaaacagactcaatcaacatcctagcggacgtcttgcctgaggagatgcccaccacagtgttgcagagcatgaagctgggggtggatgtaaaccgccacaaagaggtcattgttaaggccatttctgctgtcctgctgctgctgctcaagcactttaagttgaaccatgtctaccagtttgaatacatggcccagcacctggtgtttgccaactgcattcctttgatcctaaagttcttcaatcaaaacatcatgtcctacatcactgccaagaacagcatttctgtcctggattaccctcactgcgtggtgcatgagctgccagagctgacggcggagagtttggaagcaggtgacagtaaccaattttgctggaggaacctcttttcttgtatcaatctgcttcggatcttgaacaagctgacaaagtggaagcattcaaggacaatgatgctggtggtgttcaagtcagcccccatcttgaagcgggccctaaaggtgaaacaagccatgatgcagctctatgtgctgaagctgctcaaggtacagaccaaatacttggggcggcagtggcgaaagagcaacatgaagaccatgtctgccatctaccagaaggtgcggcatcggctgaacgacgactgggcatacggcaatgatcttgatgcccggccttgggacttccaggcagaggagtgtgcccttcgtgccaacattgaacgcttcaacgcccggcgctatgaccgggcccacagcaaccctgacttcctgccagtggacaactgcctgcagagtgtcctgggccaacgggtggacctccctgaggactttcagatgaactatgacctctggttagaaagggaggtcttctccaagcccatttcctgggaagagctgctgcagtgaggctgttggttaggggactgaaatggagagaaaagatgatctgaaggtacctgtgggactgtcctagttcattgctgcagtgctcccatcccccaccaggtggcagcacagccccactgtgtcttccgcagtctgtcctgggcttgggtgagcccagcttgacctccccttggttcccagggtcctgctccgaagcagtcatctctgcctgagatccattcttcctttacttcccccaccctcctctcttggatatggttggttttggctcatttcacaatcagcccaaggctgggaaagctggaatgggatgggaacccctccgccgtgcatctgaatttcaggggtcatgctgatgcctctcgagacatacaaatccttgctttgtcagcttgcaaaggaggagagtttaggattagggccagggccagaaagtcggtatcttggttgtgctctggggtgggggtggggtgtttctgatgttattccagcctcctgctacattatatccagaagtaattgcggaggctccttcagctgcctcagcactttgattttggacagggacaaggtaggaagagaagcttcccttaaccagaggggccatttttccttttggctttcgagggcctgtaaatatctatatataattctgtgtgtattctgtgtcatgttggggtttttaatgtgattgtgtattctgtttacattaaaaagaagcaaaaataa

SEQ ID NO: 31 - DNA, 3314 bp, Homo sapiens
  source 1..3314: mol_type = genomic DNA
gggctccgctggctcgggcgtcggatcggagccgccggggcgcgggcggcccagagctggcctgaacctccaggacttcagcccttctaggagaatctgatcccaggtgagggaggtcacttggggtgtggcccagcaggcaggcgggactccagactgggaacggaagtcagccctgctggggctgggagctgaagcatctgttccttcctcgcgggtgtgagacaaggaagagtgatcattgccacaccaggagctgaagccatggcctcaaagcctgagaagagggtggcatcgtctgtctttatcaccctggcacccccgcgccgcgatgtggccgtggcggaggaagtgaggcaggcagtttgtgaggcccggcgtggccgcccctgggaggctcctgcccccatgaagacacccgaggctggcttggcggggaggcccagcccctggacaacccctggcagagctgcagccacagtgccggctgcacctatgcagctcttcaatggaggatgcccaccccctcctcctgtcctggatggtgaggacgtgcttcctgacctggacctcctcccaccccctccaccgccccctccagtgcttctgccttctgaagaggaggctcctgctccaatgggggcctcactcattgcagacttagagcagctgcacctgtccccgcccccgcccccaccacaggccccagcggagggaccttcagtccagcccggtcccctcaggcccatggaggaagagctgccacctcccccggcagaacctgttgagaaaggggcatccacagacatctgtgccttctgccacaagaccgtgtccccccgagagctggctgtggaggccatgaagaggcagtaccatgcccagtgcttcacgtgccgcacctgccgccgccagctggctgggcagagcttctaccagaaggatgggcgacccctctgcgaaccctgctaccaggacacactggagaggtgcggcaagtgtggcgaggtggtccgggaccacatcatcagggccctgggccaggccttccacccctcctgcttcacgtgtgtgacctgcgcccggtgcattggggatgagagctttgccctgggcagccagaacgaggtgtactgcctggacgacttctacaggaaattcgcccccgtctgcagcatctgtgaaaatcccatcatccctcgggatgggaaagatgccttcaaaatcgaatgcatgggaagaaacttccatgaaaattgctacaggtgtgaggactgcaggatcctcctgtctgtcgagcccacggaccaaggctgctaccccctgaacaaccatctcttctgcaagccatgccatgtgaagcggagtgctgcggggtgctgctgagagtgcccgctgggcagtgaacagaccactagccccggctggggcccttccctgacttggtttcccttcctaacctgctcttgcacactttccttctgagcctccatggagaccagcctgcaagccggcccagcctgtccaggatacagtggggctgagcacccccaggccttccactcctctaccctctgggcaccagaaggctcctggaccatgagcttcacccccagaattccctgctgaccctgccccacttccagggaaaagctgggggaggttggacccctctcactgactagctgtctggtaggggtgctaggaccagcctcgcctgtggggttgagctgtttgaggacaaactccaaggtcccttaaaaagtgccttttagaggctgggcatggtggctcacgcttgtaatcccagcactttgggaggccaaggtgggtggatcacctgaggtcaggagttcaagaccagcctggccaacatggtgaaaccctgtctctactaaaaatacaaaaattagccaggcatggtagcaggtgcctgtaatcccagctactggggaaagctgaggcaggagaattgcttcaatctggaaggcagaggttgcagtgagattgcaccattgcattccagcctgggcaacaagagggaaactccgtctcaaaaaaaaaaaagtgccttttaggccggatgtggtggctcatgcctgtaatcccagcactttgggaggccaaggcaggcagatcacttgaggtcaggagtttgagaccagcctggccaacacggcaaaaccccgtttctactaaaaatacaaaaattagctgggtgtggtggcgtgcgcctgtaatcccagctactcaggaggctgaggcaggagcattgcttgaacccgggaggcagaggttgcagtgagctgagatggcaccactgcactccagcctgggagacacagcgagactctgtctccaaaaaaaaaagtgctttttgaaaatgttgaggttgaaatgatgggaaccaacattctttggatttagtggggagcataatagcaaacacccccttggttcgcacatgtacaggaatgggacccagttggggcacagccatggacttccccgccctggaatgtgtggtgcaaagtggggccagggcccagacccaagaggagagggtggtccgcagacaccccgggatgtcagcatcccccgacctgccttctggcggcacctcccgggtgctgtgttgagtcagcaggcatggggtgagagcctggtatatgctgggaacagggtgcaggggccaagcgttcctccttcagccttgacttgggccatgcaccccctctcccccaaacacaaacaagcacttctccagtatggtgccaggacaggtgtcccttcagtcctctggttatgacctcaagtcctacttgggccctgcagcccagcctgtgttgtaacctctgcgtcctcaagaccacacctggaagattcttcttccctttgaaggagaatcatcattgttgctttatcacttctaagacattttgtacggcacggacaagttaaacagaatgtgcttccctccctggggtctcacacgctcccacgagaatgccacaggggccgtgcactgggcaggcttctctgtagaaccccaggggcttcggcccagaccacagcgtcttgccctgagcctagagcagggagtcccgaacttctgcattcacagaccacctccacaattgttataaccaaaggcctcctgttctgttatttcacttaaatcaacatgctattttgttttcactcacttctgactttagcctcgtgctgagccgtgtatccatgcagtcatgttcacgtgctagttacgtttttcttcttacacatgaaaataaatgcataagtgttagaagctc

SEQ ID NO: 32 - DNA, 3509 bp, Homo sapiens
  source 1..3509: mol_type = genomic DNA
agaggcagcccgctcacttcccgcggaggcgctccccggcgccgcgctccgcggcagccgcctgcccccggcgctgcccccgcccgccgcgccgccgccgccgccgcgcacgccgcgccccgcagctctgggcttcctcttcgcccgggtggcgttgggcccgcgcgggcgctcgggtgactgcagctgctcagctcccctcccccgccccgcgccgcgcggccgcccgtcgcttcgcacagggctggatggttgtattgggcagggtggctccaggatgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctagctcccgggactggggggtttgtctggcatagccatgctggtagcaagagagaaaaaatcaacagcaaacaaaaccacacaaaccaaaccgtcaacagcataataaaatcccaacaactatttttatttcatttttcatgcacaacctttcccccagtgcaaaagactgttactttattattgtattcaaaattcattgtgtatattactacaaagacaaccccaaaccaatttttttcctgcgaagtttaatgatccacaagtgtatatatgaaattctcctccttccttgcccccctctctttcttccctctttcccctccagacattctagtttgtggagggttatttaaaaaaacaaaaaaggaagatggtcaagtttgtaaaatatttgtttgtgctttttccccctccttacctgaccccctacgagtttacaggtctgtggcaatactcttaaccataagaattgaaatggtgaagaaacaagtatacactagaggctcttaaaagtattgaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacatgatacattctcaagagttgcttgaccgaaagttacaaggaccccaacccctttgtcctctctacccacagatggccctgggaatcaattcctcaggaattgccctcaagaactctgcttcttgctttgcagagtgccatggtcatgtcattctgaggtcacataacacataaaattagtttctatgagtgtataccatttaaagaatttttttttcagtaaaagggaatattacaatgttggaggagagataagttatagggagctggatttcaaaacgtggtccaagattcaaaaatcctattgatagtggccattttaatcattgccatcgtgtgcttgtttcatccagtgttatgcactttccacagttggacatggtgttagtatagccagacgggtttcattattatttctctttgctttctcaatgttaatttattgcatggtttattctttttctttacagctgaaattgctttaaatgatggttaaaattacaaattaaattgttaatttttatcaatgtgattgtaattaaaaatattttgatttaaataacaaaaataataccagattttaagccgtggaaaatgttcttgatcatttgcagttaaggactttaaataaatcaaatgttaacaaaagagcatttctgttattttttttcacttaactaaatccgaagtgaatatttctgaatacgatatttttcaaattctagaactgaatataaatgacaaaaatgaaaataaaattgttttgtctgttgttataatgaatgtgtagctagtaaaaaggagtgaaagaaattcaagtaaagtgtataagttgatttaatattccaagagttgagatttttaagattctttattcccagtgatgtttacttcatttttttttttttttttgacaccggcttaagccttctgtgtttcctttgagccttttcactacaaaatcaaatattaatttaactacctttcctccttccccaatgtatcacttttctttatctgagaattcttccaatgaaaataaaatatcagctgtggctgatagaattaagttgtgtccaa

SEQ ID NO: 33 - DNA, 2501 bp, Homo sapiens
  source 1..2501: mol_type = genomic DNA
aagtcggtgccgctgccgtctctgcgttcgccatgcgtcccggggcgccagggccactctggcctctgccctggggggccctggcttgggccgtgggcttcgtgagctccatgggctcggggaaccccgcgcccggtggtgtttgctggctccagcagggccaggaggccacctgcagcctggtgctccagactgatgtcacccgggccgagtgctgtgcctccggcaacattgacaccgcctggtccaacctcacccacccggggaacaagatcaacctcctcggcttcttgggccttgtccactgccttccctgcaaagattcgtgcgacggcgtggagtgcggcccgggcaaggcgtgccgcatgctggggggccgcccgcgctgcgagtgcgcgcccgactgctcggggctcccggcgcggctgcaggtctgcggctcagacggcgccacctaccgcgacgagtgcgagctgcgcgccgcgcgctgccgcggccacccggacctgagcgtcatgtaccggggccgctgccgcaagtcctgtgagcacgtggtgtgcccgcggccacagtcgtgcgtcgtggaccagacgggcagcgcccactgcgtggtgtgtcgagcggcgccctgccctgtgccctccagccccggccaggagctttgcggcaacaacaacgtcacctacatctcctcgtgccacatgcgccaggccacctgcttcctgggccgctccatcggcgtgcgccacgcgggcagctgcgcaggcacccctgaggagccgccaggtggtgagtctgcagaagaggaagagaacttcgtgtgagcctgcaggacaggcctgggcctggtgcccgaggccccccatcatcccctgttatttattgccacagcagagtctaatttatatgccacggacactccttagagcccggattcggaccacttggggatcccagaacctccctgacgatatcctggaaggactgaggaagggaggcctgggggccggctggtgggtgggatagacctgcgttccggacactgagcgcctgatttagggcccttctctaggatgccccagcccctaccctaagacctattgccggggaggattccacacttccgctcctttggggataaacctattaattattgctactatcaagagggctgggcattctctgctggtaattcctgaagaggcatgactgcttttctcagccccaagcctctagtctgggtgtgtacggagggtctagcctgggtgtgtacggagggtctagcctgggtgagtacggagggtctagcctgggtgagtacggagggtctagcctgggtgagtacggagggtctagcctgggtgtgtatggaggatctagcctgggtgagtatggagggtctagcctgggtgagtatggagggtctagcctgggtgtgtatggagggtctagcctgggtgagtatggagggtctagcctgggtgtgtatggagggtctagcctgggtgagtatggagggtctagcctgggtgtgtacggagggtctagtctgagtgcgtgtggggacctcagaacactgtgaccttagcccagcaagccaggcccttcatgaaggccaagaaggctgccaccattccctgccagcccaagaactccagcttccccactgcctctgtgtgcccctttgcgtcctgtgaaggccattgagaaatgcccagtgtgccccctgggaaagggcacggcctgtgctcctgacacgggctgtgcttggccacagaaccacccagcgtctcccctgctgctgtccacgtcagttcatgaggcaacgtcgcgtggtctcagacgtggagcagccagcggcagctcagagcagggcactgtgtccggcggagccaagtccactctgggggagctctggcggggaccacgggccactgctcacccactggccccgaggggggtgtagacgccaagactcacgcatgtgtgacatccggagtcctggagccgggtgtcccagtggcaccactaggtgcctgctgcctccacagtggggttcacacccagggctccttggtcccccacaacctgccccggccaggcctgcagacccagactccagccagacctgcctcacccaccaatgcagccggggctggcgacaccagccaggtgctggtcttgggccagttctcccacgacggctcaccctcccctccatctgcgttgatgctcagaatcgcctacctgtgcctgcgtgtaaaccacagcctcagaccagctatggggagaggacaacacggaggatatccagcttccccggtctggggtgaggaatgtggggagcttgggcatcctcctccagcctcctccagcccccaggcagtgccttacctgtggtgcccagaaaagtgcccctaggttggtgggtctacaggagcctcagccaggcagcccaccccaccctggggccctgcctcaccaaggaaataaagactcaa

SEQ ID NO: 34 - DNA, 3326 bp, Homo sapiens
  source 1..3326: mol_type = genomic DNA
attccaggaagtttgcatagctccctggacttctgctttgcactgccctgcaggagtgggtggggaaaggaagtggctttgaggcacacagaggggcttgttgaggccaccggaggaagcttctgccaccaatatgggacctgtgcccagcctaccagaagagagcatctgaaaacatgtatcgacatggtaacccctctgcttgaagcctcacatggctccctattgccttggtgctgaacaccctatggctgaccgtggcccagcctctgcaacagctctgcctcctctccagtggtgaagacccagcctgctgagactcctcctgcagttcctcaacatgcctgcatttctgctgcctcagggcctttgcgaaggttgttccttgtaactggaatgcccttccatcccttttttattcaaaaggctgcaattttaattgaagaaagttcccttccaaggttcatgagttgcctgacttgcccaccggtttcctgcaagatcccttggcctggcacttagtgctcaggaaatatttggtgatgggccaactgagtgagaaggtgggatctggtgggaaggaaagcggaaaggtagaaattctgctcacttcctcattcccacctcccaaggaacccctggtgtccctgtggaacccgctttgggaaccggtggttcaggtcagccttttcactttgtactcaaagccacatcgcattgaagccacaggtggggcaaggtcatgcatgacctgagtctccaaatcccttcaccctgtttggttctgcaacggggattaggggagccccacgatttgttttcaaaggatgtccgggctccaggacaggatgccctgggtcacctgatgacaggtgtggtggttggaaagggcctggtttcagctccgggtacacttcctccttccttctgctgcgtggtgtggcctcttccacgtcctcagaatccagctgttactccgtccgcggcctctcagctctagggccctctgcacactggcccccccagtgtcacgggcatccagacgggatccagtgcatcctcttttagaagaaaggcctgtctccagatccccgagtccctctagcatctcccagaaggtgtcagacgcagcagtgtccagggagcggcagcagatactctgacccatggatcccctgggcccggccaagccacagtggtcgtggcgctgctgtctgaccacgctgctgtttcagctgctgatggctgtgtgtttcttctcctatctgcgtgtgtctcaagacgatcccactgtgtaccctaatgggtcccgcttcccagacagcacagggacccccgcccactccatccccctgatcctgctgtggacgtggccttttaacaaacccatagctctgccccgctgctcagagatggtgcctggcacggctgactgcaacatcactgccgaccgcaaggtgtatccacaggcagacgcggtcatcgtgcaccaccgagaggtcatgtacaaccccagtgcccagctcccacgctccccgaggcggcaggggcagcgatggatctggttcagcatggagtccccaagccactgctggcagctgaaagccatggacggatacttcaatctcaccatgtcctaccgcagcgactccgacatcttcacgccctacggctggctggagccgtggtccggccagcctgcccacccaccgctcaacctctcggccaagaccgagctggtggcctgggcagtgtccaactgggggccaaactccgccagggtgcgctactaccagagcctgcaggcccatctcaaggtggacgtgtacggacgctcccacaagcccctgccccagggaaccatgatggagacgctgtcccggtacaagttctatctggccttcgagaactccttgcaccccgactacatcaccgagaagctgtggaggaacgccctggaggcctgggccgtgcccgtggtgctgggccccagcagaagcaactacgagaggttcctgccacccgacgccttcatccacgtggacgacttccagagccccaaggacctggcccggtacctgcaggagctggacaaggaccacgcccgctacctgagctactttcgctggcgggagacgctgcggcctcgctccttcagctgggcactcgctttctgcaaggcctgctggaaactgcaggaggaatccaggtaccagacacgcggcatagcggcttggttcacctgagaggctggtgtggggcctgggctgccaggaacctcattttcctggggcctcacctgagtgggggcctcatctacctaaggactcgtttgcctgaagcttcacctgcctgaggactcacctgcctgggacggtcacctgttgcagcttcacctgcctggggattcacctacctgggtcctcactttcctggggcctcacctgctggagtcttcggtggccaggtatgtcccttacctgggatttcacatgctggcttccaggagcgtcccctgcggaagcctggcctgctggggatgtctcctggggactttgcctactggggacctcggctgttggggactttacctgctgggacctgctcccagagaccttccacactgaatctcacctgctaggagcctcacctgctggggacctcaccctggagggcactgggccctgggaactggcacccatggggccccacccatgagtgatggttctggctgatttgtttgtgatgttgttagccgcctgtgaggggtgcagagagataatcaccgcaccgtttccagatgtaatactgcaaagaaaaccgatgatgaggccgggtgcggtggctcacacctgtaatcccagcactttgggaggccgaggcaggcggatcacaaggtcaggagatcgagaccatcctggccaatatggtgaaacccgtctctactaaaaatacaaaaatttgccgggcgtggtggtgcatgcctgtaatcccagctacttgggaagctgaggcaggagaatcgcttgaaccagagagtcggaggttgcagtaagccgagatcgcgccactgcactccagcctggcgacagagcgagactctgtctcaaaaaaaaaaaaaaaaaaaaaaaaaattagctgggtgcggtggccggtgcctgtaatcccagctacccaggtggctgagacacaagaatcgcttgaacccaggaggcagatgttgtagggagccatgatcgcaccactgcactccaacctgggcgacacagtgagactgtctcaagaaaaaaaaaaaaaggggggagtgtccctttctgagtgtgcaa

SEQ ID NO: 35 - DNA, 4505 bp, Homo sapiens
  source 1..4505: mol_type = genomic DNA
aaacaggcttactgtgttcactgctggacgcctctgggcaatcttaccctcctgggaactgagagagacaatgctaggagccagaaacatgaggaatacatgagctcggcgccaatggaacatgctttcacaccgggtgcagctaaccaccacatcatacaaaactcgtcgctgacgttttagatgtataatctgattggcatggactcacagaggagggagtaagggaagagtaagaagatttcaaaggagagacacaggttgcaggttagcaggagaacaggcaagccaaatgcaaaggagccacttcagaaatgtgtcacagaaaagtgaaaatgcaacctagtggtaagtgaagaggggaagaagaaagaaaaaggaccagaaccgtgaactgaagggacagggaacagccagacgagagcttcagccatcacgaggatgatttcggaacctggagaaaatgtaagttaaatatatctacactctgatcctatctcaagagagagatattttactcatttcctggttgtgaatgatgggctcttggaagcactgtctttttagcgcgtctcttatctctgccctgatttttgtatttgtttacaatactgagttatgggagaataaacgttttctgagggcagctctgtccaatgcttcactgttagcagaagcctgtcatcagatttttgaggggaaagttttttacccaacagaaaatgcattgaaaactacccttgatgaagctacctgctatgagtacatggttcgaagccactatgtaacagaaacactctctgaagaagaggctgggttccctttagcttacacagtgaccatccacaaagacttcggcacttttgagaggctcttcagggcgatttatatgccccaaaatgtctactgtgtgcacctggatcagaaggcgacggatgcctttaaaggtgcagtgaaacagttactcagctgcttcccaaatgcttttctggcttccaagaaggagtcggttgtctatggggggatctccaggctccaggctgacctgaactgcctggaagaccttgtggcctctgaagttccctggaagtatgtcatcaacacctgcgggcaagactttcccctgaaaaccaacagggaaatagttcagtatctgaagggatttaaagggaaaaatatcacccccggagtgctgcctcctgaccacgctgttggacggactaaatacgtccaccaagaactgttaaaccacaaaaattcctacgtgattaaaacaacaaaattaaaaactcctcctcctcatgacatggtgatttactttggcacggcctacgtggctctcacaagggactttgctaacttcgtcctccaagaccagctcgcacttgacttactctcctggtccaaggacacctacagccccgacgaacatttctgggtgacactcaacaggattcccggtgttcctggctctatgccaaatgcatcctggactggaaacctcagagctataaagtggagtgacatggaagacagacacggaggctgccacggccactatgtacatggtatttgtatctatggaaacggagacttaaagtggctggttaattcaccaagcctgtttgctaacaagtttgagcttaatacctacccccttactgtggaatgcctagaactgaggcatcgcgaaagaaccctcaatcagagtgaaactgcgatacaacccagctggtatttttgagctattcatgagctactcatgactgaagggaaactgcagctgggaagaggagcctgtttttgtgagagacttttgccttcgtaatgttaaccgtttcaggaccacgtttatagcttcaggacctggctacgtaattatacttaaaatatccactggacactgtgaaatacactaacaggatggctgggtagagcaatctgggcactttggccaattttagtcttgctgtttcttgatgctcacctctatattagtttattgttaggatcaatgataaatttaaatgacctcagatctttgcaccagatactcatcatatacaaatgttttagtaaaaaagagaattgtagataatactgtctaggaaaataagaattaggtttctttgaagaaggaatcttttataacaccttaacagtcaccactgtgctcaaccagacagatagtgaaacagctttctgggtaattcaccaatttcctttaaaacataagctacctgaatggagaatacatcttgtttctgagtttcaacactagcatttttggcttactcatggacaaagttctgtatatagtataaagtcattaacaagaaacaggatatgctttaagacagaattcactgtctgttgcttcagtaaaaggacctcggggaataaaacatttctctcttatatgccagaatgtaggctggtccctatgtcatgtcttccattaagaacactaaaaagtccttgcaagaatggagatatgcattcaagagaggtgctatcacatagatctagtctgaagtctggaacactttcctcttctatgacccctctctccccagtattatcttacttgcaaaatggagaccaaattctatcctgtgaggcttttaattgcaccatagtatgctctgagtagctttacactgcctggtactgatagtagtggctcgatttttaagagccttcaattgtagatgaacatctctgttatttatccctcattcatccatccgttcattcattcagccttcaatcaacatctcttgagtgtctattatgtacaggacatgtactgagacaaaaaggaaacataagagctttttcactctaaaaatcttggcaataatgtcaacaccagaaagcctcctctggagaatcttacagagtgattgtagtttaatacaggaacacacagggctgtgtagcatgataccaggcccaggagatcagtaattacaaattaagggttaaatcagagattattcaacagagagggagaaaggaggagacagagggaggacctgttgtgttccagccattctggtattcctttatgtatctaatttcattcaaacctcacaacagtcttgtgaggcccttatataattactcccattttgcagatgaagtaactgaggcttagaaaggttaatagcaccggggaacaatttctctgggtgagaattgggactctgttgctggtcttctcagttcatttcctgaggtggatttactgagagaaggtgaaataaagccatatttagtataccagagaaggtagattttaagaatggtctcagtgttaatactgagaaaaagtcctgtcagttcagaaaaaatgtgaagtctactttagtattcctgtaatactaaaccgttgagtttctaaatatttatttattctaacaaaaagcaattactacaaatggatgacacatttaatgaacacaattttattttttttctgtaactgtgcttgttgaatgtcaatcatatttaaagggaatgactttgaagtaaaaccttttttcttgctactgaaaaaaatggagttgttttgggtggtaaagtgttaaggaatagggacagctggtcacacaaggaactcttgaaggccacatgtgaaaacctgtcacttgcacagaggccagtcccactaaggtgaccagagtgggctccaagcacaaactgccattggctatagatgggactgtgtccccccaaaattcatgtgttggagccttaaccctcaatgtgatggtatttgagatggggcctttggtaagggaagtttagatgaggtcacgagggtaggaccctcatgatgggatgagtccccttacaagacctctggcttgggccgggcgtggtggctcacacctgtaatcccaacactttgggaggccaaggcaggtagatcacttgatgccaggagttccagaccaggctggccgacatggtgaaaccccatctctactaaaaaatataaaaattagccgggctttgtggcatgtgcctgtaatcccagctatttggcaggctgaggcatgagaatcgcttgaacccaggaggtggaggttacagtgagctgagagtgccccactgcactccagcctgggtgacagagcgagactttgtcccaaaacaaaataggtgaggggatagcgaatgcactcagggtcagcagtggagtttaaaaattgtctcttttcaacttatttaaatgacagcacctgagaagaggaaccgttttacactggatgtttctcatgtagaacaagaaatctttctggaattgatgtttacatgtctgttgttggtcatctctcctgtgtcttaaatactttaatgttggaagagcatagtgtttgggctagtgggtttctgacagcccatgggaatgccctgaaactactgtatctgatgtttgttttcgatgaggttccatgttttgttttcttgggaataaattaatatattgttttcca

SEQ ID NO: 36 - DNA, 3731 bp, Homo sapiens
  source 1..3731: mol_type = genomic DNA
cttccctctagaatcctccaacatggagcctcttgcagcttacccgctaaaatgttccgggcccagagcaaaggtatttgcagttttgctgtctatagttctatgcacagtaacgctatttcttctacaactaaaattcctcaaacctaaaatcaacagcttttatgcctttgaagtgaaggatgcaaaaggaagaactgtttctctggaaaagtataaaggcaaagtttcactagttgtaaacgtggccagtgactgccaactcacagacagaaattacttagggctgaaggaactgcacaaagagtttggaccatcccacttcagcgtgttggcttttccctgcaatcagtttggagaatcggagccccgcccaagcaaggaagtagaatcttttgcaagaaaaaactacggagtaactttccccatcttccacaagattaagattctaggatctgaaggagaacctgcatttagatttcttgttgattcttcaaagaaggaaccaaggtggaatttttggaagtatcttgtcaaccctgagggtcaagttgtgaagttctggaagccagaggagcccattgaagtcatcaggcctgacatagcagctctggttagacaagtgatcataaaaaagaaagaggatctatgagaatgccattgcgtttctaatagaacagagaaatgtctccatgagggtttggtctcattttaaacattttttttttggagacagtgtctcactctgtcacccaggctggagtgcagtagtgcgttctcagctcattgcaacctctgcctttttaaacatgctattaaatgtggcaatgaaggatttttttttaatgttatcttgctattaagtggtaatgaatgttcccaggatgaggatgttacccaaagcaaaaatcaagagtagccaaagaatcaacatgaaatatattaactacttcctctgaccatactaaagaattcagaatacacagtgaccaatgtgcctcaatatcttattgttcaacttgacattttctaggactgtacttgatgaaaatgccaacacactagaccactctttggattcaagagcactgtgtatgactgaaatttctggaataactgtaaatggttatgttaatggaataaaacacaaatgttgaaaaatgtaaaatatatatacatagattcaaatccttatatatgtatgcttgttttgtgtacaggattttgttttttctttttaagtacaggttcctagtgttttactataactgtcactatgtatgtaactgacatatataaatagtcatttataaatgaccgtattataacatttgaaaaagtcttcatcattttcagtatttctctctgcattattttttagaagcaactcaaattctgtaatcataatattaccaagtaagtcagtctttatttttgcgtgaaaatcccaagtgtagaaaaaaggacatgagtataagatcatttaacttgattccttataggctgatttgggttgctaatgtattcgtactagattagccacttgggaggaattcaatgctgcatcattctgatagctgaaatagtaaaagtcaactccaaacagcctaaattctagtataaaaagggggtgatcatacaagtcctaaagtgtagatcagcagtcactgaacctcttagagtcacaaggcatttcttctcccaaagtcatgacctttatattttgttagccttataattgactaacaaagcaacctgccaagttggtgaaggttaattattgcctcatgaaatatacattcatcgcataaaagaattaaataggaagatggccaaaaatgtaaaaaagtcgtataaatttgtctaagaagaaaaagacccaatttaagtagtctgtgacttgactcccaaattctgacctgatggatgaaagtgaagatgtttcgtatggcaaattcagtaatgctacctcagtatatagttctgtatacgtatttggaccaaagttattagataaaccaaaaatatttcatgaagcatcttctgcttcagagtccatatccagccacccagctatgaatgtatttagaaagatatttggtggaggtggaatttctaccagaggtcaagaggatttgtagggatagcctcaagaatgccagatgagtgtgtcagaaagcctgctcctacctctcgccagctgcgtgactttaggcaagtcacttaagtaccttatttcagtttcctcacctgtaaattgaacaggggttgaatggaataaaaagccctttgcaactctaaaagtcaattaacacacttactaaggtgttttaagtgaccaggaaggaaatcagaactaacctggattgttagcgaagaaaacagaagccaccaacaagcctatctgtcctttgcaattttgctccaagagcctattagcaatttgaattctgagcctctaagaaaacttatgctcaggtggcccctccaagtgttggtgccagagcactcttgccttgtatcctcacctacgccttccatgaactcatagcagtgctgtggcactgcaggggtgttttatgcattcacgttaggagcatgagcagaactgcatatgccagcaccacatgggcagcagagactccccactgccagatggatgataatggagctgcactgggaacaaccaggactgtcccagtcaacatgatacagatgaacatcctgccttaagtcactagctctctccaaagccagataaagtgggtttcttcacttacatctctctaatcttggctgccttattcaacctacagactctatgtcagtgctggattttctccccttgccctttaggtaagcagagtaggcaggaggagatttgaaagcgtgtcatacatattgccttgaaagaaaggacattcaggtgtcccttcggggttctgtaacaattaggctatgtgtgctgagagagggagcggagtcatttgctgatgtgctgctacctgtaactcaaatgcctcataggaataccagcggacaaacacatagaggaaaagttgaatctttcctatattggacaatcttccttcagtcttgtttctgcatcaaaaagtctgggacacagagtgctacaggatgcctgggctaaggactgtaacagaaaaatatcaacatcagagcctggcacagtggctcacaccggtaatcccagcactttgggaggccgaggcgggtggatcacttcaggtcaggagttcgagaccatcctggccaacatggtgaaacccccatctctactaaaaatacaaaaattagccgggcacggtggcacatgcctgtaatcccagctacctgggaggctgagtcagaagaatcacttgaaaacaggaggctgaggttgcaatgagccaagattgcaccactgcactccaacctgggcaacagagcgagaccccatcttaaagaccctgtctcaaaaaagagagaaaaagaaaaagaaaaatatcaggcatcacatgaaagagtattcagctgtcgttcttgtgaaaaaggaaaaggacaaaatcaaatgcattttaactgtggtaactaattcactgacttgaagtttgctttgtttgttttctttggtgcgtagagaagaatacttcatactaactcctgatcatcttacagcagacataacaacagagtgttagagactggaataaatatgaatttggctgaaa

SEQ ID NO: 37 - DNA, 2700 bp, Homo sapiens
  source 1..2700: mol_type = genomic DNA
aggagatgtgccaaactgttaagagtggttatttctgagcagaagaatgtggatgaattccattcttcctatttttcttttcaggtctgtgcggctgctaaagaacgacccagtcaacttgcagaaattctcttacactagtgaggatgaggcctggaagacgtacctagaaaacccgttgacagctgccacaaaggccatgatgagagtcaatggagatgatgacagtgttgcggccttgagcttcctctatgattactacatgggtcccaaggagaagcggatattgtcctccagcactgggggcaggaatgaccaaggaaagaggtactaccatggcatggaatatgagacggacctcactccccttgaaagccccacacacctcatgaaattcctgacagagaacgtgtctggaaccccagagtacccagatttgctcaagaagaataacctgatgagcttggagggggccttgcccacccctggcaaggcagctcccctccctgcaggccccagcaagctggaggccggctctgtggacagctacctgttacccaccactgatatgtatgataatggctccctcaactccttgtttgagagcattcatggggtgccgcccacacagcgctggcagccagacagcaccttcaaagatgacccacaggagtcgatgctcttcccagatatcctgaaaacctccccggaacccccatgtccagaggactaccccagcctcaaaagtgactttgaatacaccctgggctcccccaaagccatccacatcaagtcaggcgagtcacccatggcctacctcaacaaaggccagttctaccccgtcaccctgcggaccccagcaggtggcaaaggccttgccttgtcctccaacaaagtcaagagtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcgctctggaggggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctgtaaggcctctcgagcatccaaaccctcacgacctgcaaggggccagcagggacgtggccccacgccacacacaacctctccacatgcctcagcgctgttacttgaatgccttccctgagggaagaggcccttgagtcacagacccacagacgtcagggccagggagagacctagggggtcccctggcctggatccccatggtatgcttgaatctgctccctgaacttcctgccagtgcctccccgtaccccaaaacaatgtcaccatggttaccacctacccagaagactgttccctcctcccaagacccttgtctgcagtggtgctcctgcaggctgcccgttaagatggtggcggcacacgctccctcccgcagcaccacgccagctggtgcggcccccactctctgtcttccttcaacttcagacaaaggatttctcaacctttggtcagttaacttgaaaactcttgattttcagtgcaaatgacttttaaaagacactatattggagtctctttctcagacttcctcagcgcaggatgtaaatagcactaacgatcgactggaacaaagtgaccgctgtgtaaaactactgccttgccactcactgttgtatacatttcttatttacgattttcatttgttatatatatatataaatatactgtatatatatgcaacattttatatttttcatggatatgtttttatcatttcaaaaaatgtgtatttcacatttcttggactttttttagctgttattcagtgatgcattttgtatactcacgtggtatttagtaataaaaatctatctatgtattacgtcacatta

SEQ ID NO: 38 - DNA, 2135 bp, Homo sapiens
  source 1..2135: mol_type = genomic DNA
atcctctcaacctacagacccagtgagccctaggccctcaccccagacccagctgctggataggacccagccccgctcccagagacaatgaccatgtttgaaaatgtcacccgggccctggccagacagctaaaccctcgaggggacctgacaccacttgacagcctcatcgacttcaagcgcttccatcccttctgcctggtgctgaggaagaggaagagcacgctcttctggggggcccggtacgtccgcaccgactacacgctgctggatgtgcttgagcccggcagctcaccttcagacccaacagacactgggaattttggctttaagaatatgctggacacccgagtggagggagatgtggatgtaccaaagacggtgaaggtgaagggaacggcagggctctcgcagaacagcactctggaggtccagacactcagtgtggctcccaaggccctggagaccgtgcaggagaggaagctggcagcagaccacccattcctgaaggagatgcaagatcaaggggagaacctgtatgtggtgatggaggtggtggagacggtgcaggaggtcacactggagcgagccggcaaggcagaggcctgcttctccctccccttcttcgccccattggggctacagggatccataaatcacaaggaggctgtaaccatccccaagggctgcgtcctggcctttcgagtgagacagctgatggtcaaaggcaaagatgagtgggatattccacatatctgcaatgataacatgcaaaccttccctcctggagaaaagtcaggagaggagaaggtcatccttatccaggcatctgatgttggggacgtacacgaaggcttcaggacactaaaagaagaagttcagagagagacccaacaagtggagaagctgagccgagtagggcaaagctccctgctcagctccctcagcaaacttctagggaagaaaaaggagctacaagaccttgagctcgcacttgaaggggctctagacaagggacatgaagtgaccctggaggcactcccaaaagatgtcctgctatcaaaggaggccgtgggcgccatcctctatttcgttggagccctaacagagctaagtgaagcccaacagaagctgctggtgaaatccatggagaaaaagatcctacccgtgcagctaaagctggtggagagcacgatggaacagaacttcctgctggataaagagggtgttttccccctgcaacctgagctgctctcctcccttggggacgaggagctgaccctcacggaggctctagtcgggctgagtggcctggaagtgcagagatcgggcccccaatatatgtgggacccagacaccctccctcgcctctgtgctctttatgcaggcctctctctccttcagcagcttaccaaggcctcctaatttgccttttacgtctgcttcatgactccctaatgccttcccaacctcgtggtgctgtgtccttaccacctaagggcatttcagagccatcagctgaagacatctgaaatctcagctggtcacccatgataaccaacttccacctgcccaaccatatatcaccccctacccctccagctccgcaacccactaagcccatctgctgatgcttaaattttctttacttttcttttctttttttttttttttttgagatggaggctcactctgtcacccaggctggagtgcagtggcttgatcttggctcactgcaacctccgcctcccaggttcaagcaattctcctgcctcagcctctcgagcagctggaattacaggcacgtgccacgacacccagctaatttttgtatttttagtagagacggggtttcaccatgttgcccaggttggtctcgaactcctggcctcaagtgatctgcccgcctcggcctcccaaagtgctgggattacaggcgtgagccaccgtgcccggcctgtgcttaaatattctaggagtagaagcactcagacttttaaaactatgagccgtttcagcaaaactgagaaaaaaagaagattaattaatttcctatcctaatccctcctacttatacttccccaaaaaacaagctttactggaattaatggaaaatattatgtcaatcttaggaa

SEQ ID NO: 39 - DNA, 5721 bp, Homo sapiens
  source 1..5721: mol_type = genomic DNA
actcttgtcagggccgcggcacatgggcggccggatgcgctgagcccggcgctgcggggccgcggagcgctggggagcagcggccgccggcgcggggaggggggtggggtgggacggcgcaccgcctccggtgctggcactaggggctggggtcggcgcggtgtcttctgcccttctgcagccgtcgacatttttttttctttctttttttcaattttgaacattttgcaaaacgaggggttcgaggcaggtgagagcatcctgcacgtcgccggggagcccgcgggcacttggcgcgctctcctgggaccgtctgcactggaaacccgaaagtttttttttaatatatatttttatgcagatgtatttataaagatataagtaatttttttcttcccttttctccaccgccttgagagcgagtacttttggcaaaggacggaggaaaagctcagcaacattttagggggcggttgtttctttcttatttctttttttaaggggaaaaaatttgagtgcatcgcgatggagaaaatgtcccgaccgctccccctgaatcccacctttatcccgcctccctacggcgtgctcaggtccctgctggagaacccgctgaagctcccccttcaccacgaagacgcatttagtaaagataaagacaaggaaaagaagctggatgatgagagtaacagcccgacggtcccccagtcggcattcctggggcctaccttatgggacaaaacccttccctatgacggagatactttccagttggaatacatggacctggaggagtttttgtcagaaaatggcattccccccagcccatctcagcatgaccacagccctcaccctcctgggctgcagccagcttcctcggctgccccctcggtcatggacctcagcagccgggcctctgcaccccttcaccctggcatcccatctccgaactgtatgcagagccccatcagaccaggtcagctgttgccagcaaaccgcaatacaccaagtcccattgatcctgacaccatccaggtcccagtgggttatgagccagacccagcagatcttgccctttccagcatccctggccaggaaatgtttgaccctcgcaaacgcaagttctctgaggaagaactgaagccacagcccatgatcaagaaagctcgcaaagtcttcatccctgatgacctgaaggatgacaagtactgggcaaggcgcagaaagaacaacatggcagccaagcgctcccgcgacgcccggaggctgaaagagaaccagatcgccatccgggcctcgttcctggagaaggagaactcggccctccgccaggaggtggctgacttgaggaaggagctgggcaaatgcaagaacatacttgccaagtatgaggccaggcacgggcccctgtaggatggcatttttgcaggctggctttggaatagatggacagtttgtttcctgtctgatagcaccacacgcaaaccaacctttctgacatcagcactttaccagaggcataaacacaactgactcccattttggtgtgcatctgtgtgtgtgtgcgtgtatatgtgcttgtgctcatgtgtgtggtcagcggtatgtgcgtgtgcgtgttcctttgctcttgccattttaaggtagccctctcatcgtcttttagttccaacaaagaaaggtgccatgtctttactagactgaggagccctctcgcgggtctcccatcccctccctccttcactcctgcctcctcagctttgcttcatgttcgagcttacctactcttccaggactctctgcttggattcactaaaaagggccctggtaaaatagtggatctcagtttttaagagtacaagctcttgtttctgtttagtccgtaagttaccatgctaatgaggtgcacacaataacttagcactactccgcagctctagtcctttataagttgctttcctcttactttcagttttggtgataatcgtcttcaaattaaagtgctgtttagatttattagatcccatatttacttactgctatctactaagtttccttttaattctaccaaccccagataagtaagagtactattaatagaacacagagtgtgtttttgcactgtctgtacctaaagcaataatcctattgtacgctagagcatgctgcctgagtattactagtggacgtaggatattttccctacctaagaatttcactgtcttttaaaaaacaaaaagtaaagtaatgcatttgagcatggccagactattccctaggacaaggaagcagagggaaatgggaggtctaaggatgaggggttaatttatcagtacatgagccaaaaactgcgtcttggattagcctttgacattgatgtgttcggttttgttgttccccttccctcacaccctgcctcgcccccacttttctagttaactttttccatatccctcttgacattcaaaacagttacttaagattcagttttcccactttttggtaatatatatatttttgtgaattatactttgttgtttttaaaaagaaaatcagttgattaagttaataagttgatgttttctaaggccctttttcctagtggtgtcatttttgaatgcctcataaattaatgattctgaagcttatgtttcttattctctgtttgcttttgaacgtatgtgctcttataaagtggacttctgaaaaatgaatgtaaaagacactggtgtatctcagaaggggatggtgttgtcacaaactgtggttaatccaatcaatttaaatgtttactatagaccaaaaggagagattattaaatcgtttaatgtttatacagagtaattataggaagttcttttttgtacagtatttttcagatataaatactgacaatgtattttggaagacatatattatatatagaaaagaggagaggaaaactattccatgttttaaaattatatagcaaagatatatattcaccaatgttgtacagagaagaagtgcttgggggtttttgaagtctttaatattttaagccctatcactgacacatcagcatgttttctgctttaaattaaaattttatgacagtatcgaggcttgtgatgacgaatcctgctctaaaatacacaaggagctttcttgtttcttattaggcctcagaaagaagtcagttaacgtcacccaaaagcacaaaatggattttagtcaaatatttattggatgatacagtgttttttaggaaaagcatctgccacaaaaatgttcacttcgaaattctgagttcctggaatggcacgttgctgccagtgccccagacagttcttttctaccctgcgggcccgcacgttttatgaggttgatatcggtgctatgtgtttggtttataatttgatagatgtttgactttaaagatgattgttcttttgtttcattaagttgtaaaatgtcaagaaattctgctgttacgacaaagaaacattttacgctagattaaaatatcctttcatcaatgggattttctagtttcctgccttcagagtatctaatcctttaatgatctggtggtctcctcgtcaatccatcagcaatgcttctctcatagtgtcatagacttgggaaacccaaccagtaggatatttctacaaggtgttcattttgtcacaagctgtagataacagcaagagatgggggtgtattggaattgcaatacattgttcaggtgaataataaaatcaaaaacttttgcaatcttaagcagagataaataaaagatagcaatatgagacacaggtggacgtagagttggcctttttacaggcaaagaggcgaattgtagaattgttagatggcaatagtcattaaaaacatagaaaaatgatgtctttaagtggagaattgtggaaggattgtaacatggaccatccaaatttatggccgtatcaaatggtagctgaaaaaactatatttgagcactggtctctcttggaattagatgtttatatcaaatgagcatctcaaatgttttctgcagaaaaaaataaaaagattctaataaaatgtattctcttgtgtgccaggagaggtttcagaaacctacctcgtcttacaaatttaaacactttggagtctgtacaggtgccttatatgtaggtcattgtcacgatacacacacacgaacactccctctggactggctgcctctccatccagggcagttaactagcaaacaaggcagatctgcttcatggagcgggaggccatggcttgactctgagtgatttgggtcaaccggagtcagacgcatgtctgcacgctgcagctattatgagagtccctttgtcatttttcaccttttcatcctaagcatctttcagagattaattatttggccattaacaatgaatccaaatcatatcatactgacatcatctagacatgatttggaaggaacagcttaggacctcctgatgaggtcacattgttgtttcttttaactagacttggcaaagaaaggcaaaaattgaccagcctatctttctgctggtgctgccttaaggaggtagtttgttgaggggagggctgtagatcattacttctttctcttcaggaagtggccactttgaaccattcaaataccacattaggcaagactgtgataggccttttgtcttcaaatacaacaggcctccactgacccatccctcaaagcagaaggaccctttgaggagagtacagatgggattccacagtggggtgggtggaatggaaacctgtactagaccacccagaggttccttctaacccactggtttggtggggaactcacagtaattccaaatgtacaatcagatgtctagggtctgttttcggaagaagcaagaattatcagtggcaccctccccactgcccccagtgtaaaacaatagacattctgtgaaatgcaaagctattctttggtttttctagtagtttatctcattttaccctattcttcctttaaggaaaactcaatctttatcacagtcaattagagcgatcccaaggcatgggaccaggcctgcttgcctatgtgtgatggcaattggagatctggatttagcactggggtctcagcaccctgcaggtgtctgagactaagtgatctgccctccaggtggcgatcaccttctgctcctaggtacccccactggcaaggccaaggtctcctccacgttttttctgcaattaataatgtcatttaaaaaatgagcaaagccttatccgaatcggatatagcaactaaagtcaatacattttgcaggaggctaagtgtaagagtgtgtgtgtgtgtgtgtgcgtgcatgtgtgtgtgtgtgtatgtgtgtgaataagtcgacataaagtctttaattttgagcaccttaccaaacataacaataatccattatccttttggcaacaccacaaagatcgcatctgttaaacaggtacaagttgacatgaggttagtttaattgtacaccatgatattggtggtatttatgctgttaagtccaaacctttatctgtctgttattcttaatgttgaataaactttgaattttttcctttctttcatgtatttttattaacagttggctagcaatggtattctgttcccacctcggtagcaaagagaccatttgtagagattattacctagataataaaatgataatactatataattagtaa

SEQ ID NO: 40 - DNA, 3624 bp, Homo sapiens
  source 1..3624: mol_type = genomic DNA
acttcccgcttgggcgcccggacggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctcttta

SEQ ID NO: 41 - DNA, 6046 bp, Homo sapiens
  source 1..6046: mol_type = genomic DNA
acagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaaaacaaacaacattaaaaaattgagaacaagtatggaaagaatgaaagatcaaggaaaaaagaataccaagttacatttcgttaaggtgcttatgatcttagaactatgcaacctaataggtttgaaactgtttacctgagagagaacaaaaagagagacttttttgtattggaagtaatctgattaatttttattttcttcaaggagagatacttgaaaggaatatgtttgtccatctgttggatccaaacatttctatattttgtaaatgttgttgttgttttttttttaatcgtttactatttgcactacaatggtgtttgacctgtctaatccttatttaacaagtattttctttggttgggggtgggggtggggtttaagagctgcacttaatgtgagctataaaagaactgctacagcacacaaaatagctatttttattattataattataattattattattattttgtaccttaaaaaatagacacatacaccaaagacatttgtgtgagcctttaaacagtctgtctgtggttggtatcattcaccatcaatgagtcaggggttgggattcaaggttgagtagtgtggattgtgttcaggcttaaaagacctgagaagtttggtttttgactccttttacatccatgaaacaggacatttcatactggatgtacagtagttgtacactgttggatatcaagttcaatcaaattcatggaactacatgcttgtatgtgtatatatacattgcttgtgcatatgcatatctgtatgtatatatacatgtattgtaccatgtccatacacattttaagcacttcaggctgtcattttttaatgttcttaaagcaatgaatgtttgtgtgcaaaacacagtatttttaagaaggataggctatagtttttgcttttactctgaactaggtgggcgcatttcaaaaattcggatgggaaaaagcctggaaattccagtgaatattcagcaaggccctctttcattgtacagggatcaaatttcctcctcttttttgtgccccctcccacttctacaagttatcccctgtggggaaaacaggatgataatcaaaactctgggctgatgtttttccaacttagtgtctattggaatcaatcttaaatcagaagctttttcagaaaaataatatttaggccagaattagagttgagtgtattttttaaaaatgattaaggcttggttgtgagaaatattacctgtaccagctgggaaaaataatgtcatcactaactaaaagataattaatttgagagaaagtgttaagagagggagagtaaggaagagaacagttaagaggaggcagaggtgagggcagtagtaaaaatctctaaaattttaatttacagccaaaattcttcatgtgtaaatttgtattgattcagatgcagaaatgaaaaaaaaacacctttgttttataaatatcaaagtacatgcttaaagccaagtttttatctagtttattctagtacttagcttgcctggaatagctaataaattattcatgtatgtgcttttgaaaatccagagccctatttttacacacttgtgtgaagttggcaaacattttgaaaaatggaaaaaagtttctaataattgggaacaattacattaattaatattttgtaaaatattgaagcttttagccctatgtcaatttgtagattaaaataaattaattataggaaaggaagataacagtgagaaaccaaacattacaaaaggtggtttagctctccttgaaaaatatactaagttggtatactataacacttggctatatgtaggcaatgtcactactgggcaaatacacttactgtgttctagaggcagccctttcttatgcagaaaatacaatacgcactgcatgagaagcttgagagtggattctaatccaggtctgtcgaccttggatatcatgcatgtgggaaggtgggtgtggtgagaaaagttttaaggcaagagtagatggccatgttcaactttacaaaatttcttggaaaactggcagtattttgaactgcatcttctttggtaccggaacctgcagaaacagtgtgagaaattaagtcctggttcactgcgcagtagcaaagatggtcaaggccatggaaaaagcagaaatttaccaagaaagctgatacccatgtatagttcccactcatctcaaatacatctgctatctttttaagctaagtcctagacatatcggggataacatgggggttgattagtgaccacagttatcagaagcagagaaatgtaattccatattttatttgaaacttattccatattttaattggatattgagtgattgggttatcaaacacccacaaactttaattttgttaaatttatatggctttgaaatagaagtataagttgctaccattttttgataacattgaaagatagtattttaccatctttaatcatcttggaaaatacaagtcctgtgaacaaccactctttcacctagcagcatgaggccaaaagtaaaggctttaaattataacatatgggattcttagtagtatgtttttttcttgaaactcagtggctctatctaaccttactatctcctcactctttctctaagactaaactctaggctcttaaaaatctgcccacaccaatcttagaagctctgaaaagaatttgtctttaaatatcttttaatagtaacatgtattttatggaccaaattgacattttcgactattttttccaaaaaagtcaggtgaatttcagcacactgagttgggaatttcttatcccagaagaccaaccaatttcatatttatttaagattgattccatactccgttttcaaggagaatccctgcagtctccttaaaggtagaacaaatactttctatttttttttcaccattgtgggattggactttaagaggtgactctaaaaaaacagagaacaaatatgtctcagttgtattaagcacggacccatattatcatattcacttaaaaaaatgatttcctgtgcaccttttggcaacttctcttttcaatgtagggaaaaacttagtcaccctgaaaacccacaaaataaataaaacttgtagatgtgggcagaaggtttgggggtggacattgtatgtgtttaaattaaaccctgtatcactgagaagctgttgtatgggtcagagaaaatgaatgcttagaagctgttcacatcttcaagagcagaagcaaaccacatgtctcagctatattattatttattttttatgcataaagtgaatcatttcttctgtattaatttccaaagggttttaccctctatttaaatgctttgaaaaacagtgcattgacaatgggttgatatttttctttaaaagaaaaatataattatgaaagccaagataatctgaagcctgttttattttaaaactttttatgttctgtggttgatgttgtttgtttgtttgtttctattttgttggttttttactttgttttttgttttgttttgttttgttttgcatactacatgcagttctttaaccaatgtctgtttggctaatgtaattaaagttgttaatttatatgagtgcatttcaactatgtcaatggtttcttaatatttattgtgtagaagtactggtaatttttttatttacaatatgtttaaagagataacagtttgatatgttttcatgtgtttatagcagaagttatttatttctatggcattccagcggatattttggtgtttgcgaggcatgcagtcaatattttgtacagttagtggacagtattcagcaacgcctgatagcttctttggccttatgttaaataaaaagacctgtttgggatgta

SEQ ID NO: 42 - DNA, 4683 bp, Homo sapiens
  source 1..4683: mol_type = genomic DNA
ccggacagcaccaggaagcccggcaggagaattctgccagatatcaggacagatggggaacagcagaagccgtgtggggcggagcttttgttcacagtttcttcctgaggaacaggcagagattgatcaattgtttgatgctctgtcatcagataaaaacagcccgaatgtctcatccaaatccttctctctgaaggcactacagaaccacgtcggggaagctcttcccccagagatggtcaccaggctgtatgatggcatgcggagggtcgacctgacagggaaggcgaagggacccagtgagaacgtgtcccaggagcagttcacagcatccatgtcccacctgttgaaaggaaactccgaggagaagagtctcatgattatgaaaatgatttctgccacagaaggtcccgtgaaggccagagaagtccaaaagtttacagaggatctggttggctctgtggtgcacgtgctaagccacagacaggagctgagaggctggactgggaaggaagccccagggcccaacccccgggtgcaggtgctggctgctcagctgctctctgagatgaagctgcaagatggcaagagacttctggggccccagtggctggactatgactgtgaccgagctgtgatcgaggactgggtgttcagggtcccccatgtggccatattcctgagtgtggtcatttgcaagggctttctcgtcctgtgctcgtctcttgatctgactaccctggtccctgagcgtcaagtggaccagggcaggggttttgagagcatcctggatgtcctctctgtcatgtacatcaacgcccagctgcctcgggagcagcggcaccgctggcgcctgctcttttcgtctgagctccatggacacagcttctcccagctctgtggccacatcactcaccgggggccctgtgtggctgtcctcgaggaccatgacaagcatgtgttcggtgggtttgcctcttgctcttgggaggtgaagcctcagtttcaaggggacaatagatgcttcctgttctccatctgccccagcatggctgtgtacacacacacgggctacaacgaccactacatgtacttgaaccatggacagcagacgatcccgaacggactgggtatgggggggcagcacaattactttgggctttgggtggatgttgattttgggaaaggacacagcagagccaagcccacgtgcaccacgtacaacagcccgcagctgtcggctcaggagaacttccagtttgataagatggaggtgtgggcggttggagacccctcagaggagcagttggccaagggcaacaagagcatcctggatgcggaccctgaggcccaggccctgctggagatcagtgggcattcgcgccacagcgaagggctccgggaagtcccggacgatgaatgaggagccgcctgagccttcctggagccaggacctggattcctctgggtagagggcaccgcctgcagcccctcttccctcccacatggtttaactgtaatagcgtaccgcacgggtcacagcctggtagtcccacatgtgccaagaagacgccctctgaagccatgtcctgactcatgggtggatctcccagaaatagatgttttttaagttttcttttttgccctacagggtcaccctcaggatctctccaagaaatcttgtgtcaaattagtaactcagtcttatactttgttcttttttaccaacgaaatcctaagcagctgagacctttcagacccccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagggaattccgctggcattagcattgctgggatctcaacttctctattgaggagggggttcctcccgctggggttcatatggcctcactcaggccatccctgttagaaggtggtacctgatggtctgttctgacgggtgagggacagaggcatctggatgaaaatctcgacgctttctgggaacagcttccaatggcatgggagttctgtatttaaaagcatgaactgtgtataaaatggggaaatggaggcaggagactttgaggtaacatggtctggtggtggcatgagtcctcagaaggtatttgtaataaggttccacacccagctctctcagctgtttttaaatgaatgtgtgtgaggaacagatgggaaagttgggagatctgtctacagagaagcaaagttgtggttctcttgctaacttcaaggtgagggacattgggcaccctaagtttgggaacttggttgataaatacgtatatggtccattccataaatcagtggtgagtgactggcctgggttctagacctctgggaaccagcacctgagtcacagctgtctaggcctcggtgctggcctgggttctagatctctgggaaccagtgcctgagtcacagctgtcagtgcagccatttgcccagggctgctcccgagggggatgatgggaaattcagcagtgtagactcactttaaacaagctccggtgatcctgaaatgctgaagatcgtgtaggtgggttgtggggtcagcagagctgccatctgcccacgtctggaaaacaacacacggtgagtcaccggttggccatgagatctccccacttaaaggtgctgtgagcttgtctctaagatatatacctcttccttttgtcttttgctgtaagctttgaccttttgcagatctgatgaaaatacaacctcttattgtatagtttgccttgattataagccatagtaaatcgagctgttcgcatttttgcaggccttgcattttctactgggaggttcatcaaaccttccacttagcaatagccctgactcaggcagaatgcctcataaattagccttcgaaagaaaagtgcacgctcagacagtgtgtggaggggcaggagcgtgatgcagacaaggacttagtcatgactgattttccttagccagtttgtaatggcttatggggaacattgtgagcttcctacctgacatgttggcagaagtagaatattaggatcctcaagcatctctaccgtaagtccaaagaatttaggtccctattgtcactttgttttgcaatgttttttataaaaatgcaattaaatacctcactattaaactgccatgatgcctcagttaaaaaaaaaattaaatgtccgtcagtgtctgtgccttggtgaccattgattgctgcttctgcccaggccgctgcaaaggttaggactttgtccactcacatgctttccaccctgagcttgttctaaggagagtgcagatcacgaatgcacctgcttccaggcagtgcacttagagaaagggcactggcccgggcttgggagcctgggggatgcagctctctctgtgggcttctcttggtgcacggttctgcctctggacactcaaacctttttagggcgtgtgcagggctggggccagtggacctgacagtaccttacctggctctgagaaactcgcaatcctagtttgcacaaatgaatagtttgttcttttgtgaaaccagaatgagaattcaggttctaaggactagaggtctccctacaaatgaagacttatctcagggccctagaaagtgctaggaggaagaggttaaaaaaaaaatttgtccgcgttgcaaactattataaatgattgccacattgcaagctattattttctcccaccatattttttaattaaaaaaaagtctggggccgggtgcggtggctcacgcctgtaatcccagcactttgggaggccggggtaggcggatcacctgaggtcggaagttcgagaccagcctgaccaacatggagaaaccccatctctactaaaaatacaaaattagctgggcatggtggcacatgcctgtaatctcagctactcaggaggctgaggtagtagaattgcttgaaccagggaggcagaggttgtggtgagccgagatcgtgccattgcactctagcctgggcaacaagagtgaaactcagtttcaagaaaaaaaaaaaaggctgggtgtgatggctcacgcctggaatcccagcattttgggagcttggggcaggccgattgcttgagcccaggagttcaagaccagcctgggcaacatggcgaaaacccatctctacaaaaaaaaatacgaaaattaaccaggcatggtaacacatgcctgtggtcccagctacttgggaggctgaggtgtgaggatcatttaggcctggaaggttgaggctgctataagctgtgattgtgccactgcactccagcctgggcaacacagcaagaccctatgtttaaaaaaaattgtgtacctggtttag

SEQ ID NO: 43 - DNA, 3349 bp, Homo sapiens
  source 1..3349: mol_type = genomic DNA
agtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 44 - DNA, 2302 bp, Homo sapiens
  source 1..2302: mol_type = genomic DNA
gtcctgcttctcctccctctcgcctccagcctctcacactctcctaagccctctcatctcctggaaccatggccagcacatccaccaccatcaggagccacagcagcagccgccggggtttcagtgccaactcagccaggctccctggggtcagccgctctggcttcagcagcatctccgtgtcccgctccaggggcagtggtggcctgggtggcgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcactgtcaaccagagtctcctgactcccctcaacctgcaaattgaccccgccatccagcgggtgcgggccgaggagcgtgagcagatcaagaccctcaacaacaagtttgcctccttcatcgacaaggtgcggttcctagagcagcagaacaaggttctggacaccaagtggaccctgctgcaggagcagggcaccaagactgtgaggcagaacctggagccgttgttcgagcagtacatcaacaacctcaggaggcagctggacaacatcgtgggggaacggggtcgtctggactcggagctgagaaacatgcaggacctggtggaggacctcaagaacaaatatgaggatgaaatcaacaagcgcacagcagcagagaatgaatttgtgactctgaagaaggatgtggatgctgcctacatgaacaaggttgaactgcaagccaaggcagacactcttacagatgagatcaacttcctgagagccttgtatgatgcagagctgtcccagatgcagacccacatctcagacacatccgtggtgctatccatggacaacaaccgcaacctggacctggacagcatcatcgctgaggtcaaggcccaatatgaggagattgctcagaggagcagggctgaggctgagtcctggtaccagacaaagtacgaggagctgcagatcacagcaggcagacatggggacgacctgcgcaacaccaagcaggagattgctgagatcaaccgcatgatccagaggctgagatctgagatcgaccacgtcaagaagcagtgtgccaacctacaggccgccattgctgatgctgagcagcgtggggagatggccctcaaggatgctaagaacaagctggaagggctggaggatgccctgcagaaggccaagcaggacctggcccggctgctgaaggagtaccaggagctgatgaacgtcaagctggccctggatgtggagatcgccacctaccgcaagctgctggagggcgaggagtgcaggctgaatggcgaaggcgttggacaagtcaacatctctgtagtgcagtccaccgtctccagtggctatggcggtgccagcggtgtcggcagtggcttaggcctgggtggaggaagcagctactcctatggcagtggtcttggcgttggaggcggctttagttccagcagcggcagagccactgggggtggcctcagctctgttggaggcggcagttccaccatcaagtacaccaccacctcctcctccagcaggaagagctacaagcactgaagtgctgccgccagctctcagtcccacagctctcaggcccctctctggcagcagagccctctcctcaggttgcttgtcctcccctggcctccagtctcccctgccctcccgggtagagctgggatgccctcacttttcttctcatcaatacctgttccactgagctcctgttgcttaccatcaagtcaacagttatcagcactcagacatgcgaatgtcctttttagttcccgtattattacaggtatctgagtctgccataattctgagaagaaaatgacctatatccccataagaactgaaactcagtctaggtccagctgcagatgaggagtcctctctttaattgctaaccatcctgcccattatagctacactcaggagttctcatctgacaagtcagttgtcctgatcttctcttgcagtgtccctgaatggcaagtgatgtaccttctgatgcagtctgcattcctgcactgctttctctgctctctttgccttcttttgttctgttgaataaagcatattgagaatgtgaa

SEQ ID NO: 45 - DNA, 2591 bp, Homo sapiens
  source 1..2591: mol_type = genomic DNA
gtaggactccggccttggtggcgggtggctggcggttccgttaggtctgagggagcgatggcggtacgcgcgttgaagctgctgaccacactgctggctgtcgtggccgctgcctcccaagccgaggtcgagtccgaggcaggatggggcatggtgacgcctgatctgctcttcgccgaggggaccgcagcctacgcgcgcggggactggcccggggtggtcctgagcatggaacgggcgctgcgctcccgggcagccctccgcgcccttcgcctgcgctgccgcacccagtgtgccgccgacttcccgtgggagctggaccccgactggtcccccagcccggcccaggcctcgggcgccgccgccctgcgcgacctgagcttcttcgggggccttctgcgtcgcgctgcctgcctgcgccgctgcctcgggccgccggccgcccactcgctcagcgaagagatggagctggagttccgcaagcggagcccctacaactacctgcaggtcgcctacttcaagatcaacaagttggagaaagctgttgctgcagcacacaccttcttcgtgggcaatcctgagcacatggaaatgcagcagaacctagactattaccaaaccatgtctggagtgaaggaggccgacttcaaggatcttgagactcaaccccatatgcaagaatttcgactgggagtgcgactctactcagaggaacagccacaggaagctgtgccccacctagaggcggcgctgcaagaatactttgtggcctatgaggagtgccgtgccctctgcgaagggccctatgactacgatggctacaactaccttgagtacaacgctgacctcttccaggccatcacagatcattacatccaggtcctcaactgtaagcagaactgtgtcacggagcttgcttcccacccaagtcgagagaagccctttgaagacttcctcccatcgcattataattatctgcagtttgcctactataacattgggaattatacacaggctgttgaatgtgccaagacctatcttctcttcttccccaatgacgaggtgatgaaccaaaatttggcctattatgcagctatgcttggagaagaacacaccagatccatcggcccccgtgagagtgccaaggagtaccgacagcgaagcctactggaaaaagaactgcttttcttcgcttatgatgtttttggaattccctttgtggatccggattcatggactccagaagaagtgattcccaagagattgcaagagaaacagaagtcagaacgggaaacagccgtacgcatctcccaggagattgggaaccttatgaaggaaatcgagacccttgtggaagagaagaccaaggagtcactggatgtgagcagactgacccgggaaggtggccccctgctgtatgaaggcatcagtctcaccatgaactccaaactcctgaatggttcccagcgggtggtgatggacggcgtaatctctgaccacgagtgtcaggagctgcagagactgaccaatgtggcagcaacctcaggagatggctaccggggtcagacctccccacatactcccaatgaaaagttctatggtgtcactgtcttcaaagccctcaagctggggcaagaaggcaaagttcctctgcagagtgcccacctgtactacaacgtgacggagaaggtgcggcgcatcatggagtcctacttccgcctggatacgcccctctacttttcctactctcatctggtgtgccgcactgccatcgaagaggtccaggcagagaggaaggatgatagtcatccagtccacgtggacaactgcatcctgaatgccgagaccctcgtgtgtgtcaaagagcccccagcctacaccttccgcgactacagcgccatcctttacctaaatggggacttcgatggcggaaacttttatttcactgaactggatgccaagaccgtgacggcagaggtgcagcctcagtgtggaagagccgtgggattctcttcaggcactgaaaacccacatggagtgaaggctgtcaccagggggcagcgctgtgccatcgccctgtggttcaccctggaccctcgacacagcgagcgggacagggtgcaggcagatgacctggtgaagatgctcttcagcccagaagagatggacctctcccaggagcagcccctggatgcccagcagggcccccccgaacctgcacaagagtctctctcaggcagtgaatcgaagcccaaggatgagctatgacagcgtccaggtcagacggatgggtgactagacccatggagaggaactcttctgcactctgagctggccagcccctcggggctgcagagcagtgagcctacatctgccactcagccgaggggaccctgctcacagccttctacatggtgctactgctcttggagtggacatgaccagacaccgcaccccctggatctggctgagggctcaggacacaggcccagccacccccaggggcctccacaggccgctgcatgacagcgatacagtacttaagtgtctgtgtagacaaccaaagaataaatgattcatggtttttttta

SEQ ID NO: 46 - DNA, 4833 bp, Homo sapiens
  source 1..4833: mol_type = genomic DNA
acttctccctgagccctccccgcgcgctccctgcctccccgcggccgcgggagaggagcccggccagggggaggcgcggagcgggccggccgcccggccttcctctccctcgctcctcctctccgcctcaacttgctccggcctcctcgctcctgcctgagttcggggcctggtctgggcttcgtcctggggctgggaagtttctgcgctccccgctgtccagtctgcatctccgcgcctcccgccgggcccccgcggtcccctcccgcccgcttcccccgctgccctccctcgctgccccctaccctctcccggggctcccggccctctactcccttcgggcgcgggcccctcgccccgcccgggcgcgccccccctcccccgggtcctgcggccccggatgcccgggccccgaggggctgctggcggcctggcccctgagatgcgcggggcgggggcggcggggctgctggcgctgctgctgctgctgctgctgctgctgctgggcctgggcggcagggtcgagggggggccggccggcgagcggggcgcaggcgggggcggggcgctggcccgcgagcgcttcaaggtggtctttgcgccggtgatctgcaagcggacctgtctcaagggccagtgtcgggacagttgtcagcagggctccaacatgacgctcatcggagagaacggccacagcacagacacgctcacgggctccggcttccgcgtggtggtgtgccctctcccctgcatgaatggcggccagtgctcctcgcgaaaccagtgcctgtgtcccccggacttcactgggcgcttctgccaggtgcccgcaggaggagccggtgggggtaccggcggctcaggccccggcctgagcaggacaggggccctgtccacaggggcgctgccgcccctggctccggagggcgactctgtggccagcaagcacgccatctacgccgtccaggtgatcgctgaccctcctgggcccggggaggggcctcctgcccagcacgcagccttcctggtgcccctaggcccgggacagatctcagcagaagtgcaggccccgccccccgtggtgaatgtgcgcgtccatcacccgcccgaggcctcagtccaggtgcaccgcattgagagctcgaacgccgagagcgcagccccctcccagcacctgctgccgcaccccaagccctcgcacccccggccgcccacccagaagcccctgggccgctgctttcaggacactctgcccaagcagccgtgtggcagcaaccccctccccggcctcaccaagcaggaagactgctgcggtagcatcggcactgcctggggccagagcaagtgccacaagtgtccccagctgcagtacacaggagtgcagaagccagggcctgtacgtggggaagtgggcgctgactgtccccagggctacaagaggcttaacagcacccactgccaggacatcaacgagtgcgcaatgccgggcgtgtgtcgccatggtgactgcctcaacaaccctggctcctatcgctgtgtctgcccacctggccatagtttaggcccctcccgtacacagtgcattgcagacaaaccggaggagaagagcctgtgtttccgcctggtgagccctgagcaccagtgccagcacccactgaccacccgcctgacccgccagctctgctgctgcagtgtcggcaaggcctggggcgcgcggtgtcagcgctgcccaacagatggcaccgctgcgttcaaggagatctgcccagctgggaagggataccacattctcacctcccaccagacgctcaccattcagggcgagagtgacttttcccttttcctgcaccctgacgggccacccaagccccagcagcttccggagagccctagccaggctccaccacctgaggacacagaggaagagagaggggtgaccacggactcaccggtgagtgaggagaggtcagtgcagcagagccacccaactgccaccacgactcctgcccggccctaccccgagctgatctcccgtccctcgcccccgaccatgcgctggttcctgccggacttgcctccttcccgcagcgccgtagagatcgctcccactcaggtcacagagactgatgagtgccgactgaaccagaacatctgtggccacggagagtgcgtgccgggcccccctgactactcctgccactgcaaccccggctaccggtcacatccccagcaccgctactgcgtggatgtgaacgagtgcgaggcagagccctgtggcccggggaggggcatctgcatgaacaccggcggctcctacaattgccactgcaaccgcggctaccgcctgcacgtgggcgccggggggcgctcgtgcgtggacctgaacgaatgcgccaagccccacctgtgcggcgacggcggcttctgcatcaactttcccggtcactacaagtgcaactgctaccccggctaccggctcaaagcctcccggcctcctgtgtgcgaagacatcgacgagtgccgggacccaagctcttgcccggatggcaaatgcgagaacaagcccgggagcttcaagtgcatcgcctgtcagcctggctaccgcagccaggggggcggggcctgtcgcgacgtgaacgagtgcgccgagggcagcccctgctcgcctggctggtgcgagaacctcccgggctccttccgctgcacctgtgcccagggctacgcgcccgcgcccgacggccgcagttgcttggatgtggacgagtgtgaggctggggacgtgtgtgacaatggcatctgcagcaacacgccaggatctttccagtgtcagtgcctctctggctaccatctgtccagggaccggagccactgcgaggacattgatgagtgtgacttccctgcagcctgcattgggggtgactgcatcaataccaatggctcctacagatgtctttgcccccaggggcatcggctggtgggtggcaggaaatgccaagacatagatgagtgcagccaggacccgagcctgtgccttccccatggggcctgcaagaaccttcagggctcctatgtgtgtgtctgcgatgagggcttcactcccacccaggaccagcacggttgtgaggaggtggagcagccccaccacaagaaggagtgctacctgaacttcgatgacacagtgttctgcgacagcgtattggccaccaacgtgacccagcaggagtgctgctgctctctgggggccggctggggcgaccactgcgaaatctacccctgcccagtctacagctcagccgagttccacagcctctgcccagacggaaagggctacacccaggacaacaacatcgtcaactacggcatcccagcccaccgtgacatcgacgagtgcatgttgttcgggtcggagatttgcaaggagggcaagtgcgtgaacacgcagcctggctacgagtgctactgcaagcagggcttctactacgacgggaacctgctggaatgcgtggacgtggacgagtgcctggacgagtccaactgccggaacggagtgtgtgagaacacgcgcggcggctaccgctgtgcctgcacgccccctgccgagtacagtcccgcgcagcgccagtgcctgagcccggaagagatggacgtggacgagtgccaggacccggcagcctgccgccctggccgctgcgtcaacctgccgggctcctaccgctgcgagtgtcgcccgccctgggtgcccgggccctccggccgcgattgccagctccccgagagcccggccgagcgtgccccggagcggcgcgacgtgtgctggagccagcgcggagaggacggcatgtgcgctggccccctggccgggcctgccctcaccttcgacgactgctgctgccgccagggccgcggctggggcgcccaatgccgaccgtgcccgccgcgcggcgcggggtcccattgcccgacatcgcagagcgagagcaattccttctgggacacaagccccctgctgttggggaagcccccaagagatgaggacagttcagaggaggattcagacgagtgtcgctgcgtgagtggccgctgcgtgccgcggccgggcggcgccgtgtgcgagtgtcccggcggcttccagctcgacgcctcccgcgcccgctgcgtggatatcgacgagtgccgagagctgaaccagcgcgggctgctgtgcaagagcgagcgctgcgtgaacaccagcggctccttccgctgcgtctgcaaagccggcttcgcgcgcagccgcccgcacggggcctgcgttccccagcgccgccgctgacgccgccgacgccgccctcggcccagacctcggtgatcactgagggatttccgcgagctcggcctcacttctgccccgacttgtggctcggacccagggaccttcagggcccgcagaccctcccggcgccttgagacccgaggcgcccctaccggcccccctccccggttagcgggcggttgtaaggtctccggcgggcgctgcctgccttcctcccagagggtgtttcctagaaactgataaatcagatcgtgcctctttacccttggctttcgaagcaaattgatgttcacgtctgacgtgggcgcgggctgcgcagggcggcgccagaccccagccgcctcccaggggctagactgagcccggcacaaggggtgtgaaatagaatttattgtggctctgattatgtacacgtgagatggcctggctgggccggccgggctcacatggtttgtacaataaatacatctgtggggcgggctctccgcagccgggaagggccaccgccacggttcagtcca

SEQ ID NO: 47 - DNA, 3375 bp, Homo sapiens
  source 1..3375: mol_type = genomic DNA
gttgggccgaggccgggactgggccggcgccgggcggggaacgggttcgcgaccgcagccgagagaccccgggcgaccggcacctccgagactcgcgggccacctgcctcgaccttccccggagcgcccccgcctccgagtcgctacttgccgggccgggccgggccgggcgtgatgcgccgcgggacccctgtcctggccactggccgccgccgccgccgccgctgagaccccgagacccccagtgacgccgcagccatggagagcggcccgcgtgcggagctgggggcgggcgcacccccagctgtggtcgccaggacccccccagagccaagaccttctccagaaggtgacccttcccccccaccaccaccaatgtcagccctggtccccgacactcccccggacacccctcctgccatgaagaatgccactagctctaagcagctcccactggaaccagagagcccctcagggcaggtcgggcctaggccagcccccccgcaggaagagtccccttcctctgaagcaaagagcagaggacccaccccaccagccatgggcccacgggatgccagacctcctcgaaggagcagccagccatctccaacagcagtgccagcctccgacagccctcccaccaagcaagaggtgaagaaggcaggagagagacacaagctggcaaaggagcggcgagaagagcgggccaagtacctggcggccaagaaggcagtgtggctggagaaggaggagaaggccaaggcgctgcgggagaagcagctccaggagcgccggcgccggctggaggagcaacgtcttaaagccgagcaacgccgtgcagccctggaggaacggcagcggcagaagctcgagaaaaacaaggagcgctatgaagcagccatccaacggtcagtgaagaagacgtgggccgaaatccggcagcagcgctggtcctgggcaggggccctgcaccacagctctccaggacataagaccagtgggagcaggtgctccgtgtcggcagttaacctgcccaaacacgtggactctataatcaacaagcggctctcaaagtcctctgccacgctctggaactcccccagtagaaatcgcagcctgcagctgagcgcatgggagagcagcatcgtggatcgtctgatgacgcccactctctccttccttgctcggagtcgcagcgcggtcacactgccccgcaacggccgggaccagggtaggggctgcgaccctgggagaggccccacgtggggccgggcaggggccagcctggcgcgcgggccgcaacccgaccgcactcatccctctgcagccgtgccggtgtgcccgcgctcggcctccgccagccccctgacgccgtgcagcgtcacccgaagcgtgcaccgctgcgcccccgccggtgagcgcggggagcgccgcaagcccaacgccgggggcagccccgctccggtgcgccgccggccggaggcctcgccggtgcagaaaaaggagaagaaggacaaggagcgggaaaacgagaaggagaagagtgccctagcccgggagcgcagcctcaagaagcgccagtcgctgcccgcctccccacgtgcccgcctctctgccagcaccgcctctgagctcagccccaaatccaaggccaggccatcctctccctccacatcctggcacaggcctgcctccccctgccccagcccagggccaggccacactctgcctccaaagccaccgtccccccgaggcaccactgcatcccccaaggggcgggttcggaggaaggaggaggcaaaggagagccccagcgccgcagggcccgaggacaagagccagagcaagcgcagggccagtaacgagaaggagtcagcagccccagcctcaccggcaccttcgccggcgccctcgcccaccccagccccgccccagaaggagcagccccccgcggagacccctacagacgctgctgtcttgacctcacccccagcccctgctcccccggtgacccctagcaaaccaatggccggcaccacagaccgagaagaagccactcggctcttggctgagaagcggcgccaggcccgggagcagcgggagcgcgaggagcaggagcggaggctgcaggcagaaagggacaagcgaatgcgagaggagcagctggcacgggaggccgaggcccgggcggagcgggaggcggaggcccggaggcgggaggagcaggaggcacgagagaaggcgcaggccgagcaggaggagcaggagcggctgcagaagcagaaagaggaggccgaagctcggtcgcgggaagaggcggagcggcagcgtctggagcgggaaaagcacttccagcagcaggagcaagagcggcaagagcgcagaaagcgtctggaggagatcatgaagaggactcggaagtcagaagtttctgaaaccaagcagaagcaggacagcaaggaggccaacgccaacggttccagcccagagcctgtgaaagctgtggaggctcggtccccagggctgcagaaggaggctgtgcagaaagaggagcccatcccacaggagcctcagtggagtctcccaagcaaggagttgccagcgtccctggtgaatggcctgcagcctctcccagcacaccaggagaatggcttctccaccaacggaccctctggggacaagagtctgagccgaacaccagagacactcctgccctttgcagaggcagaagccttcctcaagaaagctgtggtgcagtccccgcaggtcacagaagtcctttaagagggtttgccttggatccgggcacagttgtgagggctcctctgcatcacctaccaggatgtctggaggagaaaaagacagaacaaagatggaagtggcctgggcccctgggggtgggtcctctctgttgtttttaatctgcaccttatagactgatgtctctttggccggagccagatctgcccctcagtgcattcgtgtgctcgcacgcgcagacatcccttctcccccatacacacatatacactcacagcctctctggcctcttcccttggggaggggccacctgtagtatttgccttgatttggtggggtacagtggatgtgaatactgtaaatagcttgtgctcagactcctctgcgtggagagggtgggtgcaggaggcagaccctccccccaaagccccctggggagatcttcctctctctatttaactgtaactgagggggatcccaggtctggggatgggggacaccttgggccacaggatactggttgcttcaggggtacccatgccccctgccctcgcctggaatcagtgttactgcatctgattaaatgtctccagaaataaagaataattctgccaa

SEQ ID NO: 48 - DNA, 4566 bp, Homo sapiens
  source 1..4566: mol_type = genomic DNA
attctggtcggaatccacctctccgcctgtgcaacacacactttacacacgcacggggactgcaagcgggcagcatcgatcgtggctcctttaagacaaactcagacagacattttttttaaccctccttctctaatctccttccagtgcagcacttgcaaagagggagagagagggagagaaagaaagagagagagagaagagagaaactgattaggaattaggactgattcaagggaagcgagcgctagggctttgtgcatttgaatattaacatttgaggtgttctgaccagaagaagacagagcggatgatcattcattcaccacgttgacaacctcgcctgtgattgacagctggagtggcagaaagccatgagatttggtagttgggtctgaggggcgctcttttttttccttttctttctttctttctttttttttttttaaactgatttttgggggagagaagatctgcttttttttgcccccgctgctgtcttggaaacggagcgcttttatgctcagtgactcgggcgctttgcttcaggtcccgtagaccgaagatctgggaccagtagctcacgttgctggagacgttaagggatttttcgtcgtgcttttttttttttttttttttttttccgggggagtttgaatatttgtttcttttcacactggccttaaagaggatatattagaagttgaagtaggaagggagccagagaggccgatggcgcaaaggtacgacgatctaccccattacgggggcatggatggagtaggcatcccctccacgatgtatggggacccgcatgcagccaggtccatgcagccggtccaccacctgaaccacgggcctcctctgcactcgcatcagtacccgcacacagctcataccaacgccatggcccccagcatgggctcctctgtcaatgacgctttaaagagagataaagatgccatttatggacaccccctcttccctctcttagcactgatttttgagaaatgtgaattagctacttgtaccccccgcgagccgggggtggcgggcggggacgtctgctcgtcagagtcattcaatgaagatatagccgtgttcgccaaacagattcgcgcagaaaaacctctattttcttctaatccagaactggataacttgatgattcaagccatacaagtattaaggtttcatctattggaattagagaaggtacacgaattatgtgacaatttctgccaccggtatattagctgtttgaaagggaaaatgcctatcgatttggtgatagacgatagagaaggaggatcaaaatcagacagtgaagatataacaagatcagcaaatctaactgaccagccctcttggaacagagatcatgatgacacggcatctactcgttcaggaggaaccccaggcccttccagcggtggccacacgtcacacagtggggacaacagcagtgagcaaggtgatggcttggacaacagtgtagcttcccccagcacaggtgacgatgatgaccctgataaggacaaaaagcgtcacaaaaagcgtggcatctttcccaaagtagccacaaatatcatgagggcgtggctgttccagcatctaacacacccttacccttctgaagaacagaaaaagcagttggcacaagacacgggactcaccatccttcaagtgaacaattggtttattaatgcccggagaagaatagtgcagcccatgatagaccagtccaaccgagcagtaagtcaaggaacaccttataatcctgatggacagcccatgggaggtttcgtaatggacggtcagcaacatatgggaattagagcaccaggacctatgagtggaatgggcatgaatatgggcatggaggggcagtggcactacatgtaaccttcatctagttaaccaatcgcaaagcaagggggaaggctgcaaagtatgccaggggagtatgtagcccggggtggtccaatgggtgtgagtatgggacagccaagttatacccaaccccagatgcccccccatcctgctcagctgcgtcatgggccccccatgcatacgtacattcctggacaccctcaccacccaacagtgatgatgcatggaggaccgccccaccctggaatgccaatgtcagcatcaagccccacagttcttaatacaggagacccaacaatgagtggacaagtcatggacattcatgctcagtagcttaagggaatatgcattgtctgcaatggtgactgatttcaaatcatgttttttctgcaatgactgtggagttccattcttggcatctactctggaccaaggagcatccctaattcttcatagggacctttaaaaagcaggaaataccaactgaagtcaatttgggggacatgctaaataactatataagacattaagagaacaaagagtgaaatattgtaaatgctattatactgttatccatattacgttgtttcttatagattttttaaaaaaaatgtgaaatttttccacactatgtgtgttgtttccatagctcttcacttcctccagaagcctccttacattaaaaagccttacagttatcctgcaagggacaggaaggtctgatttgcaggatttttagagcattaaaataactatcaggcagaagaatctttcttctcgcctaggatttcagccatgcgcgcgctctctctctttctctctcttttcctctctctccctctttctagcctggggcttgaatttgcatgtctaattcatttactcaccatatttgaattggcctgaacagatgtaaatcgggaaggatgggaaaaactgcagtcatcaacaatgattaatcagctgttgcaggcagtgtcttaaggagactggtaggaggaggcatggaaaccaaaaggccgtgtgtttagaagcctaattgtcacatcaagcatcattgtccccatgcaacaaccaccaccttatacatcacttcctgttttaagcagctctaaaacatagactgaagatttatttttaatatgttgactttatttctgagcaaagcatcggtcatgtgtgtattttttcatagtcccaccttggagcatttatgtagacattgtaaataaattttgtgcaaaaaggactggaaaaatgaactgtattattgcaatttttttttgtaaaagtagcagtttggtatgagttggcatgcatacaagatttactaagtgggataagctaattatactttttgttgtggataaacaaatgcttgttgatagcctttttctatcaagaaaccaaggagctaattattaataacaatcattgcacactgagtcttagcgtttctgatggaaacagtttggattgtataataacgccaagcccagttgtagtcgtttgagtgcagtaatgaaatctgaatctaaaataaaaacaagattatttttgtcatgctgactccactgcttgaaaaatttgtttactgccccccagatttttaaagattaatacagtaaatataaaaattaattttggctccctaagccatatatgtatttgacaattttaaccgcaaagtaagttgtttaataataaccactgatttctttaagctgacttaatgaactcctaatatcagcaaatttgaggcctaaaggcactaaactaactctagactcagaattacatccaacagaattactcatctaatatcagtgaaattattcttgcacataaaggcaaacctaagtacaaagttaagtcttttactaaaggatgttacctaggatgagcagtatatgtttattaggaaattaactacatgaattgaagagaccagacttcaaaatctaatttttataaatatgctctatgttctcattggataaaactggttattaaccaattttccagaacagctgtacaagatttctgcatggcagccggctaaatggtagaaaataatatgtttaagctggaatagcttataattttatttaaataaaattgctgctataaaaagtgcttcccaaagctaaggaaaatatacaaatattttaattaaggcaaattcgttttaaaaaataagcctttcagtagtgattgctttgtaaacaaaggatgggtcggaggagagagagccttaaactcaagtctgacattcaggcccaagtcaccctataaaccggcccttagcaattctattttctattcgaaaagagaaaccagctgtgggttggctttactaggtgatgtgtgattgactgactcacctgctggagtagcaatgcattctcaccttttgctgatggggccttgtttctaaacacgtggatgcgcagagagcaaggctcagcctactgcagccatctctggcagcgcttctacccctccccagcccatagatgggattgtttaaatctcccttgttgacctagtggcaattcttcctctacctaaatagtcgatgtgagtgaaatcattctcttttgataggtggttgctagcagttaacaaccatttatttactttaacaattaataataaactttataaactgtc

SEQ ID NO: 49 - DNA, 6487 bp, Homo sapiens
  source 1..6487: mol_type = genomic DNA
agtcgagccccggccggctgcgcggcgcgctgggtgcatgagggggctgctccggagcgacggcggctgcagctggagccaggcgctcgcccgtccgccggttggctcgccgggacctcgcgcaccggcggcagagtcccttgcgtggattggcaagcgacgccccacctgccccgagctcaccattttctttcgcgctggctgcagctgacccggcgaagggagccgaccgggccctgggctggaggtaaaaccccacggaaagaacatgaggttcccttggaaatcattcaagaggaagatggaaggggctgtttagaagagcttaaaagctacaggctgtaagactggggcctgagtgctggcagtggaaaacactgttgggctgtgatctctccctgaaaagttccaggtgccttttcgcttcctgcaaaagaaaggaagcgaaagagaagaccatgtccatagccctgaagcaggtattcaacaaggacaagaccttccgacccaagaggaaatttgaacctggcacacagaggtttgagctgcacaaacgggctcaggcatccctcaactcgggtgtggacctgaaggcggctgtgcagttgcccagtggggaggaccagaatgactgggtggcagtacatgtggtggacttcttcaatcggatcaacctcatctatggcaccatctgtgagttctgcaccgagcggacctgtcctgtgatgtcagggggccccaaatatgagtatcggtggcaggatgatctcaagtataagaagccaacagcgctgccagctccccagtacatgaaccttcttatggattggattgaggttcagatcaacaacgaggaaatatttccaacatgcgtgggtgttcccttcccaaagaacttccttcagatctgcaagaagatcctgtgccgccttttccgggtctttgtccacgtctatatccaccacttcgaccgggtcattgtgatgggtgcagaggcccatgtcaacacctgctacaaacacttctattactttgtcacagagatgaacctcatagaccgcaaggagctagagcctttgaaagaaatgacgagcaggatgtgtcactaatgctccacctcaccctttggaagaaaggaaagctgtttcctcctggtgccctgagcgggcaggaggtggaccaccctggctgaaatgacacacctactcccaggaacagcagaggtggaggcaagcagtgactcctgagagacattccccactcactttgtgtgctcttaaccttctgagtgctgctagcccagacctgtggacgaggcagaccacaacgtgaaagaaggaccagccccttgaccgttctggctggggaattgtccacgaggaagcctctgcacttccacacatggcacagttctgcctgtgacctgccgcctaagctttactggaattcaggttttgagactgagatgcgtgttcgtatttttccacttatctgtcttgtcagctggccgacttctctgtgattggttttttaagtgccgggtgaattttggacctctggatgtgcagcaagtttttatgcaataagccttcctttcaggtctctaaaagctcctgctctgatctgtggtttaacactgtgcagggctgtggagctctgagacacctgaacccctacccatcccctgcacctccctactctccctgccgaggcgtccatagcatttccctaataaatagttttatcagggacactccatggggtggcttgtctctgccccaaacagggtcttcacgttctaactgcagggaagagactggttactagcgtaaagctgacaagttaccagttcacctgatcagaatgtattttttataaccaccatcattccttgtctcttcagtggaagatattctttcctgtttcccagaagagagaaataaaagtcctaaataactaagaatgatcagcgggagcgttgggcatgattactgcagtgtttgttctttattaaagacagggagtcgtggctgtctctacataatactaacatttcagtgaaaaaagtacaattggttatttggtacgtgtagatttaacacccagacggctgacttgtaaccctccccactagccttctgagcatttaaacccagccgtcattctctccccactccacgctccccactcctctgccacgttatgttacggcacaaaattaatttctgcccctgtgattgggccacggttgccaaggtaacagtggagctggagctaacttccttctttcatcctttccaatttttctattccagaagacagtcagaataatgcattctgtactacatcctgccttttgaaacctaaatgagttttcgttgatgaaatgttgccttctctgattcattcacaaaactgtggtggttctgaccacctgtgacgaggggggtcataatacttccagtgatccttttaatttagcaaaatatttgtcggtggagggaagtagataagaatgtattagtgtattttaaagtaataatgattaaagaataactaaatgactctgattttgttttatcaatcataatgtgttaaagccctctgtggctgccacaaaagactagaatcatgaaatctttgtccaagctaagaaagggctgtctctgtgggtacagtggcattctctgttgacctaaccaagcagcctgaaccttgctttgtgtctcgtaaaggtcatctcacggaatccaattgctgctacctcaccatgttgctgtgccctggtcaacaataaacatacttttgtcccccttcctaagtaactttgagaggacaagataggcaaagttttttccctacatctcttttggccaaaagaatatctgaccatcacagagatccagagaacagaaacaagagcccctaagtcccagcctctgcaatacacagttaccagatgggaatgcagcacagtggcacagagaggtaactacggagcccatgccctggaggaactctgtagctgtgcatcatggaacgccacaggatttttacattaaaaagttgaatatgttcctgcatttcagcagtgcataatcgagcaggaggctggaatctggagacctttgttattcattctttttaaggttattttttattaactgtgcatatgaaatttccactcaatttaaaaatccaaatctttttaagcttcttggaagaatttcctcctgcttatattgttaattgcactaatgaactcttctagggtaaattctggcaaactaccctttttttttttttaatcacagaatattcctgttcccacagaactttggcacattctgaagaaaatctctgcagtcaatttttatctcctctgcttgactgcatctaactgttcatctctgttataaatggagcttcagccctctgggtcagagactgacaaccctccttcttcttcctaacctgtcctactctccttctttttctgctttcattttaattttgggaaattttaaagtaaaagtttcaaagttgaaagattatagtgagtacccttatgtcctttagtgcatcagttgttaatgttttgccttgttttcttttcttccttttttttttttaagagaaggggtctctccctattttacccaagctggccttgaaccccttgcctcaagtgagcttcaaccttagcctcctgagtagccaggactatggcatgtaccaccgcacctgcctcacattttctttctcactgtaaatacacattcttattcttgaaccttctgaaaataagttgcagacatcatgaaaatgtaaaatacttcatcatattctttcttttaataagagcattgtcctataaatctgcaatattttcacgcccatgaaactgaacactgatactgcataaatctaatatatgatccatattcaaaattctccagttgtccccaaaatatcctttatagctgtttatttttaaatccaggatccaatcaagaattatacgttatatgtagttgtcattctcttttgtttcctttaattgattagagttgcccttttgtgtgtgtctgagtggtgggtggtggggtggtgtttgtgtatgatgtgcagtgtgtgtgtgtgtggtgtgtctttcatgacactgacattattaaagagttcaggcaagtttgtggaatgtcccacaatctagacttttttactgattgtttcctcattacgagatacaggttacattttttttcccaagactgctacatgtgtgatgttgtttccttactactgcctcacctcaggaagcacataatgtctacttgtctcttttctgatattaggactgatcaggtgttgtctgcctgatccaatcattatcaagttccataattcattattaagctctgaaattcaggacacatacatttatagagtgtgaggctgtgcatacgtacattcatcccttaatgttctcaaggaggcccaggatccccaaaatattataagcattgcagtcagatgatatagcactagcaaagcattcatccctttccatataacaggagggaggggaagaaggaagtttccatgtgcaggcaatacgtgagagcatgggcaagtgaggatttgtatgctccctgtagccagaggtgaaatattcaacgcttttattttttctcccgtcttcaatgacctcccatttggacccatagtttatttatagagagcacacactccctaattgtcagatggtttttttgggaaaactcttagatagtacagtgagttgtgaccctccaaaggatcacaagtacataaacagatacaaagtatttatgtggtattaaaatttcatggtgatggtgcaagggagagatgattaggaagaaaatgtcttacacggctgtaattgagaaatactgatctagagtcatttcctgcttcttaaattgttcgttttctcttccctgggtattttctcatttccaaagacccagagtctcagagccaggttaagtggaagaaactttgagaggtttcttaatcaattccttctatctctcaaaccatgccagtaacttccttggtgaaaaaatctatactcctctcacagttcttcgggcatggaaattctatagcattctttggtataaccaacccactgaaccctccacaacatcttgaattccattcaactcgagctgaagtattctttttgagtttcagtatatcttttctagtgtcccacttttagaaaggtccaaagatgatatataataagtgaaaatacaattaatcagagctttatttgcatatccattaaatatctgaaggctattaccagttgtgcttcagcctcctttcccttgcaattctcccagggtggcatcctctacggagtcttgttgctccattgttgaccactgatgtaactcaacgtgaaaccttcccaaagctctccacatcactctctgttatggctgtgaccagcagtgaacatgaaaagaacttatttcatgccttctccaaactatattgtttctttgctaagtccagacctgctttttctacatttataggttttctattttaatcgccatgcaaaattctttatcatactcaatcattttggtttatcttcctgacttcttacctttaacatccctgttaaagattacactgttgttgtttcaaaaaaaaaaaaaaaaaaaccttaattggcaaaacattaagggacttgaatagaattacacttatctttttgtgctgatttatgtcaatccatcattctggttattgatggaagcaaattgcttcctatgttctctaaaccttatgtcccttccatactccatgagtaccacactgggagaaaacaaaagcaaaaagattgtgggaaaagtatagccattatctttgaggaaatgtgtaccaaggcacaatcattaaaaggagttggaggcatcatttggttgacactgttgtcattcttgttctgatcattttggaccttgaagaaattggtgattctctcctagaattagacaaacaaagtgtgtttggaaataatgattgttttcctgccttaaaaaatatattaacagaaagcttttataacaggctgtttccctctggacaggtattaattctgagtaagaattttcagtgactacataaggatttgtgtaacttatgaaggaagagtccatttctaatcaaataattcgcctgttttactagcttatagtgatctgatttcagaattttcctgtatcttttttacatacatcagaaaaagaaatgtttactatatttttggttccatttatgattgtattaagcatttgactataaggaaaactaacaattaaatcaattagaaaagcaacataaaattaaatgatatttaggaaatcagttatatgtgagcttgggtattcaaatgtcacaaataaaaagcatataaccatta

SEQ ID NO: 50 - DNA, 2493 bp, Homo sapiens
  source 1..2493: mol_type = genomic DNA
agttctgtggagcagcggtggccggctaggatgggctgtctctggggtctggctctgccccttttcttcttctgctgggaggttggggtctctgggagctctgcaggccccagcacccgcagagcagacactgcgatgacaacggacgacacagaagtgcccgctatgactctagcaccgggccacgccgctctggaaactcaaacgctgagcgctgagacctcttctagggcctcaaccccagccggccccattccagaagcagagaccaggggagccaagagaatttcccctgcaagagagaccaggagtttcacaaaaacatctcccaacttcatggtgctgatcgccacctccgtggagacatcagccgccagtggcagccccgagggagctggaatgaccacagttcagaccatcacaggcagtgatcccagggaagccatctttgacaccctttgcaccgatgacagctctgaagaggcaaagacactcacaatggacatattgacattggctcacacctccacagaagctaagggcctgtcctcagagagcagcgcctcttccgacagcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccgtcacgggcctcagagagcagcgcctcttccgacggcctccatccagtcatcaccccgtcacgggcctcagagagcagcgcctcttccgacggcccccatccagtcatcaccccgtcacgggcctcagagagcagcgcctcttccgacggcccccatccagtcatcaccccctcatggtccccgggatctgacgtcactctcctcgctgaagccctggtgactgtcacaaacatcgaggttattaattgcagcatcacagaaatagaaacaacgacttccagcatccctggggcctcagacacagatctcatccccacggaaggggtgaaggcctcgtccacctccgatccaccagctctgcctgactccactgaagcaaaaccacacatcactgaggtcacagcctctgccgagaccctgtccacagccggcaccacagagtcagctgcacctgatgccacggttgggaccccactccccactaacagcgccacagaaagagaagtgacagcacccggggccacgaccctcagtggagctctggtcacagttagcaggaatccccttgaagaaacctcagccctctctgttgagacaccaagttacgtcaaagtctcaggagcagctccggtctccatagaggctgggtcagcagtgggcaaaacaacttcctttgctgggagctctgcttcctcctacagcccctcggaagccgccctcaagaacttcaccccttcagagacaccgaccatggacatcgcaaccaaggggcccttccccaccagcagggaccctcttccttctgtccctccgactacaaccaacagcagccgagggacgaacagcaccttagccaagatcacaacctcagcgaagaccacgatgaagcccccaacagccacgcccacgactgcccggacgaggccgaccacagacgtgagtgcaggtgaaaatggaggtttcctcctcctgcggctgagtgtggcttccccggaagacctcactgaccccagagtggcagaaaggctgatgcagcagctccaccgggaactccacgcccacgcgcctcacttccaggtctccttactgcgtgtcaggagaggctaacggacatcagctgcagccaggcatgtcccgtatgccaaaagagggtgctgcccctagcctgggcccccaccgacagactgcagctgcgttactgtgctgagaggtacccagaaggttcccatgaagggcagcatgtccaagcccctgaccccagatgtggcaacaggaccctcgctcacatccaccggagtgtatgtgtggggaggggcttcacctgttcccagaggtgtccttggactcaccttggcacatgttctgtgtttcagtaaagagagacctgatcacccatctgtgtgcttccatcctgcattaaaattcactcagtgtggccca

SEQ ID NO: 51 - DNA, 16756 bp, Homo sapiens
  source 1..16756: mol_type = genomic DNA
gcacttggttcgggccagccgcctgaggggacgggctcacgtctgctcctcacactgcagctgctgggccgtggagcttccccagggagccagggggacttttgccgcagccatgaagggggcacgctggaggagggtcccctgggtgtccctgagctgcctgtgtctctgcctccttccgcatgtggtcccaggaaccacagaggacacattaataactggaagtaaaactgctgccccagtcacctcaacaggctcaacaacagcgacactagagggacaatcaactgcagcttcttcaaggacctctaatcaggacatatcagcttcatctcagaaccaccagactaagagcacggagaccaccagcaaagctcaaaccgacaccctcacgcagatgatgacatcaactcttttttcttccccaagtgtacacaatgtgatggagacagctcctccagatgaaatgaccacatcatttccctccagtgtcaccaacacactcatgatgacatcaaagactataacaatgacaacctccacagactccactcttggaaacacagaagagacatcaacagcaggaactgaaagttctaccccagtgacctcagcagtctcaataacagctggacaggaaggacaatcacgaacaacttcctggaggacctctatccaagacacatcagcttcttctcagaaccactggactcggagcacgcagaccaccagggaatctcaaaccagcaccctaacacacagaaccacttcaactccttctttctctccaagtgtacacaatgtgacagggactgtttctcagaagacatctccttcaggtgaaacagctacctcatccctctgtagtgtcacaaacacatccatgatgacatcagagaagataacagtgacaacctccacaggctccactcttggaaacccaggggagacatcatcagtacctgttactggaagtcttatgccagtcacctcagcagccttagtaacatttgatccagaaggacaatcaccagcaactttctcaaggacttctactcaggacacaacagctttttctaagaaccaccagactcagagcgtggagaccaccagagtatctcaaatcaacaccctcaacaccctcacaccggttacaacatcaactgttttatcctcaccaagtggattcaacccaagtggaacagtttctcaggagacattcccttctggtgaaacaaccacctcatccccttccagtgtcagcaatacattcctggtaacatcaaaggtgttcagaatgccaacctccagagactctactcttggaaacacagaggagacatcactatctgtaagtggaaccatttctgcaatcacttccaaagtttcaaccatatggtggtcagacactctgtcaacagcactctcccccagttctctacctccaaaaatatccacagctttccacacccagcagagtgaaggtgcagagaccacaggacggcctcatgagaggagctcattctctccaggtgtgtctcaagaaatatttactctacatgaaacaacaacatggccttcctcattctccagcaaaggccacacaacttggtcacaaacagaactgccctcaacatcaacaggtgctgccactaggcttgtcacaggaaatccatctacagggacagctggcactattccaagggtcccctctaaggtctcagcaataggggaaccaggagagcccaccacatactcctcccacagcacaactctcccaaaaacaacaggggcaggcgcccagacacaatggacacaagaaacggggaccactggagaggctcttctcagcagcccaagctacagtgtgactcagatgataaaaacggccacatccccatcttcttcacctatgctggatagacacacatcccaacaaattacaacggcaccatcaacaaatcattcaacaatacattccacaagcacctctcctcaggaatcaccagctgtttcccaaaggggtcacactcaagccccgcagaccacacaagaatcacaaaccacgaggtccgtctcccccatgactgacaccaagacagtcaccaccccaggttcttccttcacagccagtgggcactcgccctcagaaattgttcctcaggacgcacccaccataagtgcagcaacaacctttgccccagctcccaccggggatggtcacacaacccaggccccgaccacagcactgcaggcagcacccagcagccatgatgccaccctggggccctcaggaggcacgtcactttccaaaacaggtgcccttactctggccaactctgtagtgtcaacaccagggggcccagaaggacaatggacatcagcctctgccagcacctcacctgacacagcagcagccatgacccatacccaccaggctgagagcacagaggcctctggacaaacacagaccagcgaaccggcctcctcagggtcacgaaccacctcagcgggcacagctaccccttcctcatccggggcgagtggcacaacaccttcaggaagcgaaggaatatccacctcaggagagacgacaaggttttcatcaaacccctccagggacagtcacacaacccagtcaacaaccgaattgctgtccgcctcagccagtcatggtgccatcccagtaagcacaggaatggcgtcttcgatcgtccccggcacctttcatcccaccctctctgaggcctccactgcagggagaccgacaggacagtcaagcccaacttctcccagtgcctctcctcaggagacagccgccatttcccggatggcccagactcagaggacaagaaccagcagagggtctgacactatcagcctggcgtcccaggcaaccgacaccttctcaacagtcccacccacacctccatcgatcacatccactgggcttacatctccacaaaccgagacccacactctgtcaccttcagggtctggtaaaaccttcaccacggccctcatcagcaacgccacccctcttcctgtcacctacgcttcctcggcatccacaggtcacaccacccctcttcatgtcaccgatgcttcctcagtatccacaggtcacgccacccctcttcctgtcaccagcccttcctcagtatccacaggtcacaccacccctcttcctgtcaccgacacttcctcagaatccacaggtcacgtcacccctcttcctgtcaccagcttttcctcagcatccacaggtgacagcacccctcttcctgtcactgacacttcctcagcatccacaggtcacgtcacccctcttcctgtcaccagcctttcctcagcatccacaggtgacaccacccctcttcctgtcactgacacttcctcagcatccacaggtcacgccacctctcttcctgtcaccgacacttcctcagtatccacaggtcacaccacccctcttcctgtcaccgacacttcctcagcatccacaggtcacgccacctctcttcctgtcaccgacacttcctcagtatccacaggtcacaccacccctcttcatgtcactgatgcttcctcagcatccacaggtcaggccacccctcttcctgtcaccagcctttcctcagtatccacaggtgacaccacgcctcttcctgtcactagcccttcctcagcatccacaggtcacgccacccctcttcttgtcaccgacacttcctcagcatccacaggacacgccacccctcttcctgtcaccgacgcttcctcagtgtccacagatcacgccacctctcttcctgtaaccatcccttccgcagcatccacaggtcacaccacccctcttcctgtcaccgacacttcctcagcatccacaggtcaggccacctctcttcttgtcaccgacacttcctcagtatccacaggtgacaccacgcctcttcctgtcactagcacttcctcagcatccacaggtcacgtcactcctcttcatgtcaccagcccttcctcagcatccacaggtcacgccacccctcttcctgtcaccagcctttcctcagcatccacaggtgacaccatgcctcttcctgtcactagcccttcctcagcatccacaggtgacaccacccctcttcctgtcaccgacgcttcctcagtatccacaggtcacaccacccctcttcatgtcactgatgcttcctcagcatccacaggtcaggccacccctcttcctgtcaccagcctttcctcagtatccacaggtgacaccacgcctcttcctgtcactagcccttcctcagcatccacaggtcacgccacccctcttcttgtcaccgacacttcctcagcatccacaggacacgccacccctcttcctgtcaccgacgcttcctcagtgtccacagatcacgccacctctcttcctgtaaccatcccttccgcagcatccacaggtcacaccacccctcttcctgtcaccgacacttcctcagcatccacaggtcaggccacctctcttcttgtcaccgacacttcctcagtatccacaggtgacaccacgcctcttcctgtcactagcacttcctcagcatccacaggtcacgtcactcctcttcatgtcaccagcccttcctcagcatccacaggtcacgccacccctcttcctgtcaccagcctttcctcagcatccacaggtgacaccatgcctcttcctgtcactagcccttcctcagcatccacaggtgacaccacccctcttcctgtcaccgacgcttcctcagtatccacaggtcacaccacccctcttcctgtcaccagcccttcctcagcatctacaggtcacaccacccctcttcctgtcaccgacacttcctcagcatccaaaggtgacaccacccctcttcctgtcaccagcccttcctcagcatctacaggtcacaccacccctcttcctgtcaccgacacttcctcagcatccacaggtgacaccacccctcttcctgtcaccaatgcttcctcattatccacaggtcacgccacccctcttcatgtcaccagcccttcctcagcatccacaggtcacgccacccctcttcctgtcaccagcacttcctcagcatccaccggtcacgccacccctcttcctgtcaccggcctttcctcagctaccacagatgacaccacccgtcttcctgtcaccgacgtttcctcggcatccacaggtcaggccacccctcttcctgtcaccagcctttcctcagtatccacaggtgacaccacgcctcttcctgtcactagcccttcctcagcatccacaggtcacgccagccctcttcttgtcactgacgcttcctcagcatccacaggtcaggccacccctcttcctgtcaccgacacttcctcagtatccacagctcacgccaccccacttcctgtcaccggcctttcttcagcttccacagatgacaccacccgtcttcctgtcaccgacgtttcctcggcatccacaggtcaggccatccctcttcctgtcaccagcccttcctcagcatccacaggtgacaccacccctcttcctgtcaccgacgcttcctcagcatccacaggtgacaccacctctcttcctgtcaccatcccttcctcagcatcttcaggtcacaccacctctcttcctgtcaccgacgcttcctcagtgtccacaggtcacgccacctctcttcttgtcaccgacgcttcctcagtatccacaggtgacaccacccctcttcctgtcaccgacactaactcagcatccacaggtgacaccacccctcttcatgtcaccgacgcttcctcagtatccacaggtcacgccacctctcttcctgtcaccagcctttcctcagcatccacaggtgacaccacgcctcttcctgtcactagcccttcctcagcatcctcaggtcacaccacccctcttcctgtcaccgacgcttcctcagtacccacaggtcacgccacctctcttcctgtcaccgacgcttcctcagtgtccacaggtcacgccacccctcttcctgtcaccgacgcttcctcagtgtccacaggtcatgccacccctcttccggtcaccgacacttcctcagtatctacaggacaggccacccctcttcctgtcaccagcctttcctcagcatccactggtgacaccacgccgcttcctgtcaccgatacttcctcagcatccacaggtcaggacacccctcttcctgtcaccagcctttcctcagtatccacaggtgacaccacgcctcttcctgtcactaacccttcctcagcatccacaggtcacgccacccctcttcttgtcaccgacgcttcctcaatatccacaggtcacgccacctctcttcttgtcaccgacgcttcctcagtatccacaggtcacgccaccgctcttcatgacaccgatgcttcctcattatccacaggggacaccacccctcttcctgtcaccagcccttcctcaacatccacaggtgacaccacccctcttcctgtcaccgaaacttcctcagtatccacaggtcacgccacctctcttcctgtcaccgacacttcctcagcatccacaggtcacgccacctctcttcctgtcaccgacacttcctcagcatccacaggtcacgccacccctcttcctgtcaccgacacttcctcagcatccacaggtcaggccacccctcttcctgtcaccagcccttcctcagcatccacaggtcacgccatccctcttcttgtcaccgacacttcctcagcatccacaggacaggccacccctcttcctgtcaccagcctttcctcagcatccacaggtgacaccacccctcttcctgtcaccgacgcttcctcagtgtccacaggtcacgccacctctcttcctgtcaccagcctttcctcagtatccacaggtgacaccactcctcttcctgtcactagcccttcctcagcatccacaggtcacgccacccctcttcatgtcaccgacgcttcctcagcatccacaggtcacgccacccctcttcctgtcaccagcctttcctcagcatccacaggtgacaccacgcctcttcctgtcactagcccttcctcagcatccacaggtcacgccacccctcttcatgtcaccgacgcttcctcagtatccacaggtgacaccacccctcttcctgtcaccagctcttcctcagcatcctcaggtcacaccacccctcttcctgtcaccgacgcttcctcagcatccacaggtgacaccacccctcttcctgtcaccgacacttcctcagcatccacaggtcacgccacccatcttcctgtcaccggcctttcctcagcttccacaggtgacaccacccgtcttcctgtcaccaacgtttcctcggcatccacaggtcatgccacccctcttcctgtcaccagcacttcctcagcatccacaggtgacaccacccctcttcctggcaccgacacttcctcagtatccacaggtcacaccacccctcttcttgtcaccgacgcttcgtcagtatccacaggtgacaccacccgtcttcctgtcaccagcccttcctcagcatctacaggtcacaccacccctctacctgtcaccgacactccctcagcatccacaggtgacaccacccctcttcctgtcaccaatgcttcctcattatccacacgtcacgccacctctcttcatgtcaccagcccttcctcagcatccacaggtcacgccacctctcttcctgtcaccgacacttccgcagcatccacaggtcacgccacccctcttcctgtcaccagcacttcctcagcatccacaggtgacaccacccctcttcctgtcaccgacacttactcagcatccacaggtcaggccacccctcttcctgtcaccagcctttcctcagtatccacaggtgacaccacgcctcttcctgtcactagcccttcctcagcatccacaggtcacgccactcctcttcttgtcaccgacgcttcctcagcatccacaggtcaggccacccctcttcctgtcaccagcctttcctcagtatccacaggtgacaccacgcctcttcctgtcactagcccttcctcagcatccaccggtcatgccacctctcttcctgtcaccgacacttcctcagcatccacaggtgacaccacctctcttcctgtcaccgacacttcctcagcatacacaggtgacaccacctctcttcctgtcaccgacacttcctcatcatccacaggtgacaccacccctcttcttgtcaccgagacttcctcagtatccacaggtgacaccacccctcttcctgtcaccgacacttcctcagcatccacaggtcacgccacccctcttcctgtcaccaacacttcctcagtatccacaggtcacgccacccctcttcatgtcaccagcccttcctcagcatccacaggtcacaccacccctcttcctgtcaccgacgcttcgtcagtgtccacaggtcacgccacctctcttcctgtcaccgacgcttcctcagtgttcacaggtcatgccacctctcttcctgtcaccatcccttcctcagcatcctcaggtcacaccacccctcttcctgtcaccgacgcttcctcagtgtccacaggtcacgccacctctcttcctgtcaccgacgcttcctcagtgtccacaggtcatgccacccctcttcctgtcaccgacgcttcctcagtgtccacaggtcacgctacccctcttcctctcaccagcctttcctcagtatccacaggtgacaccacgcctcttcctgtcaccgacacttcctcagcatccacaggtcaggccacccctcttcctgtcaccagcctttcctcagtatccacaggtgacaccacccctcttcctgtcaccgacacttcctcagcatccacaggtcacgccacctctcttcctgtcaccgacacttcctcagcatccacaggtcacgccacccctcttcctgacaccgacacttcctcagcatccacaggtcacgccacccttcttcctgtcaccgacacttcctcagcatccataggtcacgccacctctcttcctgtcaccgacacttcctcaatatccacaggtcacgccacccctcttcatgtcaccagcccttcctcagcatccaccggtcacgccaccccgcttcctgtcaccgacacttcctcagcatccacaggtcacgccaaccctcttcatgtcaccagcccttcctcagcatccaccggtcacgccaccccgcttcctgtcaccgacacttcctcagcatccacaggtcacgccacccctcttcctgtcaccagcctttcctcagtatccacaggtgacaccacgcctcttcctgtcactagcccttcctcagcatccacaggtcacaccacccctcttcctgtcaccgacacttcctcagcatccacaggtcaggccaccgctcttcctgtcaccagcacttcctcagcatccacaggtgacaccacccctcttcctgtcaccgacacttcctcagcatccacaggtcaggccacccctcttcctgtcaccagcctttcctcagtatccacaggtgacaccacgcctcttcctgtcactagcccttcctcagcatccacaggtcacgccactcctcttcttgtcaccgacgcttcctcagcatccacaggtcaggccacccctcttcctgtcaccagcctttcctcagtatccacaggtgacaccacgcctcttcctgtcactagcccttcctcagcatccaccggtcatgccacctctcttcctgtcaccgacacttcctcagcatccacaggtgacaccacctctcttcctgtcaccgacacttcctcagcatacacaggtgacaccacctctcttcctgtcaccgacacttcctcatcatccacaggtgacaccacccctcttcttgtcaccgagacttcctcagtatccacaggtcacgccactcctcttcttgtcaccgacgcttcctcagcatccacaggtcacgccacccctcttcatgtcaccagcccttcctcagcatccacaggtgacaccacccctgtgcctgtcaccgacacttcctcagtatccacaggtcacgccacccctcttcctgtcaccggcctttcctcagcttccacaggtgacaccacccgtcttcctgtcaccgacatttcctcggcatccacaggtcaggccacccctcttcctgtcaccaacacttcctcagtatccacaggtgacaccatgcctcttcctgtcactagcccttcctcagcatccacaggtcacgccacccctcttcctgtcaccagcacttcctcagcatccaccggtcacgccacccctgttcctgtcaccagcacttcctcagcatctacaggtcacaccacccctcttcctgtcaccgacacttcctcagcatccacaggtgacaccacccctcttcctgtcaccagcccttcctcagcatctacaggtcacaccacccctcttcatgtcaccatcccttcctcagcatccacaggtgacaccagcactcttcctgtcaccggcgcttcctcagcatccaccggtcacgccacccctcttcctgtcaccgacacttcctcagtatccaccggtcacgccacgcctcttcctgtcaccagcctttcctcagtatccacaggtgacaccacccctcttcctgtcaccgacgcttcctcggcatccacaggtcaggccacccctcttcctgtcaccagcctttcctcagtatccacaggtgacaccacccctcttcttgtcaccgacgcttcctcagtatccacaggtcacgccacccctcttcctgtcaccgacacttcctcagcatccacaggtgacaccacccgtcttcctgtcacggacacttcctcagcatccacaggtcaggccacccctcttcctgtcaccagcctttcctcagtatccacaggtgacaccacccctcttcttgtcaccgacgcttcctcagtatccacaggtcacgccacccctcttcctgtcaccgacacttcctcagcatccacaggtgacaccacccgtcttcctgtcacggacacttcctcagcatccacaggtcaggccacccctcttcctgtcaccatcccttcctcatcatcctcaggtcacaccacccctcttcctgtcaccagcacttcctcagtatctacaggtcacgtcacccctcttcatgtcaccagcccttcctcagcatccacaggtcacgtcacccctcttcctgtcaccagcacttcctcagcatccacaggtcacgccacccctcttcttgtcaccgacgcttcctcagtgtccacaggtcacgccacgcctcttcctgtcaccgacgcttcctcagcatccacaggtgacaccacccctcttcctgtcaccgacacttcctcagcatccacaggtcaggccacccctcttcctgtcaccagcctttcctcagtatccacaggtgacaccacccctcttcctgtcaccgacgcttcctcagcatccacaggtcacgccacccctcttcctgtcaccatcccttcctcagtatccacaggtgacaccatgcctcttcctgtcactagcccttcctcagcatccacaggtcacgccacccctcttcctgttaccggcctttcctcagcttccacaggtgacaccacccctcttcctgtcaccgacacttcctcagcatccacacgtcacgccacccctcttcctgtcaccgacacttcctcagcttccacagatgacaccacccgtcttcctgtcaccgacgtttcctcggcatccacaggacatgccacccctcttcctgtcaccagcacttcctcagcatccacaggtgacaccacccctcttcctgtcaccgacacttcctcagtatccacaggtcacgccacctctcttcctgtcaccagccgttcctcagcatccacaggtcacgccaccccccttcctgtcaccgacacttcctcagtatccacaggtcacgccacccctcttcctgtcaccagcacttcctcagtatctacaggtcacgccacccctcttcctgtcaccagcccttcctcagcatccacaggtcacgccacccctgttcctgtcaccagcacttcctcagcatccacaggtgacaccacccctcttcctgtcaccaatgcttcctcattatccacaggtcacgccacccctcttcatgtcaccagcccttcctcagcatccagaggtgacaccagcactcttcctgtcaccgatgcttcctcagcatccaccggtcacgccacccctcttcctctcaccagcctttcctcagtatccacaggtgacaccacgcctcttcctgtcaccgacacttcctctgcatccacaggtcaggccacccctcttcctgtcaccagcctttcctcagtatccacaggtgacaccacgcctcttcctgtcaccatcccttcctcagcatcctcaggtcacaccacctctcttcctgtcaccgacgcttcctcagtgtccacaggtcacggcacccctcttcctgtcaccagcacttcctcagcatccacaggtgacaccacccctcttcctgtcaccgacacttcctcagcatccacaggtcacgccacccctcttcctgtcaccgacacttcctcagcatccacaggtcacgccacccctcttcctgtcaccagcctttcctcagtatccacaggtcacgccacccctcttgctgtcagcagtgctacctcagcttccacagtatcctcggactcccctctgaagatggaaacaccaggaatgacaacaccgtcactgaagacagacggtgggagacgcacagccacatcaccaccccccacaacctcccagaccatcatttccaccattcccagcactgccatgcacacccgctccacagctgcccccatccccatcctgcctgagagaggagtttccctcttcccctatggggcaggcgccggggacctggagttcgtcaggaggaccgtggacttcacctccccactcttcaagccggcgactggcttcccccttggctcctctctccgtgattccctctacttcacagacaatggccagatcatcttcccagagtcagactaccagattttctcctaccccaacccactcccaacaggcttcacaggccgggaccctgtggccctggtggctccgttctgggacgatgctgacttctccactggtcgggggaccacattttatcaggaatacgagacgttctatggtgaacacagcctgctagtccagcaggccgagtcttggattagaaagatgacaaacaacgggggctacaaggccaggtgggccctaaaggtcacgtgggtcaatgcccacgcctatcctgcccagtggaccctcgggagcaacacctaccaagccatcctctccacggacgggagcaggtcctatgccctgtttctctaccagagcggtgggatgcagtgggacgtggcccagcgctcaggcaacccggtgctcatgggcttctctagtggagatggctatttcgaaaacagcccactgatgtcccagccagtgtgggagaggtatcgccctgatagattcctgaattccaactcaggcctccaagggctgcagttctacaggctacaccgggaagaaaggcccaactaccgtctcgagtgcctgcagtggctgaagagccagcctcggtggcccagctggggctggaaccaggtctcctgcccttgttcctggcagcagggacgacgggacttacgattccaacccgtcagcataggtcgctggggcctcggcagtaggcagctgtgcagcttcacctcttggcgaggaggcgtgtgctgcagctacgggccctggggagagtttcgtgaaggctggcacgtgcagcgtccttggcagttggcccaggaactggagccacagagctggtgctgccgctggaatgacaagccctacctctgtgccctgtaccagcagaggcggccccacgtgggctgtgctacatacaggcccccacagcccgcctggatgttcggggacccccacatcaccaccttggatggtgtcagttacaccttcaatgggctgggggacttcctgctggtcggggcccaagacgggaactcctccttcctgcttcagggccgcaccgcccagactggctcagcccaggccaccaacttcatcgcctttgcggctcagtaccgctccagcagcctgggccccgtcacggtccaatggctccttgagcctcacgacgcaatccgtgtcctgctggataaccagactgtgacatttcagcctgaccatgaagacggcggaggccaggagacgttcaacgccaccggagtcctcctgagccgcaacggctctgaggtctcggccagcttcgacggctgggccaccgtctcggtgatcgcgctctccaacatcctccacgcctccgccagcctcccgcccgagtaccagaaccgcacggaggggctcctgggggtctggaataacaatccagaggacgacttcaggatgcccaatggctccaccattcccccagggagccctgaggagatgcttttccactttggaatgacctggcagatcaacgggacaggcctccttggcaagaggaatgaccagctgccttccaacttcacccctgttttctactcacaactgcaaaaaaacagctcctgggctgaacatttgatctccaactgtgacggagatagctcatgcatctatgacaccctggccctgcgcaacgcaagcatcggacttcacacgagggaagtcagtaaaaactacgagcaggcgaacgccaccctcaatcagtacccgccctccatcaatggtggtcgtgtgattgaagcctacaaggggcagaccacgctgattcagtacaccagcaatgctgaggatgccaacttcacgctcagagacagctgcaccgacttggagctctttgagaatgggacgttgctgtggacacccaagtcgctggagccattcactctggagattctagcaagaagtgccaagattggcttggcatctgcactccagcccaggactgtggtctgccattgcaatgcagagagccagtgtttgtacaatcagaccagcagggtgggcaactcctccctggaggtggctggctgcaagtgtgacgggggcaccttcggccgctactgcgagggctccgaggatgcctgtgaggagccgtgcttcccgagtgtccactgcgttcctgggaagggctgcgaggcctgccctccaaacctgactggggatgggcggcactgtgcggctctggggagctctttcctgtgtcagaaccagtcctgccctgtgaattactgctacaatcaaggccactgctacatctcccagactctgggctgtcagcccatgtgcacctgccccccagccttcactgacagccgctgcttcctggctgggaacaacttcagtccaactgtcaacctagaacttcccttaagagtcatccagctcttgctcagtgaagaggaaaatgcctccatggcagaagtcaacgcctcggtggcatacagactggggaccctggacatgcgggcctttctccgcaacagccaagtggaacgaatcgattctgcagcaccggcctcgggaagccccatccaacactggatggtcatctcggagttccagtaccgccctcggggcccggtcattgacttcctgaacaaccagctgctggccgcggtggtggaggcgttcttataccacgttccacggaggagtgaggagcccaggaacgacgtggtcttccagcccatctccggggaagacgtgcgcgatgtgacagccctgaacgtgagcacgctgaaggcttacttcagatgcgatggctacaagggctacgacctggtctacagcccccagagcggcttcacctgcgtgtccccgtgcagtaggggctactgtgaccatggaggccagtgccagcacctgcccagtgggccccgctgcagctgtgtgtccttctccatctacacggcctggggcgagcactgtgagcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagactggaaaagggaaggtgactgaaggctgtcaggattcttcaaggagaatgaatactgggaatcaagacaagactataccttatccataggcgcaggtgcacagggggaggccataaagatcaaacatgcatggatgggtcctcacgcagacacacccacagaaggacactagcctgtgcacgcgcgcgtgcacacacacacacacacacacgagttcataatgtggtgatggccctaagttaagcaaaatgcttctgcacacaaaactctctggtttacttcaaattaactctatttaaataaagtctctctgactttttgtgtct

SEQ ID NO: 52 - DNA, 4839 bp, Homo sapiens
  source 1..4839: mol_type = genomic DNA
agtcgactgccggagactcgggaggctgagctttcctcggcctgagccagccagaccccgggcaccgcgctcacccctcttcgccgccacgtccgcgaaggcctcacgcgcgaggccaggcgaggccccgaggcgcccaccacttcacgacaccggagcgaaccgggcgccagaggctgcgacccccctgccccgaatcctgccggtgggagtggctgcatttgaaccaaacggccttcgcgggcagcagccgtcgccccgcagtcccggggctcccaagggcctgtgaccgacgccgccctccgcgtcttcgtccccgaagccccgggaaccatccgccctcgggagaccatgctgcagatgcgaggaaagccgtttcctggaacatcggaattctaaccccagggtgaaggactcacgacaggcgaggggcagacatgctgaattccacgggcgaactggagttttcgaacgaagaagatcccgagatcatctcccaactcacttccctgcctctgtccggtgggaaaagctcagctggtgtgcccgaaaaaacgggctatccggactccgtttatgtcatggcagccaacatttttcagggtattcgaatcgaaaagtcggcacagaaagtcttaatcaagtatgggaatgaacccctgcggtccttgtccgagtctgaggatcagtcctttcagcgtttgtcttatgagctggctttcagtgccctgaaatatcaagatattttggaaactatattgatagacagctgtatcttcccaagtaccacaataccagatcatttgagcagtcttattattgtgatgctatatgatttccaagatagaaaatttcaaactcgtgtcctttctgataatgaagagcccatatcagaagttcaagaagtagagaaccttcttaacagttttaagataaaattggctgcagcattggcaagatgtcgaatcaagcatgatgccctttcaatttaccacatccttccagaaacagttaggaaacaggaactaagggcctccactttaccactttatgcttggataaatacttgtaaaatcagccctgaagaagtttataataatttgaagagaagaggctataataaagtcaaatctgtattgcatattgatgataaagtctttgctgtggatcaacattgctatgatgtcttaatttttccatctcatcttaaaaatgatcttataaatatagatcttttcaaagattacaaacttatatttcaggacaaatctcgaagtcttgctgtccattctgtaaaggctttattaaatatggatgatgatgtcttaatggtcaatacaggctcatggtacacagtttcccacatgtcaattttaacaaataataatacctcaaaagtatttgtgtgtggagtacaatcacaagctaaggatcctgacttgaagacccttttcacaaaaataggatgtaaaaatattgaaatacttcatgagaaatttattaacattgaatcaaaggatcacaggttacagaaagttaaagtgattttgctgctacctcgttgttcaggactgggtgttagtaatccagtagaatttattttaaatgaacatgaagatacagaattccttaaagatcactctcaaggaggcatctcagtggacaaacttcacgttcttgctcaacagcagtatgaacagctaacacatgcaatgaaatttactaaagctcaagcagttgtttactgcacatgttcagtttttccagaagaaaatgaagctgttgttaagaaagcactggaatttcaagaccttgggaataaaggacaaccttacaggcttagtcctcctgttcttccactgtgctccttaaaggaaattcaattgtctactgataaatttttcagaatggaaccatctgaaattaccaatggttgttttctttctattttaacaagggagcgggacccttctgagacagtgtctgtgaatgatgttttggcccgagctgcagccaagggtctgctggatgggattgagttgggtaaatcatcaaaacgggagaagaagaagaaaaaatcaaaaacatcattgacaaaaggtgccactactgataatggcatccaaatgaaaattgctgagttcctgaatcgagaaactaaagccagtgctaatctatcagagactgtaacaaaaccacctcttccccagaaaaatactgctcaagtgggggcttcctcacagaccagaaaacccaacaagctggccccccatcctgcagtgcctgcatttgtgaagaacacttgtccctccagaccgcgtgaacggcagacacacttcttaagacctcggccagaagacagaatggttgctctgaaacccatcaagattgttctgcctccagtctttatgccattttcaagtccccaagggatcagatctcggatgccaactcaacatttgtactgtcgttgggttgcacccaaggcacttgtgcccacctgccttcccacacactcactatccagaaaagaggaaaagcctaaagatgacacaccttcctccctactcaggcctcctcggcgatggctttgattgtcttgtgttttttataggggccaaagagcagttgattttttttcaaagtctagtatttctctgaagattctacatctctacacaagatattcattcttttggtcacctagggatcttctaagtgtgatattactttcagagaattcagacaagtgagaaacaataatgtaggagtcagcaaagcagaattcagagacttcagccaatcactgctgctctgagaggatccagttagagactcagtatcagcggtcagaacttatctcactcctgtgaactttcaggctggacttaaagctgccaagtttcccctgcagggaaggaaacactgcctcccttcagcaggtagctcattagaaagccaaacaggcaaacgatcctggcctctcccgccagctgaccgctcttcagcatccatgcggttgtagtcgtgactttctcagtcacgatcaagggtgattttttcttaaatatcaagctgttctttgaacagggaatgaacatgagtttttgtaacgtgactgaagttgagtttaagtaggaagcgcaggaagttcccaagtgccaggtgtgtgtagctcagagttccttttacagtgaggtgtctctcactgggggagcttccaggatcctgagcagactggacacaatcatctctcccttcctctatgtcaagcactgttacaaaagactgtgagcaaatttccatctaaatattaataattctgaagaagaggcaaaactgttgaatgcaagcgatacctattgttgaagaaacccacaaatttctgattctaagatcaggggatacaacaaaatctacaagtcatttcaaatagcacacaggaatcaaactttggtaaatcatttctgaggcacaattaaatatattgtagcactatgttaattaattatattaaatgtcgattcatcttgaatgtattctcaattgcctaccaaaaattggtatgattatcatttctgggtctactgatttttcatcatggcaacagaaattgtcattaaatagaattaagatacaactatatatcttattatttaaaaacattcatagacatattcataaagtttgaatttaaagttttatttttgcaattcagttgtatagaaatgttacataaattcctaaatgctcaattcaggtggcatgatttttagaatattatcttcttcaagatgaatacttaggtctacttcagctctgatattctatttttactttcttacttgaagtgagaaaagagaatggtgaattgtattatagcggtaatacattatttttattttagcttagatttacatttatcagtaaaacgtgtcagtgggctgggtgcagtggctcacatctgtaatctcagcagtttgggaggctgaggtgggcacgccacttgagcaccggagttccagacgagcctggacaacatggcgaaaccccaaaaccccatctctacaaaaaatacaaaaattagctgggtgtggtgggacacgcctgtagtcccagctacttaggaggctgaggtacgaggatctcttgagtccaggaggtgcaggttgcagtgagccaatattgccccactgcactccagcctgggaagcagagtgagaccttgcctcaaaaaaaaaaaaaaaaaaaaaaaagtgtcaatgaagaaaggaaacaagactttttatagttgaaccaggcttattgtattttaaatccctgaagaagaattcttcatgttatcactcttgtacagaaagatataaagtgctcagcttattttgctgattttgaaaatcaaacactatctccttgccctaaaattatagcgtgtggctcatcctcttatcccacccagatttctatctggttggttagggtagtccatgcctcaaggaaggcggtgcagcttaatttaacatattgttcagtaaagaatcccattgtgtatcataagaatcccttatccttttcgggccctccaccataccagactgaaaagtattccattgtccagaagtagctggtcttgagatttttattttcaagtgtattttctgtgatgttgtctcattgttatggaatagtttaggataattttctgatctctacagtagcatgtgaagtcacaatttgttgccacttacattgattatctcctaacatgcatttggcactaaattatttttctctttagaagtatatgaagattctgaattaaaccattcattaattaca

SEQ ID NO: 53 - DNA, 8666 bp, Homo sapiens
  source 1..8666: mol_type = genomic DNA
cttcgccagcagtagcagaggcggcggcggcggctcccggaattgggttggagcaggagcctcgctggctgcttcgctcgcgctctacgcgctcagtccccggcggtagcaggagcctggacccaggcgccgccggcgggcgtgaggcgccggagcccggcctcgaggtgcataccggacccccattcgcatctaacaaggaatctgcgccccagagagtcccgggagcgccgccggtcggtgcccggcgcgccgggccatgcagcgacggccgccgcggagctccgagcagcggtagcgcccccctgtaaagcggttcgctatgccggggccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggcttccgtgagacacaagatggcgcatagtgtgctcggacacagttttgtcttcgtaggttgtgatgatagcactggtttgtttctcaagcgctatccacagaacctttgtcaacttcagttgaaaagaggtggattcatgtccagagctcatttcggggtcaggtgggaaagccaagaacttggaaaagataagacaagctataaattcggaggcaagtttcttttacaatgaacttttcagatctcacttccctccgacccctaacttccatgcccacccgtccttttaactgtgcaagcaaaattgtgcatggtcttcgtcgattaataccttgtgtgcagacactactgctccagacgtcgtttccctgataggtagagcagatccataaaaaggtatgacttatacaattaggggaagctaatggagtttattagctgagtatcaatgtctctgcgttgtacggtggtgatgggttttaatgaatatggaccctgaagcctggaaatcctcatccacgtcgaacccacaggactgtgggaagggcagaatcaatccctaagggaaaggaaacctcaccctgagggcatcacatgcactcatgttcagtgtacacaggtcaagtcccttgctctgggctctagttgggagagtggtttcattccaagtgtactccattgtcagtatgctgtttttgtttccttcactccattcaaaaagtcaaaatacaaaatttggcacagcatgccaacgggaggctgtgcccagaccaagcactggaagtgtgcttctaggcatagtcattggttttgcaaaaagagggctcaaatttaaatagaaatttacagctatttgaatggtcagatataccaagaaagaaaaatatttctgttcctcaagaaaacttgctaccctctgtgaggggaattttgctaaacttgacatctttataacatgagccagattgaaagggagtgattttcattcatcttaggtcatgttatttcatatttgtttctgaaggtgcgatagctctgttttaggttttgcttgcgcctgttaattactggaacaccttatttttcattaaaggctttgaaagccaattctcaaaaattcaaaagtgcaaattaacagaacaaaaggaaatccagtagcaactgcagtcaagcgagggagttgacaagataaaccttacgtccattcaagttatatgctggcctatgagagatgagagttgggtcgtttgttctctttgttgatgattttaaaaaaaccctctagaatacacataataacataatgaaagccatatctccatgatatatatgtgcacatatatatacatacatgtgcatgtatgtatcatattaaggacccatggtactcttaaaacactgtagaactctgtgacgcagtaaggaaggggcagatttgtacaaaaacttttctagattccatcagcaaaaaccaacacaggtttgtcacgctgcatgtctggccagctaatctcgggggaaaagctacaagttatttattttattttaagagaataaagtaggtaataatttaagggatcaaattcaaggaggaatgtgcaattttagagcaaagatttgtttaaggcaaatgagactttgggagcatcccattccagttttgtcttttttttctctgaaagaaaaaagcaaaaaataaaataaaattccacttataccttctgacaagtccctaaaggtcttgaaataaaaggttctatgcaagtgcaaagttttatagttatttttattgctgattattactattactatctctgttgtcttaagagtatgtgctgatttcagagacatctcaaattgaaagaatatcagattgcttttaaagtagctgaacgagccacagaatatctgaaattattcattgttgttcctccaccaccccctttctcatggtctgatttttagaagagtggcatcctcgttctaaaatgtaatgatcaccaaatacggccttccatcaaatttgtgaaaactacaacagtataacagtgacaaacctaattctctagcccaaacctggtctgacaatcatttccatttagaagtcattgaatagttttccaaacactttccatgtgtgttagcaaattattcctattttgtgtagatgaggacgttgagactcagagacattcagaggcacgctagaggtctccagcctagcttccagcaccattgggactgaatccaagtactctcactctgaacttcgtggttctgtccactagagactctaatatgcaaacaagcagttcaggaaagaaagcatgctaacacattcatgaagcagtatatgaagttagaagaacaaaaggaaatacaggagatgacaagcaactgagatattgtgatatataatcatgctcttagcttcagctaaattcagctaaattcttgtacactgaaccaatgtcataatcaggcttatttagaaaacactttgaactatgctataaaagattatatcagaattcatattatacatgtgttcacatcagcgctacctgtgatgttttcatgtatttatgtatgtgttataaatacttgatttatacatatacaaatgcacatacgtagtgtgtttgtgtgtttatgtataaatttataggcacacaataatagaggtaattataagtaggatgcggtatgaataatttgcttaaaatatgctaaataaccaaaactgtttaacgtcatgttgctgttagtgcttccatactccacgtgggtaggactacatcacacttttcaactctgtgcagtactgcatgggtggaagacatatttaagataatgtgcttcccaaaacaactgaataaaagccatcccactacattgagtgctttctctggctccttgcaaagaaagatactttttgtaatggtccaggaaaggaacattgctttctttttgtctttcagcacatttgtattatgctcaccttgtctctgtctcactgtgacccctttacacttgagttcagagttcaagcattccaaatataaattggaatgttggcagcccagtggcttgaaggccaatgatgagcagtccaagaccccacagcgagatgagcaactcttaggaattcccacatcctagagtgaatgcaccaactaacagtatagaatgctgtccttttcaaagcgtcctaacagcaggattacctggtcaagtatggactttctttgaatctttcttttcacaaattggactgcctgtaataccaataacattgttgtatctaactaaataaatgactgcatatacacacataccctcaattctcttgcttccccattttctttttcatcccctgtctcaggacttttattttcaatgttgacctttggtttggccatatatcactgttataggaaatctcatgagaggaatggctagtgacccaactctccaaatgtctaagttagtagttacagctgattttttatgatgcataattggaatgtggagcctctgaggttgtgatagcttgtacatgaatttcaaatgtcattctaaagaatgaggggtgggagggatttatagttagaaacgacagtgcaggaaggggtattttcttgttgtcagggctggaatgaatcactgctgctcaagtcaaaggttcttgaatatccttagtttttgcatttcccctccttttcctttgacctttatttatttaattatgtatttatttatttatttatatacttttgctccattcagcacaaacacaaagcaaagcaaaaaaaaaaatatatatatatatatctgtatatgtgttgtaggcaaaacactgtgaatttcacaacaaccaccaccaagcaactattttgccatcttaacatacatctcaggagacgaaatgagaaaagatggggatgtcattttttagtctatgcgtttgaggccaggtccatgtttatttatttctttagtctatgcattaatgaaaatgatcctgagtggaggttagctgaacgttcaatgtactggagcaagcatcataaaagctgctagtagccatgtgtttgaacaggaaaaatattacagaaaatgaaatgtaaaggcctatatcttgcagcttgtatatcttactattgcttaaaaaatgtataaagcagctggaaatgttttaaatacaaggtctttgaattaaatgtggattttaaatatgtaatcccttgacaaatgaccaaattatggtgaactattgctccctgcgttctttgatcattacctatgacttacaaatctgcctggagatgtggacattctgcatttgcttctgtatctggagagatgtttgtatatatccaggccgtatacacacacatttccatatctctctacagatatatttccccttcaatcgtgacctggtatttggaactctccttttcatttggcttatcttccttttaatgtgatgtctctgtgctaatacttaccagttcttgttttgcaatctgttttgaggtccattgctttactaagacccactgcatcttggctgatttcaaagtgacacctgaatacagtgtttaaaaaaaaaaaagttttgtttgtaaatcatgtgaccagcttctctcaacctgacatggaaagtctcttgtactacagtgtatttaataaaaatgatgtcttacaataaataacatactccaaaa

SEQ ID NO: 54 - DNA, 3178 bp, Homo sapiens
  source 1..3178: mol_type = genomic DNA
agccctcgcccagcagcccgcgccgccaccgcctcttccctccccgtgtccggtccccgtgcgtcccgaggctccccgccgcccgtcccggcgcgcaccgcgggcgtctgtccgaacgccttccagccacctgagccctcctgcgggcgactcgctcagctagcccgtgcccgcctccaccttctccgtcatcccctcttccttgcgtccggctctccactggggctgcacagtcgagggctgctcgcgtcgggaaggagatgcccagagtctctggggcgcaccctcccgtcccgctcagccgcacccagctttagaaggtgctctcagcagccactttcgggctctagcgaggacaccctctcgcagaagtccttgccgagaccccccgccccagccattctctgaaggggctgaggacactcttatcgcgcccctcatggccaagcctcggctgctagttctctacttcgctctgattgtggttccggcctgggtgtccagcattgtcctcacagggacaagcgagcccccagatgcgcagacagtggcgcctgcggaggacgagactctgcaaaacgaggcggacaaccaggagaacgttttatctcagttgctgggggactatgacaaggtcaaggctatgtctgagggctcggactgtcagtgcaagtgtgtggtgagacccctgggccgggatgcctgccagaggatcaatgcgggggcctccaggaaggaagacttctataccgtggaaaccatcacctcaggctcgtcgtgcaagtgtgcctgtgtagcacccccatcggccctcaatccctgcgagggagacttcaggctccagaagctgcgggaggcagacagccaggacttgaagctctccacaatcatagacatgttggaaggagcgttctatggcctggatctcctgaagctacattcagtcaccaccaaactggtggggcgagtggataaactggaggaggaagtgtctaaaaacctcaccaaggaaaatgaacaaatcaaagaggacatggaagaaattcgaaccgagatgaataagcgaggcaaagaaaattgctctgaaaacatcctagatagcatgccagacatccgctcagccctgcagagggatgcagcagcagcctacgcccacccagagcagtatgaagagcggtttctgcaggaagaaaccgtgtcccagcagatcaactccatcgaacttctgcagacgcgacccctggctctgcctgaggtggtgaagtcacagcggcccctgcagaggcaggtccacctgagaggccggccggcctcccagcccactgtcatccggggcatcacctactataaagccaaggtctctgaagaagagaatgacattgaagagcagcaagatgagtttttcagcggtgacaatggagtggatttgctgattgaagatcagctcctgagacacaacggcctgatgaccagtgtcacccggaggcctgcagccacccgtcagggacacagcactgctgtgacaagcgacctgaacgctcggaccgcaccctggtcctcagcactgccacagccctcgacctcagatcccagcatcgccaaccatgcctcagtgggaccaacactccaaacaacctcggtgtctccagatcccacaagggagtcagtcctgcagccttctcctcaggtaccagccaccactgtggcccacacagccacccagcaaccagcagccccagctcctccggcagtgtctcccagggaggcattgatggaagctatgcacacagtcccagtgcctcccaccacagtcagaacagactcgctggggaaagatgctcctgctgggtggggaacaacccctgccagccccacgctgagccccgaagaagaagatgacatccggaatgtcataggaaggtgcaaggacactctctccacaatcacggggccgaccacccagaacacatatgggcggaatgaaggggcctggatgaaggaccccctggccaaggatgagcggatttacgtaaccaactattactacggcaacaccctggtagagttccggaacctggagaacttcaaacaaggtcgctggagcaattcctacaagctcccgtacagctggatcggcacaggccacgtggtatacaatggcgccttctactacaatcgcgccttcacccgcaacatcatcaagtacgacctgaagcagcgctacgtggctgcctgggccatgctgcatgacgtggcctacgaggaggccaccccctggcgatggcagggccactcagacgtggactttgctgtggacgagaatggcctatggctcatctacccggccctggacgatgagggcttcagccaggaggtcattgtcctgagcaagctcaatgccgcggacctgagcacacagaaggagaccacatggcgcacggggctccggaggaatttctacggcaactgcttcgtcatctgtggggtgctgtatgccgtggatagctacaaccagcggaatgccaacatctcctacgctttcgacacccacaccaacacacagatcgtccccaggctgctgttcgagaatgagtattcctatacgacccagatagactacaaccccaaggaccgcctgctctatgcctgggacaatggccaccaggtcacttaccatgtcatctttgcctactgacacccttgtccccacaagcagaagcacagaggggtcactagcaccttgtgtgtatgtgtgtgcgcgcacgtgtgtgtaggtgggtatgtgttgtttaaaaatatatattattttgtataatattgcaaatgtaaaatgacaatttgggtctatttttttatatggattgtagatcaatccatacgtgtatgtgctggtctcatcctccccagtttatatttttgtgcaaatgaacttctccttttgaccagtaaccaccttccttcaagccttcagcccctccagctccaagtctcagatctcgaccattgaaaaggtttcttcatctgggtcttgcaggaggcaggcaacaccaggagcagaaatgaaagaggcaagaaagaagtgctatgtggcgagaaaaaaagttttaatgtattggagaagttttaaaaaacccagaaaaacgctttttttttttaataaagaagaaatttaaaatca

SEQ ID NO: 55 - DNA, 1748 bp, Homo sapiens
  source 1..1748: mol_type = genomic DNA
gactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgct

SEQ ID NO: 56 - DNA, 1771 bp, Homo sapiens
  source 1..1771: mol_type = genomic DNA
gcgcgacctgggtgccatggcggcagcggcggtgacaggccagcggcctgagaccgcggcggccgaggaggcctcgaggccgcagtgggcgccgccagaccactgccaggctcaggcggcggccgggctgggcgacggcgaggacgcaccggtgcgtccgctgtgcaagccccgcggcatctgctcgcgcgcctacttcctggtgctgatggtgttcgtgcacctgtacctgggtaacgtgctggcgctgctgctcttcgtgcactacagcaacggcgacgaaagcagcgatcccgggccccaacaccgtgcccagggccccgggcccgagcccaccttaggtcccctcacccggctggagggcatcaaggtggggcacgagcgtaaggtccagctggtcaccgacagggatcacttcatccgaaccctcagcctcaagccgctgctcttcgaaatccccggcttcctgactgatgaagagtgtcggctcatcatccatctggcgcagatgaaggggttacagcgcagccagatcctgcctactgaagagtatgaagaggcaatgagcactatgcaggtcagccagctggacctcttccggctgctggaccagaaccgtgatgggcaccttcagctccgtgaggttctggcccagactcgcctgggaaatggatggtggatgactccagagagcattcaggagatgtacgccgcgatcaaggctgaccctgatggtgacggagtgctgagtctgcaggagttctccaacatggaccttcgggacttccacaagtacatgaggagccacaaggcagagtccagtgagctggtgcggaacagccaccatacctggctctaccagggtgagggtgcccaccacatcatgcgtgccatccgccagagggtgctgcgcctcactcgcctgtcgcctgagatcgtggagctcagcgagccgctgcaggttgttcgatatggtgaggggggccactaccatgcccacgtggacagtgggcctgtgtacccagagaccatctgctcccataccaagctggtagccaacgagtctgtacccttcgagacctcctgccgctacatgacagtgctgttttatttgaacaacgtcactggtgggggcgagactgttttccctgtagcagataacagaacctacgatgaaatgagtctgattcaggatgacgtggacctccgtgacacacggaggcactgtgacaagggaaacctgcgtgtcaagccccaacagggcacagcagtcttctggtacaactacctgcctgatgggcaaggttgggtgggtgacgtagacgactactcgctgcacgggggctgcctggtcacgcgcggcaccaagtggattgccaacaactggattaatgtggaccccagccgagcgcggcaagcgctgttccaacaggagatggcccgccttgcccgagaagggggcaccgactcacagcccgagtgggctctggaccgggcctaccgcgatgcgcgcgtggaactctgagggaagagttagccccggttcccagccgcgggtcgccagttgcccaagatcaggggtccggctgtccttctgtcctgctgcagactaaaggtctggccaatgtcttgccccaccccgccagccgcgatacggcgcagttcctatattcatgttatttattgtgtactgactccatctgccccgtcaaataaaaaaccacaaggttcgagccgccgggcccgacaaactccgggtcggcgaaacaga

SEQ ID NO: 57 - DNA, 3895 bp, Homo sapiens
  source 1..3895: mol_type = genomic DNA
gccatctttcctccgctccgaacacacggagcccggggccgcacagccgccgctccgccgccgccttctcccggcctgaggaagggcctactctgccgccgccgccgcccgcccgctccagccgccgccgccgccgccaccgccctccaggctccgggacccggcccgcgccaccgcccccgtgcgcgccccgccgccgccgccttcgccttcgccttttgtttcctccgctccggcgcccccgccccggctcgcgctttgcaggggacgcagcgcgcgcccccagcgggcccgggaaaagccgcggcgcgcgcgcgcgcctgcgcggcggacccctccttctcctccccgcgtgcgcgtgcccttcttggctgcgcgccggcgccgcctggcgggcgggaggggaggtggcaggcgcgtttgcaggaggggcgcacctcttcgctcgcgcacccccccggaaggtagaccgggaaggggaggcgggcgggcggagaggagagagtggcgcgcagtccagcgagggcgggggttggctatgtggggggtggtgcaccccgcagtctagacagtctgatccgggctgggggcgtgtacactcggcgcacctgcgagactacagagcctcgggccggcacgtgtggggagtgtggacacgtctgctgcgccccgcttctcgctgctgaggggaagggagggggcgggcaggtgcagcggccgggctagtgggagggggcggcggccatggagcgggtgaacgacgcttcgtgcggcccgtctggctgctacacataccaggtgagcagacacagcacggagatgctgcacaacctgaaccagcagcgcaaaaacggcgggcgcttctgcgacgtgctcttgcgggtaggcgacgagagcttcccagcgcaccgcgccgtgctggccgcctgcagcgagtactttgagtcggtgttcagcgcccagttgggcgacggcggagctgcggacgggggtccggctgatgtagggggcgcgacggcagcaccaggcggcggggccgggggcagccgggagctggagatgcacactatcagctccaaggtatttggggacattctggacttcgcctacacttcccgcatcgtggtgcgcttggagagctttcccgaactcatgacggccgccaagttcctgctgatgaggtcggttatcgagatctgccaggaagtcatcaaacagtccaacgtacagatcctggtaccccctgcccgcgccgatataatgctctttcgcccccctgggacctcggacttgggcttccctttggacatgaccaacggggcagccttggcagccaacagcaatggcatcgccggcagcatgcagccagaggaggaggcagctcgggcggctggtgcagccattgcaggccaagcctctttgcctgtgttacctggggtggaccgcttgcccatggtggctggacccctatccccccaactgctgacttccccattccccagtgtggcatccagtgcccctcccctgactggcaagcgaggccggggccgcccaaggaaggccaacctgctggactcaatgtttgggtccccagggggcctgagggaggcaggcatccttccatgcggtctatgtggtaaggtgttcactgatgccaaccggctccggcagcacgaggcccagcacggtgtcaccagcctccagctgggctacatcgaccttcctcctccgaggctgggtgagaatgggctacccatctctgaagaccccgacggcccccgaaagaggagccggaccaggaagcaggtggcttgtgagatctgcggcaagatcttccgtgatgtgtatcatcttaaccggcacaagctgtcccactctggggagaagccctactcctgccctgtgtgtgggttgcggttcaagagaaaagaccgcatgtcctaccatgtgcggtcccatgatgggtccgtgggcaagccttacatctgccagagctgtgggaaaggcttctccaggcctgatcacttgaacggacatatcaagcaggtgcacacttctgagcggcctcacaagtgtcagacctgcaatgcttcttttgccacccgagaccgtctgcgctcccacctggcctgtcatgaagacaaggtgccctgccaggtgtgtgggaagtacttgcgggcagcatacatggcagaccacctgaagaagcacagcgaggggcccagcaacttctgcagtatctgtaaccgaggtttctcctctgcctcctacttaaaggtccatgttaaaacccaccacggtgttccccttccccaggtctccaggcaccaggagcccatcctgaatgggggagcagcgttccactgcgccaggacctatggcaacaaagaaggccagaaatgctcacatcaggatccgattgagagctctgactcctatggtgacctctcagatgccagcgacctgaagacgccagagaagcagagtgccaatggctctttctcctgcgacatggcagtccccaaaaacaaaatggagtctgatggggagaagaagtacccatgccctgaatgtgggagcttcttccgctctaagtcctacttgaacaaacacatccagaaggtgcatgtccgggctctcgggggccccctgggggacctgggccctgcccttggctcacctttctctcctcagcagaacatgtctctcctcgagtcctttgggtttcagattgttcagtcggcatttgcgtcatctttagtagatcctgaggttgaccagcagcccatggggcctgaagggaaatgaggcagctgctgtgtccccacggaaacaaccatctggggactgctgggaaatgctgtgaatgcggagggaagtgatgtttgggttctgtagctgagagatttttattcatttttaactgccccccaaccccactccaactccttctccaccacccattctcccaatggtctttagaaatagattttcatctgatattctgcagaaatatcaatgagacttggtatgggacaggggcagaaaacactacataggcctccaaggcaaaaccagtcccagtttctttaatgggaagaagctggaattcctggtgctcaattcttagtgaccccaatcctatacccaaatctatgatattctgggacctcagtgattttggtcccctcccacttctctagttcgtcatcctcccttcccatatccttcaaaagaaccacactagggtctccacctacttatacaatgcggatgcccaactgtttttaaggaagccagaagcatcccatggaccatggggtgagtgtcctccaagagccccctgagctcagccctctgcctggagggctccagacctttctgagccctgcttggaggcgagcattttcactgctaggacaagctcagctgttgaggacacccccaccccaaatttcagttcttacgtgattttaaccattcaacatgctgttgggttttaattctctaattattattattattgttattattttttaggaccagttgtagtgaattgctactgaaagctatcccaggtgatacagagctctttgtaaaccgcagtcacacattagggttagtattaaactttgtttagatgtaccataattaacttggctagttgattgtttgaagtctatggaagaaatagttttatgcaaaattttaaaaaatgccagtctggtcagggaagtagggggtttcaatgctgttgggaaccaggaaggtgggacagccggcaggtagggacattgtgtacctcagttgtgtcacatgtgagcaagcccaggttgaccttgtgatgtgaattgatctgatcagactgtattaaaaatgttagtacattactcta

SEQ ID NO: 58 - DNA, 7087 bp, Homo sapiens
  source 1..7087: mol_type = genomic DNA
aaacaaaagcaggaggctgacggggctgctggagtttgcagagacacggaggaggagagagcctgtgctttgcctgccgccgcggctgggggagatctggcttttgcaacagcccaccccctttgagatcactctggcccggagtgggggtggggggcagcggggggtggggggggaaagtttgcattgcaatccccctgccttcctctcctttctcccgatcaatgcatatttgcaaaaggattaagccacagatttaagcgccgggagcccatttctgccttgcaaaggagaccggactgaaaaacctaaagccagctctgatttcttttcgccaagtgggaaggtggtttatttttcttgctttttggagtcaacacccttccccaccagcccttatccccaccctcaccccgcaaccccttcacgccccctccccctccccctcctcatcctcccaccatcctctaaagaggcaaagggattttttttttcttttggtcttcttttttcccccttccctgtttatcctgaaaaggatttgaagacaagcttgaaggataaaaagccttggtgcttcccaggagccgagccgaggagcagaagaggaagagccgggggctgccgtagcctttggagatggacgagcagcccaggctgatgcattcccatgctggggtcgggatggccggacaccccggcctgtcccagcacttgcaggatggggccggagggaccgagggggagggcgggaggaagcaggacattggagacattttacagcaaattatgaccatcacagaccagagtttggatgaggcgcaggccagaaaacatgctttaaactgccacagaatgaagcctgccttgtttaatgtgttgtgtgaaatcaaagaaaaaacagttttgagtatccgaggagcccaggaggaggaacccacagacccccagctgatgcggctggacaacatgctgttagcggaaggcgtggcggggcctgagaagggcggagggtcggcggcagcggcggcagcggcggcggcttctggaggggcaggttcagacaactcagtggagcattcagattacagagccaaactctcacagatcagacaaatctaccatacggagctggagaaatacgagcaggcctgcaacgagttcaccacccacgtgatgaatctcctgcgagagcaaagccggaccaggcccatctccccaaaggagattgagcggatggtcagcatcatccaccgcaagttcagctccatccagatgcagctcaagcagagcacgtgcgaggcggtgatgatcctgcgttcccgatttctggatgcgcggcggaagagacggaatttcaacaagcaagcgacagaaatcctgaatgaatatttctattcccatctcagcaacccttaccccagtgaggaagccaaagaggagttagccaagaagtgtggcatcacagtctcccaggtatcaaactggtttggaaataagcgaatccggtacaagaagaacataggtaaatttcaagaggaagccaatatttatgctgccaaaacagctgtcactgctaccaatgtgtcagcccatggaagccaagctaactcgccctcaactcccaactcggctggttcttccagttcttttaacatgtcaaactctggagatttgttcatgagcgtgcagtcactcaatggggattcttaccaaggggcccaggttggagccaacgtgcaatcacaggtggatacccttcgccatgttatcagccagacaggaggatacagtgatggactcgcagccagtcagatgtacagtccgcagggcatcagtgctaatggaggttggcaggatgctactaccccttcatcagtgacctcccctacagaaggccctggcagtgttcactctgatacctccaactgatctcccagcaatcgcatcccggctgaccctgtgccccagttggggcaggggcaggagggagggtttctctcccaacgctgaagcggtcagactggaggtcgaagcaatcagcaaacacaataagagtctccttctcttctcttctttgggatgctatttcagccaatctggacacttctttatactctcttcccttttttttctgggtagaagccacccttccctgcctccagctgtcagcctggttttcgtcatcttccctgcccctgtgcctctgtcctagactcccggggtccccgccctctctcatatcactgaaggatattttcaacaattagaggaatttaaagaggaaaaaaattacaaagaaaataataaaagtgtttgtacgttttcatgctggtggtttgaggagccaaatttacctcactcgaatccctcactccctatgttaacaggcaatccttctctgtttctcttattactctcactacctcttagcaggaatactccacattgccctattcattccaggcctccctgcttcctcttgctcttcctccctggggacagtactgattggaacactttcctcctcttccttcctagccccagctattcactggggactgtcatagctgggattctaaaggtgccacatttttcagtttcatctccactaggttggttcccgggcaggaagtcaggcagcagggaaggacacgggaacagcaggtggagaattcctacagtctttcttaccctgctagcaatagctctcagtttcagaggcacagtctttggagaccattcagcactgagaaagcaatatttagaacctattgcaaaactgggcctgagttaggcatggtgatgaatgcatcagcaaggaatagaaagttcttatcgtgaaacccttcaacctcaactatgccttcatagacacacacgttcatgcacatgtaggcacatgtaccatctcacatcttcactttcccgagatgccatatacaattacctacattaataactgtagcactatgccttttgagcccgagagagggaattagtgactctaagtgaaggtcactgacacagagaagcagtatgtgtctggggcttccaggacctgcaggcccactagcgtgcacttaccagaatggcatacacaggacctgatcatgaggaagaccaggtttccagtgtaaactactcttgttcccaccacctctggagcactcagggagccccatacagtacttacaatgtctttaatggacttgattctgtttaattttttgttttatattaggcacactgtattaattttccaaaatgttataccacactatgttcttggtcctgacctattgctctggaggaaagagttgtataagaacgtggctcatgtgaacttttgctagcttcatttgaggacctgagaatcatggggaaagggaaggtaatgttttcattgaaatcatcacagtgatttttattccctgggaacacagcgtgtactaaaaatacatgagaaaatagcatgtatatgaaagctattctcaaaagtcacctgagctcaccatcttcatagccaaccctaccagttataaagatggcagctctatcacttgattaagtgggaggtggtcaaatattttggtgcctcattttcttcatctgtgagatgggaactgttatgcctggcttactaagagtcttgtgagagactgagaagttgattttgttcatatccaatctgtaaatgcgaagtcaggggaagtaatgtccctgaaataaacgggttcatgccatctagggacaataaatggttttcttgttgtaacttctggttaatatcagtaccttgatgtcatcaccgtgatgacaaagagaagagttattgttgatcttcttggttttggtctgtctcttttcttaggataaagaaaaacttccaaactagaaaaacaggccctggttcccttagtttgcacttgaacccaatatgttgccttgtacatacttggtccctgtcacattgactgcttgggaggcttccagggagaagtatgagaccctgaggggtgagaatgggcagctagcaagaacatggaaattctgcttggcactacagtcataaatagaaaacactgtgtgtgctcaggggagcaggggatgccactgaagaaactcaagggaatgtgtatttgaaggaaatgcaaaaactaagtatttagcaaaatgaaattatgccttgatgactaaaaggcactagaaaggttgtgtctactaacttcagccctaatcagaacagatgcctagaaggagcatttttgtgacaacttcatagtgattagaatcagtggagaactccatcttagtggcaggaatataatgaaactacccacgcaagaacatggttgaatcacatttgcttgacttagggcaaagtacgaaagagagacaaaagggttctcttggaaacaagaagagtgactccagatgtggcctgaataattgccatgttaagttaatgcaaaagatcagaacagggctacatttgcacaggcagtttctctccgggccgtagttttcactgatgatcacctttcacagcattttccccaaccagcatttcacttagtcttctctatacccagcacctcccccggcacccccggcaagcccactatcacttccgacttccaacgtggcatccgtgagatctgtccacattaggcgaagcaggagaacactgagagcagcaggatgggtttggaaagagcatgcctctggaaacacagcttcctgggaattcacatgaggccagtcctacagagagcaagatgcaccccaggatttcttcattttctaatagatgtgggagtgctccattttccccgacagcgaatttcccctgagaaacgatactagaccctgggtttgcccaccttgtaactcttccttatctcctccttttcatccctaatccatcctccctctggcatggaattgacgcccgtgcagtacatttgccaagtggcaccttctttcaatttatgttttattttgctatggtggtgattctttatttgctggttgtcttttctcacacatctttctctctgtctctctctttcctgctctttgtttttctgcccagaaaaacctgacttcgataccaaaaaagatgaaactacagaaactcaaatttaaaaaaaactttaaaagaaacaaaaaaatactcaacgattctttcagctttattaacattttccattgtttcttgcgacttgtgtctcgttctttgtagtattgatgatgaacatttgataatgaatgttcttgtatattcagataaagaaaaaaaaaaccaaaaaagcggtctgaatttaatagtgtttataataaaaattttaaaaatgaccctcatagcacgcaaaacaggatggggaatttcccctcttctttctgtgacaatgcgcatcattcctgcattagtttttaacaccagactacctacattcatcatttccctcatttttcttttattttcttgcatttgtgaattagttcaagaatgctagaaaagtgtcgagttgtgcacatccatttcttgtttcacaatgtttaaaagtgacagtaattcattttgtaaactaaaaaaaaaaaaaaaaaggttggaatagtgagcataataggtacaacctaacacattattatgtttattaactttgagacccagaaataaattcttttcttttcttgattcttgctcttaaaaatacaaaaaaaaaaatgttttgttttgtgttatttttggtttgtttattggggggctttttttaattgtcaggattatgatcttgctgtttttcttcaatatgtatacaaggtgatgtgaaaagatgacttgggcagaggagtaagaacaagtaggcttgttcttctactttgcttcagaattcagttaatgccaaaagcgaagatcaagcccatgttgatgtctcgttgctcacctgcatttccagagagtgtgacactcatgcagtccctgagaaaaataaaatcagggacatacttctccttttagccttttaaaaattcaaaaacgtttagtccaagggaactttttatgctatcaggaaaggtttttgctgtttttgattctgattatcacagccaagtactttgttttatttctccctaattaataactacattccatgaggcctcttccaaccaaagaggccttttcttccaggagagtcccgcaggagatgctggtatgatgggcaccattggttaagtaaactacatgcaggaagaagtccttggggccagtctgccagctgagtcctggttttggatgaagagttaatgagatattgggccaggctcaatgctgtagttttaatgctaagaggttacgtttacttcacagagtacacctcttagtaacctctgacttaggcagctgcttaaagcaaattgcaaaactggcttgatttggaatgtttttattagaggaaaaaagaaagccatattatctggaaaaaaattcattttaaataccatcattcaacaaattatgttcagaaagtggtcagaacttaagcaagaaaagtaaagaaagaatgcagaattgtggagcaatgctttaggaaatatttctacctgaacacttgtactcttgaagtcacaacaaaataatgatgagcttttcacatcacctttatggtttcaatccctagctcaaagcttcctggaatcttttattttttgtaaacttttttttcttttgttaaaataaataaaacattcaatgtttttctccttttctctcttattacttctttcctttggcattttcaatttgaaatgctttcctttggttgttggttttattctccccctacccctccccttttcttattattcagaatataaacctgcaaagctctgctctgttttggttttgaaagtttaagcttttctgcttctgtgagagcacaggcttctgtcccttttgattccaactgaacttttgtgttctctaatgatactaacacggtgtaggttttacagtctcctaatttgtactggtaatgcatattccaaataaatagtttcttttgttgcaaaaaaaaat

SEQ ID NO: 59 - DNA, 1516 bp, Homo sapiens
  source 1..1516: mol_type = genomic DNA
gattatcctgctgctgccgccaccgctgctgctgctctgcaaaattcagctgctgcctctgtcttgaggaccccagcgcctttcccccggggccatgctgcctgcagccacagcctccctcctggggcccctcctcactgcctgcgccctgctgccttttgcccagggccagacccccaactacaccagacccgtgttcctgtgcggaggggatgtgaagggggaatcaggttacgtggcaagtgaggggttccccaacctctacccccctaataaggagtgcatctggaccataacggtccccgagggccagactgtgtccctctcattccgagtcttcgacctggagctgcaccccgcctgccgctacgatgctctggaggtcttcgctgggtctgggacttccggccagcggctcggacgcttttgtgggaccttccggcctgcgcccctagtcgcccccggcaaccaggtgaccctgaggatgacgacggatgagggcacaggaggacgaggcttcctgctctggtacagcgggcgggccacctcgggcactgagcaccaattttgcggggggcggctggagaaggcccagggaaccctgaccacgcccaactggcccgagtccgattaccccccgggcatcagctgttcctggcacatcatcgcgcccccggaccaggtcatcgcgctgaccttcgagaagtttgacctggagccggacacctactgccgctatgactcggtcagcgtgttcaacggagccgtgagcgacgactcccggaggctggggaagttctgcggcgacgcagtcccgggctccatctcctccgaagggaatgaactcctcgtccagttcgtctcagatctcagtgtcaccgctgatggcttctcagcctcctacaagaccctgccgcggggcactgccaaagaagggcaagggcccggccccaaacggggaactgagcctaaagtcaagctgccccccaagtcccaacctccggagaaaacagaggaatctccttcagcccctgatgcacccacctgcccaaagcagtgccgccggacaggcaccttgcagagcaacttctgtgccagcagccttgtggtgactgcgacagtgaagtccatggttcgggagccaggggagggccttgccgtgactgtcagtcttattggtgcttataaaactggaggactggacctgccttctccacccactggtgcctccctgaagttttacgtgccttgcaagcagtgcccccccatgaagaaaggagtcagttatctgctgatgggccaggtagaagagaacagaggccccgtccttcctccagagagctttgtggttctccaccggcccaaccaggaccagatcctcaccaacctaagcaagaggaagtgcccctctcaacctgtgcgggctgctgcgtcccaggactgagacgcaggccagccccggcccctagccctcaggccttctttcttatccaaataaatgtttcttaatgaggaa

SEQ ID NO: 60 - DNA, 5754 bp, Homo sapiens
  source 1..5754: mol_type = genomic DNA
acaaactaaactcattgaattagaacagcacttgtccatctcaaagaggataaattattttttgctatccaccccagctggaatttagaccagacttacaggctttctgctccttccttcagtgaagaagagtctccagggtccctctagtgcaggtcaaagaagtccagtccagctgagttcagctccctagggcctccccagccagtctcctggaccaagagctggctttgcaagaaaacatcctactaaagccctgggaaaagagacaaatggtgaagaggagaatgaaaggttgcaggagggagctcctgttttagccctgccacctacctgggggccctctggctaccagtgtccttggaggggcttccttgctgcttctcccactaaggagctctggagccttaggaccatggacgctctcaataggaaccaaataggccctggatgccagacccagaccatggtgcagaaaggacccttggacctgatcgagacaggcaaagggctgaaagtgcaaacggacaaaccccacctggtgagcctgggcagtgggcgactcagcacagccatcaccctcctgccgctggaggaagggaggacggtgattggctctgcagccagagacatctcactacagggcccaggcctggctccagagcactgctacatcgagaacctgcggggcaccctcaccctctacccctgtggcaatgcctgcactattgatgggctccctgtccggcagcctacccggctcactcagggctgcatgttgtgcctgggtcagtccaccttccttcgctttaaccacccggctgaagccaagtggatgaaaagcatgattccagcagggggccgagcccctgggcccccctacagccctgttcctgcagaatcagaaagtctggtaaatgggaaccacaccccacagactgcaacacggggaccctctgcctgtgccagccacagttccctggtgagctctattgagaaggacctgcaagagatcatggactcactggtgctagaggagcctggagctgctggcaagaagcctgccgcaacctctccactgtcaccgatggctaatggtgggcgctacctgctgtctcccccaaccagccccggcgccatgtctgtgggctccagctatgagaacacctctccagccttctctccactctcttcaccagccagcagtggaagctgtgccagtcactcacccagtgggcaagagccaggaccttctgtgcccccgctggtacctgcccgttcctccagctaccatctggccctacagcccccacagtcccgcccaagtggtgctcgctccgagagtcctcggctgagcaggaaagggggccatgagaggcctcccagccctggcctccggggtctgctgacagacagccctgcagctactgtcttggcggaggcccggagagccactgagagcccccggctgggtgggcagctgcctgtggtggccatcagcctgagtgaatacccagcttctggtgctctcagtcaacccaccagcattcctggcagccccaagtttcagcctccagtccctgctccccgaaacaagattggcacactccaggaccgccctcccagccctttccgtgagcctccaggcagtgagcgggtgctaacaaccagcccctcacgccaactggtgggccgaacattttcagatgggttagccacccgtaccctgcagcctcctgagagtccccgcctgggccggcggggcctggacagtatgcgagaactaccccccttaagtccatctctgtcccggcgagctctctccccgctgcccacccggaccaccccagatcccaagctaaacagggaagtggcagagagtcctcggccccggcgctgggcagcccatggggcttcaccagaggacttctccctgacgctgggggcacggggccgtaggacacggagcccctcacccacactgggtgagtctctggcaccccacaagggcagcttcagtggcaggctgagcccagcctacagtctgggctctcttactggggcttcaccctgccagagtccctgtgtccagaggaagctctccagcggggacttgcgggtgcctgtcacaagggagcggaaaaatagcatcacagagatcagtgacaatgaggacgacctcctggagtaccaccggcgacagcgccaagagcggctccgggagcaggagatggagaggctggaacgccagcgcctggagaccatcctgaacctgtgtgccgaatacagccgggctgatgggggacctgaggctggggagcttcccagcattggggaggccaccgcagcattggcactggcaggccggaggccctcacgaggccttgcaggggcctctgggcggagcagcgaggagcctggcgttgccacccaacgcctatgggagagtatggagcgctcagatgaggaaaatctcaaggaggagtgcagcagcactgagagcacccagcaggagcacgaagatgcacctagcaccaagctccagggagaggtgctagccctggaagaagagcgggctcaggtgctggggcacgtggagcagctcaaggtccgtgtgaaggagctagagcagcagctgcaggagtcagcccgagaggccgaaatggagcgggcactgctgcagggagagagggaggcagagcgggcactgctgcagaaggagcagaaggcagtggatcagctgcaggagaagctggtggccttggagacaggcatccagaaggagagggacaaggaggcagaggccctggagactgagacaaagctctttgaggacttggagttccagcagttggagcgggagagccgcgtggaggaggagcgcgagctggccggccaggggctgctccggagcaaggctgagctgctccgcagcatcgccaagaggaaggagcgcctggccatcctggacagtcaggctgggcagatccgggctcaggccgtgcaggaatcagaacgcctggcccgggacaagaatgcctccttacagctgctgcaaaaggagaaggagaagctgactgtgctggaaaggagataccactcactcacagggggcaggcctttcccgaagaccacatcgaccctcaaagagatggagaagctgctgctccctgctgtagacttagagcagtggtaccaggagctgatggccgggctggggactggccccgctgcagcctcccctcactcttctcccccgcctctgcccgccaaagcttcccgtcagctgcaggtttaccgctccaagatggatggcgaggccaccagcccccttccccggacccgcagcggccccctcccctcctcctctggctcttcctcctcctcctcccagctcagcgtggctaccctggggcgtagcccctccccaaagagcgctctactcacccagaatggcacgggcagccttcctcgcaacctggcagccacactgcaggacatcgagaccaagcgccaactagctctgcagcagaagggacaacaagtgattgaagagcagcggcggcgactggctgagctgaagcagaaagcggcagctgaggcacagtgccagtgggatgcccttcacggggcagcacccttcccagcgggcccctcgggcttcccccctctcatgcaccactctatcctacaccacctgcctgcggggcgggagcgtggggaggagggtgagcacgcctatgatacgctgagtctggagagctctgacagcatggagaccagcatctccaccgggggcaactcggcctgctcccctgacaacatgtccagcgcgagtggtctggacatggggaagatcgaggagatggagaagatgctgaaagaggctcatgcagagaagaaccggctcatggagtcgagggagcgggagatggagctgcggcggcaggccctggaggaggagcggcggaggcgtgagcaggtagaacggaggctgcagagtgagagtgcccggaggcagcagctggtcgagaaggaggtcaagatgcgggagaaacaattttcccaggcacgacccctgacccgctacctgccaatccggaaggaggactttgacctgaagacacatattgagtcatcgggccatggtgttgatacctgcctgcacgtggtgctcagcagcaaggtctgccgtggctacttggtcaagatgggcggcaagattaaatcatggaagaagcgctggtttgtcttcgaccggctcaagcgcaccctttcctattatgtggacaagcatgagacgaagctgaagggagtcatctatttccaggccattgaggaagtgtactacgaccacctgcgcagtgcagccaagaagaggtttttccgcttcactatggtgactgagagcccgaacccagccctcaccttctgcgtaaagacccatgaccggctgtactacatggtggccccatctgcagaggccatgcgtatctggatggatgtcattgtcacaggggctgagggctacactcagttcatgaactaactgccgtgggcctcctggcagagcacaactggggcttttgtataagaagactttaatattctgtaaggagcttggtcctgtgagtttctgggctctggcctcctgaagaaccagccagaagaagaaaagtagaggtggctttgctgcctcctgggagcccagaacttgcagtaaccctttagggtcctgccccaggcccagccagggctgaggagctgtcacagagagggcctcagctctgacctgacacctgctctccccagcctgttttctcttttctaaaagacaaattatggtaccataagctgccaaagatcccctcctgcctcagacccctttgccaggggctttgggggctgagcagagccacatccagagtggggtaatagctcaggcggcccgcttcccatttctcaaaccccgctctgccccattgttctcctttcccttatactttttattaccttgctcaagggccagagatctcaagtgtcaaccttgaggtcccagctccatcccctagttgcagactcatcaccatggttaccatagtgactgcttcattgccatggttacatactaattgctgcagctctgtggcccagcccactgcttcagctgtgggccatctgagggtacgtgccatcatctctccagcccaggcccctgggcatctcatgctggggggaagggactgaatacctttttccttccccctgcctgtgtcttcagccctgatgcacaggctgccagccccccagtccagccctctcccttccactggtgccttgcttagagccagaagggatgaagccgggggatctatggaacagaggaggagcgatgcagttgggagaggaagctagaagggttatggttggagttctgtacagtgttgagtttccgacagggaaagaggattcctccaatgctcctagagagaaagcctgagcaggagatgatgcagcagaggggaagggccctgtggtgccgccgcccttccttcagcctccgaagggtgatggaaatggagagtggaggaccaggcctccagctgtctggcctcgcccttcacgccttaacactaagcccacctcccctgctctccttcccagcattgagcccttggttgcctgggcccaggctgggggttttcagtatttgtaagcatttcagcagaacaataaagcctttggactacggaa

SEQ ID NO: 61 - DNA, 1308 bp, Homo sapiens
  source 1..1308: mol_type = genomic DNA
ctcctctaggccgccggccgcgaagcgctgagtcacggtgaggctactggacccacactctcttaacctgccctccctgcactcgctcccggcggctcttcgcgtcacccccgccgctaaggctccaggtgccgctaccgcagcgtgagtacctggggctcctgcaggggtccactagccctccatcctctacagctcagcatcagaacactctctttttagactccgatatggggtcctccaagaaagttactctctcagtgctcagccgggagcagtcggaaggggttggagcgagggtccggagaagcattggcagacccgagttaaaaaatctggatccgtttttactgtttgatgaatttaaaggaggtagaccaggaggatttcctgatcatccacatcgaggttttgaaacagtatcctacctcctggaagggggcagcatggcccatgaagacttctgtggacacactggtaaaatgaacccaggagatttgcagtggatgactgcgggccggggcattctgcacgctgagatgccttgctcagaggagccagcccatggcctacaactgtgggttaatttgaggagctcagagaagatggtggagcctcagtaccaggaactgaaaagtgaagaaatccctaaacccagtaaggatggtgtgacagttgctgtcatttctggagaagccctgggaataaagtccaaggtttacactcgcacaccaaccttatatttggacttcaaattggacccaggagccaaacattcccaacctatccctaaagggtggacaagcttcatttacacgatatctggagatgtgtatattgggcccgatgatgcacaacaaaaaatagaacctcatcacacagcagtgcttggagaaggtgacagtgtccaggtggagaacaaggatcccaagagaagccactttgtcttaattgctggggagccattaagagaaccagttatccaacatggtccatttgtgatgaacaccaatgaagagatttctcaagctattcttgatttcagaaacgcaaaaaatgggtttgaaagggccaaaacctggaaatcaaagattgggaactagtggaaagcggaagagcaggtcttgatgtgtcctagaattttgccatttctgagattgagccattgaaggcattccatttctaaagcttatttagccggtgcttctaaagaattccacactaacgtgataacatggtttttgtaacaataaatgtaggatatttcctggcacatgcaaataaacctaatcattgtttctttaaaaa

SEQ ID NO: 62 - DNA, 1472 bp, Homo sapiens
  source 1..1472: mol_type = genomic DNA
aagaatttaccggcaggcaggtctggtcccagcagaggagacccaggggtcccagagctgggctggcgggaggcgtaatccggcggggtgagggttgatcgaagagccccgcgcgcagctgccgctcacagccccttcccgagtgcagagcgggcagagaagtccactgcttttaaggccctgcactgaaaatgcaagctcaggcgccggtggtcgttgtgacccaacctggagtcggtcccggtccggccccccagaactccaactggcagacaggcatgtgtgactgtttcagcgactgcggagtctgtctctgtggcacattttgtttcccgtgccttgggtgtcaagttgcagctgatatgaatgaatgctgtctgtgtggaacaagcgtcgcaatgaggactctctacaggacccgatatggcatccctggatctatttgtgatgactatatggcaactctttgctgtcctcattgtactctttgccaaatcaagagagatatcaacagaaggagagccatgcgtactttctaaaaactgatggtgaaaagctcttaccgaagcaacaaaattcagcagacacctcttcagcttgagttcttcaccatcttttgcaactgaaatatgatggatatgcttaagtacaactgatggcatgaaaaaaatcaaatttttgatttattataaatgaatgttgtccctgaacttagctaaatggtgcaacttagtttctccttgctttcatattatcgaattcgaatttcctggcttataaactttttaaattacatttgaaatataaaccaaatgaaatattttactgataagattcttcatgcttctttgctctccttaaaatgtctttttcactagttagttccaagggtcagtctcataattttgttcttatactttgatttcctttttctttttttttttttttaatagagttgggggatctttctttctttttttttttttttttttgagatagagtctcgctctgttgcccaggctggagtgcagtggcgtgatctcagctcactgcaacctccacctcctggatacaagcgattctgctgcctcagtctcccgagtagctgggattacaggttcccgccaccatgcccagctaatttttgtatttttagtagagacggggtttcaccatcttggtcaggctggtctcgaactcctgacctcgtgatccacctgcctcggcatcacaaaatgctgggattacaggcatgagccaccgcgtccagcctagagttgggggtctttctatgttgcccaggctggtcttgaactccgggcctctagcaagcctctcatctcagcctcccaaggtgctggaattacaggtgtgagccactgcaccatactttggtttcttagcaggagattttggttcacagtaacccagctctctttcatctaagaactttagatgagaaaaaaaaaaaa

SEQ ID NO: 63 - DNA, 3443 bp, Homo sapiens
  source 1..3443: mol_type = genomic DNA
gtgggctccggctgcagctccggtctgctctcttggctccggaacccccgcgggcgctggctccgtctgccagggatgacggcgacccctgagagcctcttccccactggggacgaactggactccagccagctccagatggagtccgatgaggtggacaccctgaaggagggagaggacccagccgaccggatgcacccgtttctggccatctatgagcttcagtctctgaaagtgcaccccttggtgttcgcacctggggtccctgtcacagcccaggtggtgggcaccgaaagatataccagcggatccaaggtgggaacctgcactctgtattctgtccgcttgactcacggcgacttttcctggacaaccaagaagaaataccgtcattttcaggagctgcatcgggacctcctgagacacaaagtcttgatgagtctgctccctctggctcgatttgccgttgcctattctccagcccgagatgcaggcaacagagagatgccctctctaccccgggcaggtcctgagggctccaccagacatgcagccagcaaacagaaatacctggagaattacctcaaccgtctcttgaccatgtctttctatcgcaactaccatgccatgacagagttcctggaagtcagtcagctgtcctttatcccggacttgggccgcaaaggactggaggggatgatccggaagcgctcaggtggccaccgtgttcctggcctcacctgctgtggccgagaccaagtttgttatcgctggtccaagaggtggctggtggtgaaggactccttcctgctgtacatgtgcctcgagacaggtgccatctcatttgttcagctctttgaccctggctttgaggtgcaagtggggaaaaggagcacggaggcacggcacggcgtgcggatcgatacctcccacaggtccttgattctcaagtgcagcagctaccggcaggcacggtggtgggcccaagagatcactgagctggcacagggcccaggcagagacttcctacagctgcaccggcatgacagctacgccccaccccggcctgggaccttggcccggtggtttgtgaatggggcaggttactttgctgctgtggcagatgccatccttcgagctcaagaggagattttcatcacagactggtggttgagtcctgaggtttacctgaagcgtccggcccattcagatgactggagactggacattatgctcaagaggaaggcggaggagggtgtccgtgtgtctattctgctgtttaaagaagtggaattggccttgggcatcaacagtggctatagcaagagggcgctgatgctgctgcaccccaacataaaggtgatgcgtcacccagaccaagtgacgttgtgggcccatcatgagaagctcctggtggtggaccaagtggtagcattcctggggggactggaccttgcctatggccgctgggatgacctgcactaccgactgactgaccttggagactcctctgaatcagctgcctcccagcctcccaccccgcgcccagactcaccagccaccccagacctctctcacaaccaattcttctggctgggcaaggactacagcaatcttatcaccaaggactgggtgcagctggaccggcctttcgaagatttcattgacagggagacgacccctcggatgccatggcgggacgttggggtggtcgtccatggcctaccggcccgggaccttgcccggcacttcatccagcgctggaacttcaccaagaccaccaaggccaagtacaagactcccacatacccctacctgcttcccaagtctaccagcacggccaatcagctccccttcacacttccaggagggcagtgcaccaccgtacaggtcttgcgatcagtggaccgctggtcagcagggactctggagaactccatcctcaatgcctacctgcacaccatcagggagagccagcacttcctctacattgagaatcagttcttcattagctgctcagatgggcggacggttctgaacaaggtgggcgatgagattgtggacagaatcctgaaggcccacaaacaggggtggtgttaccgagtctacgtgcttttgcccttactccctggcttcgagggtgacatctccacgggcggtggcaactccatccaggccattctgcactttacttacaggaccctgtgtcgtggggagtattcaatcctgcatcgccttaaagcagccatggggacagcatggcgggactatatttccatctgcgggcttcgtacacacggagagctgggcgggcaccccgtctcggagctcatctacatccacagcaaggtgctcatcgcagatgaccggacagtcatcattggttctgcaaacatcaatgaccggagcttgctggggaagcgggacagtgagctggccgtgctgatcgaggacacagagacggaaccatccctcatgaatggggcagagtatcaggcgggcaggtttgccttgagtctgcggaagcactgcttcggtgtgattcttggagcaaatacccggccagacttggatctccgagaccccatctgtgatgacttcttccagttgtggcaagacatggctgagagcaacgccaatatctatgagcagatcttccgctgcctgccatccaatgccacgcgttccctgcggactctccgggagtacgtggccgtggagcccttggccacggtcagtccccccttggctcggtctgagctcacccaggtccagggccacctggtccacttccccctcaagttcctagaggatgagtctttgctgcccccgctgggtagcaaggagggcatgatccccctagaagtgtggacatagttgaggcccccgtcagggagaggtcaccagctgctgtgccccaccacgtctggctccctgccccttaaccccaaggactgagggcagtgccctttgagatctggggaggcaggcattcctgaagggaactagaggtgttacagaggacccttacgtgagaaatagctgaaaagggcactcccaaccctgggctggggaggaggagagagtcccagagctcatcccccctgctgcccagtgcaaaccacttctccatgctgcaaaggagaagcacagctcctgccagggtgagcagggtcaagcctcttattccaggagaagggggctctgccccaggccctactacccattgttcccttcctcttcctgcccttgaaccccctccctgtcccagggccctcccagcccattgctgccaaggtggagggaaggataaagccacttctggcttcagcccccaccaggggaaggaaggagggcacattaactccctccaccagcctgctgacagacactaactttgtatccgttcaataagcatttcataaataaaggtgtagaaaagg

SEQ ID NO: 64 - DNA, 3734 bp, Homo sapiens
  source 1..3734: mol_type = genomic DNA
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 65 - DNA, 6102 bp, Homo sapiens
  source 1..6102: mol_type = genomic DNA
aggcggcttccgcccgccccaacagcgcgcacgcggctaccgagctggaggaggcggcgggcgcgagacccggaatgcgcagggcccccgcctcgccccccccagcccgggccgcggcccccgccttccccgcagtcgtcccgcactcggtgcccgccccccgaggccggcggctgctcccactcggggccgttgctgcttgtgccgtgagcgccgcccagccattgtccccgtcgctccgtcagccgcgccggaccgcgcaccaggaggcgagagcgcgcatggggagcctctgttgatgccgccgccgcgccgccctccgaggctgcgtcccgggaagcccggctccccgagcgctccggcctggcccggtgccccggacctgagtgcgtccccatggaggcgcccgggctggcccaggcggccgcggcggagagcgactcccgcaaggtggcggaggagacccccgacggggcgcccgcgctctgccccagccctgaggcgctgtcgccggagccgcctgtgtacagcctgcaggactttgacacgctggccaccgtgggcactgggacgttcgggcgggtgcacctggtgaaggagaagacagccaagcatttcttcgccctcaaggtgatgagcattcccgacgtcatccgcctaaagcaggagcaacacgtacacaatgagaagtctgtcctgaaggaagtcagccacccgttcctcatcaggctgttctggacgtggcatgacgagcgcttcctctacatgctcatggagtacgtgccgggcggcgagctcttcagctacctgcgcaaccgggggcgcttctccagcaccacggggctcttctactctgcagagatcatctgtgccatcgagtacctgcactccaaagagatcgtctacagggacttgaagccagagaacatcctgctggatagggatggccacattaagctcacggactttgggttcgccaagaagctggtagacaggacttggaccctctgtggaacacccgagtacctagcccccgaagtcattcagagcaagggccacggaagggccgtggactggtgggccctcggcatcctgatattcgagatgctttcggggtttcctccgttttttgatgacaacccgtttggcatttatcagaaaattcttgcaggcaaaatagatttccccagacatttggatttccatgtaaaagacctcattaagaaactgctcgtggttgacagaacaaggcgattaggaaacatgaagaacggggcgaatgatgtgaagcatcatcggtggttccgctccgtggactgggaagctgttccgcagagaaaactgaagcctcccatcgtgcccaagatagctggtgacggcgacacttccaacttcgaaacttaccctgagaatgactgggacacagccgcgcccgtgccgcagaaggatttagaaatcttcaagaatttctgaggacaggagctcacatctggaagaaacaggaagattggaatctgcctggaacaaagaactgcacctaagcagaccagaagcaaaatgtcttcttcacggcataaggacatctccacttttctctgtacctgtgtgtatagaaatagattagagcacagttgaaattcatggaactggcattatttaagcaactggaattccacactgtaggaaggttttgaaaattgtttggttgtagattttatcttatcctttagtgttgtgttcctactgtgatgtcttggtttttgtcatagacttaagtttataagtttgaactggacttgttcgattataaccacaaattgtgtgtgtgtgtgtgtgtgtgtgtgtgtgtgtgtgtgtgtgtgtatgcctgtgtgtatatatagaagtcattatggcagatgcacagaaattgtgcagtgatgtaaatgttcatactttacagagcctataatttttatttttcaatttgttttttcaaaaatctcttctcggggacaacatctgaagggtatgttgcatgcattaaaaaaaatcatctcacatgcattttatagttttggggaagaaaatatcatggggaggtctaccttcagtatctttagtgcttcttacctggtaacttgagactttaaaagaagaaacaaagaggggaagatatgggagcgaatttattccaagaatctacaatgacattgaagttgttggaggaatgtactgtatttaaaaaaaccttctgtgacacattcaaaaatttcatctgagctggatgcagtggcttgttcctatagtcccagcactttgggaggctgaggtgggtggattgcttgagcccaggagttggagaccagtctgggaaacgtggtgagacctcatctctacaaaatacaaaaaaattagccgggcatggtggcacgtgagtgtagtctcagctactcaggaggctgagatgagaggatcacttgagcctggggaggtccaggccgcagtgatccgagatcacaccactgcattccagcctgggtgacagagtgagaccctgtccaaaaaaaaaaaaaaaaaaaaagaaggcaatggctttgtgcttttgaagatggattgaagagaacgtccaccttaaggctttaaaagacagtgaagctgggtgcggtggtgcactcctgtaaccctgggactttgggaagctgaggcaggaagattgagcctaggagttcgagaccgacctgggcagcatagcgagaccccatctctatataaaaaaaaagatgttaaaacagtgaaattacagaaacagaacatagccaccctttttcatcagtgaccttttcttcacgtggaggtcaactcagtaggtgagaataattagtaggttcagcagaataaactctttacaattaataactcagtggaaaatgatgttcagatggtgaaatgtggaaatgttttagacaactctattttcagcctgtgcttctcactcaccatcctgttgggattaacagttgagggccatcgctgcctaaacatttagcgtgcggtttcccatcagttttaccgtgaatgtgaaaaggtgaaactggtgctgacttcggcagcaggtatactaaaattggaatgacacagaggagattagcatggccccctgcgcgaggaagatctgcagattcatgcagcattccatatttttttaagaaaaggttacactgtgcaccgacatgtggagctagaaggaaaagcttttaagtctcatgtttgctgactataaaaataaatcatagtaataatattgaacattctactttaaaaattattaataaaaccataaatgaaacacttgagcacactttaaaaagtacataaatccatgagtccatatgcctatttttaggttgtctgttttattatccaggatttcatactcagatgagaattagcatcaggaaagcctgaaatgccttaattgtgaactgatatgcaggaaaatgaaatatgcaagcacattggtgaccttgttctttacagacgtcgtgatcagcctccagccatacagaacaaatgagaaattagaagcgattccaagaacgtgtgatggtgtgaaacccactttggaccatcacacacatttcagcatttgaagtgacacaagctacctggtaactgcccatggcggggtcatagtttaaatggcccaatgtcatgtagaggttaatttaatcaaaatgagctataaacatggagcaatcactgtataattttaaaatagtttacctggccaggcatggtggctcacgcctgtaatcccagcactttgggaggccgaggcaggtggatcacctgaggtcaggagttcgagaccagcctggccaacatggcaaaacccccatctctactaaaatacaaaaattaggtgtggtggcgggcacctgtaatcctggctactcgggaggctgaggcaggagaatcgcttgaacccgggaggcagaggctgcagtaagccgagatcgtgccactgcactccagcctgggccacagagcaagactccgtctcaaaacataaaaataaaaaaaatacaaatagctgacccgggtgggcattccaaaatgtccgcatggacgatcacgaaacaatcacggtgcgaaatgtccgcatggatgatcacgaaacaatcacggtgcagaatgtccgtatggatgatcatgacaaacaatcactgcgcaaaatgtggaggaaaattcattaccataatcacaacatttgtatcagaaaaacattacaaatttactttgcttcatgtaattttacgagttggcaatattttcttaaattggaatactctaagagaataggacatggatatgttacaagaagaagtgtattggggcaagtcttgtttttctgaagggaaatgccaccggcctgctttcataggcattgataaagccaggaaggaaataaagtcacaaccaaatagtatacactgtctctactgaattcttctaattccttcgcaacagaagtctaaatcctgcttagcctaaacatatctatagccagcgatgggcaaattatatttgctgtcagcatgatagaataagaagcagaaaccccccagcagtggggtaagattgcgcgcttcatcccctcaaaatgcagttcagccccactccctctccatgcccatcaaacaactgcacaatcacttgttactgaatttcccattcagcgttttgtttctttctgtccatacttaaagaccaaatcatttgtttgctttgaagaaaaagcaaaaacaagaacaaaaacaccaaatcatttgaaatcactgtccattaaaaaaaatcaccagtggaactaaacagttgagtggtttgggatgtgttcgccatttctgtgtttgacctacatggcctctaattctttggaccattgtaactgtgaaactatggcttaatactgttaaagaccccaaggctccactctgtagccaggtgcacctcaggctgcacaaataccccaccagaatgtcacaatcattcattgctccagctactgagtgaacagggtgcaagacagcactggcttgggttgtatttttgcagccagttaactttgcatcaggtctacacagtccttgcagcctttgatttctccagttgtagagatttgaaatggggtgcccgggttcctctcgtttttccttcaatctacctagcatgcattttacacacattggaggagtcgtatgcactgtttttctgttgctgatggaggcaagcaggccttctaattagtctaacttaaacggatgggattcacttaacaaaagcaagaagaaaagcacacgaacaggtgtcttaatgcatttggatagtttctttgcggggtgtgtgtgtgtgtgtgtgtgtgtgtgtgtgtgtgtgtgtgtgtgtgtcttggtgaaggtgaggggctgtattttcaccaagccttttcataatcaagtttgctttgcaaaccttatggccttgcaccccgcagaacagtcccttcctaatagcagggatgctctgtcatggttttctgtaaattatcgtgtgttgggaagttctgttgaggcttagtttgatcttccatggtggacacgtctgttctgtatgtaaaaggcattacaattgtgttttagcagatgagacttgaagcctttccacagtccttgtgctctgagatggctgttgagctctgctcaactgtcgtagctgaattctttctttgcgctgaacactgggcagcctcaccatgttgccaacgtgcttctggggcccctgtcactgccgccggatgccgcgcacacaggcagagtgccttggcaggtttgtgcaccccttggcgagccagaactgggaaccccgcggggtgaccccttctcatggggggtcggggaggggagtgcctatttttaatcctgtctgtttgttgcacaatggaaatcactgtgatttgtacatatgccctaggaaaattttactgctgtctaatttatgtaataatactgttgattccaggtttgtttaataaaactttgtatcttttcagaagtctgagtttcatctgcttctgtctccaaa

SEQ ID NO: 66 - DNA, 2153 bp, Homo sapiens
  source 1..2153: mol_type = genomic DNA
agcactatccttgcttaggcctgagccacagaagctgctcagggactatccatggcctccccgtggccaacttgagtctgctctgcagctttaggcccgacttggaaggcccatgggctgcagatgaggaaactgaggtccagacagccgaagagtggtagtgtccaggacacacaactgggccggcggcggcggctgccccgagggacgcggccctaggcggtggcgatggggaccgcccggatcgcacccggcctggcgctcctgctctgctgccccgtgctcagctccgcgtacgcgctggtggatgcagatgacgtcatgactaaagaggaacagatcttcctgctgcaccgtgctcaggcccagtgcgaaaaacggctcaaggaggtcctgcagaggccagccagcataatggaatcagacaagggatggacatctgcgtccacatcagggaagcccaggaaagataaggcatctgggaagctctaccctgagtctgaggaggacaaggaggcacccactggcagcaggtaccgagggcgcccctgtctgccggaatgggaccacatcctgtgctggccgctgggggcaccaggtgaggtggtggctgtgccctgtccggactacatttatgacttcaatcacaaaggccatgcctaccgacgctgtgaccgcaatggcagctgggagctggtgcctgggcacaacaggacgtgggccaactacagcgagtgtgtcaaatttctcaccaatgagactcgtgaacgggaggtgtttgaccgcctgggcatgatttacaccgtgggctactccgtgtccctggcgtccctcaccgtagctgtgctcatcctggcctactttaggcggctgcactgcacgcgcaactacatccacatgcacctgttcctgtccttcatgctgcgcgccgtgagcatcttcgtcaaggacgctgtgctctactctggcgccacgcttgatgaggctgagcgcctcaccgaggaggagctgcgcgccatcgcccaggcgcccccgccgcctgccaccgccgctgccggctacgcgggctgcagggtggctgtgaccttcttcctttacttcctggccaccaactactactggattctggtggaggggctgtacctgcacagcctcatcttcatggccttcttctcagagaagaagtacctgtggggcttcacagtcttcggctggggtctgcccgctgtcttcgtggctgtgtgggtcagtgtcagagctaccctggccaacaccgggtgctgggacttgagctccgggaacaaaaagtggatcatccaggtgcccatcctggcctccattgtgctcaacttcatcctcttcatcaatatcgtccgggtgctcgccaccaagctgcgggagaccaacgccggccggtgtgacacacggcagcagtaccggaagctgctcaaatccacgctggtgctcatgcccctctttggcgtccactacattgtcttcatggccacaccatacaccgaggtctcagggacgctctggcaagtccagatgcactatgagatgctcttcaactccttccagggattttttgtcgcaatcatatactgtttctgcaatggcgaggtacaagctgagatcaagaaatcttggagccgctggacactggcactggacttcaagcgaaaggcacgcagcgggagcagcagctatagctacggccccatggtgtcccacacaagtgtgaccaatgtcggcccccgtgtgggactcggcctgcccctcagcccccgcctactgcccactgccaccaccaacggccaccctcagctgcctggccatgccaagccagggaccccagccctggagaccctcgagaccacaccacctgccatggctgctcccaaggacgatgggttcctcaacggctcctgctcaggcctggacgaggaggcctctgggcctgagcggccacctgccctgctacaggaagagtgggagacagtcatgtgaccaggcgctgggggctggacctgctgacatagtggatggacagatggaccaaaagatgggtggttgaatgatttcccactcagggctggggccaagaggaaaaacagggaaaaaaagaaaaaaaaaagaaaaaggaaaaggaa

SEQ ID NO: 67 - DNA, 5596 bp, Homo sapiens
  source 1..5596: mol_type = genomic DNA
acagagcgcaggcgccggcggaggaggcaggcgggagggggccgactggcgcggctctggagagcgctcctccgccccgcgcgccgtccctgcgccccgctccgcccgcgcctctctcccagcgccgcgcctcgggcgtaaagagcgcgcccctcgcaccgcagccagtgccgaccgcagcagcccagccccagccttcctccgcctccgcctctctctcctcctcctcctccgccgccggacacgcagccgcaggccgggaccgggacgcagctggggagtcagggacgcgcgcagccagcccttccccctccggctcccgcaccgccggccgcctcccctcgccctcctactctcccctccctgctccttcgctttttcctcctcctcctctcccggccccggctgccagcaccatgtccgcagggggagattttgggaatccactgagaaaattcaagttggtgttcttgggggagcagagcgtcgggaagacgtctctgattacgaggttcatgtacgacagcttcgacaacacataccaggcaaccattgggattgacttcttgtcaaaaaccatgtacttggaggaccgcacggtgcgactgcagctctgggacacagctggtcaggagaggttccgcagcctgatccccagctacatccgggactccacggtggctgtggtggtgtacgacatcacaaatctcaactccttccaacagacctctaagtggatcgacgacgtcaggacagagaggggcagtgatgttatcatcatgctggtgggcaacaagacggacctggctgataagaggcagataaccatcgaggagggggagcagcgcgccaaagaactgagcgtcatgttcattgagaccagtgcgaagactggctacaacgtgaagcagctttttcgacgtgtggcgtcggctctacccggaatggagaatgtccaggagaaaagcaaagaagggatgatcgacatcaagctggacaaaccccaggagcccccggccagcgagggcggctgctcctgctaatgcagagccgacctgtggcttcccatgacactccttgcttgttgtgttgcttcctattggctagcttcctaaggggggagggaaccgagttatcaagatgggaggatttttcttttctctctgtctttaggagtagggtgggatggggagggaggctgggcatcagggatcacatcactcttaacggctgttacttaaacaactattttttggtttggttgtaatatattgtactttattaagattgccaaaaactgttaaaatttaaaaaaaatttaaatcatgtgtatacaattttttgccagataaaaatgtagtcatttttatttgaaagatgtgctttttgtttttgtatatttgtaaacttatagagaaccttttccacacacctcctccttcctgttctctttgaaccattcatcacctctgccttcctcctatccccagcccaataaattaaaacaattaactgagcaaattaattaggcttcagtctggggccatctggccccactctctaggccctactccagttaaatcaaacattggttgaacacatcagcctctgaaaaggtagctctgactcttgtctttccatggccagagtgggtcgtcaacctcgtgtggagctgagaccaaggtctgtgggctctaggcagcccggcagcagcccccagaggaactgctcccacctctccttctctttccctgaaaagccttgaagggggttgagggtacaggcaaccttgtgttctctgtgccctcagggactggcagagcaagaggccatcagaaggacgaggtgctgttggcaattgcagctgcgtggtgcgtggtttatagactggggtgccatgtgtacttctgaaaatattttaaccagcccacccagcagtcagtacacatgcctgggccaagtctaagaaactcagtgtgtgatgttgggcagtgtgccatcctgaagccttcctcctggatgtcagggaagctggaccacctggagttgatcctctgtgatggggaggcgggggggggggggggtgttgtctgcagaaccaccaccttgaaagatccatgttcttagagcctcagctgctaatattgttgagtgctttctggaaaagctgatttgttgctatgttttcaccccagtggcatggcgcagttgaagtctttgtggacatatctttgtggctactgtggctgcgctgtggtcacctgaggatgtggttcaggtggggtctccagaaggctgcttgcccccagagagccatctcttccatttttgattcattcctgagaatggcttagtcctgagaatctgcaaagtctctgaagagaactctctcttcccactaagtgatataacctacttggccagcattccaaatagactcagactcactgccctgagcctgcacagagccaggtgcaatgggcagtgagtctggccaaaatgtaaaccccaccctccctgctgtcattggctctccctccaggctgctgctgggcatgtcatagatgggatgaatgaccatctggggtatggtgggctgaactccacaaggtcgttggaatgaattttgaaacttctcagaaattaatctgaaatggcaggcagtaagtgcagcagaatctggcaggactgatatttgtgcagtgggcctgccacacaccctctgcctaggaggctgtatgccagagctcatctctagcaaggaatttatcttcaggtgaagaagagaggaagcaggatcccactgagcacctcttgcagaaggcaaggaccaatgtttgaacgtgtttcagatgtcatgtgtttagtttttatatgtgtgtacataattatatatagagatagacttgtgtatacaccttccacaaacacatcctcacggagatacatgtgctcaatgtatattcttgtttctcctgctctcaagttggaacttgcattgtgataagttttggaaattcaaaagtactgcaacgtgttgaaataagaaatacttaatattgtcaaataacttgtgattgtcttgccatcaataaaataatgcatagttgtgttaaactacaggcaaacatagcccacgggaatatctccaccatttctctatgtactttatatgcacgggtgtgcatgcctgtgtgggccagtgtccccccaccatgggcacacccccaagtggtgcttgttggaagagtgcttacttacctcgagagaatttacaggagtccacatgacagtgtcctgcctggttggcagcgggggccatcacctgtggcctctgagaccgccccatttacaggaatctgcactgggtctcagtgaatgaacgtatggtagatttttattttcttggcactaaagattagcttttttaaaaaaacatcccataaaacagtagaaactcagttcccttctcaggggcacacacatccaaggcaccgtggactgtgtttccatcctggcctagaggagcagtaaaagatagcatttggtttgacatggccttagtgagtcctgcctttcctagcatgcacacaacatcctctctttgagtttgggttctccccagccccttggcctctgaggctgagggtggcttcctcccatcctcccatctcagtggttccatggcgctcccttcggcacgtgcggtttccgcccctcccttcctggggtgtggaaaggcagccagacctctttttcagaagttgttggttccctgatttttgtcttgagcaggcaccatccctgtccccttgcttgcaggtagaattggcacagagttaaggttccataataaactgttaggtgctcttgaagaagatgtcatggcattgcctcagcaccccagcaagagcattgcatctttgctgaatccagatgggttagcagagggcaaattggaggattcctttgaggaaatgttctcaattgagcaattcagtccctctccccaaagactagacgtgacctggcagattagcaggatcctcttggccttcttttttggggatggtgggagtgaagggggtgaatcttaaaagcttatcaacaaagctcctgttctgcaggcatcaggaaagttgagtgtaagatggaaaaagcaaacagcctatttacaagtggaaacacattggttctgatcactcctaacttgctgggtgatcatacatacatcaacatgaaggtgtgacatcagaatcatgagcaccatggctgcagttgtgtgatccaaggctagttggcttggtagctgttggcagcagtttcccttggtcagaggcctccttgctatctatcgctgctctcttcttgtcatccttaaacattcagtcatgttcccaggtctgaggggagcaatggccagaccagtttcagtatctctggcaacagaaacaaggattgaaatggtggtagttaagagtgcagacacatctggggtgcaacgtctgcatcatcctggtggctagccagtgcggggaggccctgccctgggataccagttgtgacaataaagggatggccctcctggcttcagcatcctgcaggtagagcagcctcattccttcacaagatggaaggcaggtgaggatgtcaggatgagatgggaccaggtagccgaggacagtgaggcatgtctgagccacttgaggggcagctgaacagaaggatgtctgtgtgcatcaaccgagcagattgtgcgcacactcgtttccgaactgcctctccccaaagaatgtgaggcattgtcaccttgttacagctatgtgaagcagagctgtcacaacttttcctgtctacaggcaggaaaaccaggatggagaagggcaacttgcttattaagatcacccagcaagttaggagtgatcagaaccagtgaagtttccatttttcatccattgctgccctgtgtgtgaacaaatgtgactccatatttattacacagcaaggactgctgtgtgtgggtgtgttcctcagaccatctctactctttttggttaggacactaggttcttcccagcatatgagtgagaatattggtggtattttgggctcctctactgcacaagggccagtccccaagtctactctgattgattgtcatgggcacccacacagatcaggacaccctttgaatttttgtttgacatgaactatttaggtgttccaaaaccttgggatctcaatgtgcaaactccttggactccagacccctgtctggttggcactcccagcagtgtgtggtttcatcaggaaatccttgcagcaggggtttagatggttccttcaggaggtgggaagcctccatagcattgaaggccagcctgcatctcgcggtgtgtgtccacttctcccctcactttcccctcacccatccctttaaagtgattggctgtgaggagcacagagggtggcaattccatctgttatgggctcagaagacatagccgccatgttgatttcacattaaactgtgtaaaatgtggaaaagactcagcatgttggtcaaacaataaactgtcctaattctcaaaacaaa

SEQ ID NO: 68 - DNA, 10577 bp, Homo sapiens
  source 1..10577: mol_type = genomic DNA
gcagagacgcctggcgcacccgcgggagcggagccgtggcgcgctcgccccggacgccggccgcccctccgctcgccctactgagcgagcggcccggggcgccgaggggtccgcgccgcgcggggcgcaccgccctggccgccatgtgctcccagctctggttcctgacggaccggcgcatccgcgaggactacccgcaggtgcagatcctgcgcgccctccggcagcgctgctccgagcaggacgtgcgcttccgggcggtgcttatggaccagatcgccgtcaccatcgtcggcggccacctcggcctccagctaaaccagaaggccctcaccactttcccggatgtggtgcttgtacgggtacccacaccctcagtgcagtcagacagtgacatcactgtcctgcgacacctggagaagctgggctgccggttggtcaatcgcccacagagcatcttaaattgcatcaacaaattctggacgttccaagaactggctggacatggggtccccatgccagacaccttctcctatggtgggcatgaagacttttcaaaaatgattgatgaagctgagcccctgggctacccagtcgtggtgaagagcacacgaggccaccggggaaaagctgtttttctggcaagagataaacatcacctctctgacatctgccatctgatccgccacgatgtgccctacctgttccagaagtacgtgaaggagtcccatggaaaggacatccgggtggtggtggtagggggccaggtcataggctctatgcttcgctgctccactgatggacggatgcagagcaactgctctctcggtggcgtgggcgtcaagtgtccgctgacagaacaaggcaagcagttggctattcaggtgtccaacatcctaggcatggacttctgtggcattgatctccttatcatggacgatggctcctttgtggtgtgtgaggcaaatgctaatgttggcttcctagcctttgaccaggcatgcaacttagatgtgggtgggatcattgcagactataccatgtccttgctgccaaataggcagactggaaagatggctgtcctcccaggactgtcgagtccaagggagaagaacgagccggatggctgtgcttcagctcagggagttgcagagagcgtctataccatcaacagtgggtctacctctagtgaaagtgagcctgaactgggagagatccgggattcctcagcaagcacaatgggggccccaccctccatgctgcccgaacctggctacaacattaacaacaggattgcttctgagttaaaacttaagtgaattcctgctttttggcagcatttaaaccaaatcctactgcttccctagtagttttgagtgaataaaatctggactaatgtgatttcatttgcacagaaactagaaatcccatctgggcactcagcattttttctaacgatgatttaagcaaatggcctagctttgtggtttttacaaagacaaatataaaaacactcaagaacaacgtcccgactgatcagtatgagactgatgtctgctgtgagcacgtggatattacggctgacgctaaggcactgactctgctgttgcttctgacttttagcagtagaacccattagctcaaaatggctcttggaattatgtactcaaaagcaaccacgggatggaagcatgtgcagacgaggtggaatgtgactgcagtaataggttcttcactaagtgtgtgtgtgatgcttggaaaagacacagttcaaacactattagaggatcgatctctgctccccatcaatcaccatcttgaccatatttctccgacactcaggatatggtgttttagggagcttcctcattagcattttcagtgaagcactttgtagaaagtgagattgaacgttctttgacttcacaggttggcaaatgtcctgctttgtaactgggcctttctacaaattgctttgccactctgaatttatagaccctgcttcaactaacgtcagctctagcagcttcttgcacctttgctgcctttggcctcagctggaaatgcacttcaatgagctagtggccactcagtttatttcttaagcgtgcactagaatctaacaccatggactttgtttttcaatggactctgttcacttgtcatccagaagccagtgggtttttaccatagaaagctacttggagatcatcattcttcccctcctctactgaaactttgtgtactgcttagctgtagggggttttcttgtatgagtgggtgttggaggtctattgagtagatgggagccattggaaacactccaatccccttgaaactctacttttatctaagatccagacacctgtccttacccacttttccaaacggggaagactcaggtatcaggaattacccattgcacattttgcgggggggggggctaatgtagacatgacaccaagtgcttttcattttaattctctaagcagtagatgagatctttcccttgggagaaatcacagagatggagttttccatttctgcttatacgaaatatgactgaaaatgaacaaagccacctttttaaccaaatacactagaacatatgcactcaagagttagcataattggagttattttcttgatgttgcatgtgccaagaacacttagggttcccctttgggattacctttgaagccatgcaaggcaagaggcctcacaactttatagtcagaccttgtgttttatccaacccctcctcctgcccaccaagatcatcctgagctgcttcacccattttatttagcagacttattttctaatgctctttagctgttaaaaaaaaaaaatcattcctcaaaaaatagatctacccttaaaggacaaagatttaccaacactgaagacccttacaagttgacagtttctaaagagggttctaggaatgttctgtgtaaggtcagcttcactggaataaacatacatcttcccaaggtgactacttcaaaggcaacactacccattcgggtgcagaaattaccgtacttactctgccataaatgtaaaatgtgtataaagttaagctattgcattttttcataacacatttaaatagaaattggcttttaaagtattgtagttaaaaccagatctcaccaagtaatagttgtgcaattgacaaatgtttggtgccatgtaatggagatgtcacactacacagttgtagtttaaatgcagtccaacataaggtgcatgccactgaactgtaaccctagtaatgtgtcttgtacagttgaaaaataatacttcacctttatataggttaagatatgtaccttatctttaccactcagggtggctttaaaagacggacagctttaggtttgttctcactggaactggttacactttctgtgcaaacttaaccaagtgatgatcgtctggaagctgcattccctcatttccactgtttggtgtgaatctagtctccccactttatctaaaaggaggctagcagggccccagcctgagcagagcagctagagagtgcttccagattcctttgccaaaggaggcccattttccagctgtgagtgacatctgtagtcattggcttcattgcctggcactagccctgtctagaagtggagaaagctggatcacagctggaccacaaccagccccaatccaggatttcccataccatgctggctgtgggctctgtgagcctcagccaggtggccaggctatacttgtcacagctggccacaggcctgctgaaggaacatggggtgtagagccagagcctggttctggttctccgcttgaacagctgacctcgtgcaatcacaggaactctgcgtctccatgtttcactcagatagagaaaatgcctcctttgtttacctctttgaagctgaagtaagtgagactggtgcatgtaaattaatggtagtagagcacctcccacatctgattttataacagtttcccatcactccttaaagaaaaaggccaggccgggcgtggtggttcacgcctgtaatctcagcacttcgggaggccgaggcgggtggatcatgaggtcaggagattgagaccattctggctaacacagtgaacaaaaaattagtcgggcatggtggcgggcgcctgtagtcccagctacttgggaggctgaggcaggagaattgcttgaaccttggaggtggaggctgcagtgagccaagattatgccactgcactccagcctgggtgacagagcgagactccgtctcaaaaaaaaaaaaaaaaagccacacattcttggcattagcacaccaaacagtggtagggtaccctgagaaaggaatgcagcttctcagaggcctcacttccagccagggaaacttaagacctgaaaggaataaggctctttgtgtattcagacagccttctggatgtatcgtatataacctgaaactctccactcttgtataccaactaacaaagtcttccttatgtaacatagattgcagtgtctgtatatggtcttcccattatctggtagtagcatcttgaaactggtcaaaacttgaatgccaatagctcagtcactcagaatatacgattctgcacttgggaaaaatgtcttcatgctgtttttctgatcgggtatggggttgccagaatgaggatttggggctttaggtctttcggatctcagaaacaatataagtagatcataagccactagtgtcacaatttcaggcccattgcttccagctaaaaggagattccatgaccatggggaccaggagaggaggaaagggaaaagggaaaggtctggaatagttttgtgaaagaaataaaaaattgggatgatttggaattctagtaggaaggaggaaaaaatggctcttctgtgggctgttttaatgaaagaatcatgaatatgtattgatcagaggtagatgtctttgaagctgttctgcataaagataatgagatgcatggatttccatccaccctggagaagtgggggagatgtgcttcctcccttctttaaatcctgaggaatgctggtcttgagaacactgaatgcaaatctgaggaagatgtaagcggctggctggggagttttaggaccacccaaataaaagctgaccctcccagaggctaatgaaccacagtgctgtgtgcaggcatagccttagcaggtcagtttaaggagtcaggtcagagaataaatccaaccttagtcaagtcactacagtttaatattttcctttgcaactggaggtaagcctgtttgccattagtctgagtggatgtcttgggaaggcccctgaaaatagccatatttgtcactgtccctaaagtgaatgttcatagatctgggactgtttttgactgtacctctgagactcaagatcaggacagattgttactttaaaatgttacctggaagacgattcttctaagggctataatgttataatcttttcctaaaactaaatcatgcctccattccaggtctatggctagactctgcagccctccctcctaaaaccacagggctgaatgtggaaactttcttcttttaagcctttccatttccttctctaggtacagctcctgtgccaatatgcactgtctcaagccctgtgctggcattccaacctggtttggtaattttgctgtgaggttcatgttctctggggctccagggcaacactaagaaagtcgcttatgatccccatttgagaccatggaattagcattttggagaccagtctagtcccccatttcataagtagggaatcttacttagttccagaggccatgttcacttgctagtcaatagcaaggctggactcaggcagtctgattcctcatccactttggcatactgccagaggtagctccatccctccctggaatctagcacatgctcccatcctgggcctaccctccgcctcctccccttctgatgagcctctgttctggatcaacacggtgttgatgtgccagctgtcaagggttcagcacttggatggcttctaggtgagagccccaaaggcatgtatatttccatgatgcaagctggctcatggcccgcaccgtctttagaccagccaagaccatttaactctaccccacactttcagtggtgggatgtgaggaagaaagcccatgccaagctaactgaaagcttatttggctccaattcggctgatgttccctcactgcagaatgtcctggaaaccaagggtttgcagctcctaaacctattgcattaggcacacccaagaagaaatcctgttcgatgcacatgctccagtttcaatcagcaacaaggtcaaaagtttccccccactttctgttccacagtgcgttccccttgcagccagacattaggcacagattcatccctattgtaacccaaatgtcagctgagggctccttgcctcttgacatccagagaggtctgataactgggctctgtttctgaacctctagatttctgtgatttctcaaagacttatatttttgcagccactgctgactccacacttctgctacctcattttaaatagcgtcttatccctgcaatgcatcagctgagacatgaagctcagtttcctcgcccatttccacatagcaaaattcaagcgagtgagtgtgtgtgcctgcgtgtgtctcagtgacgtgggaaggaatggcagatgctacaacagtgcagactcggcaccccatttatagaatgttctttttatatactatggaggaatctccactcaccttccactccacgtggtccctgtctcctgtccttgggacagagcagttccatttatattctcacatacaaccagatggatagccattccttttcttaatttgattcctgccaacaccctaaccgtcatgacttcaccttttaccatgggtggcttattaaaaacattttcctggacgaagacagttccctccccaagcggaattcgagtgccatctcttaaggtgatgcatttttaattcccactgcacaagccaggcatcacaatgaccatattcaccactcgttgtcaggggtacaccttttggaaggcagcgtgggctaatgtgaatggtactagtctgagtcagatccagttcaaatccaggtaacttaaagcttgtgtgacccagggacactttacttcaacttgttaaaccttaattttgtcacctataaatgtgatacataaacaaacttgaatgtgtcgcacttaaccacatgcctggctttccataggttcctttcctcttgcttcccccactgtgagcaacacaagcatcgccttcttgtgatcatggctcagtgccaagggataaaagctggcagacagcagctttacagctgggaaaaagagctgctgggggcagggctggcggtagctcaagcatgctgtggttcaggctaatgactggaaggtcccatccatccttgagattttatgacgctatgtgcaagatctagcgaacgtatttggctaagaaaaatcctggaggagagactgcctcagacaactggggggctggaaagccgacctggaaagcagcctgaccttggatgatttagcacctgggttttttggctcttcctgtattaggtttgctggcttcgcatcaaatggaaggaggtggtgccagggacgtgaattaaacctgtcaaatagagggtgaggggagtgcagaaggctgggggaaggaggggctgtgtggggaactctgatagtctgacagcaccatctggacttggcagggatgacagccctgagatcagatggaactaaccaataaagctgctaaaattctgctgtcctgggcctatccggagtgggactcccgtaatctcctatttagaaggaacactgatctgcaaggggagagctggggcccttaatcattctgcagagctgataacggtcactgtggaggcaggactaggcgggtgcctggtttgcttagtcccaccttgaggtcccgagttccagtggtgtgccgaagtggtttgcactggcccatgagagtggattgttgaatttccaggaattttgtaagctggttggtaaacacagccacagtgaccattagagattatattaacctgcaattaaataaagtatgttaaaaacagtaataaatactcaaaactcatcacttcctaattattttactattatctatgttcttgaggatactgacatctattgtattggtatggtgtgctacaatataatggcgtgctattgcacatctcttcccaacctgaacattcagtgacatcacatcagtagctggggaatcggccctattgagagtatttacaccacagagattggcaaacactctatatcaagacccccctccccgagagctggttgttaaacatttgtcagcacaccactgctgctgggagacagagagaagaaactgatacacgtgagcactgaaacatcttagttccagggaggtagcatttgtgttgtatttctcagaagcaagtgtcctagtgtaatgtgcacaagtggcatgaagtttaacacctaaaagactgcttctctgcttgttctgtctaacttctgtgatttcaacacttgagaaagatgtttactttgctactaaacttattggagtgatttgtaaagttcactgtcacctgtgggcagtagttcagctaaattccctccaggaaagtggactgtagcttttcctgcctaactggcaaaatgaaaatatgctgaaaagaatacatcttaactaggcaccagaatagctcatttccaagtggtttatacagcaataacagtgttaacttctccaacagagacgaaatcatttataaatgattgttttgctgtttttgctttttattaaaattattactatggaacagaatgctttgtttgcctttgtctccaaaattctgtgccttttggtggttcttgtaaagtaagaatcacatgctgaatctacattttattattttcatatcataaaatgaggaagagccttcacagaagaaacttcaccctattcacttgcacaccagctgcgttctgaaaaactataattttcctttcaaacgcagagtgccgtctgaattgtaatactgccaactctcaactgtcaagatgatggaggaaaatactaatgcagatggcgtgagcttgtcacgggcctgctcagcgtgctctcccgggctgacattcgaattaagattcaaatagcaagtgagagctgaaagctgtttaaaatggctgtgtggtgcagtaaggggtctcaggggattgaaagaatctgtgtactcaccagaagaactctgacaggtcaggcagggccctcaggacttgtgtttgcctgacatttcaagaggcttcctgggagaagcggtgataagaccctgggggactaacagtcaaaacaaggctcaagacccttgaactaaggctattccccagcttctgagggtcagggagtccaaattgaggtcagagcatcaaagggatagtgcttgattgctgcagccgaggatgatttggggtatgacattatgtaccattcatattgtccttgaacccaaacactggggaatgtggcctatgtttctttcttcttctcttacatgaaaatcgtcaaactacacaaaagtggagagaatagtaaactccccagaaaaggacgtttccatttgcctttaccacatataaaaagattagcagtaatcccttgataccatctaattcccagtccgtattcaaattcccccattgtctcaaagagaactgcatttagctccatggtagaccagcgtatttggtgagaaaggaagtactttggaaaggttgctgatggcttccctaccctcttgacggtggctctggcagaaaccatgctgcaggggcacaggtggagagatgttcaggctggctgcagatgcctttccaagttgtgggatcttgggatttcctctcctcaaagaataagcctgtgacacttccccttctacttcccagagttacatggtgtccctctaggactctcccagcttcatgctgctggcacccacttgtctatcacgctggtatccgctgcttttcctgcagctgctgctttcactaatggcacttcttaaatctggtgagaggcttctctcaaaaatgatcgctcttccactaaccttaggtctactgtactcttggtgaatcacagctctttagagctcctcaagggcctaagcatcttttgttagaaaattctggctgaaattctcatgagatgggtcagggaaccttggagtggcagcacttgtgtcacatctaaaggcactcctctgactaaagagaagtggcagctgagcacactgcctgcccagggattttcttctgctctaaaggacattattaagacaactggtctgtactgtattgcctgatagtatcatatcaatgtcaatttcctcattttggtgattgttctaataagaatacgtaagaatgtgtccttgtttttagaaaatacacattaaatatcgagggatagaggta

SEQ ID NO: 69 - DNA, 3156 bp, Homo sapiens
  source 1..3156: mol_type = genomic DNA
acagctgtgtttggctgcagggccaagagcgctgtcaagaagacccacacgcccccctccagcagctgaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggatgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatctgtctccaagaccttggcctctccttggaggacctttaggtcaaactccctagtctccacctgagaccctgggagagaagtttgaagcacaactcccttaaggtctccaaaccagacggtgacgcctgcgggaccatctggggcacctgcttccacccgtctctctgcccactcgggtctgcagacctggttcccactgaggccctttgcaggatggaactacggggcttacaggagcttttgtgtgcctggtagaaactatttctgttccagtcacattgccatcactcttgtactgcctgccaccgcggaggaggctggtgacaggccaaaggccagtggaagaaacaccctttcatctcagagtccactgtggcactggccacccctccccagtacaggggtgctgcaggtggcagagtgaatgtcccccatcatgtggcccaactctcctggcctggccatctccctccccagaaacagtgtgcatgggttattttggagtgtaggtgacttgtttactcattgaagcagatttctgcttccttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccgtatctcaggaagtccagccacttgactggcacacacccctccggacatccagcgtgacggagcccacactgccaccttgtggccgcctgagaccctcgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagtggacacgtgaggggggggtgggtgagagagacaggcagctcggattcaactaccttagataatatttctgaaaacctaccagccagagggtagggcacaaagatggatgtaatgcactttgggaggccaaggcgggaggattgcttgagcccaggagttcaagaccagcctgggcaacataccaagacccccgtctctttaaaaatatatatattttaaatatacttaaatatatatttctaatatctttaaatatatatatatattttaaagaccaatttatgggagaattgcacacagatgtgaaatgaatgtaatctaatagaagcctaatcagcccaccatgttctccactgaaaaatcctctttctttggggtttttctttctttcttttttgattttgcactggacggtgacgtcagccatgtacaggatccacaggggtggtgtcaaatgctattgaaattgtgttgaattgtatgctttttcacttttgataaataaacatgtaaaaatgtttcaaaaaaataataaaataaataaatacgaa

SEQ ID NO: 70 - DNA, 2102 bp, Homo sapiens
  source 1..2102: mol_type = genomic DNA
aagagtagaatcgtgtcgcggctcgagagcgagagtcacgtcccggcgctagcccagcccgacccagaatgaaaaaggcaggcattgacctccctctgaggcagtttccaggcccaccgtggtgcacgcaaaccacttcctggccatgcgctccctcctgcttctcagcgccttctgcctcctggaggcggccctggccgccgaggtgaagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctagggctcgtgtcgctgggcggcaaggcgaccacggcgtcgcaggccaaggcagtgctgagcgccgagcagctgcgcgacgaggaggtgcacgccggcctgggcgagctgctgcgctcactcagcaactccacggcgcgcaacgtgacctggaagctgggcagccgactgtacggacccagctcagtgagcttcgctgatgacttcgtgcgcagcagcaagcagcactacaactgcgagcactccaagatcaacttccgcgacaagcgcagcgcgctgcagtccatcaacgagtgggccgcgcagaccaccgacggcaagctgcccgaggtcaccaaggacgtggagcgcacggacggcgccctgctagtcaacgccatgttcttcaagccacactgggatgagaaattccaccacaagatggtggacaaccgtggcttcatggtgactcggtcctataccgtgggtgtcatgatgatgcaccggacaggcctctacaactactacgacgacgagaaggaaaagctgcaaatcgtggagatgcccctggcccacaagctctccagcctcatcatcctcatgccccatcacgtggagcctctcgagcgccttgaaaagctgctaaccaaagagcagctgaagatctggatggggaagatgcagaagaaggctgttgccatctccttgcccaagggtgtggtggaggtgacccatgacctgcagaaacacctggctgggctgggcctgactgaggccattgacaagaacaaggccgacttgtcacgcatgtcaggcaagaaggacctgtacctggccagcgtgttccacgccaccgcctttgagttggacacagatggcaacccctttgaccaggacatctacgggcgcgaggagctgcgcagccccaagctgttctacgccgaccaccccttcatcttcctagtgcgggacacccaaagcggctccctgctattcattgggcgcctggtccggcctaagggtgacaagatgcgagacgagttatagggcctcagggtgcacacaggatggcaggaggcatccaaaggctcctgagacacatgggtgctattggggttgggggggaggtgaggtaccagccttggatactccatggggtgggggtggaaaaacagaccggggttcccgtgtgcctgagcggaccttcccagctagaattcactccacttggacatgggccccagataccatgatgctgagcccggaaactccacatcctgtgggacctgggccatagtcattctgcctgccctgaaagtcccagatcaagcctgcctcaatcagtattcatatttatagccaggtaccttctcacctgtgagaccaaattgagctaggggggtcagccagccctcttctgacactaaaacacctcagctgcctccccagctctatcccaacctctcccaactataaaactaggtgctgcagcccctgggaccaggcacccccagaatgacctggccgcagtgaggcggattgagaaggagctcccaggaggggcttctgggcagactctggtcaagaagcatcgtgtctggcgttgtggggatgaactttttgttttgtttcttccttttttagttcttcaaagatagggagggaagggggaacatgagcctttgttgctatcaatccaagaacttatttgtacattttttttttcaataaaacttttccaatgaca

SEQ ID NO: 71 - DNA, 3095 bp, Homo sapiens
  source 1..3095: mol_type = genomic DNA
attccggcacctcccttaggcgccagggacagccgagcgttacctggtcccgggcagcggagttctttacccaccccagttctggttctgacgccctagctcattccgcaaatttagggcttgggtctggcttgttcccctccggctcgaaccacctcttctctgagccgagccagctaccggggctcctggaattgccacccctccctgggcacccttgaggcctccgtggagggacgtcacggggcagagcgggacgtgagcctgagtttgctgcaggcgtgctctgtgtggtggctgggttctgccaatccccgtgcccaccgggtgggcgcggccgggaagctcctgcccctccctgctggtcggcgtcacgcgtgacgtcccgcgtgatggctgggagggcccggcggcgacagcggaggcagagaggaaggcggttctgagagcttcagagagcgatggaaagcaaaatgggtgaattgcctttagacatcaacatccaggaacctcgctgggaccaaagtactttcctgggcagagcccggcactttttcactgttactgatcctcgaaatctgctgctgtccggggcacagctggaagcttctcggaacatcgtgcagaactacagggccggcgtggtgaccccagggatcaccgaggaccagctgtggagggccaagtatgtgtatgactccgccttccatccggacacaggggagaaggtggtcctgattggccgcatgtcagcccaggtgcccatgaacatgaccatcactggctgcatgctcacattctacaggaagaccccaaccgtggtgttctggcagtgggtgaatcagtccttcaatgccattgttaactactccaaccgcagtggtgacactcccatcactgtgaggcagctggggacagcctatgtgagtgccaccactggagctgtggccacggccctgggactcaaatccctcaccaagcacctgccccccttggtcggcagatttgtgccctttgcagcagtggcagctgccaactgcatcaacatccccctgatgaggcagagagagctgcaggtgggcatcccggtggctgatgaggcaggtcagaggcttggctactcggtgactgcagccaagcagggaatcttccaggtggtgatttcaagaatctgcatggcgattcctgccatggccatcccaccactgatcatggacactctggagaagaaagacttcctgaagcgccgcccctggctgggggcacccctgcaggtgggactggtgggcttctgcctggtatttgcaacccccctgtgctgtgccctattcccccagaagagctccatacacataagcaacctggaaccagagctgagagctcagatccatgagcaaaaccccagcgttgaagtggtctactacaacaaggggctttgaggagggtcagcctctgtcccctccctcacttccttgggctgctgctttagtggagtcatgtcacccctaccacttggctatctgcctagcactgggcaggggccttggtgggcagatggcaattgagggtagcaacctattagggtgggggagggacctccataaggcttttcctcccttctctggtttcaaagatcagagcacataacccctcctgtgcttgagtgtccatgcatatacatacatgatacacatgtgtatgtgtacattgggtcctgaaagctagaagcaggcatgctagcctagtatgttctgacatctggcttcccttctcagcctcatgtccacctgcctgccagccaggctacaggtgtgacttccttctctaaactgttacaccagccaagttatttttgatggcacctcatcccttctagaaataggaggagccccaggatctcaggacagagacttatagacactagtaggacaaagcgggctgaatccttcaggtttctgatacctagctccccaagctgactgggctggcagaggagaacatgttgagacaagggaggcaggggacttatgcatccctcagtgccatcccttgtatcctggaatagctccatttcccctcctcctctctaccagacaaaggagtgcctgtgtcctgtactgccctcgctgtctcccccaccaccctacttgacagcgtgggcatcttcaggcacagccttgggagttcctggtgtgctctgacatcatgacctcaaatctaaatcctccaatcccaactccctttcccaaacaaaaagccacagaggcagagcaagcattcccctttaagagcttccactgcaccccctcccaagggacacagcggtaggaatggtgcttaaactccacaggtatcagagagggtgtaactaggacatcctcaagggcagctaggccccgaatgtacaatgttaagacagggaattttgtgttccattgacttttttttttttttttaatggagtttcactattttgcccaggctggagtgcgatggtgcgatcttggctcactgcaacctctgcctcctgggttcaagtgattctcttgcctcagtctcccgagtagtggaaattacaggtgtgtgctaccacatcttgctagttttgtatttttagcagagatgggggtttcaccatgttggccaggctagtctcgaactcctgacctcaggtgatccacctgccttggcctcccaaagcactgggattacaagcatgagccactgtgcccagcctgttccactgacatttcttagacattcagcaaaacccccaccttaacctcttttctttcttgagggttggtcctgtccccacctccaccctcccaccccctggaagaggaagggcccgggcatcagtggctagtccaaataaaatatgggcttggggatggaatgggtggtggtaagttcacagagtgtagttagatcccaactcccatgacctctggcttcagtggtgggtggggcagggcagatgaaagggcttcagtgggaacctctgagagcattttcctgttccccctatcaaccgcccccagtgataacatctgtgaagccagccattactcaataaactgcaaacttgtcta

SEQ ID NO: 72 - DNA, 2616 bp, Homo sapiens
  source 1..2616: mol_type = genomic DNA
atggacggcgagagcgaggtggatttttctagcaacagcataacccctttgtggcggaggcggtcgattcctcagccccaccagcttctgggccggagcaagccgaggccccagtcctaccagagccccaacgggttactaattacggatttcccggtggaggacggagggacgctctccgcagcgcagattcccgcccaggtgcccaccgcctcggacagcaggacggtacataggagccccctgcttctgggcgcccagcggagagcggtggccaatggtgggacggcatccccggagtacagggctgcctctcctcgacttcgacggcccaagtcacccaagctccccaaagcggtgcctggcggctccccgaaatccccagcaaatggcgcggtgaccttgcctgcgccgccgccgccgccggttctgcgccccccgcggactcctaacgcgcccgccccctgcacccccgaggaggaccttactgggttgactgccagcccggtgccttcgcccactgcaaatggccttgccgctaataacgactctcctgggtcaggttcgcagtccggccggaaggcaaaggaccccgaacgggggctctttcctgggccccagaaaagttcttcggaacaaaaactccccctccaaaggctgccctcccaggagaacgagctcctcgagaatccttccgtggttttgagtacaaacagccccgccgccctcaaagtggggaagcagcagatcattccgaagagtctggcctcggaaattaaaataagtaaatccaacaatcaaaatgtggagccccacaagagactcctcaaggtgcgcagcatggtggagggcctaggaggacccctgggtcacgcaggggaggagagtgaggtcgataacgacgtggatagcccagggtctctgcggagaggcttgcggtccacgtcttatcgcagggcagtggtcagtggctttgattttgacagtcctaccagctcgaagaagaagaacagaatgtcccagcctgttctgaaagtggtgatggaagacaaggagaagttttccagtctgggaaggataaagaaaaaaatgctgaaaggacaaggaacatttgatggggaagaaaatgctgtcctgtatcaaaactacaaggaaaaggcccttgacattgattctgatgaagagtcagagcccaaagaacagaagtcagatgaaaaaattgtgattcaccataagccattgagatccacatggagccaactctctgcggtgaaaagaaagggattatctcagacagtaagccaggaggaaagaaagagacaagaggctatctttgaagtcatatcctctgaacattcatatttactcagcttggagatcttgatacgaatgtttaaaaattctaaagaactgagtgatacaatgactaaaaccgagaggcaccatcttttctccaatattacagatgtctgtgaggcaagcaaaaagttctttatagagttggaagcaagacatcagaataatatcttcatagatgacataagtgacattgtggaaaaacacacagcatccacatttgacccatatgtgaaatactgcacaaatgaagtctaccaacaacgaacactacaaaaattgttagctaccaatccatcctttaaggaagtattgtcaaggattgagtcccatgaagactgtaggaacttacccatgatctcttttctcattctccccatgcagagggtgacccgccttcccctgctgatggatactatctgtcaaaaaacacctaaggactctccgaagtatgaagtctgcaaaagagccttgaaggaagttagcaagttggttcgactatgcaatgagggcgcccggaagatggaaaggactgagatgatgtacacaattaactcccagctggaatttaaaattaagccttttcctttagtctcctcttcccggtggttggtaaaaagaggtgaattgacagcctatgttgaagacactgtgcttttctcaagaaggacatccaaacagcaagtctacttctttctctttaacgatgtgctcattatcaccaagaagaagagtgaagaaagttacaacgtcaatgattattccttaagagatcagctattggtggaatcttgtgacaatgaagagcttaattcttctccagggaagaacagctccacaatgctctattcaagacagagctctgccagtcacctctttactctgacagtccttagtaaccacgcgaatgagaaagtggagatgctactaggagctgagacgcagagcgagcgagcccgctggataactgccctgggacacagcagcgggaagccgcctgcagaccgaacctcactgacccaggtggaaatcgttaggtcatttactgctaagcagccagatgaactctccctgcaggtggctgacgtcgtcctcatctatcaacgtgtcagcgatggctggtatgagggggaacgactacgagatggagaaagaggctggtttcctatggaatgtgccaaggagataacatgtcaagctacaattgataagaatgtggagagaatgggacgcttgctaggactggagaccaacgtgtag

SEQ ID NO: 73 - DNA, 2707 bp, Homo sapiens
  source 1..2707: mol_type = genomic DNA
gtaatacctaagagggaagtggcttcatttcagtggctgacttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctataaaaatgtaaatgcaaggtcacacatattaatgacagcctgttgtattaatgatggctccaggtcagtgtctggagtttcattccatcccagggcttggatgtcaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacatgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaatacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcacatacaaaaa

SEQ ID NO: 74 - DNA, 1723 bp, Homo sapiens
  source 1..1723: mol_type = genomic DNA
gaactgactcggagcgaggagacccgagcgagcagacgcggccctggcgcccgccctgcgcactcaccatggcgatgcatttcatcttctcagatacagcggtgcttctgtttgatttctggagtgtccacagtcctgctggcatggccctttcggtgttggtgctcctgcttctggctgtactgtatgaaggcatcaaggttggcaaagccaagctgctcaaccaggtactggtgaacctgccaacctccatcagccagcagaccatcgcagagacagacggggactctgcaggctcagattcattccctgttggcagaacccaccacaggtggtatttgtgtcactttggccagtctctaatccatgtcatccaggtggtcatcggctacttcatcatgctggccgtaatgtcctacaacacctggattttccttggtgtggtcttgggctctgctgtgggctactacctagcttacccacttctcagcacagcttagctggtgaggaacgtgcaggcactgaggctggagggacatggagccccctcttccagacactatacttccaactgccctttcttctgatggctattcctccaccttattcccagcccctggaaactttgagctgaagccagcacttgctccctggagttcggaagccattgcagcaaccttccttctcagccagcctacgtagggcccaggcatggtcttgtgtcttaagacagctgctgtgaccaaagggagaatggagataacaggggtggcagggttactgagcccatgacaatgcttctctgtgactcaaaccaggaatttccaaagatttcaagccagggagaagggttcttggtgatgcagggcatggaacctggacaccctcagctctcctgctttgtgccttatctacaggagcatcgcccattggacttcctgacctcttctgtctttgagggacagagaccaagctagatcctttttctcacctttctgcctttggaacacatgaagatcatctcgtctatggatcatgttgacaaactaagttttttttatttttcccattgaactcctagttggcaattttgcacattcatacaaaaaaatttttaatgaaatgatttcattgattcatgatggatggcagaaactgctgagacctatttccctttcttggggagagaataagtgacagctgattaaaggcagagacacaggactgctttcaggctcctggtttattctctgatagactgagctccttccaccagaaggcactgcctgcaggaagaagatgatctgatggccgtgggtgtctgggaagctcttcgtggcctcaatgccctcctttatcctcatctttcttctatgcagaacaaaaagctgcatctaataatgttcaatacttaatattctctatttattacttactgcttactcgtaatgatctagtggggaaacatgattcattcacttaaaatactgattaagccatgggcaggtactgactgaagatgcaatccaaccaaagccattacattttttgagttagatgggactctctggatagttgaacctcttcactttataaaaaaggaaagagagaaaatcactgctgtatactaaatacctcacagattagatgaaaagatggttgtaagctttgggaattaaaaacaaacaaatacattttagtaaatatatatttttaaatagtc

SEQ ID NO: 75 - DNA, 1993 bp, Homo sapiens
  source 1..1993: mol_type = genomic DNA
agacgccgcgcggggcggggattggtctgtggtcctctctcggctcctcgcggctcgcggcggccgacggttcctgggacacctgcttgcttggcccgtccggcggctcagggcttctctgctgcgctcccggttcgctggacgggaagaagggctgggccgtcccgtcccgtccccatcggaaccccaagtcgcgccgctgacccgtcgcagggcgagatgagcgcggacgcagcggccggggcgcccctgccccggctctgctgcctggagaagggtccgaacggctacggcttccacctgcacggggagaagggcaagttgggccagtacatccggctggtggagcccggctcgccggccgagaaggcggggctgctggcgggggaccggctggtggaggtgaacggcgaaaacgtggagaaggagacccaccagcaggtggtgagccgcatccgcgccgcactcaacgccgtgcgcctgctggtggtcgaccccgagacggacgagcagctgcagaagctcggcgtccaggtccgagaggagctgctgcgcgcccaggaagcgccggggcaggccgagccgccggccgccgccgaggtgcagggggctggcaacgaaaatgagcctcgcgaggccgacaagagccacccggagcagcgcgagcttcggcctcggctctgtaccatgaagaagggccccagtggctatggcttcaacctgcacagcgacaagtccaagccaggccagttcatccggtcagtggacccagactccccggctgaggcttcagggctccgggcccaggatcgcattgtggaggtgaacggggtctgcatggaggggaagcagcatggggacgtggtgtccgccatcagggctggcggggacgagaccaagctgctggtggtggacagggaaactgacgagttcttcaagaaatgcagagtgatcccatctcaggagcacctgaatggtcccctgcctgtgcccttcaccaatggggagatacagaaggagaacagtcgtgaagccctggcagaggcagccttggagagccccaggccagccctggtgagatccgcctccagtgacaccagcgaggagctgaattcccaagacagccccccaaaacaggactccacagcgccctcgtctacctcctcctccgaccccatcctagacttcaacatctccctggccatggccaaagagagggcccaccagaaacgcagcagcaaacgggccccgcagatggactggagcaagaaaaacgaactcttcagcaacctctgagcgccctgctgccacccagtgactggcagggccgagccagcattccaccccacctttttccttctccccaattactcccctgaatcaatgtacaaatcagcacccacatcccctttcttgacaaatgatttttctagagaactatgttcttccctgactttagggaaggtgaatgtgttcccgtcctcccgcagtcagaaaggagactctgcctccctcctcctcactgagtgcctcatcctaccgggtgtccctttgccaccctgcctgggacatcgctggaacctgcaccatgccaggatcatgggaccaggcgagagggcaccctcccttcctcccccatgtgataaatgggtccagggctgatcaaagaactctgactgcagaactgccgctctcagtggacagggcatctgttaccctgagacctgtggcagacacgtcttgttttcatttgatttttgttaagagtgcagtattgcagagtctagaggaatttttgtttccttgattaacatgattttcctggttgttacatccagggcatggcagtggcctcagccttaaacttttgttcctactcccaccctcagcgaactgggcagcacggggagggtttggctacccctgcccatccctgagccaggtaccaccattgtaaggaaacactttcagaaattcagctggttcctccaaa

SEQ ID NO: 76 - DNA, 2180 bp, Homo sapiens
  source 1..2180: mol_type = genomic DNA
agttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaatgaataatgcttattatgcatctgaagtgttattttatgtgttagattataataaagtaagctgctagtattattttta

SEQ ID NO: 77 - DNA, 2512 bp, Homo sapiens
  source 1..2512: mol_type = genomic DNA
gatggttgtctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaa

SEQ ID NO: 78 - DNA, 984 bp, Homo sapiens
  source 1..984: mol_type = genomic DNA
accagatcccagaggctgaacacctcgaccttctctgcacagcagatgatccctgagcagctgaagaccagaaaagccactaagactttctgcttaattcaggagcttagaggattcttcaaagagtgtgtccagcatcctttgaagcatgagttcttaccagcagaagcagacctttaccccaccacctcagcttcaacagcagcaggtgaaacaacccagccagcctccacctcaggaaatatttgttcccacaaccaaggagccatgccactcaaaggttccacaacctggaaacacaaagattccagagccaggctgtaccaaggtccctgagccaggctgtaccaaggtccctgagccaggctgtaccaaggtccctgagccaggttgtaccaaggtccctgagccaggctgtaccaaggtccctgagccaggttgtaccaaggtccctgagccaggctacaccaaggtccctgaaccaggcagcatcaaggtccctgaccaaggcttcatcaagtttcctgagccaggtgccatcaaagttcctgagcaaggatacaccaaagttcctgtgccaggctacacaaagctaccagagccatgtccttcaacggtcactccaggcccagctcagcagaagaccaagcagaagtaatttggtgcacagacaagcccttgagaagccaaccaccagatgctggacaccctcttcccatctgtttctgtgtcttaattgtctgtagaccttgtaatcagcacattgtcaccccaagccatagtctctctcttatttgtatcctaaaaatacgtactataaagcttttgttcacacacactctgaagaatcctgtaagcccctgaattaagcagaaagtcttcatggcttttctggtcttcggctgctcagggttcatctgaagattcgaatgaaaagaaatgcatgtttcctgctcttccctcattaaattgcttttaattcca

SEQ ID NO: 79 - DNA, 2712 bp, Homo sapiens
  source 1..2712: mol_type = genomic DNA
ctcacttccctggagccgcccgcttgcccgtcggtcgctagctcgctcggtgcgcgtcgtcccgctccatggcgctcttcgtgcggctgctggctctcgccctggctctggccctgggccccgccgcgaccctggcgggtcccgccaagtcgccctaccagctggtgctgcagcacagcaggctccggggccgccagcacggccccaacgtgtgtgctgtgcagaaggttattggcactaataggaagtacttcaccaactgcaagcagtggtaccaaaggaaaatctgtggcaaatcaacagtcatcagctacgagtgctgtcctggatatgaaaaggtccctggggagaagggctgtccagcagccctaccactctcaaacctttacgagaccctgggagtcgttggatccaccaccactcagctgtacacggaccgcacggagaagctgaggcctgagatggaggggcccggcagcttcaccatcttcgcccctagcaacgaggcctgggcctccttgccagctgaagtgctggactccctggtcagcaatgtcaacattgagctgctcaatgccctccgctaccatatggtgggcaggcgagtcctgactgatgagctgaaacacggcatgaccctcacctctatgtaccagaattccaacatccagatccaccactatcctaatgggattgtaactgtgaactgtgcccggctgctgaaagccgaccaccatgcaaccaacggggtggtgcacctcatcgataaggtcatctccaccatcaccaacaacatccagcagatcattgagatcgaggacacctttgagacccttcgggctgctgtggctgcatcagggctcaacacgatgcttgaaggtaacggccagtacacgcttttggccccgaccaatgaggccttcgagaagatccctagtgagactttgaaccgtatcctgggcgacccagaagccctgagagacctgctgaacaaccacatcttgaagtcagctatgtgtgctgaagccatcgttgcggggctgtctgtagagaccctggagggcacgacactggaggtgggctgcagcggggacatgctcactatcaacgggaaggcgatcatctccaataaagacatcctagccaccaacggggtgatccactacattgatgagctactcatcccagactcagccaagacactatttgaattggctgcagagtctgatgtgtccacagccattgaccttttcagacaagccggcctcggcaatcatctctctggaagtgagcggttgaccctcctggctcccctgaattctgtattcaaagatggaacccctccaattgatgcccatacaaggaatttgcttcggaaccacataattaaagaccagctggcctctaagtatctgtaccatggacagaccctggaaactctgggcggcaaaaaactgagagtttttgtttatcgtaatagcctctgcattgagaacagctgcatcgcggcccacgacaagagggggaggtacgggaccctgttcacgatggaccgggtgctgacccccccaatggggactgtcatggatgtcctgaagggagacaatcgctttagcatgctggtagctgccatccagtctgcaggactgacggagaccctcaaccgggaaggagtctacacagtctttgctcccacaaatgaagccttccgagccctgccaccaagagaacggagcagactcttgggagatgccaaggaacttgccaacatcctgaaataccacattggtgatgaaatcctggttagcggaggcatcggggccctggtgcggctaaagtctctccaaggtgacaagctggaagtcagcttgaaaaacaatgtggtgagtgtcaacaaggagcctgttgccgagcctgacatcatggccacaaatggcgtggtccatgtcatcaccaatgttctgcagcctccagccaacagacctcaggaaagaggggatgaacttgcagactctgcgcttgagatcttcaaacaagcatcagcgttttccagggcttcccagaggtctgtgcgactagcccctgtctatcaaaagttattagagaggatgaagcattagcttgaagcactacaggaggaatgcaccacggcagctctccgccaatttctctcagatttccacagagactgtttgaatgttttcaaaaccaagtatcacactttaatgtacatgggccgcaccataatgagatgtgagccttgtgcatgtgggggaggagggagagagatgtactttttaaatcatgttccccctaaacatggctgttaacccactgcatgcagaaacttggatgtcactgcctgacattcacttccagagaggacctatcccaaatgtggaattgactgcctatgccaagtccctggaaaaggagcttcagtattgtggggctcataaaacatgaatcaagcaatccagcctcatgggaagtcctggcacagtttttgtaaagcccttgcacagctggagaaatggcatcattataagctatgagttgaaatgttctgtcaaatgtgtctcacatctacacgtggcttggaggcttttatggggccctgtccaggtagaaaagaaatggtatgtagagcttagatttccctattgtgacagagccatggtgtgtttgtaataataaaaccaaagaaacata

SEQ ID NO: 80 - DNA, 3076 bp, Homo sapiens
  source 1..3076: mol_type = genomic DNA
agttccactggcaggggacctgcctccctgttgccaccacaagagaggaaaagttggtcaaacaggtggggaggccagagctacaagcctcgggttccctccccaccacccgtgccaggcaggcacccgggccctggcacctgctgcctgcccagaggccacccagcctcctagacaggtggctgacatggaggagctgaccatctgggaacagcacacggccacactgtccaaggacccccgccggggctttggcattgcgatctctggaggccgagaccggcccggtggatccatggttgtatctgacgtggtacctggagggccggcggagggcaggctacagacaggcgaccacatcgtcatggtgaacggggtttccatggagaatgccacctccgcgtttgccattcagatactcaagacctgcaccaagatggccaacatcacagtgaaacgtccccggaggatccacctgcccgccaccaaagccagcccctccagcccagggcgccaggactcggatgaagacgatgggccccagcgggtggaggaggtggaccagggccggggctatgacggcgactcatccagtggctccggccgctcctgggacgagcgctcccgccggccgaggcctggtcgccggggccgggccggcagccatgggcgtaggagcccaggtggtggctctgaggccaacgggctggccctggtgtccggctttaagcggctgccacggcaggacgtgcagatgaagcctgtgaagtcagtgctggtgaagaggagagacagcgaagagtttggcgtcaagctgggcagtcagatcttcatcaagcacattacagattcgggcctggctgcccggcaccgtgggctgcaggaaggagatctcattctacagatcaacggggtgtctagccagaacctgtcactgaacgacacccggcgactgattgagaagtcagaagggaagctaagcctgctggtgctgagagatcgtgggcagttcctggtgaacattccgcctgctgtcagtgacagcgacagctcgccattggaggacatctcggacctcgcctcggagctatcgcaggcaccaccatcccacatcccaccaccaccccggcatgctcagcggagccccgaggccagccagaccgactctcccgtggagagtccccggcttcggcgggaaagttcagtagattccagaaccatctcggaaccagatgagcaacggtcagagttgcccagggaaagcagctatgacatctacagagtgcccagcagtcagagcatggaggatcgtgggtacagccccgacacgcgtgtggtccgcttcctcaagggcaagagcatcgggctgcggctggcagggggcaatgacgtgggcatcttcgtgtccggggtgcaggcgggcagcccggccgacgggcagggcatccaggagggagatcagattctgcaggtgaatgacgtgccattccagaacctgacacgggaggaggcagtgcagttcctgctggggctgccaccaggcgaggagatggagctggtgacgcagaggaagcaggacattttctggaaaatggtgcagtcccgcgtgggtgactccttctacatccgcactcactttgagctggagcccagtccaccgtctggcctgggcttcacccgtggcgacgtcttccacgtgctggacacgctgcaccccggccccgggcagagccacgcacgaggaggccactggctggcggtgcgcatgggtcgtgacctgcgggagcaagagcggggcatcattcccaaccagagcagggcggagcagctggccagcctggaagctgcccagagggccgtgggagtcgggcccggctcctccgcgggctccaatgctcgggccgagttctggcggctgcggggtcttcgtcgaggagccaagaagaccactcagcggagccgtgaggacctctcagctctgacccgacagggccgctacccgccctacgaacgagtggtgttgcgagaagccagtttcaagcgcccggtagtgatcctgggacccgtggccgacattgctatgcagaagttgactgctgagatgcctgaccagtttgaaatcgcagagactgtgtccaggaccgacagcccctccaagatcatcaaactagacaccgtgcgggtgattgcagaaaaagacaagcatgcgctcctggatgtgaccccctccgccatcgagcgcctcaactatgtgcagtactaccccattgtggtcttcttcatccccgagagccggccggccctcaaggcactgcgccagtggctggcgcctgcctcccgccgcagcacccgtcgcctctacgcacaagcccagaagctgcgaaaacacagcagccacctcttcacagccaccatccctctgaatggcacgagtgacacctggtaccaggagctcaaggccatcattcgagagcagcagacgcggcccatctggacggcggaagatcagctggatggctccttggaggacaacctagacctccctcaccacggcctggccgacagctccgctgacctcagctgcgacagccgcgttaacagcgactacgagacggacggcgagggcggcgcgtacacggatggcgagggctacacagacggcgagggggggccctacacggatgtggatgatgagcccccggctccagccctggcccggtcctcggagcccgtgcaggcagatgagtcccagagcccgagggatcgtgggagaatctcggctcatcagggggcccaggtggacagccgccacccccagggacagtggcgacaggacagcatgcgaacctatgaacgggaagccctgaagaaaaagtttatgcgagtacatgatgcggagtcctccgatgaagacggctatgactggggtccggccactgacctgtgacctctcgaaggctgccagctggtccgtcctccttctccctccctggggctgggactcagtttcccatacagaacccacaaccttacctccctccgcctggtctttaataaacagagtattttcacagca

SEQ ID NO: 81 - DNA, 1982 bp, Homo sapiens
  source 1..1982: mol_type = genomic DNA
aaagggctggacgctcacccaagaaggggaccccggcctgacctctctcggaattcaaaaaaatctaaggctcagagagggaatgggagctggctcttccctctgtgtttagcatccacgttttttggcggtctggctgaaaccagcccaccctagttcgggcgccagagcaacgcagttccgagggcagatctccaaggggcggaggcagagccgcgggtggatctttaactcaagactagcatgaagagttgccttctggcctgccctgagtctcctcaaataacaacaggcccttccaccgcagccatccgcacgggaggcctcgcgattgctcggaaccatcccgcaggagttcagctgatattttctagtgtggggcgagagattttgtggagcgcatttaaggggtttttgttgtgactgctgccttgtatacatttattttctttcttggaactgggcctcgccctcctcccactgacatgatggcccagtccaaggccaatggctcgcactatgcgctgaccgccatcggcctggggatgctggtccttggggtgatcatggccatgtggaacctggtacccggcttcagcgcggccgagaagccaacagctcagggcagcaacaagaccgaggtgggtggcggcatcctcaagagcaagaccttctctgtggcctacgtgctggtcggggccggggtgatgctgctgctgctttctatctgcctgagtatcagggataagaggaagcagcggcagggcgaggacctggcccatgtccagcacccgacaggcgctgggcctcacgcccaggaggaagacagccaggaggaagaagaggaggatgaggaggctgcctcaaggtactatgttcccagctacgaggaagtgatgaacacaaactactcagaagcaaggggagaggagcagaacccgaggttgagcatctctctcccgtcctatgagtcactgacggggctcgacgagaccacccccacatccaccagggctgacgtggaggccagccctgggaacccccctgacaggcagaactctaagttggccaaacgactgaaaccgctgaaagttcgaaggattaaatctgaaaagcttcacctcaaagactttaggatcaacctcccagacaaaaacgtccctcctccctcgatagagcctttgactcctccaccgcagtatgatgaagtccaggagaaggcccccgacacccggccgcccgactgaatggccccacttgagccacgctccctcctgtctctcacacctttcacccccaagactctaacaaagccacatgagccacagttgagaagcggaggggccagctgtgcatggagccatttggatggcggcgggcggggggggattctctgtatcaggagtgactttgttgccccacacagcctcctgctgcaggtgctttggaaagagatgctgccttggagctggtgaatctgtggaccacattcaagggtgtggcacaggcatcttcccatccttttcactccgaatcgctggcgacacattctcctttccagctaggaaagggttcctcgcggctggtttagattgtggttgtttgttttgcttctactaagactgttttgtttcaaaaaggaaacaagttttgtgtttgctgtctacgctggagtcctgaactgtgggtagaaaacacgacctggctttgtagaaaggacacagggctgttttatgaactaagcggtgaggctcaggtggcggctctcgcagagcccctgatgctgttgttctttgagggcttaaggcctgatgaacgtaggcacgtgatgcataatagtcttcaatggtacacttaactagtctcttctgtgtaacagcaaaaaaaaaaaaaaaaagaagaagaaagaaaactgtaggaaatgttctttttgaaatgccatgcaatggagctttttgtaataaaatattttatatgtagta

SEQ ID NO: 82 - DNA, 3313 bp, Homo sapiens
  source 1..3313: mol_type = genomic DNA
gagccgtagacttctaatacatctagagatgtcatatagttgattagaaagttgagttcagtgggtgcagacctgcaagatcatattcttcctcctgtacatgatgtatcggacagtgggatttggcacccgaagcagaaatctgaagccatggatgattgccgttctcattgtgttgtccctgacagtggtggcagtgaccataggtctcctggttcacttcctagtatttgaccaaaaaaaggagtactatcatggctcctttaaaattttagatccacaaatcaataacaatttcggacaaagcaacacatatcaacttaaggacttacgagagacgaccgaaaatttggtgagtcaggtggatgagatatttatagattcagcctggaagaaaaattatatcaagaaccaagtagtcagactgactccagaggaagatggtgtgaaagtagatgtcattatggtgttccagttcccctctactgaacaaagggcagtaagagagaagaaaatccaaagcatcttaaatcagaagataaggaatttaagagccttgccaataaatgcctcatcagttcaagttaatgcaatgagctcatcaacaggggagttaactgtccaagcaagttgtggtaaacgagttgttccattaaacgtcaacagaatagcatctggagtcattgcacccaaggcggcctggccttggcaagcttcccttcagtatgataacatccatcagtgtggggccaccttgattagtaacacatggcttgtcactgcagcacactgcttccagaagtataaaaatccacatcaatggactgttagttttggaacaaaaatcaaccctcccttaatgaaaagaaatgtcagaagatttattatccatgagaagtaccgctctgcagcaagagagtacgacattgctgttgtgcaggtctcttccagagtcaccttttcggatgacatacgccagatttgtttgccagaagcctctgcatccttccaaccaaatttgactgtccacatcacaggatttggagcactttactatggtggggaatcccaaaatgatctccgagaagccagagtgaaaatcataagtgatgatgtctgcaagcaaccacaggtgtatggcaatgatataaaacctggaatgttctgtgccggatatatggaaggaatttatgatgcctgcaggggtgattctgggggacctttagtcacaagggatctgaaagatacgtggtatctcattggaattgtaagctggggagataactgtggtcaaaaggacaagcctggagtctacacacaagtgacttattaccgaaactggattgcttcaaaaacaggcatctaattcacgataaaagttaaacaaagaaagctgtatgcaggtcatatatgcatgagaattcaactatttagtgggtgtagtacaacaaagtgatattaaattactggatctagtaacatgaaacacacaacgtaagttatttagaatcactttaatcaaccaataatccttagccaatttataagggacttttatttgtaaagtaatggatctggcttgaaaaatacggtagagatacttagctctttaaatcacgaatgttgaagtaccagtgagactcaatacatatttttgaagatagtccatgggatttttagaatgtcgttgtcaagggtctccttttaactgagaaactttttgaactcacaaagtgttcaagaaacccttgtataattccctacatttctctcgagctcacaaatacttttttttctttttccttattcaatcagattttccaaagtacctttccaccataagaaatgaattttctacttctacacccatttgagagacaccaataaaagaaagtcatatgtaggaaacaaagtctgatagtaaaacaagccagagatcttctaactttttttagttataaaacctctaatttttggtgacttttctacacacacacacacatacagtcacatagatattcttatcctacagatataatcagaaaaatcccgattaatgaattatatatagatagatagagtgattagttaatcaaaactatatatttgttaaaaatttgggagaaggtgatggtaatttcattagtcccaaaacaccggcataagaaatttgacatttcacagatgcacttacatattttgaccacaagggggaaattttgtgcattattattgtagggttaaatgcctgaaaaaaataagcatttactaataccttgaaaaatcaaagaacattatgttcctccgtagcaagaaaaattttatgccctcgcattgctacagccaaatagcttataaggcttatggcaatgtcccaactattgcaatcataaaaccctagttttagaaaaatttgaaaataaatataaaatttgagcacctactgcatataacacatggggctataacatataacatatagtatgttttctcttttggaccactttgttatatgtggtccacaagagaaaacatactccatcagcaacgtgtctcctgaagtcattaggagagtaaggctcatgtatttcgtaatacaattaaattaaggctttatggaagcaattattctgcttggcaatgcctgtgaaatattatggtctgcaggctggggagtcatttactcatttagcccaaatataagacaaatcattggaacgatgaacttgctttataataatttccctcacatgctattgagaaggcatgaaaataatcctaaaatatctaaaagagatatgcagttcactcataaaacacttgattcataatttagactcatagaattttaaagctgagaagagattgcatgtcacataggccaatatacttatgttatgtttaagaatatgagtatattcttaactatttttattgaattcttaactatttttattgaatcatcagatctgtaaaattttaagtgagagctattgaacctgtattaagatagaaaactcttaagataagaattttaaatgacgagattcatttaggcagctggctgccaattctctgaattcacttcaagacaaagtttcttcttcatatataaatatacataggcatataaattattctatattatggtggtaaaacttaatgctgactttggatgttcagattcaggaaatgatcttagaattatgaactttgtatttttaaactatttggtgcagcatttcattttgggtcacatgtaagcagatattgatatgtcatttttaatctaataaagttggtgatcagagca

SEQ ID NO: 83 - DNA, 2645 bp, Homo sapiens
  source 1..2645: mol_type = genomic DNA
agcacataagtcaaaagctgcgtcagaaggttctaacttttgtcatcactattaccagcattgtcatcgttatcgttatcttcgtcatcatcattaccaccgttatacctgatactgccataacaatcagaacattatgtacaggcacggcatatcttcccaaagatcttggccactatggactacgatctttatttttcttggagtggcggcaatcttgggagtaaccattggtcttcttgttcattttctggcagttgagaagacttactattatcaaggtgattttcatatttctggagtcacatacaatgataattgtgaaaacgcagcttcacaagccagcacaaatctaagcaaagatattgagactaagatgttaaatgcatttcaaaattccagtatatataaggaatatgtcaaatctgaggtcatcaaacttctgcctaatgccaatggttcaaatgtgcagttacagctgaaattcaagtttcctccagcagaaggagttagcatgaggactaaaatcaaggctaaattacatcagatgttgaaaaacaacatggcatcctggaatgcagttcctgcttccattaaactcatggaaatcagcaaggctgcttctgaaatgcttaccaacaactgttgtgggagacaagtagccaacagtatcataactggcaacaaaattgtgaatggaaaaagctccctggagggggcatggccatggcaggccagcatgcaatggaaaggccgtcactactgtggagcctctctgatcagcagcaggtggctattatctgcagctcactgctttgctaagaaaaataattcaaaagattggactgtcaactttggaattgtagtaaataaaccatatatgacacggaaagtccaaaacattatttttcatgaaaattatagcagtcctgggcttcatgatgatattgcccttgtgcagcttgctgaagaagtttcttttacagagtacattcgtaagatttgtcttcctgaagccaaaatgaagctctcagaaaatgacaatgttgtagttacaggttggggaacactttatatgaatggttcatttccagtgatacttcaagaagactttttgaagattattgacaacaaaatttgcaatgcctcatatgcatactctggctttgtgactgatacaatgttatgtgctggatttatgtcaggagaagctgatgcatgtcagaatgattctggtggaccactagcttaccctgattccagaaatatctggcatcttgttggaatagtaagctggggtgatggatgtggtaaaaagaataagccaggtgtctatactcgagtgacttcttatcgcaattggattacatccaagactggactctgaaaaaaaaggaattatacaaaggaacataaagaccactgtaggctatccttatctgtggctttgggatctttttattttgattgttggtaaactaatcattttattatatataaaaatataaatgtctttattaatattttattatatctaaaacactgtccatgcatttattaatcttttaaattaatgtaagagataggaagaggccaggtgtagtggctcatgcctgtaatcctagcactttgagaggccgagataggcggattgcttgagcccaggagtttgagggcagcctgggaaacatggctaaatccgtgttcaaaaaatacaaaaattagctaggcatggtggcgtgcacctatagtcccagctattcaggaggctgaggtgggaggattgcttgagcccagcaggttgaggctgtggtgatttgtgattgcgccaccactgcactccagcctgggcaacagagtgagaccctgtctcaaaaaaaaaaaaaaaaaaaggagagaagaaaataaagaaggctaatatataatttttcttaatagctgagttgtgattcattttaccattatattcagaattgaccttaatttgttttccagaatcaaaagaaaataaaatataaattttgaaactatgagaacatatataggggatttatcaaatgagttactttttattatatcaattataatttcattttattaactggaataagtgcgaaagtgtacattactttttaacttatatgtgaaatgattcatgtacctcacaaaatcatgcaatcttgcagtgaagaaggagtatcagagctccagctacactgagtctttcaatttacagatcaagaaactgaagaccagagaagagaaatgatttggttaaagtcactctccatcatctgtgataagttgacaaatttatccttacacaaatttgtcctcattgagttgacatccaaaatttgtcaggaattaaagaatgacttttgagtggcaattatttatataacattttccattgtcttcatattcaccttttgtcatttgtttgctttctcttgcagcaaaaataatatgaggtcatcaagggcagagaactgtgtggcatctccttatagtaaaagtgcaatcatatggatggctaactttgtaatctctacaaataatttaccttatgtaattttccaaatgtaaaagtcaccaaaaacttaaaggcattaaaataaactaaaaaagtactcatttcttca

SEQ ID NO: 84 - DNA, 3250 bp, Homo sapiens
  source 1..3250: mol_type = genomic DNA
accagggtcccggctcggggtccgggctggggaggggaacctgggcgcctgggacccgccgatgccccctgccccgcccggaggtgaaagcgggtgtgaggagcgcggcgcggcaggtcatattgaacattccagatacctatcattactcgatgctgttgataacagcaagatggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaaatcaaggatgctcagtttaaggtacactgtttccatgttatgtttctacacattgctacctcagtgctcctggaaacttagcttttgatgtctccaagtagtccaccttcatttaactctttgaaactgtatcatctttgccaagtaagagtggtggcctatttcagctgctttgacaaaatgactggctcctgacttaacgttctataaatgaatgtgctgaagcaaagtgcccatggtggcggcgaagaagagaaagatgtgttttgttttggactctctgtggtcccttccaatgctgtgggtttccaaccaggggaagggtcccttttgcattgccaagtgccataaccatgagcactactctaccatggttctgcctcctggccaagcaggctggtttgcaagaatgaaatgaatgattctacagctaggacttaaccttgaaatggaaagtcatgcaatcccatttgcaggatctgtctgtgcacatgcctctgtagagagcagcattcccagggaccttggaaacagttggcactgtaaggtgcttgctccccaagacacatcctaaaaggtgttgtaatggtgaaaacgtcttccttctttattgccccttcttatttatgtgaacaactgtttgtctttttttgtatcttttttaaactgtaaagttcaattgtgaaaatgaatatcatgcaaataaattatgcaatttttttttcaaagtaaaaaaaaaa

SEQ ID NO: 85 - DNA, 3610 bp, Homo sapiens
  source 1..3610: mol_type = genomic DNA
actccggcatttgcagcagagcgaaaggtggtcgagtcctgaaggagggcctgatgtcttcatcattctcaaattcttaggacggtcgggccctggaaggaacgctctcggaattggccgcggaaaccgatctgcccgttgtgtttgtgaaacagagaaagataggcggccatggtccaaccttgaaggcttatcaggagggcagacttcaaaagctactaaaaatgaacggccctgaagatcttcccaagtcctatgactatgaccttatcatcattggaggtggctcaggaggtctggcagctgctaaggaggcagcccaatatggcaagaaggtgatggtcctggactttgtcactcccacccctcttggaactagatggggtctcggaggaacatgtgtgaatgtgggttgcatacctaaaaaactgatgcatcaagcagctttgttaggacaagccctgcaagactctcgaaattatggatggaaagtcgaggagacagttaagcatgattgggacagaatgatagaagctgtacagaatcacattggctctttgaattggggctaccgagtagctctgcgggagaaaaaagtcgtctatgagaatgcttatgggcaatttattggtcctcacaggattaaggcaacaaataataaaggcaaagaaaaaatttattcagcagagagatttctcattgccactggtgaaagaccacgttacttgggcatccctggtgacaaagaatactgcatcagcagtgatgatcttttctccttgccttactgcccgggtaagaccctggttgttggagcatcctatgtcgctttggagtgcgctggatttcttgctggtattggtttagacgtcactgttatggttaggtccattcttcttagaggatttgaccaggacatggccaacaaaattggtgaacacatggaagaacatggcatcaagtttataagacagttcgtaccaattaaagttgaacaaattgaagcagggacaccaggccgactcagagtagtagctcagtccaccaatagtgaggaaatcattgaaggagaatataatacggtgatgctggcaataggaagagatgcttgcacaagaaaaattggcttagaaaccgtaggggtgaagataaatgaaaagactggaaaaatacctgtcacagatgaagaacagaccaatgtgccttacatctatgccattggcgatatattggaggataaggtggagctcaccccagttgcaatccaggcaggaagattgctggctcagaggctctatgcaggttccactgtcaagtgtgactatgaaaatgttccaaccactgtatttactcctttggaatatggtgcttgtggcctttctgaggagaaagctgtggagaagtttggggaagaaaatattgaggtttaccatagttacttttggccattggaatggacgattccgtcaagagataacaacaaatgttatgcaaaaataatctgtaatactaaagacaatgaacgtgttgtgggctttcacgtactgggtccaaatgctggagaagttacacaaggctttgcagctgcgctcaaatgtggactgaccaaaaagcagctggacagcacaattggaatccaccctgtctgtgcagaggtattcacaacattgtctgtgaccaagcgctctggggcaagcatcctccaggctggctgctgaggttaagccccagtgtggatgctgttgccaagactgcaaaccactggctcgtttccgtgcccaaatccaaggcgaagttttctagagggttcttgggctcttggcacctgcgtgtcctgtgcttaccaccgcccaaggcccccttggatctcttggataggagttggtgaatagaaggcaggcagcatcacactggggtcactgacagacttgaagctgacatttggcagggcatcgaagggatgcatccatgaagtcaccagtctcaagcccatgtggtaggcggtgatggaacaactgtcaaatcagttttagcatgacctttccttgtggattttcttattctcgttgtcaagttttctagggttgaatttttttcttttttctccatggtgttaatgatattagagatgaaaaacgttagcagttgatttttgtccaaaagcaagtcatggctagagtatccatgcaaggtgtcttgttgcatggaagggatagtttggctcccttggaggctatgtaggcttgtcccgggaaagagaactgtcctgcagctgaaatggactgttctttactgacctgctcagcagtttcttctctcatatattcccaaaacaagtacatctgcgatcaactctagccaaatttgcccctgtgtgctacatgatggatgattattattttaaggtctgtttaggaagggaaatggctacttggccagccattgcctggcatttggtagtatagtatgattctcaccattatttgtcatggaggcagacatacaccagaaatgggggagaaacagtacatatctttctgtctttagtttattgtgtgctggtctaagcaagctgagatcatttgcaatggaaaacacgtaacttgtttaaaagtttttctggtagctttagctttatgctaaaaaaaataatgacattgggtatctatttctttctaagactacattagtaggaaaataagtcttttcatgcttatgatttagctgttttgtggtaattgctttttaaaggaagttattaatatcataagttattattaatattttgaacacaggtggatgtgaaggattttcatttaaaaaccaagtggttttgactttttctgttgaatgaacaactgtgccttgtggaatttttgcagaagtgtttatgctttgttagcatttcaacttgcattattataaagaggtattaatgcctcagttatgtgtttgtcaatgtactggctgaggattctatctcagctgtcttttctaactgtgtaggttgagttttgaacacgtgcttgtggacatcaggcctcctgccagcagttcttgaagcttctttttcattcctgctactctacctgtatttctcagttgcagcactgagtggtcaaaatacatttctgggccacctcagggaacccatgcatctgcctggcatttaggcagcagagcccctgaccgtcccccacagggctctgcctcacgtcctcatctcatttggctgtgtaaagaaatgggaaaagggaaaaggagagagcaattgaggcagttgaccatattcagttttatttatttatttttaatttgtttttttctccaagtccaccagtctctgaaattagaacagtaggcggtatgagataatcaggcctaatcatgttgtgattctcttttcttagtggagtggaatgttctatccccacaagaaggattatatcttatagacttgtcttgttcagattctgtatttacccattttattgaaacatatactaagttccatgtatttttgttacaaatcttctgaaaaaaaacaaaacaatgtgaaacattaaaattaaaaggcattaataatatccacgtgtgccttcttactgaa

SEQ ID NO: 86 - DNA, 3304 bp, Homo sapiens
  source 1..3304: mol_type = genomic DNA
cttttctccaggaagagagctgtgaccagcagcgtcccttattcgcttggccttggttcctgtttgcactggctacagcagggcactggcccctactgtcaccgccacctacacaaagaccctatctctgagcgctgcagcctactgttcagccccaggtttgaggatggatgccctggacgcttcgaagctactggatgaggagctgtattcaagacagctgtatgtgctgggctcacctgccatgcagaggattcagggagccagggtcctggtgtcaggcctgcagggcctgggggccgaggtggccaagaacttggttctgatgggtgtgggcagcctcactctgcatgatccccaccccacctgctggtccgacctggctgcccagtttctcctctcagagcaggacttggaaaggagcagagccgaggcctctcaagagctcttggctcagctcaacagagctgtccaggtcgtcgtgcacacgggtgacatcactgaggacctgctgttggacttccaggtggtggtgctgactgctgcaaagctggaggagcagctgaaggtgggcaccttgtgtcataagcatggagtttgctttctggcggctgacacccggggcctcgtggggcagttgttctgtgactttggtgaggacttcactgtgcaggaccccacagaggcagaacccctgacagctgccatccagcacatctcccagggctcccctggcattctcactctgaggaaaggggccaatacccactacttccgtgatggagacttggtgactttctcgggaattgagggaatggttgagctcaacgactgtgatccccggtctatccacgtgcgggaggatgggtccctggagattggagacacaacaactttctctcggtacttgcgtggtggggctatcactgaagtcaagagacccaagactgtgagacataagtccctggacacagccctgctccagccccatgtggtggcccagagctcccaggaagttcaccatgcccactgcctgcatcaggccttctgtgcactgcacaagttccagcacctccatggccggccaccccagccctgggatcctgttgatgcagagactgtggtgggcctggcccgggacctggaaccactgaagcggacagaggaagagccactggaagagccactggatgaggccctagtgcggacagtcgccctaagcagtgcaggtgtcttgagccctatggtggccatgctgggtgcagtagctgcccaggaagtgctgaaggcaatctccaggaagttcatgcctctggaccagtggctttactttgatgccctcgattgtcttccggaagatggggagctccttcccagtcctgaggactgtgccctgagaggcagccgctatgatgggcaaattgcagtgtttggggctggttttcaggagaaactgagacgccagcactacctcctggtgggcgctggtgccattggttgtgagctgctcaaagtctttgccctagtgggactgggggccgggaacagcgggggcttgactgttgttgacatggaccacatagagcgctccaatctcagccgtcagttcctcttcaggtcccaggacgttggtagacccaaggcagaggtggctgcagcagctgcccggggcctgaacccagacttacaggtgatcccgctcacctacccactggatcccaccacagagcacatctatggggataactttttctcccgtgtggatggtgtggctgctgccctggacagtttccaggcccggcgctatgtggctgctcgttgcacccactatctgaagccactgctggaggcaggcacatcgggcacctggggcagtgctacagtattcatgccacatgtgactgaggcctacagagcccctgcctcagctgcagcttctgaggatgccccctaccctgtctgtaccgtgcggtacttccctagcacagccgagcacaccctgcagtgggcccggcatgagtttgaagaactcttccgactgtctgcagagaccatcaaccaccaccaacaggcacacacttccctggcagacatggatgagccacagacactcaccttactgaagccagtgcttggggtcctgagagtgcgtccacagaactggcaagactgtgtggcgtgggctcttggccactggaaactctgctttcattatggcatcaaacagctgctgaggcacttcccacctaataaagtgcttgaggatggaactcccttctggtcaggtcccaaacagtgtccccagcccttggagtttgacaccaaccaagacacacacctcctctacgtactggcagctgccaacctgtatgcccagatgcatgggctgcctggctcacaggactggactgcactcagggagctgctgaagctgctgccacagcctgacccccaacagatggcccccatctttgctagtaatctagagctggcttcggcttctgctgagtttggccctgagcagcagaaggaactgaacaaagccctggaagtctggagtgtgggccctcccctgaagcctctgatgtttgagaaggatgatgacagcaacttccatgtggactttgtggtagcggcagctagcctgagatgtcagaactacgggattccaccggtcaaccgtgcccagagcaagcgaattgtgggccagattatcccagccattgccaccactacagcagctgtggcaggcctgttgggcctggagctgtataaggtggtgagtgggccacggcctcgtagtgcctttcgccacagctacctacatctggctgaaaactacctcatccgctatatgccttttgccccagccatccagacgttccatcacctgaagtggacctcttgggaccgtctgaaggtaccagctgggcagcctgagaggaccctggagtcgctgctggctcatcttcaggagcagcacgggttgagggtgaggatcctgctgcacggctcagccctgctctatgcggccggatggtcacctgaaaagcaggcccagcacctgcccctcagggtgacagaactggttcagcagctgacaggccaggcacctgctcctgggcagcgggtgttggtgctagagctgagctgtgagggtgacgacgaggacactgccttcccacctctgcactatgagctgtgacaaggcagccaccctgtcacctagctcaatggagccccggatcccaagccctgcattgtaagcccacagtaggcactcaataaatgcttgttaaaggaa

SEQ ID NO: 87 - DNA, 8324 bp, Homo sapiens
  source 1..8324: mol_type = genomic DNA
ctccccgcgcgccgggtcggagccggtgcgggagcggagccgcgcgaagccggcaggagcacgcgggagcgcggccccgcagtggcccggcccgagagagcagcgcgcaggagctggagcggcgccacggcccccaccccagcgcggacctcgcccggaactcggaccccgtcctcgaagcgtgatctctcccgcctcgcacgccctggccgccgggccgcgggcatggacggcgtccgctcgccctagatggacggcgatggcggccgccgagacgtccccggcacgctgatggagcccgggcgcggcgcggggcccgcgggcatggcggagcctcgggcgaaggcggcgcggccggggccccagcgctttctgcggcgcagcgtggtagagtcggaccaggaggagccgccgggcttggaggcagccgaggcgccgggcccgcagcccccgcagcccctgcagcgccgggtgcttctgctctgcaagacgcgccgcctcatcgcggagcgcgcccgcggacgccccgccgcccccgcgcccgcagcgctggtagcgcagccgggagcccccggagcccccgcggacgccggccccgagcccgtgggcacgcaggagcccggcccggaccccatcgcagccgctgtcgaaaccgcgcctgcccccgacggcggccccagggaggaggcggcggcgaccgtgaggaaggaggatgagggggcggccgaggcgaagcctgagcccgggcgcactcgccgggacgagcccgaagaggaggaggacgacgaggacgacctcaaggccgtggccacctctctggacggccgcttcctcaagttcgacatcgagctgggccgcggttccttcaagacggtctacaaggggctggacacggagacctgggtggaggtggcctggtgtgagctgcaggaccggaagctcaccaagctggagcggcagcggttcaaggaagaggctgagatgctgaaaggcctgcagcaccccaacatcgtgcgcttctacgacttctgggagtccagcgccaagggcaagcggtgcattgtgctggtgacggagctgatgacctcagggacgctgaagacatacctgaagcggttcaaggtgatgaagcccaaggttctccgcagctggtgccggcagatcctgaagggcctgctgttcctgcacacaaggacgccacccatcatccaccgagacctgaaatgtgacaatattttcatcaccggaccaactgggtctgtgaagattggcgacttgggcctggccactctgaaaagagcgtcatttgccaaaagtgtgataggtactcccgagttcatggcgcccgagatgtacgaggagcactacgatgagtccgtggacgtctatgcctttgggatgtgcatgctggagatggccacctcggagtacccctactcggagtgccagaatgcggcccagatctaccgcaaggtcacctgtggtatcaagccggccagctttgagaaagtgcacgatcctgaaatcaaggagattattggggagtgtatctgcaaaaacaaggaggaaaggtacgagatcaaagacctgctgagccacgccttcttcgcagaggacacaggcgtgagggtggagctcgcggaggaggaccacggcaggaagtccaccatcgccctgaggctctgggtggaagaccccaagaaactgaagggaaagcccaaggacaatggagccatagagttcaccttcgacctggagaaggagacgccggatgaggtggcccaagagatgattgagtctggattcttccacgagagtgacgtcaagatcgtggccaagtccatccgtgaccgcgtggccttgatccagtggcggcgggagaggatctggcccgcgctgcagcccaaggagcagcaggatgtgggcagcccggacaaggccaggggtccgccggtgcccctgcaggtccaggtgacctaccatgcacaggctgggcagcccgggccaccagagcccgaggagccggaggccgaccagcacctcctgccacctacgttgccgaccagcgccacctccctggcctcggacagcaccttcgacagcggccagggctctaccgtgtactcagactcgcagagcagccagcagagcgtgatgcttggctcccttgccgacgcagcgccgtccccggcccagtgtgtgtgcagcccccctgtgagcgaggggcccgtcctgccgcagagcctgccctcgctgggggcctaccagcagcccacggctgcacctggcttgccggtgggctctgtcccggcccccgcctgccctccgtccctccagcagcacttcccggatccggccatgagcttcgcccccgtgctgccgccgcccagcacccccatgcccacgggcccaggccagccagcaccccccggccagcagcctcctccgctggcccagccgacacccctgccgcaggtcctggccccacagcccgtggtccccctccagccggttcccccccacctgccaccgtacctggctccagcctcccaggtgggggcccccgctcagctgaagcccctccagatgccacaggcgcccctgcagccgcttgctcaagtccctccgcagatgcccccgattcctgttgtgccccccatcacgcccctggcgggaatcgacggcctccctccggccctcccagacctgccgaccgcgactgtgcctcccgtgccaccacctcagtatttctctccagccgtgatcttgccgagcctcgctgccccactcccccctgcgtccccagccttgcctctgcaggctgtgaagctgccccacccccctggggcgcccctggccatgccctgccggaccattgtgccaaatgcaccggccactatccccctgctggccgtagccccaccgggcgtggctgccctgtccattcattctgccgtggcccagctcccaggccaacctgtgtacccagcggccttcccacagatggcgcctactgacgtccctccttccccccatcacacggtgcagaatatgagggccacccctccacagccggcactgcctccacaacccacactgcccccacaacccgtgctgcccccgcaacccacgctgccccctcaacctgtgttgcccccgcaacccacacggccccctcaacctgtgctgcccccgcaacccatgctgcccccacaacctgtgctgcccccgcagccggcactgcctgtgcgccctgagcccctccagccccaccttcctgaacaagctgctccagctgctacaccagggagccagattctgcttggccacccagctccctatgctgtggacgtcgccgctcaggtccccaccgtgcctgtgccaccggctgcggtcctctcgccgcctctgccggaagtgctgctgcctgccgcccctgagctcctgcctcagttccccagctccctggccacggtgtctgcctctgtgcagagtgtgcccacccagactgccacacttctgccaccagcaaacccaccgctgcctggcgggcccgggatcgccagcccttgcccaactgtccagctgacggtggaaccagtccaagaggagcaggcctcacaggacaagccgcccggcctcccgcagagctgtgagagctatggaggttctgatgtcacttctggaaaagagctgagtgacagctgtgaaggcgcctttggagggggcaggctggagggcagggcagcccgaaaacaccaccgcaggtccacgcgtgcgcgctcccggcaggagagggccagccggccccggcttaccatcttgaacgtgtgcaacactggggacaagatggtggagtgccagctggagacgcacaaccacaagatggtgaccttcaagttcgacttggacggggacgcacccgatgaaattgccacgtatatggtggagcatgactttatcctgcaggccgagcgggaaacgttcatcgagcagatgaaggatgtcatggacaaggcagaggacatgctcagcgaggacacagacgccgaccgtggctccgacccagggaccagcccgccacacctcagcacctgcggcctgggcaccggggaggagagccgacaatcccaagccaacgcccccgtgtatcagcagaacgtcctgcacaccgggaagaggtggttcatcatctgtccggtggctgagcaccccgcccccgaggcccctgaatcttcgcccccacttcctctaagctccctgccgccagaagccagccaaggtccatgcagggggctcaccctgccctgtctcccatggcgcagagcagcctgtggggctgtgttcctatccttgttttctgctgaatcagctcagtcaaagcagcctccagattcagcgccctataaagaccagctgtcctcgaaggaacaacccagctttctagccagtcagcagctcctgagccaggcgggccccagcaaccctcctggggcacccccagcccctttggccccctcctcccctcctgtgactgctctgccccaagatggagcagctccagccaccagcaccatgccagagccagcgtcaggaactgccagccaggcagggggtccagggacacctcaggggctgaccagtgagctcgagacgtctcagccactagcggagactcacgaggccccgcttgctgtgcagcccctcgtggtgggcctagcaccttgcactccagctccagaggctgcctcaaccagggacgccagtgccccaagggagcccctgccacctcctgcacctgagcccagcccccacagcgggaccccacagcccgccttgggtcaacctgctcccctgcttcctgccgcagtgggggccgtcagcctggccacctcccagctcccaagcccacccctggggcccaccgtccccccacagccaccctcggccctggagtcggatggggaagggccgccccccagggtgggctttgtggacagcaccatcaagagcctggacgagaagctgcggactctgctctaccaggagcacgtgcccacctcctcagcctcagctgggacccctgtggaggtgggcgacagagacttcaccctggagcccctgagaggggaccagccccgctcagaggtctgcgggggggacctggccctgcccccagtgcctaaggaggcggtctcagggcgtgtccagctgccccagcccttggtggagaagtcagaactggcccccactcgaggggccgtgatggagcagggcacgtcctcgtcaatgacagcagagtcgtctcccaggagtatgctaggctatgacagagatggaaggcaggtggcctcagactcccatgtggtccccagcgtcccccaggatgtacctgcttttgtgagacctgcacgtgtggagcccacagacagggatggtggagaagctggagaaagctcggcagagcccccgccgagtgacatgggcacagtggggggccaggctagccacccccagacactcggcgctcgagctttggggtcccctcggaaacgtccagagcagcaggatgtcagctcaccagccaagactgtgggccgtttctcggtggtcagcactcaggacgagtggaccctggcctccccccacagcctgagatactctgccccacccgacgtctacctggacgaggccccctccagccccgacgtgaagctggcagtgcggcgggcgcagacggcctcctccatcgaggtcggcgtgggcgagcccgtgtccagcgactctggggacgagggccctcgggcgagacccccggtgcagaagcaggcgtccctgcccgtgagtggcagcgtggctggcgacttcgtgaagaaggccaccgccttcctgcagaggccttctcgggccggctcgctgggccccgagacacccagcagggtgggcatgaaggtccccacgatcagcgtgacctccttccattcccagtcgtcctacatcagcagcgacaatgattcggagctcgaggatgctgacataaagaaggagctgcagagtctgcgggagaagcacctgaaggagatctcggagctgcagagccagcagaagcaggagatcgaagctctgtaccgccgcctgggcaagccactgccccccaacgtgggcttcttccacacggcaccccccactggccgccggagaaaaaccagcaagagcaagctgaaggcaggcaagctgctaaatcccctggtgcggcagctcaaggtcgtggcctccagcacaggtcacttggctgactccagcagaggccctcccgctaaggaccctgcccaagccagtgtggggctcactgcagacagcacgggcctgagcgggaaggcagtgcagacccagcagccctgctccgtccgggcctccctgtcttcggacatctgctccggcttagccagtgatggaggcggagcgcgtggccaaggctggacggtttaccacccaacgtctgagagagtgacctataagtctagtagcaaacctcgtgctcgattcctcagtggacccgtatctgtgtccatctggtctgccctgaagcgtctctgcctaggcaaagaacacagcagtaggtcctccaccagcagcctggccccaggccctgagccaggcccccagcccgccctgcacgtccaggcgcaggtgaacaacagcaacaacaagaagggtaccttcacggacgacctgcacaagctggtggacgagtggacgagcaagacggtgggggccgcgcagctgaagcccacgctcaaccagctgaagcagacccagaagctgcaagacatggaggcccaggcaggctgggctgcccctggcgaggcgcgggctatgaccgcacctcgagcaggagtggggatgccacgtctgcccccagcgcccggccctctgtccaccacggtcattcccggagccgccccgaccctgtccgtgcccacaccagatggcgccctcggaaccgcccggagaaaccaggtgtggtttggcctccgagtcccccccaccgcctgctgtgggcacagcactcagccgcgagggggacagcgggtgggcagcaagactgcttcctttgcggcttcagaccctgttcgctcctaggttcctgtggtccacgcgccgtctccacacccacttcctatacttgagttgatggttagaaccttgtcgtcaccctgcagaagtacagtgccttgaatgccagcttttccgttccctgatgaaaagatatgttaaaaaaaattatcggaaaaggtttcatttgcaattggcttgtgcattgataatctttatttactgttttaagttgcagagatgtgaatggtttacaaatctgaagctgaagttcaatctttggttttctgttgtaaatgccttttacaaacattgaattagctaccttaagtattgaagagcttccattgctaggtgagccctgctttgtcctcagtagagtgccggttccctgggctcatccaggggctgagagacggcgggacgctggggcagggcacactggcggagctgcttgctcagtagggaatgtcagttgttgcgctgggccatgagaaatccgccagaaaacgttaggtgagcagacatgccccccatgccagtgggctgctgtgagtgaggataaggtgtgtgttgggcatagaaaccctggctgcccgcccaccctgtggagacaagtgcagctcctccagctggagaggctgccctctctcctgcccacttccctcccttctccatgatttccatggagacctgtggctctgctcacccctggcatgcagaccgctctcccgtccagccctaagcctgctctgcggagggcggggtcattcttctcctggagatttcagtgggactcgtccccagtgggcacaacacagcccttggtgggaggggaaggccccagcctcctccacctcccactggaaagcagactgcttgggactgcccagctgtgaattgtatagtttctgtacttattagaactgggtaaattattttggttcaaatctattattccatcaattcagttagaattgaattttctaggtgattatgcagaatcttctgccagggcacgatgctgtcgtaagagatttctgttctctgtactgggcccccttgccctgttccttgagtgaagtgggggctgccctcacctgtcccccttgcctgtgaatcccttccttgtacatggtggtcagtggcacggaatccccaatagattgtatatctgaaggagaaaaataaacacttttgctcgaaaa

SEQ ID NO: 88 - DNA, 5924 bp, Homo sapiens
  source 1..5924: mol_type = genomic DNA
gcctcctcggcctccgctcgcagccgccgcctccgcctccgccgggctgaggagccgggagtccgccgcgccggctcggggctgcgggatggggagttagcgccacggcggcggcagtggccgcagcgcaccccgccgccgcccaggagcccgtccagccaggggtgccgggcccgcccagcccgccccggagccaggcccgcgggcggcggcggagctgggcaggtggatgcggctggaagatggcgccctcgggcccgggcagcagcgccaggcggcggtgccggcgggtgctgtactggatcccggtggtgttcatcaccctcctgctcggctggtcctactacgcctacgccatccagctgtgcatagtgtccatggaaaacactggcgaacaagttgtgtgcctgatggcctatcatctactttttgcaatgtttgtctggtcatactggaaaactatctttacattaccaatgaatccttcaaaagaattccatctctcttatgcagagaaagatttgttggagagagagccaagaggagaagcccatcaggaagttcttaggcgagcagccaaggatcttcccatctataccaggaccatgtctggagccatccgatactgtgacagatgccaacttataaaaccagatcgctgccatcactgctccgtctgtgataaatgtattttgaagatggatcatcattgtccatgggtgaacaattgtgttggattttcaaattataagttctttctccttttcttggcttattctctgctctactgcctttttattgcggcaacagatttacagtattttatcaaattttggacaaatggcctacctgatactcaagccaagttccatattatgtttttattctttgctgcagctatgttttctgtcagcttgtcttctctgtttggctatcattgttggctagtcagcaaaaataaatctacattagaggcattcagaagtccagtatttcgacatggaacagataagaatggattcagcttgggtttcagtaaaaacatgcgacaagtttttggtgatgagaagaagtactggttgctacccattttttcaagtctaggtgatggctgctcctttccaacttgccttgttaaccaggatcctgaacaagcatctactcctgcagggctgaattccacagctaaaaatctcgaaaaccatcagtttcctgcaaagccattgagagagtcccagagccaccttcttactgattctcagtcttggacggagagcagcataaacccaggaaaatgcaaagctggtatgagcaatcctgcattaaccatggaaaatgagacttaactcttcaagcaagataaattcatactttataaaagtatcaatgctgtagatggatggaagaggcttcccacaggaaggtgccaccagtcagttgtgcctatgtccctttggctggaaatgcagaatatgaattgattagttctctccaagccattgcttaaaatataacatgttttggatccaatacacacattgttacaactaacacaaattcctattaaatattaaaagtagttctggtttattaatcaacggggaaaacatcttctccaaaaaacttggaataaatccaaggaccagtttttacccaaatatatgggtagcacagtttatcacatagaaactccattaatcatctgattttccgaatctgaaaattgagactattaagatattaggatttcagagatttcaagtcacattataatgataagcattattcataaaacttgttacctttaagaaggtggaagtggcaaaccatacttcttttttttcctctgatgtgaatccagcctcagactgagtgaactgtaataattatgaattcattacagagtccaggtggcctgcagttgaagatcatcaaccatttttgcctcacttaattccagccttttgttttctgctggaaaataagtgtggacattgaagcttgagctctcaaagcagttggctggaatacttttgtcagaatacggtacatttctattacatcagaaatatattttcatctcttcttgttaaattgggaggaaatttatgatagcaattatgaagattgttttatgacattcttttgtcagtttggctttctaaaaatctctttttagattatttctcctgttgaacatagtaaaactattgaatttctcttaagaattcctaataggtcaatagatttaccctccagtgatatctatattatttctttctcgtctcatcaaaatgatgacaggtaaactatatttttccttaaacacctattacagttaaattatgcaaatcattaaataaaaatcatacaacttttggaaagttagttcaacatgaactaaaatggcatgctatttggaaatttagtttgagataaactaaagtgtgttgatgccagaatgttcagcttcagtaaatataataagctcttgtgccttgtatgcactatttaaaaaaagttttttttatttgagtccagtataattcatgtaaatgttaacaattagaataatactctgtatgcttttttgatactgattttgagaatttaaagcagattaccttttaaaactggaccaactaagtaattggtatttaatcaaagagaaaatggtaataaacttttcaaaatctttgttaaaccaaacattcaacacaaaataaactagaaggccagaggataatggaataaaagatcattgcaattacttatccttcctaaaaatatagttttatattaattgtgcttatggaagaaacaatgtcagccaagtccattttatagtttgagtgcaattctttgaacaatagaaatatctgcagtctttcacagatttgtattatgctgaagagtttcatctgacaatctgcttcaagaaatctcagaaaatatgataacattttaactttcattttagagcacgttttggtcatttttaaaaatacctaaagtgccagaccggaacctatagctactgctagaagtcttaaaaaaaccaacagcagcacaggatgtattaagaattatatgaagtcaggtttgtttttttttttttttttttttcaaagcacagtactgttagctgtttttgtggacaggattcgattaagtattccctcttgtcaaactggaagctaggggaaaaagagggatttttatcctttactcttctagagtactgttaatgcccctttcccacagtcttttatataattaaatatatgtcaatacacattagaatcagatttgaaaaagttaaaacaatttcattgttgtaattgttccctttctgttttcatatagtgaataacctttaaagggttgttttgttttgttttgaattataggagttataatctttggagatgattgcatatctcattagatatgcaatataaatttatctgagtgaacaaagtgctaaataaatagatctacattttgtacatatttatataaaatttacctttaagtatttactttaaaaaatttaatggcttaactcgaacttgaagacacatacttcaactgtccttattgtccattaaactgataattttgatttttcttgcttttatagattttactatataggaatcaagatttaagaaattttgcattaaaaatagtgtaccaatgcttcatatacgttagttatttgctattatgtagggaagaggattgttatttcaaagatatattaaagaacagttgcatctgaatataatcatgatgcattcaatgaagttcatatccatgaattcactcctaatataccctaataaagtggttgatcacctacattatctgtctataagaaactgcctttgcactgacaatgtaaattattttaagcataaattttcctaatagtttattagagctactaatggcaaaattcatgtcttcaatgtggccataacataggtcttgggaggggtggtgagaaaataaggtatttactttgtagtagttaagtgattctgaggacaattagaaacagactataaaactcaactacactgtaatcaatagacaactattggaaatatggcatatgttgacaaacacttaactagggaaatcaaagatataaatcatgtttataaaaaattaggcacgcaatcatgtgattcatgcttcaagaaaatatatgaaaaaagtgttactccattatgttataaactagcagattttccctattttgaaatgcatgtaaacgggctcggctgattctgaaattgtactgtatcatttggtatttacttatatggtcaaatacttgtttgtaaaagtgatggttctgatacctttgatcaaacatagatacaaacctcagaaaatggtaaatgcaaagcagtttacgttgtaaatcatgatggtgtcctccagcacaaccatcccacccacacacctagactacagattctgatctgtgaatattttcaccaaagtgacatttaaacaagtggatttttcacaaataacacgtgtttaatatctttctgcagaaacgtgttcttctaaagcagaaaaccacgtggcttaatcccaaatgttttacacttcttttccacatacttagccagttcagcaccgatttcctcttatatgtggaaggagactatgttacatacctgaagtattactcttttacaaagataccattagaattttctcactgtcttggtaaaaattcctagattctactttttattgcaagtgttagcaatatgatcttgtaacgccatgaaaagcattaagtagaagccaacattaacctagattttacaactgagcaatctgttccccaccagatttcctttagaatttattaccaaaggatcttattttgttacagaacacaggaaattgttaaaccttataatgttgcaaaccatgcattttccttttctcttttccataagatttttttattgtagtagtgccctggcttgtaaacagtggacatcactgaaaggatgaaatcaagtaggcttgtttcaaagagactcctgtgaatcaactatgaatatgaagtatctcccagaacaatgttgagtgtttcaggagactctgaaacagggcacccattttttttttccttcctcaaaatagtctccttgaaatgtgagcaaagaactatgatgatgcagtccaggtatctaatagctcaatcaccaacaccgctgagaattgttcttataccagcaaaaagttcaaatacaataacctgggcaatgccaacagaagacacttttaatcatttgcagacctttcttgttgcaggaaagtgagggcaaaaacaaactggtgaaaaatgttcaaaaatagatctttttgatgttcatagccagtattcagtatttctgtattgtttacaattgggggaaaatgtctgttaaggctaatcatctgagtgatttgagtagcaattaaaccaaagggaaacttttaataagttattttaagccctggtttatggctagttcccttttgctcaggaaaaatagtactactttatggtttatgaaaacctatgatttatgttgcatcttccggggaaggtttaacagtacaaggagctgaatcctacctgaaattattaatgcttaatatattgcttctgtttgattgataaaggtgcttaatcaattgaaaaaaaaacgcataatgcttcaaggtaaaattccgttgattctgaatttaaaaagatactacagaatgcgtcagttctcctgttgtatttttctcagttatgagagcaaggctattttgtcagtttagattttgtgtctttttctgtcagtatgtatggctccctaattgaaaacatcttaaatgaactaatcaaacactta